(19) United States
(12) Patent Application Publication
(10) Pub. No.: US 2024/0084243 A1
(43) Pub. Date: Mar. 14, 2024

(54) SURFACE DISPLAYED FUSION PROTEINS

(71) Applicant: Clara Foods Co., Daly City, CA (US)

(72) Inventors: Weixi ZHONG, Daly City, CA (US); Logan Hurst, Daly City, CA (US)

(21) Appl. No.: 18/344,790

(22) Filed: Jun. 29, 2023

Related U.S. Application Data
(60) Provisional application No. 63/356,984, filed on Jun. 29, 2022.

(51) Int. Cl. C12N 1/16; C07K 14/395; C12N 9/24; C12N 15/81

(52) U.S. Cl. CPC C12N 1/16; C07K 14/395; C12N 9/2402; C12N 15/815; C12R 2001/84

(57) ABSTRACT

The present disclosure provides engineered eukaryotic cells comprising a surface displayed fusion proteins comprising a catalytic domain of an enzyme and an anchoring domain of a glycosylphosphatidylinositol (GPI)-anchored protein and methods of use.

### CROSS-REFERENCE

This application claims priority to and benefit of U.S. Provisional Application No. 63/356,984, filed Jun. 29, 2022, which is herein incorporated by reference in its entirety.

### SEQUENCE LISTING

The instant application contains a Sequence Listing which has been submitted electronically in XML format and is hereby incorporated by reference in its entirety. Said XML copy, created on Jun. 29, 2023, is named 56045US_CRF_sequencelisting.xml and is 439,995 bytes in size.

### BACKGROUND

Recombinant protein expression is a useful method for producing large quantities of animal-free proteins. In some cases, it is desirable to enzymatically modify a secreted recombinant protein and/or enzymatically modify a protein or other chemical in a culturing medium. There exists an unmet need for engineered eukaryotic cells that express surface displayed enzymes for modifying a secreted recombinant protein and/or for modifying another chemical in a culturing medium.

### SUMMARY

An aspect of the present disclosure is an engineered eukaryotic cell that expresses a surface-displayed fusion protein. The fusion protein comprising a catalytic domain of an enzyme and an anchoring domain of a glycosylphosphatidylinositol (GPI)-anchored protein, wherein the anchoring domain comprises at least about 200 amino acids and/or at least about 30% of the residues in the anchoring domain are serines or threonines.

In embodiments, the anchoring domain comprises at least about 225 amino acids, at least about 250 amino acids, at least about 275 amino acids, at least about 300 amino acids, at least about 325 amino acids, at least about 350 amino acids, at least about 375 amino acids, or at least about 400 amino acids.

In some embodiments, at least about 35% of the residues in the anchoring domain are serines or threonines, at least about 40% of the residues in the anchoring domain are serines or threonines, at least about 45% of the residues in the anchoring domain are serines or threonines, or at least about 50% of the residues in the anchoring domain are serines or threonines.

In various embodiments, the serines or threonines in the anchoring domain are capable of being O-mannosylated.

In embodiments, a fusion protein having an anchoring domain comprising at least about 325 amino acids provides greater enzymatic activity relative to a fusion protein having an anchoring domain comprising less than about 300 amino acids.

In some embodiments, a fusion protein having an anchoring domain comprising at least about 300 amino acids provides greater enzymatic activity relative to a fusion protein having an anchoring domain comprising less than about 250 amino acids.

In various embodiments, the fusion protein comprises the anchoring domain of the GPI anchored protein.

In embodiments, the fusion protein comprises the GPI anchored protein without its native signal peptide.

In some embodiments, the GPI anchored protein is not native to the engineered eukaryotic cell.

In various embodiments, the GPI anchored protein is naturally expressed by a S. cerevisiae cell and the engineered eukaryotic cell is not a S. cerevisiae cell.

In embodiments, the GPI anchored protein is selected from Tir4, Dan1, Dan4, Sag1, FIG. 2, or Sed1.

In some embodiments, the anchoring domain of the GPI anchored protein comprises an amino acid sequence that is at least 70% identical, at least 75% identical, at least 80% identical, at least 85% identical, at least 90% identical, or at least 95% identical, to one of SEQ ID NO: 1 to SEQ ID NO: 14.

In various embodiments, the anchoring domain of the GPI anchored protein comprises an amino acid sequence of one of SEQ ID NO: 1 to SEQ ID NO: 14.

In embodiments, the engineered eukaryotic cell is a yeast cell.

In some embodiments, the engineered eukaryotic cell is a Pichia species. In some cases, the Pichia species is Pichia pastoris.

In various embodiments, the engineered eukaryotic cell comprises a genomic modification that expresses the fusion protein and/or comprises an extrachromosomal modification that expresses the fusion protein.

In embodiments, the fusion protein comprises a portion of the enzyme in addition to its catalytic domain.

In some embodiments, the fusion protein comprises substantially the entire amino acid sequence of the enzyme.

In various embodiments, the enzyme catalyzes a post-translational modification of a protein secreted by the engineered eukaryotic cell, the enzyme catalyzes a reaction which removes impurities secreted by the engineered eukaryotic cell, and/or the enzyme catalyzes a reaction which allows the engineered eukaryotic cell to rely on alternate carbon sources. In some cases, the catalyzed post-translational modification comprises deglycosylation, acetylation, adenylation, alkylation, amidation, glycosylation, hydroxylation, methylation, proteolysis, or phosphorylation. The enzyme catalyzing a post-translational modification may be an endoglycosidase, e.g., endoglycosidase H. In various case, the enzyme that catalyzes a reaction that removes impurities comprises a hydrolase, a decarboxylase, an esterase, a lipase, a phosphatase, a glycosidase, a peptidase, a protease, or a nucleosidase. The enzyme that catalyzes a reaction that removes impurities may be a mannosidase. In additional cases, the enzyme that catalyzes a reaction which allows the engineered eukaryotic cell to rely on alternate carbon sources comprises a sucrase (e.g., invertase), an amylase, a cellulase, an isomaltase, a lactase, a maltase, or a sugar isomerase. The enzyme that catalyzes a reaction which allows the engineered eukaryotic cell to rely on alternate carbon sources may be a sucrase (e.g., invertase).

In embodiments, the enzyme comprises an amino acid sequence that is at least 70% identical, at least 75% identical, at least 80% identical, at least 85% identical, at least 90% identical, or at least 95% identical, to one of SEQ ID NO: 15 to SEQ ID NO: 20.

In some embodiments, the enzyme comprises an amino acid sequence of one of SEQ ID NO: 15 to SEQ ID NO: 20.

In various embodiments, the fusion protein comprises an amino acid sequence that is at least 70% identical, at least 75% identical, at least 80% identical, at least 85% identical, at least 90% identical, or at least 95% identical, to one of SEQ ID NO: 21 to SEQ ID NO: 26.

In embodiments, the fusion protein comprises an amino acid sequence of one of one of SEQ ID NO: 24 to SEQ ID NO: 26.

In some embodiments, in the fusion protein, the catalytic domain is N-terminal to the anchoring domain.

In various embodiments, the fusion protein comprises a linker between the catalytic domain and the anchoring domain.

In embodiments, the fusion protein comprises a linker having an amino acid sequence that is at least 95% identical to SEQ ID NO: 31.

In some embodiments, upon translation, the fusion protein comprises a signal peptide and/or a secretory signal.

In various embodiments, the engineered eukaryotic cell comprises two or more fusion proteins, three or more fusion proteins, or four fusion proteins. In some cases, the two or more fusion proteins comprise different enzyme types or the two or more fusion proteins comprise the same enzyme type. In various cases, the two of the three or more fusion proteins or two of the four or more fusion proteins comprise different enzyme types or two of the three or more fusion proteins or two of the four or more fusion proteins comprise the same enzyme type. In additional cases, the three of the three or more fusion proteins or three of the four or more fusion proteins comprise different enzyme types or three of the three or more fusion proteins or three of the four or more fusion proteins comprise the same enzyme type. In various cases, each of the two or more, three or more, or four fusion proteins comprise different enzyme types or each of the two or more, three or more, or four fusion proteins comprise the same enzyme type. In embodiments, the enzyme types are selected from an enzyme that catalyzes a post-translational modification of a protein secreted by the engineered eukaryotic cell, an enzyme that catalyzes a reaction which removes impurities secreted by the engineered eukaryotic cell, and/or an enzyme that catalyzes a reaction which allows the engineered eukaryotic cell to rely on alternate carbon sources.

In some embodiments, the engineered eukaryotic cell comprises a mutation in its AOX1 gene and/or its AOX2 gene.

In various embodiments, the engineered eukaryotic cell comprises a genomic modification that overexpresses a secreted recombinant protein and/or comprises an extrachromosomal modification that overexpresses a secreted recombinant protein. In some cases, the secreted recombinant protein is an animal protein, e.g., an egg protein. The egg protein may be selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

In embodiments, the genomic modification and/or the extrachromosomal modification that overexpresses the secreted recombinant protein comprises an inducible promoter. In some cases, the inducible promoter is an AOX1, DAK2, PEX11, FLD1, FGH1, DAS1, DAS2, CAT1, MDH3, HAC1, BiP, RAD30, RVS161-2, MPP10, THP3, TLR, GBP2, PMP20, SHB17, PEX8, PEX4, or TKL3 promoter. In various cases, the genomic modification and/or the extrachromosomal modification that overexpresses a secreted recombinant protein comprises an AOX1, TDH3, MOX, RPS25A, or RPL2A terminator. In further cases, the genomic modification and/or the extrachromosomal modification that overexpresses a secreted recombinant protein encodes a signal peptide and/or a secretory signal. In additional cases, the genomic modification and/or the extrachromosomal modification that overexpresses a secreted recombinant protein comprises codons that are optimized for the species of the engineered eukaryotic cell. In some cases, the secreted recombinant protein is designed to be secreted from the cell and/or is capable of being secreted from the cell.

In some embodiments, the engineered eukaryotic cell comprises an additional genomic modification comprising a knockout of a coding sequence for a cell wall protein or an additional genomic modification that overexpresses a cell wall protein. In some cases, the engineered eukaryotic cell comprises an additional genomic modification comprising a knockout of the coding sequences for more than one cell wall proteins or an additional genomic modification that overexpresses more than one a cell wall proteins. In various cases, the cell wall protein is a mannoprotein. In further cases, the cell wall protein is one or more of a CCW12 homolog, a CCW14 homolog, a CCW22 homolog, a FLO5 homolog, or a SED1 homolog. In additional cases, the cell wall protein comprises the amino acid sequence of any one of SEQ ID NO: 306 to SEQ ID NO: 319. In some cases, the additional genomic modification reduces the number of native cell wall proteins expressed by the engineered eukaryotic cell, thereby allowing additional space for localization of the surface-displayed fusion protein.

In various embodiments, the engineered eukaryotic cell comprises a further genomic modification that overexpresses a protein related to the p24 complex. In some cases, the engineered eukaryotic cell comprises a further genomic modification comprising that overexpresses more than one protein related to the p24 complex. In various cases, the protein related to the p24 complex is selected from Erp1, Erp2, Erp3, Erp5, Emp24, and Erv25. In further cases, the protein related to the p24 complex comprises the amino acid sequence of any one of SEQ ID NO: 320 to SEQ ID NO: 325. In some cases, the further genomic modification promotes trafficking of the surface-displayed fusion protein through the secretory pathway.

In embodiments, the engineered eukaryotic cell further encodes one or more additional fusion proteins comprising a catalytic domain of an enzyme and an adhesion or anchoring domain from a cell surface protein selected from Sed1p, Flo5-2, Flo11, Saccharomyces cerevisiae Flo5, CWP, and PIR with the adhesion or anchoring domain having the ability to capture exopolysaccharides and retain the additional fusion protein at the extracellular surface.

Another aspect of the present disclosure is a method for expressing a surface-displayed fusion protein comprising a catalytic domain of an enzyme and an anchoring domain of glycosylphosphatidylinositol (GPI)-anchored protein. The method comprising obtaining any herein-disclosed engineered eukaryotic cell and culturing the engineered eukaryotic cell under conditions that promote expression of the fusion protein.

In some embodiments, when the engineered eukaryotic cell comprises a genomic modification and/or an extrachromosomal modification that overexpresses a secreted recombinant protein comprises an inducible promoter, the method comprises culturing the engineered eukaryotic cell under conditions that promote expression of the fusion protein by contacting the engineered eukaryotic with an agent that activates the inducible promoter.

In various embodiments, the inducible promoter is an AOX1, DAK2, PEX11, FLD1, FGH1, DAS1, DAS2, CAT1, MDH3, HAC1, BiP, RAD30, RVS161-2, MPP10, THP3, TLR, GBP2, PMP20, SHB17, PEX8, PEX4, or TKL3 promoter. In some cases, when the inducible promoter is an AOX1, DAK2, PEX11, FLD1, FGH1, DAS2, CAT1, PMP20, SHB17, PEX8, PEX4, TKL3 or DAS1 promoter and the agent that activates the inducible promoter is methanol. In various cases, the secreted recombinant protein is designed to be secreted from the cell and/or is capable of being secreted from the cell.

Yet another aspect of the present disclosure is a population of any herein-disclosed engineered eukaryotic cells.

A further aspect of the present disclosure is a bioreactor comprising a population of any herein-disclosed engineered eukaryotic cells.

In an aspect, the present disclosure provides a composition comprising any herein-disclosed engineered eukaryotic cells and a secreted recombinant protein.

In embodiments, the secreted recombinant protein is an animal protein, e.g., an egg protein. The egg protein may be selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

In another aspect, the present disclosure provides a composition comprising any herein-disclosed engineered eukaryotic cell, a secreted recombinant protein that has been deglycosylated, and one or more oligosaccharides cleaved from the secreted recombinant protein.

In some embodiments, the secreted recombinant protein is an animal protein, e.g., an egg protein. The egg protein may be selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

In yet another aspect, the present disclosure provides a method for post-translationally modifying a secreted recombinant protein. The method comprising contacting a secreted recombinant protein with a fusion protein anchored to any herein-disclosed engineered eukaryotic cell, wherein the fusion protein comprises a catalytic enzyme that deglycosylates, acetylates, adenylates, alkylates, amidates, glycosylates, hydroxylates, methylates, or phosphorylates.

In a further aspect, the present disclosure provides a method for removing impurities secreted by an engineered eukaryotic cell. The method comprising culturing any herein-disclosed engineered eukaryotic cell under conditions that an impurity is secreted by the engineered eukaryotic cell and contacting the impurity with a fusion protein anchored to the engineered eukaryotic cell, wherein the fusion protein comprises a catalytic enzyme that cleaves the impurity, denatures the impurity, modifies the impurity, and/or detoxifies the impurity.

An aspect of the present disclosure is a method for allowing an engineered eukaryotic cell to rely on alternate carbon sources. The method comprising contacting an alternate carbon source with a fusion protein anchored any herein-disclosed engineered eukaryotic cell, wherein the fusion protein comprises a catalytic enzyme that cleaves the alternate carbon source into a carbon source that can be taken in by the cell and used as a carbon source by the cell.

In various embodiments, when the fusion protein comprises an invertase, the engineered eukaryotic cell is capable of growing on sucrose as its primary carbon source. In some cases, when the fusion protein comprises the anchoring domain is from Tir4, the engineered eukaryotic cell has increased growth when grown on sucrose as its primary carbon source relative to a eukaryotic cell that is not engineered to rely on sucrose as an alternate carbon source.

Any aspect or embodiment may be combined with any other aspect or embodiment.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 includes schematics of various surface displayed fusion proteins comprising a catalytic domain of an enzyme and an anchoring domain of a glycosylphosphatidylinositol (GPI)-anchored protein, i.e., Dan 1, Sed1, and Tir4.

FIG. 2 includes schematics of nucleic acids encoding three surface displayed fusion proteins. This example shows a full plasmid map, containing the components of FIG. 3 and commonly used plasmid vector elements.

FIG. 3 includes schematics of the three surface displayed fusion proteins. In these schematics, the enzyme is Endoglycosidase H (EndoH) and the three anchoring domains of GPI-anchored proteins are Dan 1, Sed1, and Tir4. The top map of FIG. 3 shows a plasmid map of the amino acid sequence SEQ ID 24; the middle map of FIG. 3 shows a plasmid map of the amino acid sequence of SEQ ID 26; and the bottom map of FIG. 3 shows a plasmid map of amino acid sequence of SEQ ID NO: 22.

FIG. 4 is a photograph of an SDS-PAGE gel demonstrating the ability of surface displayed EndoH—Dan1, EndoH—Sed1, or EndoH—Tir4 fusion proteins do deglycosylate an illustrative glycoprotein.

FIG. 5 illustrates the growth of P. pastoris on minimal nutrient plates containing glucose, fructose and sucrose.

FIG. 6 illustrates an exemplary schematic of a construct to express SUC2.

FIG. 7 illustrates the growth of P. pastoris strains using mannose as a sole carbon source.

FIG. 8 illustrates the growth of P. pastoris strains using glucose or sucrose as a sole carbon source. The strains labelled “_D” in FIG. 8 denote that dextrose (glucose) was used as the carbon source in the experimental condition. The strains labelled “_S” in FIG. 8 denote that sucrose was used as the carbon source in the experimental condition.

FIG. 9 illustrates the growth of P. pastoris strains using mannose as a sole carbon source.

FIG. 10 illustrates size exclusion chromatography of EPS samples. strain 8 is strain 7 after the deletion of 5 native P. pastoris mannosyltransferases.

FIG. 11 illustrates a general schematic for mannosidase surface display.

FIG. 12 illustrates size exclusion chromatography of EPS samples. By coupling the deletion of native mannosyltransferases with the expression of a surface-displayed B. thetaiotaomicron mannosidase, strain 9 is able to reduce the size of the EPS byproduct.

FIG. 13 illustrates that disruption of native mannosyltransferases is important for B. theta enzymes to recognize mannan as a substrate for cleavage. The strains with deletions and mannosidase elicits the right-shift in the EPS elution profile.

FIG. 14 illustrates another general schematic for mannosidase surface display.

FIG. 15 depicts chromatograms of background strain (strain 7) and new strain (strain 9).

### DETAILED DESCRIPTION

### Introduction

The present disclosure provides engineered eukaryotic cells comprising a surface displayed fusion protein. The fusion protein comprising a catalytic domain of an enzyme and an anchoring domain of a glycosylphosphatidylinositol (GPI)-anchored protein.

Surface displaying a catalytic domain of an enzyme provides effective and efficient means to project the catalytic domain into the extracellular space, thereby increasing the likelihood that the catalytic domain will encounter and catalyze an enzymatic reaction with its substrate, e.g., protein, lipid, carbohydrate, or other compound. In the present disclosure, an fusion protein is localized to the extracellular surface of a cell, i.e., is surface displayed. This way, the catalytic domain is unlikely to contact an intracellular, membrane-associated, or cell wall protein, thereby lowering the opportunity for the enzyme to modify, degrade, or the like a substrate needed by the cell. In one example, the enzyme is an endoglycosidase which deglycosylates glyocoproteins and removes their attached oligosaccharide; by surface displaying the fusion protein, the catalytic domain does not remove a needed oligosaccharide from a cellular glycoprotein. Instead, the surface displayed endoglycosidase primarily deglycosylates proteins found in the extracellular space, e.g., secreted recombinant proteins. Accordingly, in some embodiments, the present disclosure provides recombinant cells having the means to deglycosylate secreted glycoproteins proteins and having a reduced likelihood of undesirably deglycosylating its own intracellular, membrane bound, or cell wall glycoproteins. Additionally, since the surface displayed endoglycosidase is securely attached to the recombinant cell, it is not released into and present in a culturing medium. Thus, there is no need to separate the endoglycosidase from the secreted recombinant protein when making a generally contaminant-free recombinant protein product. In other words, the use of surface displayed endoglycosidase avoids the added expense, time, and inefficiency, as described above, that is needed to later remove the endoglycosidase when manufacturing a recombinant protein product for human or animal use, e.g., in a consumable composition. In other embodiments, the fusion protein catalyzes a reaction that cleaves a dissacharide, which would the cell would be unable to utilize as a carbon source. By cleaving the dissacharide into monosaccharides, the cell is able to use the monosaccharides even though the culturing medium did not included the monosaccharide. In further embodiments, the fusion protein expresses an enzyme, e.g., a mannosidase, that digests an impurity secreted by the cell. The herein-disclosed surface display fusion proteins are modular and can be adapted to catalyze any reaction that a user may desire.

### Surface Displayed Fusion Proteins

An aspect of the present disclosure is an engineered eukaryotic cell that expresses a surface-displayed fusion protein. The fusion protein comprising a catalytic domain of an enzyme and an anchoring domain of a glycosylphosphatidylinositol (GPI)-anchored protein, wherein the anchoring domain comprises at least about 200 amino acids and/or at least about 30% of the residues in the anchoring domain are serines or threonines.

A fusion protein is a protein consisting of at least two domains that are normally encoded by separate genes but have been joined so that they are transcribed and translated as a single unit; thereby, producing a single (fused) polypeptide.

In the present disclosure, a fusion protein comprises at least a catalytic domain of an enzyme and an anchoring domain of GPI-anchored protein. Typically, a GPI-anchored protein is a cell surface protein, e.g., which is located on the extracellular surface of the cell.

A fusion protein may further comprise linkers that separate the two domains. Linkers can be flexible or rigid; they can be semi-flexible or semi-rigid. Separating the two domains, may promote activity of the catalytic domain in that it reduces steric hindrance upon the catalytic site which may be present if the catalytic site is too closely positioned relative to an anchoring domain. Additionally, a linker may further project the catalytic domain into the extracellular space, thereby increasing the likelihood that the catalytic domain will encounter and catalyze an enzymatic reaction with its substrate, e.g., protein, lipid, carbohydrate, or other compound.

In embodiments, the anchoring domain comprises at least about 225 amino acids, at least about 250 amino acids, at least about 275 amino acids, at least about 300 amino acids, at least about 325 amino acids, at least about 350 amino acids, at least about 375 amino acids, or at least about 400 amino acids.

In some embodiments, at least about 35% of the residues in the anchoring domain are serines or threonines, at least about 40% of the residues in the anchoring domain are serines or threonines, at least about 45% of the residues in the anchoring domain are serines or threonines, or at least about 50% of the residues in the anchoring domain are serines or threonines.

In various embodiments, the serines or threonines in the anchoring domain are capable of being O-mannosylated.

In embodiments, a fusion protein having an anchoring domain comprising at least about 325 amino acids provides greater enzymatic activity relative to a fusion protein having an anchoring domain comprising less than about 300 amino acids.

In some embodiments, a fusion protein having an anchoring domain comprising at least about 300 amino acids provides greater enzymatic activity relative to a fusion protein having an anchoring domain comprising less than about 250 amino acids.

Surprisingly, it was discovered that a correlation between the length of the GPI-linked anchor protein and/or the amount of predicted 0-glycosylated serine/threonine residues and the efficiency of the displayed enzyme, e.g., EndoH.

In embodiments, the fusion protein comprises the GPI anchored protein without its native signal peptide.

In some embodiments, the GPI anchored protein is not native to the engineered eukaryotic cell.

In various embodiments, the GPI anchored protein is naturally expressed by a S. cerevisiae cell and the engineered eukaryotic cell is not a S. cerevisiae cell.

In embodiments, the GPI anchored protein is selected from Tir4, Dan1, Dan4, Sag1, FIG. 2, or Sed1.

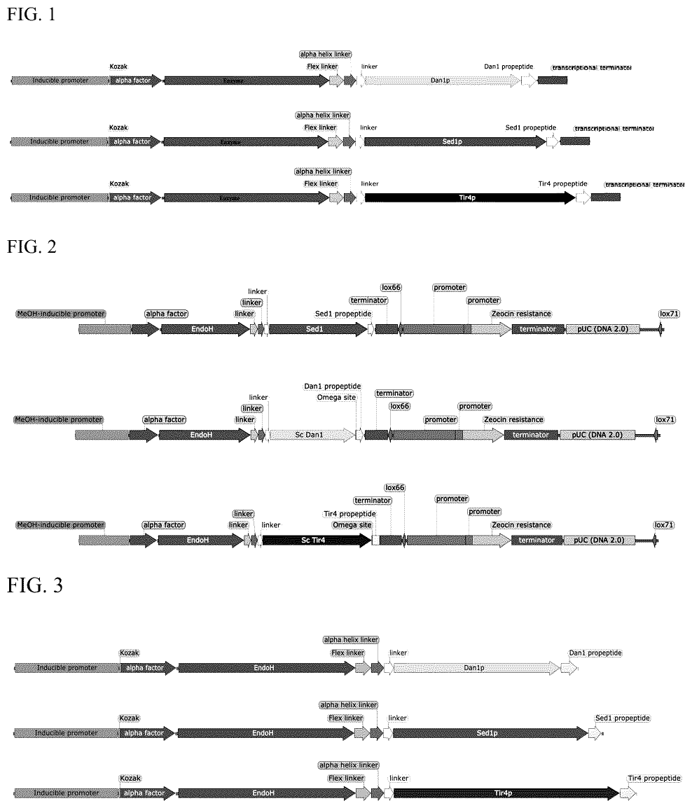

Schematic of various surface displayed fusion proteins comprising a catalytic domain of an enzyme and an anchoring domain of a glycosylphosphatidylinositol (GPI)-anchored protein, i.e., Dan 1, Sed1, and Tir4 are shown in FIG. 1.

In some embodiments, the anchoring domain of the GPI anchored protein comprises an amino acid sequence that is at least 70% identical, at least 75% identical, at least 80% identical, at least 85% identical, at least 90% identical, or at least 95% identical, to one of SEQ ID NO: 1 to SEQ ID NO: 14.

In various embodiments, the anchoring domain of the GPI anchored protein comprises an amino acid sequence of one of SEQ ID NO: 1 to SEQ ID NO: 14.

Sed1p is a major component of the Saccharomyces cerevisiae cell wall. It is required to stabilize the cell wall and for stress resistance in stationary-phase cells. See, e.g., the world wide web (at) uniprot.org/uniprot/Q01589. It is believed that Asn 318 (with respect to SEQ ID NO: 13) is the most likely candidate for the GPI attachment site in Sed1p. In some embodiments, a fusion protein comprising a Sed1p anchoring domain has a sequence having at least 95% or more sequence identity with SEQ ID NO:13 or SEQ ID NO: 14. In some cases, the sequence identity may be greater than or about 90%, 95%, 96%, 97%, 98%, 99%, or 100%. In various embodiments, the Sed1p anchoring domain of a fusion protein of the present disclosure comprises a GPI attachment site; thus, the anchoring domain may only require a short fragment of SEQ ID NO: 13 or SEQ ID NO: 14, i.e., a fragment that is 5, 10, 25, 50, 100, 200, or 300 or more amino acids in length, as long as it is capable of projecting the catalytic domain of the fusion protein into the extracellular space. In some embodiments, the anchoring domain comprises, at least, Sed1p's GPI attachment site.

Komagataella phaffii Flo5-2 is considered to be an ortholog of both Saccharomyces Flo1 and Flo5. See, e.g., the worldwide web (at) uniprot.org/uniprot/F2QXP0. The two Saccharomyces flocculation proteins are highly similar in their amino acid sequence, only significantly differing in the length of the linker portion used to extend the protein past the cell wall. The Saccharomyces flocculation proteins are cell wall proteins that participate directly in adhesive cell-cell interactions during yeast flocculation, a reversible, asexual process in which cells adhere to form aggregates (flocs) consisting of thousands of cells. The flocculation family of proteins are useful in the present disclosure, for, at least, two reasons. First, they generally extend relatively far from the cell wall and, second, it is believed that they bind and capture some exopolysaccharides. Notably, Flo5-2 has a GPI anchor site towards its C-terminus which can tether the protein to a cell's membrane. Therefore, a fusion protein comprising an anchoring domain of Flo5-2 may anchor the fusion protein to the extracellular surface of an engineered cell via its GPI anchor or by the domain's interaction with exopolysaccharides located on the extracellular surface of an engineered cell.

In some embodiments, a fusion protein comprising a Saccharomyces cerevisiae Flo5 anchoring domain has a sequence that has 95% or more sequence identity with SEQ ID NO: 335. In some cases, the sequence identity may be greater than or about 90%, 95%, 96%, 97%, 98%, 99%, or 100%. In various embodiments, the Flo5 anchoring domain of a fusion protein of the present disclosure comprises a GPI attachment site; thus, the anchoring domain may only require a short fragment of SEQ ID NO: 335, i.e., a fragment that is 5, 10, 25, 50, 100, 200, 300, 400, 500, 600, 700, 800, 900, or 1000 or more amino acids in length, as long as it is capable of projecting the catalytic domain of the fusion protein into the extracellular space. In some embodiments, the anchoring domain comprises, at least, Flo5's GPI attachment site. In some embodiments, the anchoring domain lacks Flo5's GPI attachment site yet retains the ability to capture exopolysaccharides and retain the fusion protein at the extracellular surface.

Flo11 is another GPI-anchored cell surface glycoprotein (flocculin). See, e.g., the worldwide web (at) uniprot.org/uniprot/F2QRD4. Flo11 is believed to be required for pseudohyphal and invasive growth, flocculation, and biofilm formation. It is a major determinant of colony morphology and required for formation of fibrous interconnections between cells. Like the other yeast flocculation proteins, its adhesive activity is inhibited by mannose, but not by glucose, maltose, sucrose, or galactose. Thus, use of Flo11 in a fusion protein of the present disclosure may be useful extending the fusion protein relatively far from the cell wall, and for binding and capturing some exopolysaccharides. Like, Flo5-2, Flo11 has a GPI anchor site towards its C-terminus which can tether the protein to a cell's membrane. Therefore, a fusion protein comprising an anchoring domain of Flo11 may anchor the fusion protein to the extracellular surface of an engineered cell via its GPI anchor or by the domain's interaction with exopolysaccharides located on the extracellular surface of an engineered cell. Moreover, without wishing to be bound by theory, inclusion of an anchoring domain of Flo11 may promote capture of a secreted glycoprotein for deglycosylation.

In some embodiments, a fusion protein comprising a Flo11 anchoring domain has a sequence that has 95% or more sequence identity with SEQ ID NO: 328 or SEQ ID NO: 329. In some cases, the sequence identity may be greater than or about 90%, 95%, 96%, 97%, 98%, 99%, or 100%. In various embodiments, the Flo11 anchoring domain of a fusion protein of the present disclosure comprises a GPI attachment site; thus, the anchoring domain may only require a short fragment of SEQ ID NO: 328 or SEQ ID NO: 329, i.e., a fragment that is 5, 10, 25, 50, 100, 200, 300, 400, 500, 600, 700, 800, 900, or 1000 or more amino acids in length, as long as it is capable of projecting the catalytic domain of the fusion protein into the extracellular space. In some embodiments, the anchoring domain comprises, at least, Flo1 l's GPI attachment site. In some embodiments, the anchoring domain lacks Flo1 l's GPI attachment site yet retains the ability to capture exopolysaccharides and retain the fusion protein at the extracellular surface.

When a linker is present, a fusion protein may have a general structure of: N terminus-(a)-(b)-(c)-C terminus, wherein (a) is comprises a first domain, (b) is one or more linkers, and (c) is a second domain. The first domain may comprise a catalytic domain of an enzyme and the second domain may comprise an anchoring domain of a GPI anchored protein. In some embodiments, in the fusion protein, the catalytic domain is N-terminal to the anchoring domain. The fusion protein may comprise a linker N-terminal to the anchoring domain.

Linkers useful in fusion proteins may comprise one or more sequences of SEQ ID NO: 28 to SEQ ID NO: 31. In one example, a tandem repeat (of two, three, four, five, six, or more copies) of a linker, e.g., of SEQ ID NO: 28 or SEQ ID NO: 29 is included in a fusion protein.

In embodiments, the fusion protein comprises a linker having an amino acid sequence that is at least 95% identical to SEQ ID NO: 31.

In embodiments, a fusion protein comprises a Glu-Ala-Glu-Ala (EAEA; SEQ ID NO: 27) spacer dipeptide repeat. The EAEA (SEQ ID NO: 27) is a removable signal that promotes yields of an expressed protein in certain cell types.

Other linkers are well-known in the art and can be substituted for the linkers of SEQ ID NO: 28 to SEQ ID NO: 31. For example, In embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In embodiments, the linker comprises a polypeptide. In embodiments, the polypeptide is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some cases, the linker is about 59 amino acids long.

The length of a linker may be important to the effectiveness of a surface displayed enzyme's catalytic domain. For example, if a linker is too short, then the catalytic domain of the enzyme may not project far enough away from the cell surface such that it is incapable of interacting with its substrate, e.g., protein, lipid, carbohydrate, or other compound. In this case, the catalytic domain may be buried in the cell wall and/or among other cell surface proteins or sugars. On the other hand, the linker may be too long and/or too rigid to allow adequate contact between a substrate and the catalytic domain of the enzyme.

The secondary structure of a linker may also be important to the effectiveness of a surface displayed enzyme's catalytic domain. More specifically, a linker designed to have a plurality of distinct regions may provide additional flexibility to the fusion protein. As examples, a linker having one or more alpha helices may be superior to a linker having no alpha helices.

The longer linker of (SEQ ID NO: 31) comprises three subsections: an N-terminal flexible GS linker with higher S content, a rigid linker that forms four turns of an alpha helix, and a flexible GS linker with much higher G content on its C-terminus. Linkers containing only G's and S's in repetitive sequences are commonly used in fusion proteins as flexible spacers that do not introduce secondary structure. In some cases, the ratio of G to S determines the flexibility of the linker. Linkers with higher G content may be more flexible than linkers with higher S content. The structure of the linker of SEQ ID NO: 31 is designed to mimic multi-domain proteins in nature, which often uses alpha helices (sometimes multiple) to separate as well as orient their domains spatially. In fusion proteins of the present disclosure, a complex linker, such as that of SEQ ID NO: 31 can be viewed as a multi-domain protein with the catalytic domain of an enzyme and an anchoring domain of a GPI anchored protein being separate functional domains.

In various embodiments, the fusion protein comprises a linker having an amino acid sequence that is at least 95%, 96%, 97%, 98%, 99%, or 100% identical to SEQ ID NO: 31.

In embodiments, the linker is substantially comprised of glycine and serine residues (e.g. about 30%, or about 40%, or about 50%, or about 60%, or about 70%, or about 80%, or about 90%, or about 95%, or about 96%, or about 97%, or about 98%, or about 99%, or about 100% glycines and serines).

In various embodiments, the engineered eukaryotic cell comprises a genomic modification that expresses the fusion protein and/or comprises an extrachromosomal modification that expresses the fusion protein.

In embodiments, the fusion protein comprises a portion of the enzyme in addition to its catalytic domain.

In some embodiments, the fusion protein comprises substantially the entire amino acid sequence of the enzyme.

In some embodiments, upon translation, the fusion protein comprises a signal peptide and/or a secretory signal.

In various embodiments, the engineered eukaryotic cell comprises two or more fusion proteins, three or more fusion proteins, or four fusion proteins.

In some cases, the two or more fusion proteins comprise different enzyme types or the two or more fusion proteins comprise the same enzyme type.

In various cases, the two of the three or more fusion proteins or two of the four or more fusion proteins comprise different enzyme types or two of the three or more fusion proteins or two of the four or more fusion proteins comprise the same enzyme type.

In additional cases, the three of the three or more fusion proteins or three of the four or more fusion proteins comprise different enzyme types or three of the three or more fusion proteins or three of the four or more fusion proteins comprise the same enzyme type.

In various cases, each of the two or more, three or more, or four fusion proteins comprise different enzyme types or each of the two or more, three or more, or four fusion proteins comprise the same enzyme type.

In embodiments, the enzyme types are selected from an enzyme that catalyzes a post-translational modification of a protein secreted by the engineered eukaryotic cell, an enzyme that catalyzes a reaction which removes impurities secreted by the engineered eukaryotic cell, and/or an enzyme that catalyzes a reaction which allows the engineered eukaryotic cell to rely on alternate carbon sources.

### Enzymes

In various embodiments, the enzyme (of a surface displayed fusion protein) catalyzes a post-translational modification of a protein secreted by the engineered eukaryotic cell, the enzyme catalyzes a reaction which removes impurities secreted by the engineered eukaryotic cell, and/or the enzyme catalyzes a reaction which allows the engineered eukaryotic cell to rely on alternate carbon sources.

In some cases, the catalyzed post-translational modification comprises deglycosylation, acetylation, adenylation, alkylation, amidation, glycosylation, hydroxylation, methylation, proteolysis, or phosphorylation. The enzyme catalyzing a post-translational modification may be an endoglycosidase, e.g., endoglycosidase H.

In various case, the enzyme that catalyzes a reaction that removes impurities comprises a hydrolase, a decarboxylase, an esterase, a lipase, a phosphatase, a glycosidase, a peptidase, a protease, or a nucleosidase. The enzyme that catalyzes a reaction that removes impurities may be a mannosidase.

In additional cases, the enzyme that catalyzes a reaction which allows the engineered eukaryotic cell to rely on alternate carbon sources comprises a sucrase (e.g., invertase), an amylase, a cellulase, an isomaltase, a lactase, a maltase, or a sugar isomerase. The enzyme that catalyzes a reaction which allows the engineered eukaryotic cell to rely on alternate carbon sources may be a sucrase (e.g., invertase).

In embodiments, the enzyme comprises an amino acid sequence that is at least 70% identical, at least 75% identical, at least 80% identical, at least 85% identical, at least 90% identical, or at least 95% identical, to one of SEQ ID NO: 15 to SEQ ID NO: 20.

In some embodiments, the enzyme comprises an amino acid sequence of one of SEQ ID NO: 15 to SEQ ID NO: 20.

In various embodiments, the fusion protein comprises an amino acid sequence that is at least 70% identical, at least 75% identical, at least 80% identical, at least 85% identical, at least 90% identical, or at least 95% identical, to one of SEQ ID NO: 21 to SEQ ID NO: 26.

In embodiments, the fusion protein comprises an amino acid sequence of one of one of SEQ ID NO: 24 to SEQ ID NO: 26.

The catalytic domain from an enzyme will be chosen based on the its substrate, e.g., protein, lipid, carbohydrate, or other compound, to which a catalyzed reaction is desired. As an example, if it is desired that an engineered eukaryotic cell become able to rely on alternate carbon sources, then the enzyme may be a sucrase (e.g., invertase). If it is desired that an engineered eukaryotic cell become able to remove impurities secreted by the cell, then the enzyme may be a mannosidase. And, if is desired that an engineered eukaryotic cell become able to deglycosylate proteins secreted by the cell or otherwise present in a culturing medium, the enzyme may be an endoglycosidase, e.g., endoglycosidase H.

In some embodiments, the enzyme may be a glycosyl hydrolase. For example, in some examples, the glycosyl hydrolase may be an invertase such as proteins encoded by the SUC2 or MAL1 genes which cleave a disaccharide sucrose to release glucose and fructose which can be utilized by a yeast such as P. pastoris. In some embodiments, the glycosyl hydrolase may be an invertase such as proteins encoded by the INV1, CINV1, CIN2, INVE, INVA, or SI genes which cleave a disaccharide sucrose to release glucose and fructose which can be utilized by a yeast cell. Additional non-limiting examples of glycosyl hydrolases include, but are not limited to: invertase, invertase 1, cytosolic invertase 1, Beta-fructofuranosidase, insoluble isoenzyme 2, Alkaline/neutral invertase, Alkaline/neutral invertase A, Alkaline/neutral invertase E, and Sucrase-isomaltase.

In some embodiments, the enzyme comprises an amino acid sequence with at least 75%, at least 80%, at least 85%, at least 90%, at least 92%, at least 95%, at least 97% at least 99%, or 100% sequence identity to an amino acid sequence selected from: SEQ ID NOs: 15-20, and 351-361.

In certain embodiments, the enzyme is a glycosyl hydrolase of the family GHS. In certain embodiments, the enzyme is a glycosyl hydrolase of the family GH7. In certain embodiments, the enzyme is a glycosyl hydrolase of the family GH9. Such glycosyl hydrolases are found in PCT Application Publication No.: WO2009090381, which is hereby incorporated by reference in its entirety.

### Endoglycosidases

In some embodiments, the enzyme is an endoglycosidase. A glycoprotein is a protein that carries carbohydrates covalently bound to their peptide backbone. It is known that approximately half of all proteins typically expressed in a cell undergo glycosylation, which entails the covalent addition of sugar moieties (e.g., oligosaccharides) to specific amino acids. Most soluble and membrane-bound proteins expressed in the endoplasmic reticulum are glycosylated to some extent, including secreted proteins, surface receptors and ligands, and organelle-resident proteins. Additionally, some proteins that are trafficked from the Golgi to the cell wall and/or to the extracellular environment are also glycosylated. Lipids and proteoglycans can also be glycosylated, significantly increasing the number of substrates for this type of modification. In particular, many cell wall proteins are glycosylated.

Protein glycosylation has multiple functions in a cell. In the ER, glycosylation is used to monitor the status of protein folding, acting as a quality control mechanism to ensure that only properly folded proteins are trafficked to the Golgi. Oligosaccharides on soluble proteins can be bound by specific receptors in the trans Golgi network to facilitate their delivery to the correct destination. These oligosaccharides can also act as ligands for receptors on the cell surface to mediate cell attachment or stimulate signal transduction pathways. Because they can be very large and bulky, oligosaccharides can affect protein-protein interactions by either facilitating or preventing proteins from binding to cognate interaction domains.

In general, a glycoprotein's oligosaccharides are important to the protein's function. Consequently, should a glycoprotein be deglycosylated intracellularly, once the protein has reached its final destination (if ever), and in a deglycosylated state, the protein may have a lessened and/or an absent activity.

When it is desirable to deglycosylate a recombinant glycoprotein for inclusion in composition for human or animal use (e.g., a food product, drink product, nutraceutical, pharmaceutical, or cosmetic), the recombinant glycoprotein may be contacted with an isolated endoglycosidase that is capable of cleave sugar chains from the glycoprotein. For this, the isolated endoglycosidase may be added to a culturing vessel such that the recombinant glycoprotein is deglycosylated once secreted into its culturing medium. Alternately, a recombinant glycoprotein that has been separated from its culturing medium may be subsequently incubated with the isolated endoglycosidase. Although both of these methods may have effectiveness in providing deglycosylated recombinant proteins, they both increase, at least, the time, expense, and inefficiency involved with manufacturing deglycosylated recombinant proteins. When preparing deglycosylated recombinant proteins for human or animal use, e.g., in a consumable composition, it is preferable, and in some cases, necessary due to regulatory requirements, for the final recombinant protein be free of contaminants. One such contaminant is the endoglycosidase itself. In this case, the endoglycosidase must be removed in part or completely from the final recombinant protein product. This removal would entail multiple purification steps that both increase the expense due to these additional steps and reduce the amount of recombinant protein produced, as some protein would be lost during the various purifications. Also, these purification steps would extend the time for manufacturing the recombinant protein product, thereby reducing efficiency of the process. Moreover, when a recombinant glycoprotein is combined with the endoglycosidase, either in a culturing medium or after the recombinant glycoprotein has been separated from its medium, there is no guarantee that each recombinant glycoprotein will come into contact with an endoglycosidase; to ensure sufficient deglycosylation, the glycoprotein and endoglycosidase must remain in a solution for an extended period of time. This extension of time further reduces the efficiency of the manufacturing process. Finally, purchasing the isolated endoglycosidase or manufacturing the isolated endoglycosidase in house would incur additional expenses. Together, there is an unmet need for manufacturing deglycosylated recombinant protein that is effective and efficient. The methods and systems of the present disclosure satisfy this unmet need.

An Endoglycosidase is an enzyme that releases oligosaccharides from glycoproteins or glycolipids. Unlike exoglycosidases, endoglycoidases cleave polysaccharide chains between residues that are not the terminal residue and break the glycosidic bonds between two sugar monomer in the polymer. When an endoglycosidase cleaves, it releases an oligosaccharide product.

Numerous endoglycosidases have been characterized, cloned, and/or purified. These include Endoglycosidase D, Endoglycosidase F1, Endoglycosidase F2, Endoglycosidase F3, Endoglycosidase H, Endoglycosidase Hf, Endoglycosidase S, Endoglycosidase T, Endoglycoceramidase I, O-Glycosidase, Peptide-N-Glycosidase A (PNGaseA), and PNGaseF.

Normally, an endoglycosidase comprises at least a catalytic domain which is responsible for cleaving an oligonucleotide from a glycoprotein. The endoglycosidase may also comprise domains that help recognize an oligosaccharide and/or the glycoprotein itself. The endoglycosidase may further comprise domains that help facilitate, e.g., positioning of the oligosaccharide and/or glycoprotein itself, cleavage of the oligosaccharide.

In various embodiments, a fusion protein comprises at least the catalytic domain of the endoglycosidase. In some cases, a fusion protein comprises a portion of the endoglycosidase in addition to its catalytic domain. In some embodiments, a fusion protein comprises substantially the entire amino acid sequence of the endoglycosidase.

### Endoglycosidase H

In some cases, the endoglycosidase is endoglycosidase H.

Endoglycosidase H (EndoH); Endo-beta-N-acetylglucosaminidase H (EC:3.2.1.96); DI-N-acetylchitobiosyl beta-N-acetylglucosaminidase H; Mannosyl-glycoprotein endo-beta-N-acetyl-glucosaminidase H is a highly specific endoglycosidase which cleaves asparagine-linked mannose rich oligosaccharides, but not highly processed complex oligosaccharides from glycoproteins. EndoH hydrolyzes (cleaves) the bond in the diacetylchitobiose core of the oligosaccharide between two N-acetylglucosamine (GlcNAc) subunits directly proximal to the asparagine residue, generating a truncated sugar molecule that is released intact and one N-acetylglucosamine residue remaining on the asparagine.

Variants of the known amino acid sequence of endoH may be determined by consulting the literature, e.g. Robbins et al., “Primary structure of the Streptomyces enzyme endo-beta-N-acetylglucosaminidase H.” J. Biol. Chem. 259:7577-7583 (1984); Rao et al., “Crystal structure of endo-beta-N-acetylglucosaminidase H at 1.9-A resolution: active-site geometry and substrate recognition.” Structure 3:449-457 (1995); Rao et al., “Mutations of endo-beta-N-acetylglucosaminidase H active site residue Asp130 and Glu132: activities and conformations.” Protein Sci. 8:2338-2346 (1999); the contents of which are incorporated by reference in their entirety. For example, Rao et al., (1999) teaches specific mutations that reduce (e.g., from 1.25% to 0.05% of wild-type activity) or completely obliterate enzymatic activity. Thus, a variant of endoH which comprises a substitution at Asp172 and/or Glu174 (with respect to SEQ ID NO: 20) would be understood to have undesired activity. Based on the published structural and functional analyses and routine experimentation, it could be readily determined those amino acids within endoH that could be substituted and would retain enzymatic activity and which amino acids could not be substituted.

In embodiments, the endoH that is surface displayed, e.g., is part of a fusion protein, comprises an amino acid sequence of SEQ ID NO: 19 or SEQ ID NO: 20. The amino acid sequence of SEQ ID NO: 1 lacks an N-terminal signal peptide that is present in SEQ ID NO: 20. The endoH may be a variant of SEQ ID NO: 19 or SEQ ID NO: 20. The variant may have at least or about 70%, 75%, 80%, 85%, 90%, 92%, 95%, 96%, 97%, 98%, 99%, or 100% sequence identity with one of SEQ ID NO: 19 or SEQ ID NO: 20.

In various embodiments, the fusion protein comprises an amino acid sequence that is at least 70% identical, at least 75% identical, at least 80% identical, at least 85% identical, at least 90% identical, or at least 95% identical, to one of SEQ ID NO: 21 to SEQ ID NO: 26.

In embodiments, the fusion protein comprises an amino acid sequence of one of one of SEQ ID NO: 24 to SEQ ID NO: 26.

Schematics of various surface displayed fusion proteins comprising a catalytic domain of endoH and an anchoring domain of a glycosylphosphatidylinositol (GPI)-anchored protein, i.e., Dan 1, Sed1, and Tir4 are shown in FIG. 3. Schematics of illustrative nucleic acids encoding the three surface displayed fusion proteins are shown in FIG. 2.

### Engineered Eukaryotic Cells

The present disclosure relates to engineered eukaryotic cells. These engineered cells are genetically modified to express a surface displayed fusion protein comprising a catalytic domain of an enzyme and an anchoring domain of a glycosylphosphatidylinositol (GPI)-anchored protein.

In embodiments, the engineered eukaryotic cell is a yeast cell.

In some embodiments, the engineered eukaryotic cell is a Pichia species. In some cases, the Pichia species is Pichia pastoris.

A fusion protein may be expressed by the cell by nucleic acid sequence, e.g., an expression cassette, that is stably integrated into a cell's chromosome. Alternately, a fusion protein may be expressed by the cell by an extrachromosomal nucleic acid sequence, e.g., plasmid, vector, or YAC which comprises an expression cassette. Any method for transfecting cells with suitable constructs that express the fusion protein may be used.

An expression cassette is any nucleic acid sequence that contains a subsequence that codes for a transgene and can confer expression of that subsequence when contained in a microorganism and is heterologous to that microorganism. It may comprise one or more of a coding sequence, a promoter, and a terminator. It may encode a secretory signal. It may further encode a signal sequence. In some embodiments, a nucleic acid sequence, e.g., which is expressed by a recombinant cell, may comprise an expression cassette.

The expression cassettes useful herein can be obtained using chemical synthesis, molecular cloning or recombinant methods, DNA or gene assembly methods, artificial gene synthesis, PCR, or any combination thereof. Methods of chemical polynucleotide synthesis are well known in the art and need not be described in detail herein. One of skill in the art can use the sequences provided herein and a commercial DNA synthesizer to produce a desired DNA sequence. For preparing polynucleotides using recombinant methods, a polynucleotide comprising a desired sequence can be inserted into a suitable cloning or expression vector, and the cloning or expression vector in turn can be introduced into a suitable host cell for replication and amplification. Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the expression vector. Methods for obtaining cloning and expression vectors are well-known (see, e.g., Green and Sambrook, Molecular Cloning: A Laboratory Manual, 4th edition, Cold Spring Harbor Laboratory Press, New York (2012)), the contents of which is incorporated herein by reference in its entirety.

In some cases, it is desirable for a engineered cell to express multiple copies of the fusion protein and/or to control expression of the fusion protein. Thus, a nucleic acid sequence or expression cassette may comprise a constitutive promoter, inducible promoter, and hybrid promoter. A promoter refers to a polynucleotide subsequence of nucleic acid sequence or an expression cassette that is located upstream, or 5′, to a coding sequence and is involved in initiating transcription of the coding sequence when the nucleic acid sequence or expression cassette is integrated into a chromosome or located extrachromosomally in a host cell.

Notably, in some cases, it is undesirable for a cell to excessively express the fusion protein. A primary purpose of the recombinant cells of the present disclosure is to produce the secreted recombinant proteins, e.g., for inclusion in composition for human or animal use. Should a cell express excessive amounts of the fusion protein, then the transcriptional and translational machinery dedicated to producing the fusion protein cannot be used to produce the secreted recombinant proteins. If so, the cell may become stressed and produce either less secreted recombinant proteins and/or may produce undesirable byproducts. Thus, in some embodiments, a nucleic acid encoding a fusion protein is fused to a weak promoter or to an intermediate strength promoter rather than a strong promoter.

In embodiments, the nucleic acid sequence or expression cassette comprises an inducible promoter. The inducible promoter may be an AOX1, DAK2, PEX11, FLD1, FGH1, DAS1, DAS2, CAT1, MDH3, HAC1, BiP, RAD30, RVS161-2, MPP10, THP3, TLR, GBP2, PMP20, SHB17, PEX8, PEX4, or TKL3 promoter. In some embodiments, the promoter used may have a sequence that has 95% or more sequence identity with any of SEQ ID NO: 32 to SEQ ID NO: 59. In some cases, the sequence identity may be greater than or about 90%, 92%, 95%, 96%, 97%, 98%, 99%, or 100% sequence identity with any of SEQ ID NO: 32 to SEQ ID NO: 59.

In embodiments, the nucleic acid sequence or expression cassette comprises a terminator sequence. A terminator is a section of nucleic acid sequence that marks the end of a gene during transcription. In some cases, the terminator is an AOX1, TDH3, MOX, RPS25A, or RPL2A terminator. In some embodiments, the terminator used may have a sequence that has 95% or more sequence identity with any of SEQ ID NO: 60 to SEQ ID NO: 63. In some cases, the sequence identity may be greater than or about 90%, 92%, 95%, 96%, 97%, 98%, 99%, or 100% sequence identity with any of SEQ ID NO: 60 to SEQ ID NO: 63.

Certain combinations of promoter and terminator may provide more preferred expression of the fusion protein and/or more preferred activity of the fusion protein. It is well-within the skill of an artisan to determine which combinations of promoters and terminators achieve desirability and which combinations do not.

Moreover, in some cases, the same combination of promoter and terminator may have preferred activity in one strain and have less preferred activity in another strain. Without wishing to be bound by theory, the strain difference may be due to a construct's integration into the host cell's genome or it may be due to epigenetic reasons. It is well-within the skill of an artisan to determine which strains for a certain combination of promoter and terminator achieve desirability and which strains do not.

Additionally, some combinations of promoters and terminators and certain strains perform better when cells are cultured at higher density (e.g., in bioreactors) versus low density cell cultures, as in a high throughput screen. Thus, a combination or strain may appear to be less desirable when assayed in small scale cultures, but may actually be a preferred combination or strain when cultured at higher cell density, which would be the case for commercial scale production of deglycosylated proteins. It is well-within the skill of an artisan to determine the culturing conditions that ensure certain combination of promoter and terminator and specific strains provided desirable amounts of enzymatic activity.

In some cases, the nucleic acid sequence or expression cassette encodes a signal peptide and/or a secretory signal. A signal peptide, also known as a signal sequence, targeting signal, localization signal, localization sequence, transit peptide, leader sequence, or leader peptide, may support secretion of a protein or polynucleotide. Extracellular secretion (for the purposes of surface display) of a recombinant or heterologously expressed fusion protein is facilitated by having a signal peptide included in the fusion protein. A signal peptide may be derived from a precursor (e.g., prepropeptide, preprotein) of a protein. Signal peptides may be derived from a precursor of a protein including, but not limited to, acid phosphatase (e.g., Pichia pastoris PHO1), albumin (e.g., chicken), alkaline extracellular protease (e.g., Yarrowia lipolytica XRP2), α-mating factor (α-MF, MFα1) (e.g., Saccharomyces cerevisiae), amylase (e.g., α-amylase, Rhizopus oryzae, Schizosaccharomyces pombe putative amylase SPCC63.02c (Amyl)), β-casein (e.g., bovine), carbohydrate binding module family 21 (CBM21)-starch binding domain, carboxypeptidase Y (e.g., Schizosaccharomyces pombe Cpy1), cellobiohydrolase I (e.g., Trichoderma reesei CBH1), dipeptidyl protease (e.g., Schizosaccharomyces pombe putative dipeptidyl protease SPBC1711.12 (Dpp1)), glucoamylase (e.g., Aspergillus awamori), heat shock protein (e.g., bacterial Hsp70), hydrophobin (e.g., Trichoderma reesei HBFI, Trichoderma reesei HBFII), inulase, invertase (e.g., Saccharomyces cerevisiae SUC2), killer protein or killer toxin (e.g., 128 kDa pGKL killer protein, α-subunit of the K1 killer toxin (e.g., Kluyveromyces lactis), K1 toxin KILM1, K28 pre-pro-toxin, Pichia acaciae), leucine-rich artificial signal peptide CLY-L8, lysozyme (e.g., chicken CLY), phytohemagglutinin (PHA-E) (e.g., Phaseolus vulgaris), maltose binding protein (MBP) (e.g., Escherichia coli), P-factor (e.g., Schizosaccharomyces pombe P3), Pichia pastoris Dse, Pichia pastoris Exg, Pichia pastoris Pir1, Pichia pastoris Scw, and cell wall protein Pir4 (protein with internal repeats). In some embodiments, the signal peptide used may have a sequence that has 80% or more sequence identity with any of SEQ ID NO: 64 to SEQ ID NO: 163. In some cases, the sequence identity may be greater than or about 90%, 92%, 95%, 96%, 97%, 98%, 99%, or 100% sequence identity with any of SEQ ID NO: 64 to SEQ ID NO: 163. In some cases, the signal peptide used may have a sequence that has 80% or more sequence identity with any of SEQ ID NO: 64 to SEQ ID NO: 163. In some cases, the sequence identity may be greater than or about 90%, 92%, 95%, 96%, 97%, 98%, 99%, or 100% sequence identity with any of SEQ ID NO: 64 to SEQ ID NO: 163.

In various embodiments, a fusion protein comprises an α-mating factor (α-MF, MFα1) (e.g., Saccharomyces cerevisiae) secretion signal. In some cases the alpha mating factor signal peptide and secretion signal has a sequence that has 95% or more sequence identity with SEQ ID NO: 298 or SEQ ID NO: 299. In some cases, the sequence identity may be greater than or about 90%, 92%, 95%, 96%, 97%, 98%, 99%, or 100% sequence identity with any of with SEQ ID NO: 2998 or SEQ ID NO: 299. The α-mating factor secretion signal targets a fusion protein through the secretory pathway and is removed before exiting the cell.

In some cases, a nucleic acid sequence or expression cassette encodes a selectable marker. The selectable maker may be an antibiotic resistance gene (e.g., zeocin, ampicillin, blasticidin, kanamycin, nourseothricin, chloroamphenicol, tetracycline, triclosan, ganciclovir, and any combination thereof), an auxotrophic marker (e.g., f ade1, arg4, his4, ura3, met2, and any combination thereof).

In various embodiments, a nucleic acid sequence or expression cassette comprises codons that are optimized for the species of the engineered cell, e.g., a yeast cell including a Pichia cell. As known in the art, codon optimization may improve stability and/or increase expression of a recombinant protein, e.g., a fusion protein of the present disclosure. Surprisingly, codon optimization of a nucleic acid sequence or expression cassette may improve the transfection efficiency of the nucleic acid sequence or expression cassette into the genome of a host cell. Codon utilization tables for various species of host cell are publicly available. See, e.g., the worldwide web (at) kazusa.or.jp/codon/cgi-bin/showcodon.cgi?species=4922&aa=15&style=N.

Host cells useful for expression fusion proteins of the present disclosure include but are not limited to: Arxula spp., Arxula adeninivorans, Kluyveromyces spp., Kluyveromyces lactis, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Colletotrichum spp., Colletotrichum gloeosporiodes, Endothia spp., Endothia parasitica, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculosum, Penicillium purpurogenum, Penicillium roqueforti, Pleurotus spp., Pleurotus ostreatus, Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, Trichoderma vireus, Aspergillus oryzae, Bacillus subtilis, Escherichia coli, Myceliophthora thermophila, Neurospora crassa, Pichia pastoris, Komagataella phaffii and Komagataella pastoris.

Transfection of a host cell with an expression cassette can exploit the natural ability of a host cell to integrate exogenous DNA into its chromosome. This natural ability is well documented for yeast cells, including Pichia cells. In some embodiments an additional vector and or additional elements may be designed to aide (as deemed necessary by one skilled in the art) for the particular method of transfection (e.g. CAS9 and gRNA vectors for a CRISPR/CAS9 based method).

In some cases, a host eukaryotic cell that expresses a fusion protein comprises a mutation in its AOX1 gene and/or its AOX2 gene. A deletion in either the AOX1 gene or AOX2 gene generates a methanol-utilization slow (mutS) phenotype that reduces the strain's ability to consume methanol as an energy source. A deletion in both the AOX1 gene and the AOX2 gene generates a methanol-utilization minus (mutM) phenotype that substantially limits the strain's ability to consume methanol as an energy source. Using an AOX1 mutant and/or AOX2 mutant cell is especially useful in the context of a fusion protein encoded by an expression cassette that comprises a methanol-inducible promoter, e.g., AOX1, DAK2, PEX11, FLD1, FGH1, DAS2, CAT1, PMP20, SHB17, PEX8, PEX4, TKL3 or DAS1. In this configuration, the host cell does not use methanol as an energy source, thus, when the cell is provided methanol, the methanol is primarily used to activate the methanol-inducible promoter, thereby especially activating the promoter and causing increased expression of the fusion protein.

The conditions that promote expression of the fusion protein may be standard growth conditions. However, when the engineered eukaryotic cell comprises a nucleic acid sequence that encodes the fusion protein and comprises an inducible promoter, culturing the engineered eukaryotic cell under conditions that promote expression of the fusion protein comprises contacting the cell with an agent that activates the inducible promoter. When the inducible promoter is an AOX1, DAK2, PEX11, FLD1, FGH1, DAS2, CAT1, PMP20, SHB17, PEX8, PEX4, TKL3 or DAS1 promoter the agent that activates the inducible promoter is methanol.

In some embodiments, the engineered eukaryotic cell comprises an additional genomic modification comprising a knockout of a coding sequence for a cell wall protein or an additional genomic modification that overexpresses a cell wall protein. In some cases, the engineered eukaryotic cell comprises an additional genomic modification comprising a knockout of the coding sequences for more than one cell wall proteins or an additional genomic modification that overexpresses more than one a cell wall proteins. In various cases, the cell wall protein is a mannoprotein. In further cases, the cell wall protein is one or more of a CCW12 homolog, a CCW14 homolog, a CCW22 homolog, a FLO5 homolog, or a SED1 homolog. In additional cases, the cell wall protein comprises the amino acid sequence of any one of SEQ ID NO: 306 to SEQ ID NO: 319. In some cases, the additional genomic modification reduces the number of native cell wall proteins expressed by the engineered eukaryotic cell, thereby allowing additional space for localization of the surface-displayed fusion protein.

In various embodiments, the engineered eukaryotic cell comprises a further genomic modification that overexpresses a protein related to the p24 complex. In some cases, the engineered eukaryotic cell comprises a further genomic modification comprising that overexpresses more than one protein related to the p24 complex. In various cases, the protein related to the p24 complex is selected from Erp1, Erp2, Erp3, Erp5, Emp24, and Erv25. In further cases, the protein related to the p24 complex comprises the amino acid sequence of any one of SEQ ID NO: 320 to SEQ ID NO: 325. In some cases, the further genomic modification promotes trafficking of the surface-displayed fusion protein through the secretory pathway.

Yet another aspect of the present disclosure is a population of any herein-disclosed engineered eukaryotic cells.

A further aspect of the present disclosure is a bioreactor comprising a population of any herein-disclosed engineered eukaryotic cells.

In an aspect, the present disclosure provides a composition comprising any herein-disclosed engineered eukaryotic cells and a secreted recombinant protein.

In embodiments, the secreted recombinant protein is an animal protein, e.g., an egg protein. The egg protein may be selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

In another aspect, the present disclosure provides a composition comprising any herein-disclosed engineered eukaryotic cell, a secreted recombinant protein that has been deglycosylated, and one or more oligosaccharides cleaved from the secreted recombinant protein.

In some embodiments, the secreted recombinant protein is an animal protein, e.g., an egg protein. The egg protein may be selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

Another aspect of the present disclosure is a method for expressing a surface-displayed fusion protein comprising a catalytic domain of an enzyme and an anchoring domain of glycosylphosphatidylinositol (GPI)-anchored protein. The method comprising obtaining any herein-disclosed engineered eukaryotic cell and culturing the engineered eukaryotic cell under conditions that promote expression of the fusion protein.

In some embodiments, when the engineered eukaryotic cell comprises a genomic modification and/or an extrachromosomal modification that overexpresses a secreted recombinant protein comprises an inducible promoter, the method comprises culturing the engineered eukaryotic cell under conditions that promote expression of the fusion protein by contacting the engineered eukaryotic with an agent that activates the inducible promoter.

In various embodiments, the inducible promoter is an AOX1, DAK2, PEX11, FLD1, FGH1, DAS1, DAS2, CAT1, MDH3, HAC1, BiP, RAD30, RVS161-2, MPP10, THP3, TLR, GBP2, PMP20, SHB17, PEX8, PEX4, or TKL3 promoter. In some cases, when the inducible promoter is an AOX1, DAK2, PEX11, FLD1, FGH1, DAS2, CAT1, PMP20, SHB17, PEX8, PEX4, TKL3 or DAS1 promoter and the agent that activates the inducible promoter is methanol. In various cases, the secreted recombinant protein is designed to be secreted from the cell and/or is capable of being secreted from the cell.

### Secreted Proteins

In various embodiments, the engineered eukaryotic cell comprises a genomic modification that overexpresses a secreted recombinant protein and/or comprises an extrachromosomal modification that overexpresses a secreted recombinant protein.

In some cases, the secreted recombinant protein is an animal protein, e.g., an egg protein. The egg protein may be selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

The secreted recombinant protein may have amino acid sequence of any one of SEQ ID NO: 164 to SEQ ID NO: 297. The secreted recombinant protein may be a variant of any one of SEQ ID NO: 164 to SEQ ID NO: 297. The variant may have at least or about 70%, 75%, 80%, 85%, 90%, 92%, 95%, 96%, 97%, 98%, 99%, or 100% sequence identity with one of SEQ ID NO: 164 to SEQ ID NO: 297.

In some cases, the engineered eukaryotic cell that expresses the surface display fusion protein further comprises a genomic modification that overexpresses secreted recombinant protein. Here, as a cell secretes the recombinant protein into the extracellular space, it comes in contact with a surface displayed fusion protein, which enzymatically interacts with the secreted recombinant protein.

In some cases, the secreted recombinant protein is a glycoprotein and the catalytic domain of the enzyme cleaves oligosaccharide from the secreted recombinant protein, with both the deglycosylated protein and the liberated oligosaccharide progressing into the extracellular space, e.g., the growth medium in which the eukaryotic cell is being cultured.

In alternate cases, a first engineered eukaryotic cell expresses the surface display fusion protein and a second engineered eukaryotic cell overexpresses a secreted recombinant protein.

The genomic modification that overexpresses the secreted recombinant protein may comprise a promoter (constitutive promoter, inducible promoter, and hybrid promoter) as disclosed herein; the genomic modification that overexpresses the secreted recombinant protein may comprise a terminator sequence as disclosed herein; the genomic modification that overexpresses the secreted recombinant protein may encode a secretory signal as disclosed herein; and/or the genomic modification that overexpresses the secreted recombinant protein may encode a signal sequence as disclosed herein.

In embodiments, the genomic modification and/or the extrachromosomal modification that overexpresses the secreted recombinant protein comprises an inducible promoter. In some cases, the inducible promoter is an AOX1, DAK2, PEX11, FLD1, FGH1, DAS1, DAS2, CAT1, MDH3, HAC1, BiP, RAD30, RVS161-2, MPP10, THP3, TLR, GBP2, PMP20, SHB17, PEX8, PEX4, or TKL3 promoter. In some cases, when the inducible promoter is an AOX1, DAK2, PEX11, FLD1, FGH1, DAS2, CAT1, PMP20, SHB17, PEX8, PEX4, TKL3 or DAS1 promoter and the agent that activates the inducible promoter is methanol.

A host cell may comprise a first promoter driving the expression of the fusion protein and a second promoter driving the expression of the secreted recombinant protein. The first and second promoter may be selected from the list of promoters provided herein. In some cases, the first promoter and the second promoter may be the same. Alternatively, the first and the second promoter may be different.

In various cases, the genomic modification and/or the extrachromosomal modification that overexpresses a secreted recombinant protein comprises an AOX1, TDH3, MOX, RPS25A, or RPL2A terminator.

In further cases, the genomic modification and/or the extrachromosomal modification that overexpresses a secreted recombinant protein encodes a signal peptide and/or a secretory signal.

In additional cases, the genomic modification and/or the extrachromosomal modification that overexpresses a secreted recombinant protein comprises codons that are optimized for the species of the engineered eukaryotic cell. In some cases, the secreted recombinant protein is designed to be secreted from the cell and/or is capable of being secreted from the cell.

### Additional Attachments for Surface Display

In embodiments, the engineered eukaryotic cell further encodes one or more additional fusion proteins comprising a catalytic domain of an enzyme and an adhesion or anchoring domain from a cell surface protein selected from Sed1p, Flo5-2, Flo11, Saccharomyces cerevisiae Flo5, CWP, and PIR with the adhesion or anchoring domain having the ability to capture exopolysaccharides and retain the additional fusion protein at the extracellular surface.

Sed1p is a major component of the Saccharomyces cerevisiae cell wall. It is required to stabilize the cell wall and for stress resistance in stationary-phase cells. See, e.g., the world wide web (at) uniprot.org/uniprot/Q01589. It is believed that Asn 318 (with respect to SEQ ID NO: 13) is the most likely candidate for the GPI attachment site in Sed1p. In some embodiments, a fusion protein comprising a Sed1p anchoring domain has a sequence having at least 95% or more sequence identity with SEQ ID NO:13 or SEQ ID NO: 14. In some cases, the sequence identity may be greater than or about 90%, 95%, 96%, 97%, 98%, 99%, or 100%.

Komagataella phaffii Flo5-2 is considered to be an ortholog of both Saccharomyces Flo1 and Flo5. See, e.g., the world wide web (at) uniprot.org/uniprot/F2QXP0. The Saccharomyces flocculation proteins are cell wall proteins that participate directly in adhesive cell-cell interactions during yeast flocculation, a reversible, asexual process in which cells adhere to form aggregates (flocs) consisting of thousands of cells. The lectin-like proteins stick out of the cell wall of flocculent cells and selectively bind mannose residues in the cell walls of adjacent cells. Literature on Saccharomyces Flo 1p shows that monomeric mannose added to the media can prevent flocculation, suggesting that flocculation by Flo 1p results from binding to mannose in the cell wall and free-floating mannose can compete for the binding spot. Thus, the flocculation family of proteins are useful in the present disclosure, for, at least, two reasons. First, they generally extend relatively far from the cell wall and, second, it is believed that they bind and capture some exopolysaccharides. A fusion protein comprising an anchoring domain of Flo5-2 may anchor the fusion protein to the extracellular surface of an engineered cell via its GPI anchor or by the domain's interaction with exopolysaccharides located on the extracellular surface of an engineered cell. Moreover, without wishing to be bound by theory, inclusion of an anchoring domain of Flo5-2 may promote capture of a secreted glycoprotein for deglycosylation.

In some embodiments, a fusion protein comprising a Flo5-2 anchoring domain has a sequence that has 95% or more sequence identity with SEQ ID NO: 5 or SEQ ID NO: 6. In some cases, the sequence identity may be greater than or about 90%, 95%, 96%, 97%, 98%, 99%, or 100%. In various embodiments, the Flo5-2 anchoring domain of a fusion protein of the present disclosure comprises a GPI attachment site; thus, the anchoring domain may only require a short fragment of SEQ ID NO: 5 or SEQ ID NO: 6, i.e., a fragment that is 5, 10, 25, 50, 100, 200, 300, 400, 500, 600, 700, 800, 900, or 1000 or more amino acids in length, as long as it is capable of projecting the catalytic domain of the fusion protein into the extracellular space. In some embodiments, the anchoring domain comprises, at least, Flo5-2's GPI attachment site. In some embodiments, the anchoring domain lacks Flo5-2's GPI attachment site yet retains the ability to capture exopolysaccharides and retain the fusion protein at the extracellular surface.

In some embodiments, a fusion protein comprising a Saccharomyces cerevisiae Flo5 anchoring domain has a sequence that has 95% or more sequence identity with SEQ ID NO: 335. In some embodiments, the anchoring domain lacks Flo5's GPI attachment site yet retains the ability to capture exopolysaccharides and retain the fusion protein at the extracellular surface.

Flo11 is another GPI-anchored cell surface glycoprotein (flocculin). See, e.g., the worldwide web (at) uniprot.org/uniprot/F2QRD4. Flo11 is believed to be required for pseudohyphal and invasive growth, flocculation, and biofilm formation. Like, Flo5-2, Flo11 has a GPI anchor site towards its C-terminus which can tether the protein to a cell's membrane. Therefore, a fusion protein comprising an anchoring domain of Flo11 may anchor the fusion protein to the extracellular surface of an engineered cell via its GPI anchor or by the domain's interaction with exopolysaccharides located on the extracellular surface of an engineered cell.

In some embodiments, a fusion protein comprising a Flo11 anchoring domain has a sequence that has 95% or more sequence identity with SEQ ID NO: 328 or SEQ ID NO: 329. In some cases, the sequence identity may be greater than or about 90%, 95%, 96%, 97%, 98%, 99%, or 100%. In various embodiments, the Flo11 anchoring domain of a fusion protein of the present disclosure comprises a GPI attachment site; thus, the anchoring domain may only require a short fragment of SEQ ID NO: 328 or SEQ ID NO: 329, i.e., a fragment that is 5, 10, 25, 50, 100, 200, 300, 400, 500, 600, 700, 800, 900, or 1000 or more amino acids in length, as long as it is capable of projecting the catalytic domain of the fusion protein into the extracellular space. In some embodiments, the anchoring domain lacks Flo11's GPI attachment site yet retains the ability to capture exopolysaccharides and retain the fusion protein at the extracellular surface.

A fusion protein comprising a CWP, and PIR anchoring domain may be attached to a cell wall, independent of a GPI linkage.

### Compositions

In an aspect, the present disclosure provides a composition comprising any herein-disclosed engineered eukaryotic cells and a secreted recombinant protein.

In embodiments, the secreted recombinant protein is an animal protein, e.g., an egg protein. The egg protein may be selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

In another aspect, the present disclosure provides a composition comprising any herein-disclosed engineered eukaryotic cell, a secreted recombinant protein that has been deglycosylated, and one or more oligosaccharides cleaved from the secreted recombinant protein.

In some embodiments, the secreted recombinant protein is an animal protein, e.g., an egg protein. The egg protein may be selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

Also, the present disclosure further relates to a composition comprising a secreted protein that has been deglycosylated and one or more oligosaccharides cleaved from the secreted protein.

Further, the present disclosure relates to a composition comprising a secreted protein that has been deglycosylated.

Additionally, the present disclosure relates to a composition comprising one or more oligosaccharides cleaved from a secreted protein.

These compositions may be liquid or dried. The secreted protein that has been deglycosylated and/or one or more oligosaccharides cleaved from the secreted protein may be lyophilized. In some cases, the secreted protein that has been deglycosylated and/or one or more oligosaccharides cleaved from the secreted protein are isolated, e.g., from each other and/or from a growth medium. The secreted protein that has been deglycosylated and/or one or more oligosaccharides cleaved from the secreted protein may be concentrated.

Deglycosylated proteins and/or one or more oligosaccharides cleaved from the secreted protein, as disclosed herein, may be used in a consumable composition comprising. Illustrative uses and features of such consumable compositions are described in WO 2016/077457, the contents of which is incorporated herein by reference in its entirety.

A consumable composition may comprise one or more deglycosylated proteins. As used herein, a consumable composition refers to a composition, which comprises an isolated deglycosylated protein and/or a cleaved oligosaccharide and may be consumed by an animal, including but not limited to humans and other mammals. Consumable food compositions include food products, beverage products, dietary supplements, food additives, and nutraceuticals as non-limiting examples. The consumable composition may comprise one or more components in addition to the deglycosylated protein. The one or more components may include ingredients, solvents used in the formation of foodstuff or beverages. For instance, the deglycosylated protein may be in the form of a powder which can be mixed with solvents to produce a beverage or mixed with other ingredients to form a food product.

The nutritional content of the deglycosylated protein may be higher than the nutritional content of an identical quantity of a control protein. The control protein may be the same protein produced recombinantly but not treated with a fusion protein of the present disclosure. The control protein may be the same protein produced recombinantly in a host cell which does not express a surface displayed fusion protein. The control protein may be the same protein isolated from a naturally occurring source. For instance, the control protein may be an isolated an egg white protein.

The nutritional content of a composition comprising the deglycosylated protein can be more than the nutritional content of the composition comprising a control protein. The protein content of the deglycosylated protein composition may be about 1% to 80% more than the protein content of a composition comprising a control protein. The protein content of the deglycosylated protein composition may be about 1% to 5% more than the protein content of a composition comprising a control protein. The protein content of the deglycosylated protein composition may be about 1% to 10% more than the protein content of a composition comprising a control protein. The protein content of the deglycosylated protein composition may be about 1% to 20% more than the protein content of a composition comprising a control protein. The protein content of the deglycosylated protein composition may be about 1% to 50% more than the protein content of a composition comprising a control protein. The protein content of the deglycosylated protein composition may be about 1% to 80% more than the protein content of a composition comprising a control protein. The protein content of the deglycosylated protein composition may be about 5% to 10%, 5-15%, 5-20%, 5-30%, 5-50%, 5-80% more than the protein content of a composition comprising a control protein. The protein content of the deglycosylated protein composition may be about 10% to 80%, 10-20%, 10-30%, 10-50%, 10-70%, 10-80% more than the protein content of a composition comprising a control protein. The protein content of the deglycosylated protein composition may be about 1%, 5%, 10%, 20%, 30%, 40%, 50%, 60%, 70%, or 80% more than the protein content of a composition comprising a control protein.

Protein content of a deglycosylated protein composition may be measured using conventional methods. For instance, protein content may be measured using nitrogen quantitation by combustion and then using a conversion factor to estimate quantity of protein in a sample followed by calculating the percentage (w/w) of the dry matter.

The nitrogen to carbon ratio of a deglycosylated protein be higher than the nitrogen to carbon ratio of a control protein. The nitrogen to carbon ratio of a recombinant protein may be greater than or equal to about 0.1. The nitrogen to carbon ratio of a deglycosylated protein be higher than the nitrogen to carbon ratio of a control protein. The nitrogen to carbon ratio of a recombinant protein may be greater than or equal to about 0.25. The nitrogen to carbon ratio of a recombinant protein may be greater than or equal to about 0.3. The nitrogen to carbon ratio of a recombinant protein may be greater than or equal to about 0.35. The nitrogen to carbon ratio of a recombinant protein may be greater than or equal to about 0.4. The nitrogen to carbon ratio of a recombinant protein may be greater than or equal to about 0.5.

Solubility of a deglycosylated protein may be greater than the solubility of a control protein. Solubility of a composition comprising a deglycosylated protein may be higher than the solubility of a composition comprising the control protein. Thermal stability of the deglycosylated protein may be greater than the thermal stability of a control protein.

The degree of glycosylation of the recombinant protein may be dependent on the consumable composition being produced. For instance, a consumable composition may comprise a lower degree of glycosylation to increase the protein content of the composition. Alternatively, the degree of glycosylation may be higher to increase the solubility of the protein in the composition.

### Methods

In yet another aspect, the present disclosure provides a method for post-translationally modifying a secreted recombinant protein. The method comprising contacting a secreted recombinant protein with a fusion protein anchored to any herein-disclosed engineered eukaryotic cell, wherein the fusion protein comprises a catalytic enzyme that deglycosylates, acetylates, adenylates, alkylates, amidates, glycosylates, hydroxylates, methylates, or phosphorylates.

In a further aspect, the present disclosure provides a method for removing impurities secreted by an engineered eukaryotic cell. The method comprising culturing any herein-disclosed engineered eukaryotic cell under conditions that an impurity is secreted by the engineered eukaryotic cell and contacting the impurity with a fusion protein anchored to the engineered eukaryotic cell, wherein the fusion protein comprises a catalytic enzyme that cleaves the impurity, denatures the impurity, modifies the impurity, and/or detoxifies the impurity.

An aspect of the present disclosure is a method for allowing an engineered eukaryotic cell to rely on alternate carbon sources. The method comprising contacting an alternate carbon source with a fusion protein anchored any herein-disclosed engineered eukaryotic cell, wherein the fusion protein comprises a catalytic enzyme that cleaves the alternate carbon source into a carbon source that can be taken in by the cell and used as a carbon source by the cell.

In various embodiments, when the fusion protein comprises an invertase, the engineered eukaryotic cell is capable of growing on sucrose as its primary carbon source. In some cases, when the fusion protein comprises the anchoring domain is from Tir4, the engineered eukaryotic cell has increased growth when grown on sucrose as its primary carbon source relative to a eukaryotic cell that is not engineered to rely on sucrose as an alternate carbon source.

Another aspect of the present disclosure is a method for deglycosylating a secreted glycoprotein. The method comprises contacting a secreted protein with a fusion protein anchored to any herein-disclosed engineered eukaryotic cell. By contacting a secreted protein with the fusion protein, the catalytic domain cleaves and releases an oligonucleotide from the secreted glycoprotein.

In some cases, the secreted glycoprotein is expressed by the engineered eukaryotic cell.

Notably, a fusion protein anchored to an engineered eukaryotic cell (of the present disclosure) is more effective at deglycosylating the secreted glycoprotein than an intracellular endoglycosidase, e.g., an intracellular endoglycosidase located within a Golgi vesicle. In particular, a fusion protein anchored to the surface of an engineered eukaryotic cell (of the present disclosure) is more effective at deglycosylating the secreted glycoprotein than an intracellular endoglycosidase that is linked to a membrane associating domain, e.g., a membrane associating domain that comprises an amino acid sequence of OCH1. Preferably, the amino acid sequence of OCH1 that is included in a fusion protein of the present disclosure lacks the wild-type OCH1 Golgi retention domain. This retention domain comprises at least a portion of the first 48 residues of Pichia OCH1 protein. If the Golgi retention domain of OCH1 is included in a fusion protein of the present disclosure, then it is unlikely that the fusion protein would be displayed on the exterior of the cell, as needed to be a surface displayed fusion protein of the present disclosure. In embodiments, a fusion protein having an OCH1 anchoring domain lacks the OCH1 Golgi retention domain. In some embodiments, a fusion protein having an OCH1 anchoring domain lacks at least a portion of the first 48 residues of Pichia OCH1 protein. In various embodiments, a fusion protein having an OCH1 anchoring domain lacks the first 48 residues of Pichia OCH1 protein.

A deglycosylated protein of the present disclosure can have a level of N-linked glycosylation that is reduced by at least about 10 percent (e.g., 10 percent, 20 percent, 30 percent, 40 percent, 50 percent, 60 percent, 70 percent, 80 percent, 90 percent, or 100 percent) as compared to the level of N-linked glycosylation of the same glycoprotein that is not contacted with a fusion protein of the present disclosure, including a glycoprotein contacted with an intracellular endoglycosidase.

In some cases, the secreted glycoprotein is expressed by a cell other than the engineered eukaryotic cell.

In some embodiments, the method further comprises a step of isolating the deglycosylated secreted protein, e.g., from a cleaved oligosaccharide and/or from its growth medium. In some embodiments, the method further comprises a step of drying the deglycosylated secreted protein and/or the cleaved oligosaccharides.

In various embodiments, the secreted glycoprotein is an animal protein. In some embodiments, the animal protein is an egg protein, e.g., selected from the group consisting of ovalbumin, ovomucoid, lysozyme ovoglobulin G2, ovoglobulin G3, α-ovomucin, (3-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

The glycoprotein may have amino acid sequence of any one of SEQ ID NO: 164 to SEQ ID NO: 297. The glycoprotein may be a variant of any one of SEQ ID NO: 164 to SEQ ID NO: 297. The variant may have at least or about 70%, 75%, 80%, 85%, 90%, 92%, 95%, 96%, 97%, 98%, 99%, or 100% sequence identity with one of SEQ ID NO: 164 to SEQ ID NO: 297.

Another aspect of the present disclosure is a method for deglycosylating a plurality of secreted glycoproteins. The method comprises contacting the plurality of secreted glycoproteins with a population of any herein disclosed engineered eukaryotic cells. By contacting the plurality of secreted glycoprotein with the fusion protein, the catalytic domains cleave and release oligonucleotides from the plurality secreted glycoprotein and provide a plurality of deglycosylated secreted proteins.

In some cases, substantially every secreted glycoprotein in the plurality of secreted glycoproteins is deglycosylated upon contact with the population of engineered eukaryotic cells.

Notably, the amount of deglycosylation of the secreted glycoproteins is not increased by further contacting the secreted protein with an isolated endoglycosidase.

Further, the amount of deglycosylation of the secreted glycoproteins is more than the amount obtained from a population of cells that express an intracellular endoglycosidase in addition to expressing the secreted glycoprotein.

In some embodiments, the method further comprises a step of isolating the plurality of deglycosylated secreted proteins and may further comprise a step of drying the plurality of deglycosylated secreted proteins.

In various embodiments, the secreted glycoprotein is an animal protein. In some embodiments, the animal protein is an egg protein, e.g., selected from the group consisting of ovalbumin, ovomucoid, lysozyme ovoglobulin G2, ovoglobulin G3, α-ovomucin, (3-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

The glycoprotein may have amino acid sequence of any one of SEQ ID NO: 164 to SEQ ID NO: 297. The glycoprotein may be a variant of any one of SEQ ID NO: 164 to SEQ ID NO: 297. The variant may have at least or about 70%, 75%, 80%, 85%, 90%, 92%, 95%, 96%, 97%, 98%, 99%, or 100% sequence identity with one of SEQ ID NO: 164 to SEQ ID NO: 297.

Any aspect or embodiment described herein can be combined with any other aspect or embodiment as disclosed herein.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used in the specification and claims, the singular forms “a”, “an” and “the” include plural references unless the context clearly dictates otherwise.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” mean A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

As used herein, the term “about” a number refers to that number plus or minus 10% of that number and/or within one standard deviation (plus or minus) from that number. The term “about” a range refers to that range minus 10% of its lowest value and plus 10% of its greatest value and that range minus one standard deviation its lowest value and plus one standard deviation of its greatest value.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “increased”, “increasing”, or “increase” are used herein to generally mean an increase by a statically significant amount relative to a reference level. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease in a value relative to a reference level. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level.

As used herein, the term “catalytic domain” comprises a portion of an enzyme that provides catalytic activity

The section headings used herein are for organizational purposes only and are not to be construed as limiting the subject matter described.

### REFERENCES

Ye M et al. Cell-surface Engineering of Yeasts for Whole-cell Biocatalysts. Bioprocess and Biosystems Engineering. 2021. 44:1003-1019.
Pastor-Cantizano N et al. p24 family proteins: key players in the regulation of trafficking along the secretory pathway. Protoplasma. 2016. 253(4):967-85.
Wentz A E and Shusta E V. A novel high-throughput screen reveals yeast genes that increase secretion of heterologous proteins. Appl Environ Microbiol. 2007. 73(4):1189-1198.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### Additional Embodiments

Embodiment 1: An engineered eukaryotic cell comprising a surface displayed catalytic domain of an endoglycosidase, wherein the surface displayed catalytic domain of an endoglycosidase is a portion of a fusion protein expressed by the cell.

Embodiment 2: The engineered eukaryotic cell of Embodiment 1, wherein the fusion protein further comprises an anchoring domain of a cell surface protein.

Embodiment 3: The engineered eukaryotic cell of Embodiment 1 or Embodiment 2, wherein the fusion protein comprises a portion of the endoglycosidase in addition to its catalytic domain.

Embodiment 4: The engineered eukaryotic cell of any one of Embodiments 1 to 3, wherein the fusion protein comprises substantially the entire amino acid sequence of the endoglycosidase.

Embodiment 5: The engineered eukaryotic cell of any one of Embodiments 1 to 4, wherein the endoglycosidase is endoglycosidase H.

Embodiment 6: The engineered eukaryotic cell of any one of Embodiments 1 to 5, wherein the fusion protein comprises an amino acid sequence that is at least 95%, 96%, 97%, 98%, 99%, or 100% identical to SEQ ID NO: 19 or SEQ ID NO:20.

Embodiment 7: The engineered eukaryotic cell of any one of Embodiments 1 to 6, wherein the fusion protein comprises a portion of the cell surface protein in addition to its anchoring domain.

Embodiment 8: The engineered eukaryotic cell of any one of Embodiments 1 to 7, wherein the fusion protein comprises substantially the entire amino acid sequence of the cell surface protein.

Embodiment 9: The engineered eukaryotic cell of any one of Embodiments 1 to 8, wherein the cell surface protein is selected from Sed1p, Flo5-2, or Flo11.

Embodiment 10: The engineered eukaryotic cell of any one of Embodiments 1 to 9, wherein the fusion protein comprises an amino acid sequence that is at least 95% identical to one of SEQ ID NO: 13 to SEQ ID NO: 328 and SEQ ID NO: 335.

Embodiment 11: The engineered eukaryotic cell of any one of Embodiments 1 to 10, wherein the anchoring domain stably attaches the fusion protein to the extracellular surface of the cell.

Embodiment 12: The engineered eukaryotic cell of any one of Embodiments 1 to 11, wherein upon translation the fusion protein comprises a signal peptide and/or a secretory signal.

Embodiment 13: The engineered eukaryotic cell of any one of Embodiments 1 to 12, wherein the anchoring domain is N-terminal to the catalytic domain in the fusion protein.

Embodiment 14: The engineered eukaryotic cell of Embodiment 13, wherein the fusion protein comprises a linker C-terminal to the anchoring domain.

Embodiment 15: The engineered eukaryotic cell of any one of Embodiments 1 to 12, wherein the anchoring domain is C-terminal to the catalytic domain in the fusion protein.

Embodiment 16: The engineered eukaryotic cell of Embodiment 15, wherein the fusion protein comprises a linker N-terminal to the anchoring domain.

Embodiment 17: The engineered eukaryotic cell of any one of Embodiments 1 to 16, wherein the cell surface protein is Sed1p and the endoglycosidase is endoglycosidase H.

Embodiment 18: The engineered eukaryotic cell of Embodiment 17, wherein the fusion protein comprises an amino acid sequence that is at least 95% identical to SEQ ID NO: 336 or SEQ ID NO: 337.

Embodiment 19: The engineered eukaryotic cell of any one of Embodiments 1 to 16, wherein the cell surface protein is Flo5-2 or Flo11 and the endoglycosidase is endoglycosidase H.

Embodiment 20: The engineered eukaryotic cell of Embodiment 19, wherein the fusion protein comprises an amino acid sequence that is at least 95% identical to SEQ ID NO: 338 or SEQ ID NO: 339.

Embodiment 21: The engineered eukaryotic cell of Embodiment 19, wherein the fusion protein comprises an amino acid sequence that is at least 95% identical to SEQ ID NO: 340 or SEQ ID NO: 341.

Embodiment 22: An engineered eukaryotic cell that expresses a fusion protein comprising a catalytic domain of an endoglycosidase and a portion of a cell surface protein, wherein the portion of the cell surface protein lacks its native anchoring domain.

Embodiment 23: The engineered eukaryotic cell of Embodiment 22, wherein the fusion protein comprises a portion of the endoglycosidase in addition to its catalytic domain.

Embodiment 24: The engineered eukaryotic cell of Embodiment 22 or Embodiment 23, wherein the fusion protein comprises substantially the entire amino acid sequence of the endoglycosidase.

Embodiment 25: The engineered eukaryotic cell of any one of Embodiments 22 to 24, wherein the endoglycosidase is endoglycosidase H.

Embodiment 26: The engineered eukaryotic cell of any one of Embodiments 22 to 25, wherein the fusion protein comprises an amino acid sequence that is at least 95% identical to SEQ ID NO: 19 or SEQ ID NO: 20.

Embodiment 27: The engineered eukaryotic cell of any one of Embodiments 22 to 26, wherein the fusion protein comprises substantially the entire amino acid sequence of the cell surface protein other than its native anchoring domain.

Embodiment 28: The engineered eukaryotic cell of any one of Embodiments 22 to 27, wherein the cell surface protein is Flo5-2.

Embodiment 29: The engineered eukaryotic cell of any one of Embodiments 22 to 28, wherein the fusion protein comprises an amino acid sequence that is at least 95% identical to SEQ ID NO: 330 and is capable of binding an exopolysaccharide present on the surface of the cell and thereby attaching the fusion protein to the extracellular surface of the cell for surface display.

Embodiment 30: The engineered eukaryotic cell of any one of Embodiments 22 to 29, wherein the portion of the cell surface protein that lacks its native anchoring domain is capable of adhering to an extracellular component of the cell.

Embodiment 31: The engineered eukaryotic cell of Embodiment 30, wherein the extracellular component of the cell is a protein, lipid, sugar, or combination thereof associated with extracellular surface of the cell.

Embodiment 32: The engineered eukaryotic cell of Embodiment 30 or Embodiment 31, wherein the extracellular component of the cell is an exopolysaccharide present on the extracellular surface of the cell wall.

Embodiment 33: The engineered eukaryotic cell of any one of Embodiments 22 to 32, wherein upon translation the fusion protein comprises a signal peptide and/or a secretory signal.

Embodiment 34: The engineered eukaryotic cell of any one of Embodiments 22 to 33, wherein in the fusion protein, the portion of the cell surface protein that lacks its native anchoring domain is N-terminal to the catalytic domain.

Embodiment 35: The engineered eukaryotic cell of Embodiment 34, wherein the fusion protein comprises a linker C-terminal to the portion of the cell surface protein that lacks its native anchoring domain.

Embodiment 36: The engineered eukaryotic cell of any one of Embodiments 22 to 35, wherein in the fusion protein, the portion of the cell surface protein that lacks its native anchoring domain is C-terminal to the catalytic domain.

Embodiment 37: The engineered eukaryotic cell of Embodiment 36, wherein the fusion protein comprises a linker N-terminal to the portion of the cell surface protein that lacks its native anchoring domain.

Embodiment 38: The engineered eukaryotic cell of Embodiment 34 or Embodiment 35, wherein the fusion protein further comprises a second portion of the cell surface protein that lacks its native anchoring domain.

Embodiment 39: The engineered eukaryotic cell of Embodiment 38, wherein the second portion of the cell surface protein that lacks its native anchoring domain is C-terminal to the catalytic domain.

Embodiment 40: The engineered eukaryotic cell of Embodiment 39, wherein the fusion protein comprises a second linker N-terminal to the second portion of the cell surface protein that lacks its native anchoring domain.

Embodiment 41: The engineered eukaryotic cell of any one of Embodiments 22 to 37, wherein the fusion protein comprises an amino acid sequence that is at least 95% identical to SEQ ID NO: 331 or SEQ ID NO: 332, wherein the fusion protein comprises an adhesion domain that is capable of binding an exopolysaccharide present on the surface of the cell and thereby attaches the fusion protein to the extracellular surface of the cell for surface display.

Embodiment 42: The engineered eukaryotic cell of any one of Embodiments 38 to 40, wherein the fusion protein comprises an amino acid sequence that is at least 95% identical to SEQ ID NO: 333 or SEQ ID NO: 334, wherein the fusion protein comprises an adhesion domain that is capable of binding an exopolysaccharide present on the surface of the cell and thereby attaches the fusion protein to the extracellular surface of the cell for surface display.

Embodiment 43: The engineered eukaryotic cell of any one of Embodiments 1 to 42, wherein the engineered eukaryotic cell comprises a mutation in its AOX1 gene and/or its AOX2 gene.

Embodiment 44: The engineered eukaryotic cell of any one of Embodiments 1 to 43, wherein the engineered eukaryotic cell is a yeast cell, e.g., a Pichia species.

Embodiment 45: The engineered eukaryotic cell of any one of Embodiments 1 to 44, wherein the fusion protein comprises a linker having an amino acid sequence that is at least 95% identical to SEQ ID NO: 31.

Embodiment 46: The engineered eukaryotic cell of any one of Embodiments 1 to 45, further comprising a genomic modification that overexpresses a secretory glycoprotein.

Embodiment 47: The engineered eukaryotic cell Embodiment 46, wherein the secretory glycoprotein is an animal protein, e.g., an egg protein.

Embodiment 48: The engineered eukaryotic cell Embodiment 47, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

Embodiment 49: The engineered eukaryotic cell of any one of Embodiments 1 to 45, wherein the cell lacks a genomic modification that overexpresses a secretory glycoprotein.

Embodiment 50: The engineered eukaryotic cell of any one of Embodiments 1 to 49, comprising a nucleic acid sequence that encodes the fusion protein.

Embodiment 51: The engineered eukaryotic cell of Embodiment 50, wherein the nucleic acid sequence that encodes the fusion protein is integrated into the cell's genome.

Embodiment 52: The engineered eukaryotic cell of Embodiment 50, wherein the nucleic acid sequence that encodes the fusion protein is extrachromosomal.

Embodiment 53: The engineered eukaryotic cell of any one of Embodiments 50 to 52, wherein the nucleic acid sequence comprises an inducible promoter.

Embodiment 54: The engineered eukaryotic cell of Embodiment 53, wherein the inducible promoter is an AOX1, ADH3, DAK2, PEX11, FLD1, FGH1, DAS2, CAT1, MDH3, HAC1, BiP, RAD30, RVS161-2, MPP10, THP3, TLR, GBP2, PMP20, SHB17, PEX8, or PEX4 promoter.

Embodiment 55: The engineered eukaryotic cell of any one of Embodiments 50 to 54, wherein the nucleic acid sequence comprises an AOX1, TDH3, RPS25A, or RPL2A terminator.

Embodiment 56: The engineered eukaryotic cell of any one of Embodiments 50 to 55, wherein the nucleic acid sequence encodes a signal peptide and/or a secretory signal.

Embodiment 57: The engineered eukaryotic cell of any one of Embodiments 50 to 56, wherein the nucleic acid sequence comprises codons that are optimized for the species of the engineered cell.

Embodiment 58: A method for deglycosylating a secreted glycoprotein, the method comprising contacting a secreted protein with a fusion protein anchored to an engineered eukaryotic cell of any one of Embodiments 1 to 57, thereby providing a deglycosylated secreted glycoprotein.

Embodiment 59: The method of Embodiment 58, wherein the secreted glycoprotein is expressed by the engineered eukaryotic cell.

Embodiment 60: The method of Embodiment 58 or Embodiment 59, wherein the fusion protein anchored to an engineered eukaryotic cell is more effective at deglycosylating the secreted protein than an intracellular endoglycosidase.

Embodiment 61: The method of Embodiment 60, wherein the intracellular endoglycosidase is located within a Golgi vesicle.

Embodiment 62: The method of Embodiment 60 or Embodiment 61, wherein the intracellular endoglycosidase is linked to a membrane associating domain.

Embodiment 63: The method of Embodiment 62, wherein the membrane associating domain comprises an amino acid sequence of OCH1.

Embodiment 64: The method of Embodiment 58, wherein the secreted protein is expressed by a cell other than the engineered eukaryotic cell.

Embodiment 65: The method of any one of Embodiment 58 to 64, further comprising a step of isolating the deglycosylated secreted protein.

Embodiment 66: The method of Embodiment 65, further comprising a step of drying the deglycosylated secreted protein.

Embodiment 67: The method of any one of Embodiments 58 to 66, wherein the secreted protein is an animal protein, e.g., an egg protein.

Embodiment 68: The method of Embodiment 67, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

Embodiment 69: A method for deglycosylating a plurality of secreted glycoproteins, the method comprising contacting the plurality of secreted glycoproteins with a population of engineered eukaryotic cells of any one of Embodiments 1 to 57, thereby providing a plurality of deglycosylated secreted glycoproteins.

Embodiment 70: The method of Embodiment 69, wherein substantially every secreted glycoprotein in the plurality of secreted proteins is deglycosylated upon contact with the population of engineered eukaryotic cells.

Embodiment 71: The method of Embodiment 69 or Embodiment 70, wherein the amount of deglycosylation of the secreted glycoproteins is not increased by further contacting the secreted protein with an isolated endoglycosidase.

Embodiment 72: The method of any one of Embodiments 69 to 71, wherein the amount of deglycosylation of the secreted glycoproteins is more than the amount obtained from a population of cells that express an intracellular endoglycosidase.

Embodiment 73: The method of any one of Embodiment 69 to 72, further comprising a step of isolating the plurality of deglycosylated secreted proteins.

Embodiment 74: The method of Embodiment 73, further comprising a step of drying the plurality of deglycosylated secreted proteins.

Embodiment 75: The method of any one of Embodiments 69 to 74, wherein the secreted protein is an animal protein, e.g., an egg protein.

Embodiment 76: The method of Embodiment 75, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

Embodiment 77: A method for expressing a fusion protein comprising an anchoring domain of a cell surface protein and a catalytic domain of an endoglycosidase, the method comprising obtaining the engineered eukaryotic cell of any one of Embodiments 1 to 57 and culturing the engineered eukaryotic cell under conditions that promote expression of the fusion protein.

Embodiment 78: The method of Embodiment 77, wherein when the engineered eukaryotic cell comprises a nucleic acid sequence that encodes the fusion protein and comprises an inducible promoter, culturing the engineered eukaryotic cell under conditions that promote expression of the fusion protein comprises contacting the cell with an agent that activates the inducible promoter.

Embodiment 79: The method of Embodiment 78, wherein the inducible promoter is an AOX1, DAK2, PEX11 promoter and the agent that activates the inducible promoter is methanol.

Embodiment 80: A population of engineered eukaryotic cells of any one of Embodiments 1 to 57.

Embodiment 81: A bioreactor comprising the population of engineered eukaryotic cells of Embodiment 80.

Embodiment 82: A composition comprising an engineered eukaryotic cell of any one of Embodiments 1 to 57 and a secreted glycoprotein.

Embodiment 83: The composition of Embodiment 82, wherein the secreted glycoprotein is an animal protein, e.g., an egg protein.

Embodiment 84: The composition of Embodiment 83, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

Embodiment 85: A composition comprising an engineered eukaryotic cell of any one of Embodiments 1 to 57, a secreted protein that has been deglycosylated, and one or more oligosaccharides cleaved from the secreted protein.

Embodiment 86: The composition of Embodiment 85, wherein the secreted glycoprotein is an animal protein, e.g., egg protein.

Embodiment 87: The composition of Embodiment 86, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

Embodiment 88: An engineered eukaryotic cell which expresses a surface displayed catalytic domain of endoglycosidase H, wherein the catalytic domain is directly or indirectly tethered to the exterior surface of the cell.

Embodiment 89. A surface-displayed fusion protein comprising a catalytic domain of an enzyme and an anchoring domain of a glycosylphosphatidylinositol (GPI)-anchored protein, wherein the anchoring domain comprises at least about 200 amino acids and/or at least about 30% of the residues in the anchoring domain are serines or threonines.

Embodiment 90. A polynucleotide encoding the surface-displayed fusion protein of embodiment 88.

Embodiment 91. A vector comprising a polynucleotide encoding a surface-displayed fusion protein of embodiment 88.

Embodiment 92. A host cell comprising the polynucleotide of embodiment 89 or a vector of embodiment 90.

### EXAMPLES

The following examples are included for illustrative purposes only and are not intended to limit the scope of the invention.

### Example 1: Construction and Use of a Surface Displayed EndoH—Dan1, EndoH—Sed1p, and EndoH—Tir4p Fusion Protein

This example illustrates construction and analysis of fusion protein comprising a catalytic domain of an enzyme and the anchoring domain of a GPI-linked anchor protein.

Nucleic acid sequences (similar to those shown in FIG. 2) and which encoded the surface displayed fusion proteins shown in FIG. 3 (e.g., comprising one of SEQ ID NO: 21 to SEQ ID NO: 26) were constructed and transfected into Pichia cells. Transfected cells that faithfully expressed and surface displayed the fusion protein were isolated and expanded in culture.

During translation and processing by the engineered cell, the signal peptide (MRFPSIFTAVLFAASSALA; SEQ ID NO: 66) was first cleaved off in the cell's endoplasmic reticulum. When the protein arrives in the late Golgi, the secretion signal (APVNTTTEDETAQIPAEAVIGYSDLEGDFDVAVLPFSNSTNNGLLFINTTIASIAAKEE GVSLDKR; SEQ ID NO: 298) was cleaved off. Around the same time, the propeptide on the C-term (APVNTTTEDETAQIPAEAVIGYSDLEGDFDVAVLPFSNSTNNGLLFINTTIASIAAKEE GVSLDKREAEA; SEQ ID NO: 299) was also cleaved off for the attachment of the GPI anchor. The final resultant fusion protein is as below, and include the full EndoH protein, the mature Tir4, Dan1, or Sed1 protein, plus various linker elements and having the amino acid sequence of, respectively, SEQ ID NO: 21, SEQ ID NO: 23, and SEQ ID NO: 25.

The Dan1 portion comprised 255 total amino acids with 97/98 Serine/Threonine predicted to be O-mannosylated, which totaled 38% of all residues; the Sed1 portion comprised 300 total amino acids, with 135/135 Serine/Threonine predicted to be 0-mannosylated, which totaled 45% of all residues; and the Tir4p portion comprised 345 total amino acids, with 41/147 Serine/Threonine predicted to be O-mannosylated, which totaled 41% of all residues.

The surface displayed fusion protein was incorporated into the cell membrane via a GPI anchor attached to the protein's C-terminus.

This surface displayed fusion protein was shown to be effective at deglycosylating an illustrative secreted glycoprotein (here, ovomucoid (OVD)). A high-throughput screen of cells engineered cells to express OVD and the surface displayed EndoH—Dan1, EndoH—Sed1, or EndoH—Tir4, fusion proteins was performed. In this screen, all engineered cell lines were capable of deglycosylating OVD while maintaining OVD titer.

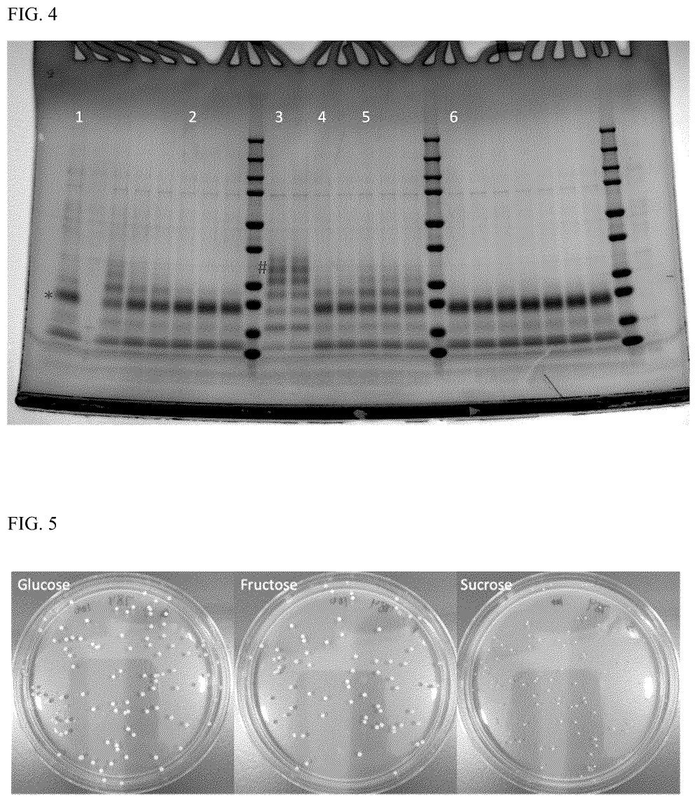

In FIG. 4, the lanes and data shown are as follows: Lane 1—control strain already contains EndoH-Sed1 (Red asterisk highlights the expected band for deglycosylated POI); Lane 2—Test strain with the EndoH-Sed1 construct added; Lane 3—Test strain that appears to have failed to transform the EndoH-Dan1 construct (Red pound symbol highlights the fully glycosylated POI—suggesting no active EndoH in this strain); Lane 4—Test strain with the EndoH-Dan1 construct added; Lane 5—Test strain with the EndoH-Dan1 construct added, but weaker deglycosylation pattern compared to Lane 4 (suggests the construct was damaged or is not expressing to the same amount as the clone in Lane 4); and Lane 6—Test strain with the EndoH-Tir4 construct added. The deglycosylation is extremely powerful in the EndoH-Tir4 constructs, suggesting the larger anchor can more effectively function on POI in the supernatant.

The anchoring domains of the GPI-linked proteins are heavily O-mannosylated on serine and threonine residues. This may facilitate covalent interactions with cell wall polysaccharides following glycosyltransferase activity of native enzymes within the cell wall. These covalent interactions may be helpful in retaining the surface-displayed fusion proteins on the cell's exterior, while still preventing their accumulation in supernatant samples that contain POI.

### Example 2: Construction and Use of a Surface Displayed Suc2—Tir4p Fusion Protein

This example illustrates construction and analysis of a fusion protein comprising a catalytic domain of an invertase and the anchoring domain of a GPI-linked anchor protein which allows an engineered eukaryotic cell to rely on alternate carbon sources.

A background strain strain 1 was used as a test strain. The genetic modifications present in strain 1 are deletion of AOX1 and AOX2. No target protein cassettes were present in this strain. strain 1 was plated on minimal nutrient plates containing Glucose, Fructose, or Sucrose. As shown in FIG. 5, the background strain was able to grow on glucose and fructose at similar rates and had similar colony sizes. The strain grew to pinprick sized colonies on sucrose and stops. It's hypothesized that the sucrose source may contain a small amount of hydrolyzed material (glucose and fructose).

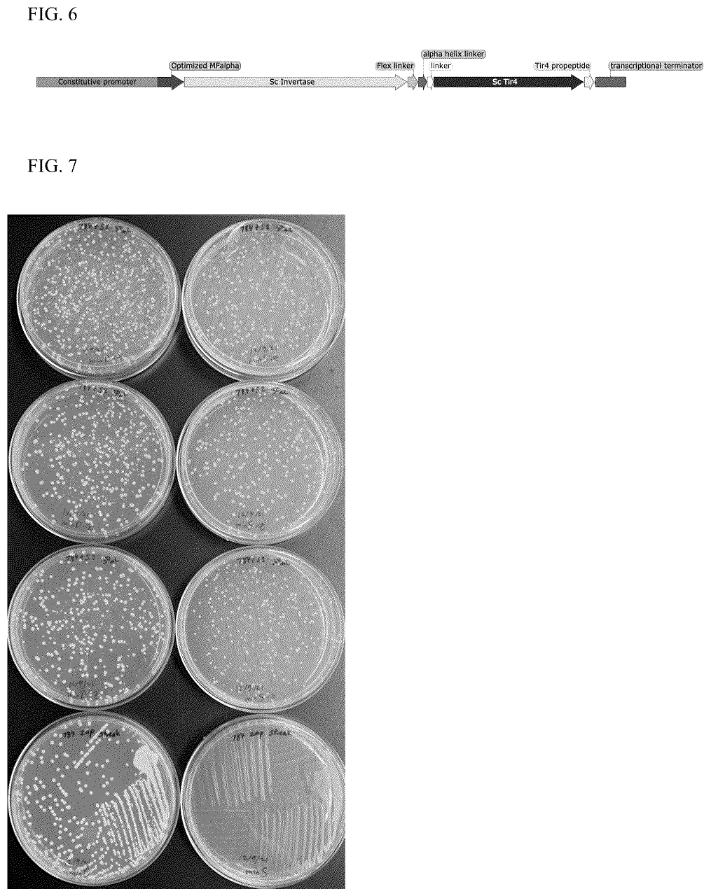

A surface displayed invertase (suc2) from Saccharomyces cerevisiae was transformed into a high performing strain (strain 2) previously transformed to express ovalbumin. The fusion protein was driven by PGcw14, a highly expressed constitutive promoter. A schematic of the DNA sequence for the expression cassette is shown in FIG. 6. An illustrative amino acid sequence for the fusion protein is shown in (SEQ ID NO: 342).

Candidates successfully producing protein under sucrose feed were able to achieve 50%+ per cell productivity when compared to the same strains under glucose feed in high throughput screening. The below table shows the growth and productivity comparisons of the same strain candidates when fed different carbon sources. Candidates were picked into sucrose-containing media and grown for 24 hours. The starter cultures were then used to inoculate equally into sucrose-containing media and glucose-containing media for high throughput screening. Eight high performing candidates are shown below. Note that the parent strain strain 2 is unable to grow and produce protein in sucrose feed, therefore all strain 2 comparisons are made to its performance in glucose.

| | | | | | Supernatant | Supernatant | | |
|---|---|---|---|---|---|---|---|---|
| | | | Supernatant | | protein | protein | | |
| | | | protein | | concentration | concentration | Productivity | Productivity |
| | | | concentration | Productivity | in sucrose vs | in glucose vs | in sucrose | in glucose |
| | OD* in | OD in | in sucrose vs | in sucrose | strain 2 in | strain 2 in | vs strain 2 | vs strain 2 |
| Candidates | sucrose | glucose | glucose¹ | vs glucose² | glucose³ | glucose⁴ | in glucose⁵ | in glucose⁶ |
| 1 | 16.76 | 14.02 | 0.81 | 0.68 | 1.09 | 1.34 | 0.77 | 1.13 |
| 2 | 17.16 | 14.2 | 0.92 | 0.76 | 1.04 | 1.13 | 0.71 | 0.93 |
| 3 | 15.8 | 13.37 | 0.79 | 0.67 | 0.99 | 1.25 | 0.74 | 1.10 |
| 4 | 16.41 | 14.29 | 1.15 | 1.00 | 0.98 | 0.85 | 0.71 | 0.70 |
| 5 | 19.29 | 17.66 | 1.15 | 1.05 | 0.87 | 0.76 | 0.53 | 0.50 |
| 6 | 16.66 | 14.59 | 0.76 | 0.66 | 0.87 | 1.14 | 0.61 | 0.92 |
| 7 | 17.04 | 13.67 | 0.67 | 0.54 | 0.75 | 1.12 | 0.52 | 0.96 |
| 8 | 16.14 | 14.45 | 0.61 | 0.55 | 0.68 | 1.11 | 0.49 | 0.90 |

In the above table, *OD, optical density, is an indirect measure of cell density in culture, thus reflecting cell growth. For reference, strain 2 achieved OD's of 1.14 in sucrose (practically no growth) and 11.76 in glucose. Column 3 is a ratio of protein concentration measured in the culture supernatant, comparing sucrose-fed culture to glucose-fed culture of the same candidate. Column 4 is a ratio of per cell productivity, comparing sucrose-fed culture to glucose-fed culture of the same candidate. Productivity was measured by protein concentration in supernatant divided by OD. Column 5 is a ratio of protein concentration measured in the culture supernatant, comparing sucrose-fed culture of new candidate to glucose-fed culture of parent strain strain 2. Column 6 is a ratio of protein concentration measured in the culture supernatant, comparing glucose-fed culture of new candidate to glucose-fed culture of parent strain strain 2. Column 7 is a ratio of per cell productivity, comparing sucrose-fed culture of new candidate to glucose-fed culture of parent strain strain 2. And, Column 8 is a ratio of per cell productivity, comparing glucose-fed culture of new candidate to glucose-fed culture of parent strain strain 2.

FIG. 7 illustrates the growth of P. pastoris strains using mannose as a sole carbon source.

All candidates grew more cell mass in sucrose feed vs glucose. Focusing on protein concentration and productivity of new strain in sucrose feed vs strain 2 in glucose feed metrics, candidates 1˜4 all perform admirably well, with similar supernatant protein concentration to parent and 71-77% productivity.

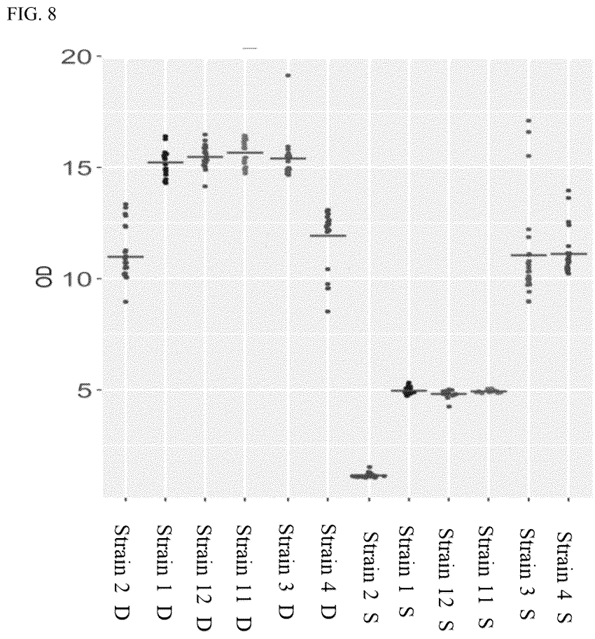

FIG. 8 illustrates the comparison of growth on glucose (D) (shown as “_D” in FIG. 8) vs sucrose (S) (shown as “_S” in FIG. 8) of various background strains and strains that were engineered to display invertase. Strain 2, strain 1, and strain 11 are background strains produced, strain 12 is a “wild-type” P. pastoris strain, and strain 3 and strain 4 express the Suc2 construct (strain 2+Suc2-Tir4). Strain 2, strain 1, and strain 11 are background strains which express rOVA, strain 12 is a “wild-type” P. pastoris strain, and strain 3 and strain 4 were engineered express the Suc2 construct (strain 2+Suc2-Tir4, i.e., the surface displayed invertase fusion protein). While almost all the strains reach OD600 values of 10 or higher when grown in glucose-containing media, only the strains the display the enzyme can reach such levels with sucrose is the main carbon source in the media. All other media components were the same, final concentrations of sugar in media was 0.5%). OD600 measures the amount turbidity of a culture, which is related to the amount of cells present in the culture and is an indicator of cell proliferation/cell growth.

### Example 3: Construction and Use of a Surface Displayed Mannosidase Fusion Protein

This example illustrates construction and analysis of a fusion protein (SEQ ID NO: 26) comprising a catalytic domain of a mannosidase and the anchoring domain of a GPI-linked anchor protein which allows an engineered eukaryotic cell to that cleaves an impurity.

Constructs were designed to disrupt beta-mannosyl transferases BMT1 and BMT2 genes (XP 002493882.1 and XP 002493883.1 respectively) in a Pichia pastoris strain. Knockouts were performed via standard Homologous Recombination (HR) methods in yeast. In summary, genes of interest (GOIs) were deleted by using linearized plasmids that had homology to genomic regions that surround the GOIs, which were transformed into yeast via standard electroporation techniques. The native HR machinery replaces the GOI with the linearized plasmid. The plasmid with antibiotic resistance can eventually be removed using the Cre/lox recombinase system leaving only a small insertion scar where the GOI initially was found.

In this example, the disruption of BMT1 and BMT2 lead to the production of a smaller exopolysaccharide. Using gel electrophoresis and the cationic dye Alcian blue (which binds to the phospho-mannan moiety via the phosphodiester bond) it was shown that disrupting the BMT1 and BMT2 genes (AT250_GQ6804781 and AT250_GQ6804782) produces a noticeable shift in the size of EPS, which strongly suggests that the EPS byproduct is a form of mannan polysaccharide.

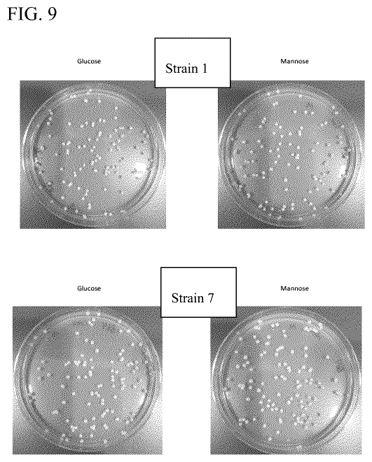

As shown in FIG. 9, Pichia species can grow with mannose as a sole carbon source, illustrating that production strains will be able to recover carbon from the EPS/mannan that is broken down.

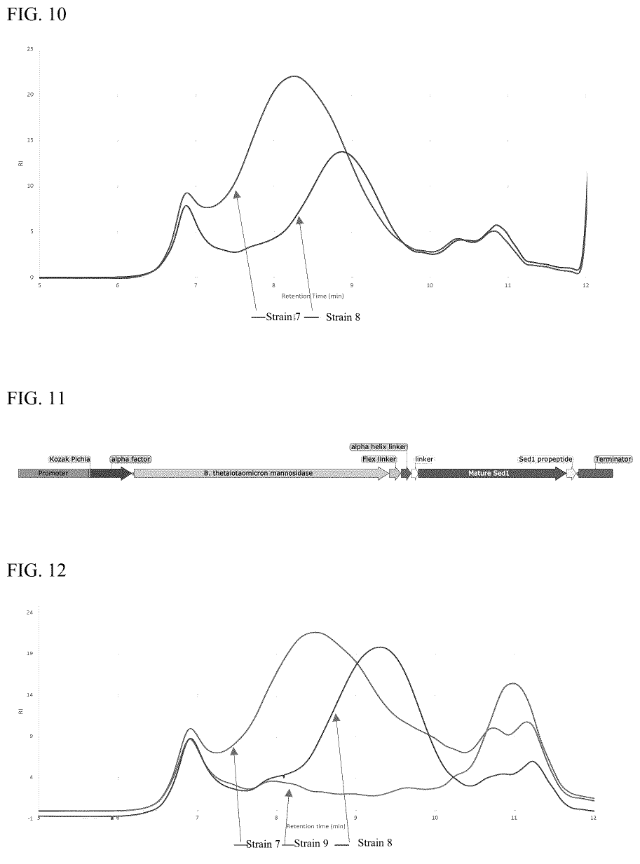

Mannan has been identified using gel electrophoresis and mass spectrometry as the polysaccharide impurity (known as EPS—extracellular polysaccharide) found in supernatants from P. pastoris strains that secrete Proteins of Interest (POIs). Mannan is produced by the sequential action of many mannosyltransferases in the Golgi apparatus. Following the attachment of the core glycan moiety to an asparagine residue, mannan polymerase I (M-pol I) extend the core structure with ˜ten alpha-1,6 mannose units using the Mnn9 catalytic subunit. Next the M-pol II complex (catalytic subunits Mnn10 and Mnn11) extends by another ˜50-100 alpha-1,6 mannose units, which creates a long, linear mannan backbone composed of alpha-1,6-linked sugars. The linear mannan backbone is the extensively decorated with alpha-1,2- and phospho-mannose branch points. These decorations are carried out by members of the MNN and KTR families of proteins—of which there are a total of ten known in P. pastoris. Finally, some species of yeast (including C. albicans and P. pastoris) produce terminal beta-1,2-linked mannose units to “cap” the mannan molecule (opposed to the terminal alpha-1,3-mannose units found in S. cerevisiae mannan), and these reactions are carried out by the BMT family of mannosyltransferases (four of these family members are found in P. pastoris, two of which have been determined to be catalytically active—BMT1/2). Following the identification of the mannosyltransferases discussed above, they were deleted to reduce the size and complexity of the mannan/EPS molecule. As is shown in the chromatogram in FIG. 10, the deletion of multiple native mannosyltransferases indeed increased the retention time of eluted EPS using size exclusion chromatography (SEC) (indicative of a decrease in the size of the molecule). Strain 8 was built from strain 7 by the sequential deletion of five native mannosyltransferases (BMT1 (SEQ ID NO: 343), BMT2 (SEQ ID NO: 344), MNN2 (SEQ ID NO: 345), MNNF1 (SEQ ID NO: 346), MNNF2 (SEQ ID NO: 347)), causing the noticeable right-shift in the EPS peak between 8 and 9 minutes.

The strain was also modified to express mannan hydrolytic enzymes (mannanases/mannosidases) which are normally expressed by the common human gut microbe Bacteroides thetaiotaomicron. Most yeasts are not known to produce enzymes that breakdown their own cell wall material, however B. theta has been shown to scavenge carbon in the form of mannose from yeast cell wall material in the human gut. Using a surface-display approach (FIG. 11) this example demonstrates that these enzymes can used to breakdown the EPS molecule produced by P. Pastoris (following the deletion of select native mannosyltransferases), once again evidenced by shifts in the elution profile of EPS following SEC analysis (FIG. 12).

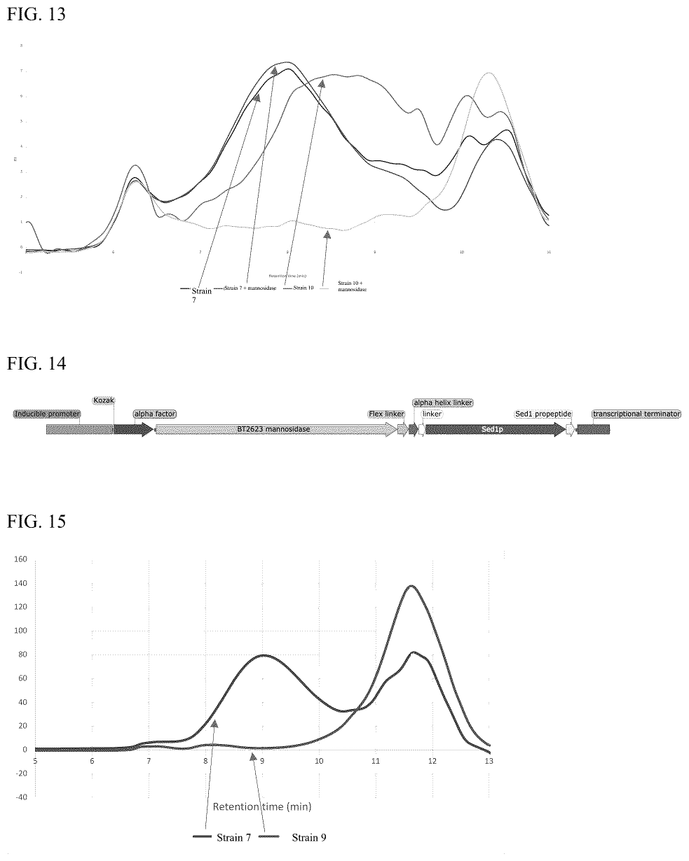

Some mannosyltransferase deletions are required for B. theta mannosidases to recognize EPS as a substrate for cleavage. In FIG. 13, it is shown that when strain 7 and strain 10 (strain 7+3 deleted mannosyltransferases) express the exact same mannosidase construct, only the strain 10+mannosidase build produces EPS which the surface-displayed enzyme can use as a substrate. The disruption of native mannosyltransferases are important for B. theta enzymes to recognize mannan as a substrate for cleavage. Only the strain with deletions and mannosidase elicits the right-shift in the EPS elution profile.

In another experiment, the construct shown in FIG. 14 was inserted in the genome of strain 10 cells, which is strain 7 with deletions to key mannosyltransferase genes XP 002490149/GQ68_02166T0 (MNN2/5 homolog 1), XP 002493883/GQ68_04782T0 (BMT1), and XP 002493882/GQ68_T0 (BMT2)] and the size of EPS byproduct was monitored using size exclusion chromatography (SEC). FIG. 15 depicts chromatograms of background strain (strain 7) and new strain (strain 9). strain 9 was produced by coupling the deletion of three native enzymes that decorate the polysaccharide byproduct with the expression of the surface-displayed mannosidase enzyme. The loss of the peak at 9 minutes suggests the byproduct has become significantly smaller compared to that produced by the background strain strain 7.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE 1
SEQUENCES

| | | |
|---|---|---|
| Tir4 from | SEQ ID NO: | QINELNVVLDDVKTNIADYITLSYTPNSGFSLDQMPAGIMDIAAQLVANPSDDSYTTLYSEV |
| Saccharomyces | 1 | DFSAVEHMLTMVPWYSSRLLPELEAMDASLTTSSSAATSSSEVASSSIASSTSSSVAPSSSEV |
| cerevisiae | | VSSSVASSSSEVASSSVASTSEATSSSAVTSSSAVSSSTESVSSSSVSSSSAVSSSEAVSSSPVS |
| | | SVVSSSAGPASSSVAPYNSTIASSSSTAQTSISTIAPYNSTTTTTPASSASSVIISTRNGTTVTET |
| | | DNTLVTKETTVCDYSSTSAVPASTTGYNNSTKVSTATICSTCKEGTSTATDFSTLKTTVTVC |
| | | DSACQAKKSATVVSVQSKTTGIVEQTENGAAKAVIGMGAGALAAVAAMLL |
| Tir4 from | SEQ ID NO: | MAYSKITLLAALAAIAYAQTQAQINELNVVLDDVKTNIADYITLSYTPNSGFSLDQMPAGI |
| Saccharomyces | 2 | MDIAAQLVANPSDDSYTTLYSEVDFSAVEHMLTMVPWYSSRLLPELEAMDASLTTSSSAA |
| cerevisiae | | TSSSEVASSSIASSTSSSVAPSSSEVVSSSVASSSSEVASSSVASTSEATSSSAVTSSSAVSSSTE |
| (underlined is signal | | SVSSSSVSSSSAVSSSEAVSSSPVSSVVSSSAGPASSSVAPYNSTIASSSSTAQTSISTIAPYNST |
| peptide, may or may | | TTTTPASSASSVIISTRNGTTVTETDNTLVTKETTVCDYSSTSAVPASTTGYNNSTKVSTATI |
| not be utilized in | | CSTCKEGTSTATDFSTLKTTVTVCDSACQAKKSATVVSVQSKTTGIVEQTENGAAKAVIGM |
| design) | | GAGALAAVAAMLL |
| Tir4 (NP_014652.1) | SEQ ID NO: | QINELNVVLDDVKTNIADYITLSYTPNSGFSLDQMPAGIMDIAAQLVANPSDDSYTTLYSEV |
| from Saccharomyces | 3 | DFSAVEHMLTMVPWYSSRLLPELEAMDASLTTSSSAATSSSEVASSSIASSTSSSVAPSSSEV |
| cerevisiae | | VSSSVAPSSSEVVSSSVAPSSSEVVSSSVASSSSEVASSSVAPSSSEVVSSSVASSSSEVASSSV |
| | | APSSSEVVSSSVAPSSSEVVSSSVASSSSEVASSSVAPSSSEVVSSSVASSTSEATSSSAVTSSS |
| | | AVSSSTESVSSSSVSSSSAVSSSEAVSSSPVSSVVSSSAGPASSSVAPYNSTIASSSSTAQTSIST |
| | | LAPYNSTTTTTPASSASSVIISTRNGTTVTETDNTLVTKETTVCDYSSTSAVPASTTGYNNST |
| | | KVSTATICSTCKEGTSTATDFSTLKTTVTVCDSACQAKKSATVVSVQSKTTGIVEQTENGA |
| | | AKAVIGMGAGALAAVAAMLL |
| Tir4 (NP_014652.1) | SEQ ID NO: | MAYSKITLLAALAAIAYAQTQAQINELNVVLDDVKTNIADYITLSYTPNSGFSLDQMPAGI |
| from Saccharomyces | 4 | MDIAAQLVANPSDDSYTTLYSEVDFSAVEHMLTMVPWYSSRLLPELEAMDASLTTSSSAA |
| cerevisiae | | TSSSEVASSSIASSTSSSVAPSSSEVVSSSVAPSSSEVVSSSVAPSSSEVVSSSVASSSSEVASSS |
| (underlined is signal | | VAPSSSEVVSSSVASSSSEVASSSVAPSSSEVVSSSVAPSSSEVVSSSVASSSSEVASSSVAPSS |
| peptide, may or may | | SEVVSSSVASSTSEATSSSAVTSSSAVSSSTESVSSSSVSSSSAVSSSEAVSSSPVSSVVSSSAG |
| not be utilized in | | PASSSVAPYNSTIASSSSTAQTSISTIAPYNSTTTTTPASSASSVIISTRNGTTVTETDNTLVTKE |
| design) | | TTVCDYSSTSAVPASTTGYNNSTKVSTATICSTCKEGTSTATDFSTLKTTVTVCDSACQAKK |
| | | SATVVSVQSKTTGIVEQTENGAAKAVIGMGAGALAAVAAMLL |
| Dan1 from | SEQ ID NO: | ASVTTTLSPYDERVNLIELAVYVSDIGAHLSEYYAFQALHKTETYPPEIAKAVFAGGDFTTM |
| Saccharomyces | 5 | LTGISGDEVTRMITGVPWYSTRLMGAISEALANEGIATAVPASTTEASSTSTSEASSAATESS |
| cerevisiae | | SSSESSAETSSNAASTQATVSSESSSAASTIASSAESSVASSVASSVASSASFANTTAPVSSTSS |
| | | ISVTPVVQNGTDSTVTKTQASTVETTITSCSNNVCSTVTKPVSSKAQSTATSVTSSASRVIDV |
| | | TTNGANKFNNGVFGAAAIAGAAALLL |
| Dan1 from | SEQ ID NO: | MSRISILAVAAALVASATAASVTTTLSPYDERVNLIELAVYVSDIGAHLSEYYAFQALHKTE |
| Saccharomyces | 6 | TYPPELAKAVFAGGDFTTMLTGISGDEVTRMITGVPWYSTRLMGAISEALANEGIATAVPAS |
| cerevisiae | | TTEASSTSTSEASSAATESSSSSESSAETSSNAASTQATVSSESSSAASTIASSAESSVASSVAS |
| (underlined is signal | | SVASSASFANTTAPVSSTSSISVTPVVQNGTDSTVTKTQASTVETTITSCSNNVCSTVTKPVS |
| peptide, may or may | | SKAQSTATSVTSSASRVIDVTTNGANKFNNGVFGAAAIAGAAALLL |
| not be utilized in | | |
| design) | | |
| Dan4 from | SEQ ID NO: | ITATTTLSPYDERVNLIELAVYVSDIRAHIFQYYSFRNHHKTETYPSEIAAAVFDYGDFTTRL |
| Saccharomyces | 7 | TGISGDEVTRMITGVPWYSTRLKPAISSALSKDGIYTAIPTSTSTTTTKSSTSTTPTTTITSTTS |
| cerevisiae | | TTSTTPTTSTTSTTPTTSTTSTTPTTSTTSTTPTTSTTSTTPTTSTTSTTPTTSTTSTTPTTSTTST |
| | | TPTTSTTSTTPTTSTTPTTSTTSTTSQTSTKSTTPTTSSTSTTPTTSTTPTTSTTSTAPTTSTTSTT |
| | | STTSTISTAPTTSTTSSTFSTSSASASSVISTTATTSTTFASLTTPATSTASTDHTTSSVSTTNAF |
| | | TTSATTTTTSDTYISSSSPSQVTSSAEPTTVSEVTSSVEPTRSSQVTSSAEPTTVSEFTSSVEPT |
| | | RSSQVTSSAEPTTVSEFTSSVEPTRSSQVTSSAEPTTVSEFTSSVEPTRSSQVTSSAEPTTVSEF |
| | | TSSVEPTRSSQVTSSAEPTTVSEFTSSVEPIRSSQVTSSAEPTTVSEVTSSVEPIRSSQVTTTEPV |
| | | SSFGSTFSEITSSAEPLSFSKATTSAESISSNQITISSELIVSSVITSSSEIPSSIEVLTSSGISSSVEP |
| | | TSLVGPSSDESISSTESLSATSTFTSAVVSSSKAADFFTRSTVSAKSDVSGNSSTQSTTFFATPS |
| | | TPLAVSSTVVTSSTDSVSPNIPFSEISSSPESSTAITSTSTSFIAERTSSLYLSSSNMSSFTLSTFT |
| | | VSQSIVSSFSMEPTSSVASFASSSPLLVSSRSNCSDARSSNTISSGLFSTIENVRNATSTFTNLS |
| | | TDEIVITSCKSSCTNEDSVLTKTQVSTVETTITSCSGGICTTLMSPVTTINAKANTLTTTETST |
| | | VETTITTCPGGVCSTLTVPVTTITSEATTTATISCEDNEEDITSTETELLTLETTITSCSGGICTT |
| | | LMSPVTTINAKANTLTTTETSTVETTITTCSGGVCSTLTVPVTTITSEATTTATISCEDNEEDV |
| | | ASTKTELLTMETTITSCSGGICTTLMSPVSSFNSKATTSNNAESTIPKAIKVSCSAGACTTLTT |
| | | VDAGISMFTRTGLSITQTTVTNCSGGTCTMLTAPIATATSKVISPIPKASSATSIAHSSASYTV |
| | | SINTNGAYNFDKDNIFGTAIVAVVALLLL |
| Dan4 from | SEQ ID NO: | MVNISIVAGIVALATSAAAITATTTLSPYDERVNLIELAVYVSDIRAHIFQYYSFRNHHKTET |
| Saccharomyces | 8 | YPSEIAAAVFDYGDFTTRLTGISGDEVTRMITGVPWYSTRLKPAISSALSKDGIYTAIPTSTST |
| cerevisiae | | TTTKSSTSTTPTTTITSTTSTTSTTPTTSTTSTTPTTSTTSTTPTTSTTSTTPTTSTTSTTPTTSTTS |
| (underlined is signal | | TTPTTSTTSTTPTTSTTSTTPTTSTTSTTPTTSTTPTTSTTSTTSQTSTKSTTPTTSSTSTTPTTST |
| peptide, may or may | | TPTTSTTSTAPTTSTTSTTSTTSTISTAPTTSTTSSTFSTSSASASSVISTTATTSTTFASLTTPAT |
| not be utilized in | | STASTDHTTSSVSTTNAFTTSATTTTTSDTYISSSSPSQVTSSAEPTTVSEVTSSVEPTRSSQVT |
| design) | | SSAEPTTVSEFTSSVEPTRSSQVTSSAEPTTVSEFTSSVEPTRSSQVTSSAEPTTVSEFTSSVEP |
| | | TRSSQVTSSAEPTTVSEFTSSVEPTRSSQVTSSAEPTTVSEFTSSVEPIRSSQVTSSAEPTTVSE |
| | | VTSSVEPIRSSQVTTTEPVSSFGSTFSEITSSAEPLSFSKATTSAESISSNQITISSELIVSSVITSSS |
| | | EIPSSIEVLTSSGISSSVEPTSLVGPSSDESISSTESLSATSTFTSAVVSSSKAADFFTRSTVSAKS |
| | | DVSGNSSTQSTTFFATPSTPLAVSSTVVTSSTDSVSPNIPFSEISSSPESSTAITSTSTSFIAERTS |
| | | SLYLSSSNMSSFTLSTFTVSQSIVSSFSMEPTSSVASFASSSPLLVSSRSNCSDARSSNTISSGLF |
| | | STIENVRNATSTFTNLSTDEIVITSCKSSCTNEDSVLTKTQVSTVETTITSCSGGICTTLMSPVT |
| | | TINAKANTLTTTETSTVETTITTCPGGVCSTLTVPVTTITSEATTTATISCEDNEEDITSTETEL |
| | | LTLETTITSCSGGICTTLMSPVTTINAKANTLTTTETSTVETTITTCSGGVCSTLTVPVTTITSE |
| | | ATTTATISCEDNEEDVASTKTELLTMETTITSCSGGICTTLMSPVSSFNSKATTSNNAESTIPK |
| | | AIKVSCSAGACTTLTTVDAGISMFTRTGLSITQTTVTNCSGGTCTMLTAPIATATSKVISPIPK |
| | | ASSATSIAHSSASYTVSINTNGAYNFDKDNIFGTAIVAVVALLLL |
| Sag1 from | SEQ ID NO: | ININDITFSNLEITPLTANKQPDQGWTATFDFSIADASSIREGDEFTLSMPHVYRIKLLNSSQT |
| Saccharomyces | 9 | ATISLADGTEAFKCYVSQQAAYLYENTTFTCTAQNDLSSYNTIDGSITFSLNFSDGGSSYEY |
| cerevisiae | | ELENAKFFKSGPMLVKLGNQMSDVVNFDPAAFTENVFHSGRSTGYGSFESYHLGMYCPNG |
| | | YFLGGTEKIDYDSSNNNVDLDCSSVQVYSSNDFNDWWFPQSYNDTNADVTCFGSNLWITL |
| | | DEKLYDGEMLWVNALQSLPANVNTIDHALEFQYTCLDTIANTTYATQFSTTREFIVYQGRN |
| | | LGTASAKSSFISTTTTDLTSINTSAYSTGSISTVETGNRTTSEVISHVVTTSTKLSPTATTSLTIA |
| | | QTSIYSTDSNITVGTDIHTTSEVISDVETISRETASTVVAAPTSTTGWTGAMNTYISQFTSSSF |
| | | ATINSTPIISSSAVFETSDASIVNVHTENITNTAAVPSEEPTFVNATRNSLNSFCSSKQPSSPSS |
| | | YTSSPLVSSLSVSKTLLSTSFTPSVPTSNTYIKTKNTGYFEHTALTTSSVGLNSFSETAVSSQG |
| | | TKIDTFLVSSLIAYPSSASGSQLSGIQQNFTSTSLMISTYEGKASIFFSAELGSIIFLLLSYLLF |
| Sag1 from | SEQ ID NO: | MFTFLKIILWLFSLALASAININDITFSNLEITPLTANKQPDQGWTATFDFSIADASSIREGDEF |
| Saccharomyces | 10 | TLSMPHVYRIKLLNSSQTATISLADGTEAFKCYVSQQAAYLYENTTFTCTAQNDLSSYNTID |
| cerevisiae | | GSITFSLNFSDGGSSYEYELENAKFFKSGPMLVKLGNQMSDVVNFDPAAFTENVFHSGRST |
| (underlined is signal | | GYGSFESYHLGMYCPNGYFLGGTEKIDYDSSNNNVDLDCSSVQVYSSNDFNDWWFPQSY |
| peptide, may or may | | NDTNADVTCFGSNLWITLDEKLYDGEMLWVNALQSLPANVNTIDHALEFQYTCLDTIANT |
| not be utilized in | | TYATQFSTTREFIVYQGRNLGTASAKSSFISTTTTDLTSINTSAYSTGSISTVETGNRTTSEVIS |
| design) | | HVVTTSTKLSPTATTSLTIAQTSIYSTDSNITVGTDIHTTSEVISDVETISRETASTVVAAPTST |
| | | TGWTGAMNTYISQFTSSSFATINSTPIISSSAVFETSDASIVNVHTENITNTAAVPSEEPTFVN |
| | | ATRNSLNSFCSSKQPSSPSSYTSSPLVSSLSVSKTLLSTSFTPSVPTSNTYIKTKNTGYFEHTAL |
| | | TTSSVGLNSFSETAVSSQGTKIDTFLVSSLIAYPSSASGSQLSGIQQNFTSTSLMISTYEGKASI |
| | | FFSAELGSIIFLLLSYLLF |
| FIG. 2 from | SEQ ID NO: | QIVFYQNSSTSLPVPTLVSTSIADFHESSSTGEVQYSSSYSYVQPSIDSFTSSSFLTSFEAPTETS |
| Saccharomyces | 11 | SSYAVSSSLITSDTFSSYSDIFDEETSSLISTSAASSEKASSTLSSTAQPHRTSHSSSSFELPVTA |
| cerevisiae | | PSSSSLPSSTSLTFTSVNPSQSWTSFNSEKSSALSSTIDFTSSEISGSTSPKSLESFDTTGTITSSY |
| | | SPSPSSKNSNQTSLLSPLEPLSSSSGDLILSSTIQATTNDQTSKTIPTLVDATSSLPPTLRSSSMA |
| | | PTSGSDSISHNFTSPPSKTSGNYDVLTSNSIDPSLFTTTSEYSSTQLSSLNRASKSETVNFTASI |
| | | ASTPFGTDSATSLIDPISSVGSTASSFVGISTANFSTQGNSNYVPESTASGSSQYQDWSSSSLP |
| | | LSQTTWVVINTTNTQGSVTSTTSPAYVSTATKTVDGVITEYVTWCPLTQTKSQAIGVSSSISS |
| | | VPQASSFSGSSILSSNSSTLAASNNVPESTASGSSQYQDWSSSSLPLSQTTWVVINTTNTQGS |
| | | VTSTTSPAYVSTATKTVDGVITEYVTWCPLTQTKSQAIGISSSTISATQTSKPSSILTLGISTLQ |
| | | LSDATFKGTETINTHLMTESTSITEPTYFSGTSDSFYLCTSEVNLASSLSSYPNFSSSEGSTATI |
| | | TNSTVTFGSTSKYPSTSVSNPTEASQHVSSSVNSLTDFTSNSTETIAVISNIHKTSSNKDYSLT |
| | | TTQLKTSGMQTLVLSTVTTTVNGAATEYTTWCPASSIAYTTSISYKTLVLTTEVCSHSECTP |
| | | TVITSVTATSSTIPLLSTSSSTVLSSTVSEGAKNPAASEVTINTQVSATSEATSTSTQVSATSAT |
| | | ATASESSTTSQVSTASETISTLGTQNFTTTGSLLFPALSTEMINTTVVSRKTLIISTEVCSHSKC |
| | | VPTVITEVVTSKGTPSNGHSSQTLQTEAVEVTLSSHQTVTMSTEVCSNSICTPTVITSVQMRS |
| | | TPFPYLTSSTSSSSLASTKKSSLEASSEMSTFSVSTQSLPLAFTSSEKRSTTSVSQWSNTVLTN |
| | | TIMSSSSNVISTNEKPSSTTSPYNFSSGYSLPSSSTPSQYSLSTATTTINGIKTVYTTWCPLAEK |
| | | STVAASSQSSRSVDRFVSSSKPSSSLSQTSIQYTLSTATTTISGLKTVYTTWCPLTSKSTLGAT |
| | | TQTSSTAKVRITSASSATSTSISLSTSTESESSSGYLSKGVCSGTECTQDVPTQSSSPASTLAYS |
| | | PSVSTSSSSSFSTTTASTLTSTHTSVPLLPSSSSISASSPSSTSLLSTSLPSPAFTSSTLPTATAVSS |
| | | STFIASSLPLSSKSSLSLSPVSSSILMSQFSSSSSSSSSLASLPSLSISPTVDTVSVLQPTTSIATLT |
| | | CTDSQCQQEVSTICNGSNCDDVTSTATTPPSTVTDTMTCTGSECQKTTSSSCDGYSCKVSET |
| | | YKSSATISACSGEGCQASATSELNSQYVTMTSVITPSAITTTSVEVHSTESTISITTVKPVTYT |
| | | SSDTNGELITITSSSQTVIPSVTTIITRTKVAITSAPKPTTTTYVEQRLSSSGIATSFVAAASSTW |
| | | ITTPIVSTYAGSASKFLCSKFFMIMVMVINFI |
| FIG. 2 from | SEQ ID NO: | MNSFASLGLIYSVVNLLTRVEAQIVFYQNSSTSLPVPTLVSTSIADFHESSSTGEVQYSSSYS |
| Saccharomyces | 12 | YVQPSIDSFTSSSFLTSFEAPTETSSSYAVSSSLITSDTFSSYSDIFDEETSSLISTSAASSEKASS |
| cerevisiae | | TLSSTAQPHRTSHSSSSFELPVTAPSSSSLPSSTSLTFTSVNPSQSWTSFNSEKSSALSSTIDFTS |
| (underlined is signal | | SEISGSTSPKSLESFDTTGTITSSYSPSPSSKNSNQTSLLSPLEPLSSSSGDLILSSTIQATTNDQT |
| peptide, may or may | | SKTIPTLVDATSSLPPTLRSSSMAPTSGSDSISHNFTSPPSKTSGNYDVLTSNSIDPSLFTTTSE |
| not be utilized in | | YSSTQLSSLNRASKSETVNFTASIASTPFGTDSATSLIDPISSVGSTASSFVGISTANFSTQGNS |
| design) | | NYVPESTASGSSQYQDWSSSSLPLSQTTWVVINTTNTQGSVTSTTSPAYVSTATKTVDGVIT |
| | | EYVTWCPLTQTKSQAIGVSSSISSVPQASSFSGSSILSSNSSTLAASNNVPESTASGSSQYQD |
| | | WSSSSLPLSQTTWVVINTTNTQGSVTSTTSPAYVSTATKTVDGVITEYVTWCPLTQTKSQAI |
| | | GISSSTISATQTSKPSSILTLGISTLQLSDATFKGTETINTHLMTESTSITEPTYFSGTSDSFYLC |
| | | TSEVNLASSLSSYPNFSSSEGSTATITNSTVTFGSTSKYPSTSVSNPTEASQHVSSSVNSLTDF |
| | | TSNSTETIAVISNIHKTSSNKDYSLTTTQLKTSGMQTLVLSTVTTTVNGAATEYTTWCPASSI |
| | | AYTTSISYKTLVLTTEVCSHSECTPTVITSVTATSSTIPLLSTSSSTVLSSTVSEGAKNPAASEV |
| | | TINTQVSATSEATSTSTQVSATSATATASESSTTSQVSTASETISTLGTQNFTTTGSLLFPALS |
| | | TEMINTTVVSRKTLIISTEVCSHSKCVPTVITEVVTSKGTPSNGHSSQTLQTEAVEVTLSSHQ |
| | | TVTMSTEVCSNSICTPTVITSVQMRSTPFPYLTSSTSSSSLASTKKSSLEASSEMSTFSVSTQSL |
| | | PLAFTSSEKRSTTSVSQWSNTVLTNTIMSSSSNVISTNEKPSSTTSPYNFSSGYSLPSSSTPSQY |
| | | SLSTATTTINGIKTVYTTWCPLAEKSTVAASSQSSRSVDRFVSSSKPSSSLSQTSIQYTLSTAT |
| | | TTISGLKTVYTTWCPLTSKSTLGATTQTSSTAKVRITSASSATSTSISLSTSTESESSSGYLSKG |
| | | VCSGTECTQDVPTQSSSPASTLAYSPSVSTSSSSSFSTTTASTLTSTHTSVPLLPSSSSISASSPS |
| | | STSLLSTSLPSPAFTSSTLPTATAVSSSTFIASSLPLSSKSSLSLSPVSSSILMSQFSSSSSSSSSLA |
| | | SLPSLSISPTVDTVSVLQPTTSIATLTCTDSQCQQEVSTICNGSNCDDVTSTATTPPSTVTDTM |
| | | TCTGSECQKTTSSSCDGYSCKVSETYKSSATISACSGEGCQASATSELNSQYVTMTSVITPSA |
| | | ITTTSVEVHSTESTISITTVKPVTYTSSDTNGELITITSSSQTVIPSVTTIITRTKVAITSAPKPTT |
| | | TTYVEQRLSSSGIATSFVAAASSTWITTPIVSTYAGSASKFLCSKFFMIMVMVINFI |
| Sed1 from | SEQ ID NO: | QFSNSTSASSTDVTSSSSISTSSGSVTITSSEAPESDNGTSTAAPTETSTEAPTTAIPTNGTSTEA |
| Saccharomyces | 13 | PTTAIPTNGTSTEAPTDTTTEAPTTALPTNGTSTEAPTDTTTEAPTTGLPTNGTTSAFPPTTSL |
| cerevisiae | | PPSNTTTTPPYNPSTDYTTDYTVVTEYTTYCPEPTTFTTNGKTYTVTEPTTLTITDCPCTIEKP |
| | | TTTSTTEYTVVTEYTTYCPEPTTFTTNGKTYTVTEPTTLTITDCPCTIEKSEAPESSVPVTESK |
| | | GTTTKETGVTTKQTTANPSLTVSTVVPVSSSASSHSVVINSNGANVVVPGALGLAGVAMLF |
| | | L |
| Sed1 from | SEQ ID NO: | MKLSTVLLSAGLASTTLAQFSNSTSASSTDVTSSSSISTSSGSVTITSSEAPESDNGTSTAAPT |
| Saccharomyces | 14 | ETSTEAPTTAIPTNGTSTEAPTTAIPTNGTSTEAPTDTTTEAPTTALPTNGTSTEAPTDTTTEA |
| cerevisiae | | PTTGLPTNGTTSAFPPTTSLPPSNTTTTPPYNPSTDYTTDYTVVTEYTTYCPEPTTFTTNGKT |
| (underlined is signal | | YTVTEPTTLTITDCPCTIEKPTTTSTTEYTVVTEYTTYCPEPTTFTTNGKTYTVTEPTTLTITD |
| peptide, may or may | | CPCTIEKSEAPESSVPVTESKGTTTKETGVTTKQTTANPSLTVSTVVPVSSSASSHSVVINSN |
| not be utilized in | | GANVVVPGALGLAGVAMLFL |
| design) | | |
| Saccharomyces | SEQ ID NO: | SMTNETSDRPLVHFTPNKGWMNDPNGLWYDEKDAKWHLYFQYNPNDTVWGTPLFWGH |
| cerevisiae SUC2 | 15 | ATSDDLTNWEDQPIAIAPKRNDSGAFSGSMVVDYNNTSGFFNDTIDPRQRCVAIWTYNTPE |
| (without peptides that | | SEEQYISYSLDGGYTFTEYQKNPVLAANSTQFRDPKVFWYEPSQKWIMTAAKSQDYKIEIY |
| are cleaved off post- | | SSDDLKSWKLESAFANEGFLGYQYECPGLIEVPTEQDPSKSYWVMFISINPGAPAGGSFNQY |
| translationally) | | FVGSFNGTHFEAFDNQSRVVDFGKDYYALQTFFNTDPTYGSALGIAWASNWEYSAFVPTN |
| | | PWRSSMSLVRKFSLNTEYQANPETELINLKAEPILNISNAGPWSRFATNTTLTKANSYNVDL |
| | | SNSTGTLEFELVYAVNTTQTISKSVFADLSLWFKGLEDPEEYLRMGFEVSASSFFLDRGNSK |
| | | VKFVKENPYFTNRMSVNNQPFKSENDLSYYKVYGLLDQNILELYFNDGDVVSTNTYFMTT |
| | | GNALGSVNMTTGVDNLFYIDKFQVREVK |
| Saccharomyces | SEQ ID NO: | MLLQAFLFLLAGFAAKISASMTNETSDRPLVHFTPNKGWMNDPNGLWYDEKDAKWHLYF |
| cerevisiae SUC2 | 16 | QYNPNDTVWGTPLFWGHATSDDLTNWEDQPIAIAPKRNDSGAFSGSMVVDYNNTSGFFN |
| (including peptides | | DTIDPRQRCVAIWTYNTPESEEQYISYSLDGGYTFTEYQKNPVLAANSTQFRDPKVFWYEPS |
| that are cleaved off | | QKWIMTAAKSQDYKIEIYSSDDLKSWKLESAFANEGFLGYQYECPGLIEVPTEQDPSKSYW |
| post-translationally) | | VMFISINPGAPAGGSFNQYFVGSFNGTHFEAFDNQSRVVDFGKDYYALQTFFNTDPTYGSA |
| UniProtKB-P00724 | | LGIAWASNWEYSAFVPTNPWRSSMSLVRKFSLNTEYQANPETELINLKAEPILNISNAGPWS |
| (INV2_YEAST) | | RFATNTTLTKANSYNVDLSNSTGTLEFELVYAVNTTQTISKSVFADLSLWFKGLEDPEEYLR |
| | | MGFEVSASSFFLDRGNSKVKFVKENPYFTNRMSVNNQPFKSENDLSYYKVYGLLDQNILEL |
| | | YFNDGDVVSTNTYFMTTGNALGSVNMTTGVDNLFYIDKFQVREVK |
| Pichia angusta | SEQ ID NO: | MTIESQEPWWKSAVVYQVWPASFKDSNGDGIGDLNGITSELDHIKSLGTDVIWLSPHYASP |
| MAL1 (including | 17 | LDDMGYDISDYNAINPQFGTMEDMDRLLAEIKKRDMRLILDLVINHTSSEHAWFKESRSSR |
| peptides that are | | DNPKRDWYIWKDNANNWLSFFSGSAWSYDEKTKQYYLRLFAETQPDLNWENPKTREAIY |
| cleaved off post- | | KSALEFWYEKGVSGFRIDTAGLYSKVQTFEDAPVTFPGEKYQPAGPLINSGPRIHEFHKEMY |
| translationally) | | EKVTSRYDAMTVGEVGHCSKADALKYVSAKEKEMNMMFLFDTVDVGSDKSDRFRYKGF |
| UniProtKB- | | TLTDFKDAIINQSNFIFDDETGELNDAWSTVFIENHDQPRCVTRFGNTSNKLFWSRSAKMLA |
| Q9P8G8 | | LLQTTLTGTLFVYQGQEIGMTNVSPKWDISEYLDINTINYWNAFNETEHSDEEKAELLKIIN |
| (Q9P8G8_PICAN) | | LLARDNARTPVQWDSSENGGFGGKPWMRINDNYKDINVASQKEDPDSVLNFYRNAIKTRK |
| | | HYSETLIFGRFEVQDYDNQEIFYYTKTSNKGQKKMAVVLNFTDREVEYPIPQGKLLLSNIAN |
| | | NITGKLQPYEGRLIEVN |
| Saccharomyces | SEQ ID NO: | MLLQAFLFLLAGFAAKISASMTNETSDRPLVHFTPNKGWMNDPNGLWYDAKEGKWHLYF |
| cerevisiae SUC1 | 351 | QYNPNDTVWGLPLFWGHATSDDLTHWQDEPVAIAPKRKDSGAYSGSMVIDYNNTSGFFN |
| (invertase 1) | | DTIDPRQRCVAIWTYNTPESEEQYISYSLDGGYTFTEYQKNPVLAANSTQFRDPKVFWYEPS |
| Unitprot Accession: | | KKWIMTAAKSQDYKIEIYSSDDLKSWKLESAFANEGFLGYQYECPGLIEVPSEQDPSKSHW |
| P10594 | | VMFISINPGAPAGGSFNQYFVGSFNGHHFEAFDNQSRVVDFGKDYYALQTFFNTDPTYGSA |
| | | LGIAWASNWEYSAFVPSNPWRSSMSLVRPFSLNTEYQANPETELINLKAEPILNISSAGPWS |
| | | RFATNTTLTKANSYNVDLSNSTGTLEFELVYAVNTTQTISKSVFADLSLWFKGLEDPEEYLR |
| | | MGFEVSASSFFLDRGNSKVKFVKENPYFTNRMSVNNQPFKSENDLSYYKVYGLLDQNILEL |
| | | YFNDGDVVSTNTYFMTTGNALGSVNMTTGVDNLFYIDKFQVREVK |
| Kluyveromyces lactis | SEQ ID NO: | MLKLLSLMVPLASAAVIHRRDANISAIASEWNSTSNSSSSLSLNRPAVHYSPEEGWMNDPN |
| INV1 | 352 | GLWYDAKEEDWHIYYQYYPDAPHWGLPLTWGHAVSKDLTVWDEQGVAFGPEFETAGAF |
| (invertase) | | SGSMVIDYNNTSGFFNSSTDPRQRVVAIWTLDYSGSETQQLSYSHDGGYTFTEYSDNPVLDI |
| Unitprot Accession: | | DSDAFRDPKVFWYQGEDSESEGNWVMTVAEADRFSVLIYSSPDLKNWTLESNFSREGYLG |
| Q9Y746 | | YNYECPGLVKVPYVKNTTYASAPGSNITSSGPLHPNSTVSFSNSSSIAWNASSVPLNITLSNS |
| | | TLVDETSQLEEVGYAWVMIVSFNPGSILGGSGTEYFIGDFNGTHFEPLDKQTRFLDLGKDY |
| | | YALQTFFNTPNEVDVLGIAWASNWQYANQVPTDPWRSSMSLVRNFTITEYNINSNTTALVL |
| | | NSQPVLDFTSLRKNGTSYTLENLTLNSSSHEVLEFEDPTGVFEFSLEYSVNFTGIHNWVFTD |
| | | LSLYFQGDKDSDEYLRLGYEANSKQFFLDRGHSNIPFVQENPFFTQRLSVSNPPSSNSSTFDV |
| | | YGIVDRNIIELYFNNGTVTSTNTFFFSTGNNIGSIIVKSGVDDVYEIESLKVNQFYVD |
| Cyberlindnera jadinii | SEQ ID NO: | MSLTKDASEDQEDIKSLTMNTSLVDSSIYRPLVHLTPPVGWMNDPNGLFYDSSESTYHVYY |
| INV1 (invertase) | 353 | QYNPNDTIWGLPLYWGHATSDDLLTWDHHAPAIGPENDDEGIYSGSIVIDYDNTSGFFDDS |
| Unitprot Accession: | | TRPEQRIVAIYTNNLPDVETQDIAYSTDGGYTFEKYENNPVIDVNSTQFRDPKVIWYEETEQ |
| 094224 | | WVMTVAKSQEYKIQIYTSDNLKDWSLASNFSTKGYVGYQYECPGLFEATIENPKSGDPEK |
| | | KWVMVLAINPGSPLGGSINEYFVGDFNGTEFIPDDDATRFMDTGKDFYAFQAFFNAPENRS |
| | | IGVAWSSNWQYSNQVPDPDGYRSSMSSIREYTLRYVSTNPESEQLILCQKPFFVNETDLKV |
| | | VEEYKVSNSSLTVDHTFGSSFANSNTTGLLDFNMTFTVNGTTDVTQKDSVTFELRIKSNQS |
| | | DEAIALGYDYNNEQFYINRATESYFQRTNQFFQERWSTYVQPLTITESGDKQYQLYGLVDN |
| | | NILELYFNDGAFTSTNTFFLEKGKPSNVDIVASSSKEAYHRGPAD |
| Oryza sativa japonica | SEQ ID NO: | MELAVGAGGMRRSASHTSLSESDDFDLSRLLNKPRINVERQRSFDDRSLSDVSYSGGGHGG |
| (rice) CINV1 | 354 | TRGGFDGMYSPGGGLRSLVGTPASSALHSFEPHPIVGDAWEALRRSLVFFRGQPLGTIAAFD |
| (invertase) Unitprot | | HASEEVLNYDQVFVRDFVPSALAFLMNGEPEIVRHFLLKTLLLQGWEKKVDRFKLGEGAM |
| Accession: Q69T31 | | PASFKVLHDSKKGVDTLHADFGESAIGRVAPVDSGFWWIILLRAYTKSTGDLTLAETPECQ |
| | | KGMRLILSLCLSEGFDTFPTLLCADGCCMIDRRMGVYGYPIEIQALFFMALRCALQLLKHD |
| | | NEGKEFVERIATRLHALSYHMRSYYWLDFQQLNDIYRYKTEEYSHTAVNKFNVIPDSIPDW |
| | | LFDFMPCQGGFFIGNVSPARMDFRWFALGNMIAILSSLATPEQSTAIMDLIEERWEELIGEM |
| | | PLKICYPAIENHEWRIVTGCDPKNTRWSYHNGGSWPVLLWLLTAACIKTGRPQIARRAIDL |
| | | AERRLLKDGWPEYYDGKLGRYVGKQARKFQTWSIAGYLVAKMMLEDPSHLGMISLEEDK |
| | | AMKPVLKRSASWTN |
| Arabidopsis thaliana | SEQ ID NO: | MEGVGLRAVGSHCSLSEMDDLDLTRALDKPRLKIERKRSFDERSMSELSTGYSRHDGIHDS |
| Alkaline/neutral | 355 | PRGRSVLDTPLSSARNSFEPHPMMAEAWEALRRSMVFFRGQPVGTLAAVDNTTDEVLNYD |
| invertase CINV1 | | QVFVRDFVPSALAFLMNGEPDIVKHFLLKTLQLQGWEKRVDRFKLGEGVMPASFKVLHDP |
| INVA | | IRETDNIVADFGESAIGRVAPVDSGFWWIILLRAYTKSTGDLTLSETPECQKGMKLILSLCLA |
| UnitProt Accession | | EGFDTFPTLLCADGCSMIDRRMGVYGYPIEIQALFFMALRSALSMLKPDGDGREVIERIVKR |
| No.: Q9LQF2 | | LHALSFHMRNYFWLDHQNLNDIYRFKTEEYSHTAVNKFNVMPDSIPEWVFDFMPLRGGYF |
| | | VGNVGPAHMDFRWFALGNCVSILSSLATPDQSMAIMDLLEHRWAELVGEMPLKICYPCLE |
| | | GHEWRIVTGCDPKNTRWSYHNGGSWPVLLWQLTAACIKTGRPQIARRAVDLIESRLHRDC |
| | | WPEYYDGKLGRYVGKQARKYQTWSIAGYLVAKMLLEDPSHIGMISLEEDKLMKPVIKRSA |
| | | SWPQL |
| Arabidopsis thaliana | SEQ ID NO: | MSAIYLLRKISTKTPSRFHRSLFFSTFSKDSPPDLSRTTSIRHLSSSQRFVSSSIYCFPQSKILPN |
| Alkaline/neutral | 356 | RFSEKTTGISVRQFSTSVETNLSDKSFERIHVQSDAILERIHKNEEEVETVSIGSEKVVREESE |
| invertase A, | | AEKEAWRILENAVVRYCGSPVGTVAANDPGDKMPLNYDQVFIRDFVPSALAFLLKGEGDI |
| mitochondrial INVE | | VRNFLLHTLQLQSWEKTVDCYSPGQGLMPASFKVRTVALDENTTEEVLDPDFGESAIGRV |
| UnitProt Accession | | APVDSGLWWIILLRAYGKITGDFSLQERIDVQTGIKLIMNLCLADGFDMFPTLLVTDGSCMI |
| No.: UnitProt | | DRRMGIHGHPLEIQSLFYSALRCSREMLSVNDSSKDLVRAINNRLSALSFHIREYYWVDIKK |
| Accession No.: | | INEIYRYKTEEYSTDATNKFNIYPEQIPPWLMDWIPEQGGYLLGNLQPAHMDFRFFTLGNF |
| Q9FXA8 | | WSIVSSLATPKQNEAILNLIEAKWDDIIGNMPLKICYPALEYDDWRIITGSDPKNTPWSYHN |
| | | SGSWPTLLWQFTLACMKMGRPELAEKALAVAEKRLLADRWPEYYDTRSGKFIGKQSRLY |
| | | QTWTVAGFLTSKLLLANPEMASLLFWEEDYELLDICACGLRKSDRKKCSRVAAKTQILVR |
| Arabidopsis thaliana | SEQ ID NO: | MAASETVLRVPLGSVSQSCYLASFFVNSTPNLSFKPVSRNRKTVRCTNSHEVSSVPKHSFHS |
| Alkaline/neutral | 357 | SNSVLKGKKFVSTICKCQKHDVEESIRSTLLPSDGLSSELKSDLDEMPLPVNGSVSSNGNAQ |
| invertase E, | | SVGTKSIEDEAWDLLRQSVVFYCGSPIGTIAANDPNSTSVLNYDQVFIRDFIPSGIAFLLKGE |
| chloroplastic INVE | | YDIVRNFILYTLQLQSWEKTMDCHSPGQGLMPCSFKVKTVPLDGDDSMTEEVLDPDFGEA |
| UnitProt Accession | | AIGRVAPVDSGLWWIILLRAYGKCTGDLSVQERVDVQTGIKMILKLCLADGFDMFPTLLVT |
| No.: Q9FK88 | | DGSCMIDRRMGIHGHPLEIQALFYSALVCAREMLTPEDGSADLIRALNNRLVALNFHIREY |
| | | YWLDLKKINEIYRYQTEEYSYDAVNKFNIYPDQIPSWLVDFMPNRGGYLIGNLQPAHMDFR |
| | | FFTLGNLWSIVSSLASNDQSHAILDFIEAKWAELVADMPLKICYPAMEGEEWRIITGSDPKN |
| | | TPWSYHNGGAWPTLLWQLTVASIKMGRPELAEKAVELAERRISLDKWPEYYDTKRARFIG |
| | | KQARLYQTWSIAGYLVAKLLLANPAAAKFLTSEEDSDLRNAFSCMLSANPRRTRGPKKAQ |
| | | QPFIV |
| Oryza sativa japonica | SEQ ID NO: | MGVLGSRVAWAWLVQLLLLQQLAGASHVVYDDLELQAAATTADGVPPSIVDSELRTGYH |
| (rice) Beta- | 358 | FQPPKNWINDPNAPMYYKGWYHLFYQYNPKGAVWGNIVWAHSVSRDLINWVALKPAIEP |
| fructofuranosidase, | | SIRADKYGCWSGSATMMADGTPVIMYTGVNRPDVNYQVQNVALPRNGSDPLLREWVKPG |
| insoluble isoenzyme | | HNPVIVPEGGINATQFRDPTTAWRGADGHWRLLVGSLAGQSRGVAYVYRSRDFRRWTRA |
| 2 CIN2 | | AQPLHSAPTGMWECPDFYPVTADGRREGVDTSSAVVDAAASARVKYVLKNSLDLRRYDY |
| Unit Prot Accession | | YTVGTYDRKAERYVPDDPAGDEHHIRYDYGNFYASKTFYDPAKRRRILWGWANESDTAA |
| No.: Q0JDC5 | | DDVAKGWAGIQAIPRKVWLDPSGKQLLQWPIEEVERLRGKWPVILKDRVVKPGEHVEVTG |
| | | LQTAQADVEVSFEVGSLEAAERLDPAMAYDAQRLCSARGADARGGVGPFGLWVLASAGL |
| | | EEKTAVFFRVFRPAARGGGAGKPVVLMCTDPTKSSRNPNMYQPTFAGFVDTDITNGKISLR |
| | | SLIDRSVVESFGAGGKACILSRVYPSLAIGKNARLYVFNNGKAEIKVSQLTAWEMKKPVM |
| | | MNGA |
| Rattus norvegicus | SEQ ID NO: | MAKKKFSALEISLIVLFIIVTAIAIALVTVLATKVPAVEEIKSPTPTSNSTPTSTPTSTSTPTSTS |
| (rat) | 359 | TPSPGKCPPEQGEPINERINCIPEQHPTKAICEERGCCWRPWNNTVIPWCFFADNHGYNAESI |
| Sucrase-isomaltase, | | TNENAGLKATLNRIPSPTLFGEDIKSVILTTQTQTGNRFRFKITDPNNKRYEVPHQFVKEETG |
| intestinal | | IPAADTLYDVQVSENPFSIKVIRKSNNKVLCDTSVGPLLYSNQYLQISTRLPSEYIYGFGGHI |
| Si Gene UnitProt | | HKRFRHDLYWKTWPIFTRDEIPGDNNHNLYGHQTFFMGIGDTSGKSYGVFLMNSNAMEVF |
| Accession No .: | | IQPTPIITYRVTGGILDFYIFLGDTPEQVVQQYQEVHWRPAMPAYWNLGFQLSRWNYGSLD |
| P23739 | | TVSEVVRRNREAGIPYDAQVTDIDYMEDHKEFTYDRVKFNGLPEFAQDLHNHGKYIIILDP |
| | | AISINKRANGAEYQTYVRGNEKNVWVNESDGTTPLIGEVWPGLTVYPDFTNPQTIEWWAN |
| | | ECNLFHQQVEYDGLWIDMNEVSSFIQGSLNLKGVLLIVLNYPPFTPGILDKVMYSKTLCMD |
| | | AVQHWGKQYDVHSLYGYSMAIATEQAVERVFPNKRSFILTRSTFGGSGRHANHWLGDNT |
| | | ASWEQMEWSITGMLEFGIFGMPLVGATSCGFLADTTEELCRRWMQLGAFYPFSRNHNAEG |
| | | YMEQDPAYFGQDSSRHYLTIRYTLLPFLYTLFYRAHMFGETVARPFLYEFYDDTNSWIEDT |
| | | QFLWGPALLITPVLRPGVENVSAYIPNATWYDYETGIKRPWRKERINMYLPGDKIGLHLRG |
| | | GYIIPTQEPDVTTTASRKNPLGLIVALDDNQAAKGELFWDDGESKDSIEKKMYILYTFSVSN |
| | | NELVLNCTHSSYAEGTSLAFKTIKVLGLREDVRSITVGENDQQMATHTNFTFDSANKILSIT |
| | | ALNFNLAGSFIVRWCRTFSDNEKFTCYPDVGTATEGTCTQRGCLWQPVSGLSNVPPYYFPP |
| | | ENNPYTLTSIQPLPTGITAELQLNPPNARIKLPSNPISTLRVGVKYHPNDMLQFKIYDAQHKR |
| | | YEVPVPLNIPDTPTSSNERLYDVEIKENPFGIQVRRRSSGKLIWDSRLPGFGFNDQFIQISTRL |
| | | PSNYLYGFGEVEHTAFKRDLNWHTWGMFTRDQPPGYKLNSYGFHPYYMALENEGNAHG |
| | | VLLLNSNGMDVTFQPTPALTYRTIGGILDFYMFLGPTPEIATRQYHEVIGFPVMPPYWALGF |
| | | QLCRYGYRNTSEIEQLYNDMVAANIPYDVQYTDINYMERQLDFTIGERFKTLPEFVDRIRK |
| | | DGMKYIVILAPAISGNETQPYPAFERGIQKDVFVKWPNTNDICWPKVWPDLPNVTIDETITE |
| | | DEAVNASRAHVAFPDFFRNSTLEWWAREIYDFYNEKMKFDGLWIDMNEPSSFGIQMGGK |
| | | VLNECRRMMTLNYPPVFSPELRVKEGEGASISEAMCMETEHILIDGSSVLQYDVHNLYGWS |
| | | QVKPTLDALQNTTGLRGIVISRSTYPTTGRWGGHWLGDNYTTWDNLEKSLIGMLELNLFGI |
| | | PYIGADICGVFHDSGYPSLYFVGIQVGAFYPYPRESPTINFTRSQDPVSWMKLLLQMSKKVL |
| | | EIRYTLLPYFYTQMHEAHAHGGTVIRPLMHEFFDDKETWEIYKQFLWGPAFMVTPVVEPFR |
| | | TSVTGYVPKARWFDYHTGADIKLKGILHTFSAPFDTINLHVRGGYILPCQEPARNTHLSRQN |
| | | YMKLIVAADDNQMAQGTLFGDDGESIDTYERGQYTSIQFNLNQTTLTSTVLANGYKNKQE |
| | | MRLGSIHIWGKGTLRISNANLVYGGRKHQPPFTQEEAKETLIFDLKNMNVTLDEPIQITWS |
| Oryctolagus | SEQ ID NO: | MAKRKFSGLEITLIVLFVIVFIIAIALIAVLATKTPAVEEVNPSSSTPTTTSTTTSTSGSVSCPSE |
| cuniculus (Rabbit) | 360 | LNEVVNERINCIPEQSPTQAICAQRNCCWRPWNNSDIPWCFFVDNHGYNVEGMTTTSTGLE |
| Sucrase-isomaltase, | | ARLNRKSTPTLFGNDINNVLLTTESQTANRLRFKLTDPNNKRYEVPHQFVTEFAGPAATETL |
| intestinal | | YDVQVTENPFSIKVIRKSNNRILFDSSIGPLVYSDQYLQISTRLPSEYMYGFGEHVHKRFRHD |
| Si Gene UnitProt | | LYWKTWPIFTRDQHTDDNNNNLYGHQTFFMCIEDTTGKSFGVFLMNSNAMEIFIQPTPIVT |
| Accession No .: | | YRVIGGILDFYIFLGDTPEQVVQQYQELIGRPAMPAYWSLGFQLSRWNYNSLDVVKEVVRR |
| P07768 | | NREALIPFDTQVSDIDYMEDKKDFTYDRVAYNGLPDFVQDLHDHGQKYVIILDPAISINRRA |
| | | SGEAYESYDRGNAQNVWVNESDGTTPIVGEVWPGDTVYPDFTSPNCIEWWANECNIFHQE |
| | | VNYDGLWIDMNEVSSFVQGSNKGCNDNTLNYPPYIPDIVDKLMYSKTLCMDSVQYWGKQ |
| | | YDVHSLYGYSMAIATERAVERVFPNKRSFILTRSTFAGSGRHAAHWLGDNTATWEQMEW |
| | | SITGMLEFGLFGMPLVGADICGFLAETTEELCRRWMQLGAFYPFSRNHNADGFEHQDPAFF |
| | | GQDSLLVKSSRHYLNIRYTLLPFLYTLFYKAHAFGETVARPVLHEFYEDTNSWVEDREFLW |
| | | GPALLITPVLTQGAETVSAYIPDAVWYDYETGAKRPWRKQRVEMSLPADKIGLHLRGGYII |
| | | PIQQPAVTTTASRMNPLGLIIALNDDNTAVGDFFWDDGETKDTVQNDNYILYTFAVSNNNL |
| | | NITCTHELYSEGTTLAFQTIKILGVTETVTQVTVAENNQSMSTHSNFTYDPSNQVLLIENLNF |
| | | NLGRNFRVQWDQTFLESEKITCYPDADIATQEKCTQRGCIWDTNTVNPRAPECYFPKTDNP |
| | | YSVSSTQYSPTGITADLQLNPTRTRITLPSEPITNLRVEVKYHKNDMVQFKIFDPQNKRYEVP |
| | | VPLDIPATPTSTQENRLYDVEIKENPFGIQIRRRSTGKVIWDSCLPGFAFNDQFIQISTRLPSEY |
| | | IYGFGEAEHTAFKRDLNWHTWGMFTRDQPPGYKLNSYGFHPYYMALEDEGNAHGVLLLN |
| | | SNAMDVTFMPTPALTYRVIGGILDFYMFLGPTPEVATQQYHEVIGHPVMPPYWSLGFQLCR |
| | | YGYRNTSEIIELYEGMVAADIPYDVQYTDIDYMERQLDFTIDENFRELPQFVDRIRGEGMRY |
| | | IIILDPAISGNETRPYPAFDRGEAKDVFVKWPNTSDICWAKVWPDLPNITIDESLTEDEAVNA |
| | | SRAHAAFPDFFRNSTAEWWTREILDFYNNYMKFDGLWIDMNEPSSFVNGTTTNVCRNTEL |
| | | NYPPYFPELTKRTDGLHFRTMCMETEHILSDGSSVLHYDVHNLYGWSQAKPTYDALQKTT |
| | | GKRGIVISRSTYPTAGRWAGHWLGDNYARWDNMDKSIIGMMEFSLFGISYTGADICGFFN |
| | | DSEYHLCTRWTQLGAFYPFARNHNIQFTRRQDPVSWNQTFVEMTRNVLNIRYTLLPYFYT |
| | | QLHEIHAHGGTVIRPLMHEFFDDRTTWDIFLQFLWGPAFMVTPVLEPYTTVVRGYVPNAR |
| | | WFDYHTGEDIGIRGQVQDLTLLMNAINLHVRGGHILPCQEPARTTFLSRQKYMKLIVAADD |
| | | NHMAQGSLFWDDGDTIDTYERDLYLSVQFNLNKTTLTSTLLKTGYINKTEIRLGYVHVWGI |
| | | GNTLINEVNLMYNEINYPLIFNQTQAQEILNIDLTAHEVTLDDPIEISWS |
| Homo sapiens | SEQ ID NO: | MARKKFSGLEISLIVLFVIVTIIAIALIVVLATKTPAVDEISDSTSTPATTRVTTNPSDSGKCPN |
| Sucrase-isomaltase, | 361 | VLNDPVNVRINCIPEQFPTEGICAQRGCCWRPWNDSLIPWCFFVDNHGYNVQDMTTTSIGV |
| intestinal | | EAKLNRIPSPTLFGNDINSVLFTTQNQTPNRFRFKITDPNNRRYEVPHQYVKEFTGPTVSDTL |
| Si Gene | | YDVKVAQNPFSIQVIRKSNGKTLFDTSIGPLVYSDQYLQISTRLPSDYIYGIGEQVHKRFRHD |
| UnitProt Accession | | LSWKTWPIFTRDQLPGDNNNNLYGHQTFFMCIEDTSGKSFGVFLMNSNAMEIFIQPTPIVTY |
| No .: P14410 | | RVTGGILDFYILLGDTPEQVVQQYQQLVGLPAMPAYWNLGFQLSRWNYKSLDVVKEVVR |
| | | RNREAGIPFDTQVTDIDYMEDKKDFTYDQVAFNGLPQFVQDLHDHGQKYVIILDPAISIGRR |
| | | ANGTTYATYERGNTQHVWINESDGSTPIIGEVWPGLTVYPDFTNPNCIDWWANECSIFHQE |
| | | VQYDGLWIDMNEVSSFIQGSTKGCNVNKLNYPPFTPDILDKLMYSKTICMDAVQNWGKQY |
| | | DVHSLYGYSMAIATEQAVQKVFPNKRSFILTRSTFAGSGRHAAHWLGDNTASWEQMEWSI |
| | | TGMLEFSLFGIPLVGADICGFVAETTEELCRRWMQLGAFYPFSRNHNSDGYEHQDPAFFGQ |
| | | NSLLVKSSRQYLTIRYTLLPFLYTLFYKAHVFGETVARPVLHEFYEDTNSWIEDTEFLWGPA |
| | | LLITPVLKQGADTVSAYIPDAIWYDYESGAKRPWRKQRVDMYLPADKIGLHLRGGYIIPIQE |
| | | PDVTTTASRKNPLGLIVALGENNTAKGDFFWDDGETKDTIQNGNYILYTFSVSNNTLDIVCT |
| | | HSSYQEGTTLAFQTVKILGLTDSVTEVRVAENNQPMNAHSNFTYDASNQVLLIADLKLNLG |
| | | RNFSVQWNQIFSENERFNCYPDADLATEQKCTQRGCVWRTGSSLSKAPECYFPRQDNSYS |
| | | VNSARYSSMGITADLQLNTANARIKLPSDPISTLRVEVKYHKNDMLQFKIYDPQKKRYEVP |
| | | VPLNIPTTPISTYEDRLYDVEIKENPFGIQIRRRSSGRVIWDSWLPGFAFNDQFIQISTRLPSEY |
| | | IYGFGEVEHTAFKRDLNWNTWGMFTRDQPPGYKLNSYGFHPYYMALEEEGNAHGVFLLN |
| | | SNAMDVTFQPTPALTYRTVGGILDFYMFLGPTPEVATKQYHEVIGHPVMPAYWALGFQLC |
| | | RYGYANTSEVRELYDAMVAANIPYDVQYTDIDYMERQLDFTIGEAFQDLPQFVDKIRGEG |
| | | MRYIIILDPAISGNETKTYPAFERGQQNDVFVKWPNTNDICWAKVWPDLPNITIDKTLTEDE |
| | | AVNASRAHVAFPDFFRTSTAEWWAREIVDFYNEKMKFDGLWIDMNEPSSFVNGTTTNQCR |
| | | NDELNYPPYFPELTKRTDGLHFRTICMEAEQILSDGTSVLHYDVHNLYGWSQMKPTHDAL |
| | | QKTTGKRGIVISRSTYPTSGRWGGHWLGDNYARWDNMDKSIIGMMEFSLFGMSYTGADIC |
| | | GFFNNSEYHLCTRWMQLGAFYPYSRNHNIANTRRQDPASWNETFAEMSRNILNIRYTLLPY |
| | | FYTQMHEIHANGGTVIRPLLHEFFDEKPTWDIFKQFLWGPAFMVTPVLEPYVQTVNAYVPN |
| | | ARWFDYHTGKDIGVRGQFQTFNASYDTINLHVRGGHILPCQEPAQNTFYSRQKHMKLIVA |
| | | ADDNQMAQGSLFWDDGESIDTYERDLYLSVQFNLNQTTLTSTILKRGYINKSETRLGSLHV |
| | | WGKGTTPVNAVTLTYNGNKNSLPFNEDTTNMILRIDLTTHNVTLEEPIEINWS |
| B. thetaiotaomicron | SEQ ID NO: | MKKVIKKYFFLALAIIMYSCNEDEKYDILERYTPETITSDELAPVLNLQAQYMDSNSEIVLVT |
| mannosidase | 18 | WMNPEDDFLSKVEISCCSANDNLLGEPVLLDAVSTKVGSYQTSLSVEERGYVKIVAINEKG |
| | | VRSEARTAEILSSQQDFVYRADCLMSSVIELFFGGRYNAWNENYPNATGPYWDGIAAVWG |
| | | QGAAYSGFVTMYKVTKETNNEKLRAKYAEKEETFLNSIDIFLNNGSGRKSFAYGTYIGPND |
| | | ERYYDDNVWIGIEMANLYELTGNEVYLQHANTVWNFILEGIDDVTGGGVYWKEGAVSKH |
| | | TCSTAPAAVMALKLYQLSKNESYLELAKSLYSYCKDVLQDPNDYLFYDNVRLSDPSDKNSE |
| | | LKVSKDKFTYNSGQPMLAAAMLYRITKEEQFLKDAQNIAQSIYKKWFKNYHSSILDRDIMI |
| | | LSDPNTWFNAVMFRGFVELYKIDKNDVYVKAVKNTMEHAWQSNCRNRLTNLMSDDYAG |
| | | DKKEGKWNIKTQGAFVEIFSLIGELEQLGCFQE |
| mature EndoH seq | SEQ ID NO: | APAPVKQGPTSVAYVEVNNNSMLNVGKYTLADGGGNAFDVAVIFAANINYDTGTKTAYL |
| only without its | 19 | HFNENVQRVLDNAVTQIRPLQQQGIKVLLSVLGNHQGAGFANFPSQQAASAFAKQLSDAV |
| native signal peptide | | AKYGLDGVDFDDEYAEYGNNGTAQPNDSSFVHLVTALRANMPDKIISLYNIGPAASRLSY |
| | | GGVDVSDKFDYAWNPYYGTWQVPGIALPKAQLSPAAVEIGRTSRSTVADLARRTVDEGY |
| | | GVYLTYNLDGGDRTADVSAFTRELYGSEAVRTP |
| endoH (with signal | SEQ ID NO: | MFTPVRRRVRTAALALSAAAALVLGSTAASGASATPSPAPAPAPAPVKQGPTSVAYVEVN |
| peptide underlined) | 20 | NNSMLNVGKYTLADGGGNAFDVAVIFAANINYDTGTKTAYLHFNENVQRVLDNAVTQIR |
| | | PLQQQGIKVLLSVLGNHQGAGFANFPSQQAASAFAKQLSDAVAKYGLDGVDFDDEYAEY |
| | | GNNGTAQPNDSSFVHLVTALRANMPDKIISLYNIGPAASRLSYGGVDVSDKFDYAWNPYY |
| | | GTWQVPGIALPKAQLSPAAVEIGRTSRSTVADLARRTVDEGYGVYLTYNLDGGDRTADVS |
| | | AFTRELYGSEAVRTP |
| EndoH-Tir4 fusion | SEQ ID NO: | APAPVKQGPTSVAYVEVNNNSMLNVGKYTLADGGGNAFDVAVIFAANINYDTGTKTAYL |
| (partial ORF, without | 21 | HFNENVQRVLDNAVTQIRPLQQQGIKVLLSVLGNHQGAGFANFPSQQAASAFAKQLSDAV |
| peptides that are | | AKYGLDGVDFDDEYAEYGNNGTAQPNDSSFVHLVTALRANMPDKIISLYNIGPAASRLSY |
| cleaved off post- | | GGVDVSDKFDYAWNPYYGTWQVPGIALPKAQLSPAAVEIGRTSRSTVADLARRTVDEGY |
| translationally) | | GVYLTYNLDGGDRTADVSAFTRELYGSEAVRTPGSSGSSGSSGSSGSSGSSGSSGSSEAAAR |
| | | EAAAREAAAREAAARGGGGSGGGGSGGGGSQINELNVVLDDVKTNIADYITLSYTPNSGF |
| | | SLDQMPAGIMDIAAQLVANPSDDSYTTLYSEVDFSAVEHMLTMVPWYSSRLLPELEAMDA |
| | | SLTTSSSAATSSSEVASSSIASSTSSSVAPSSSEVVSSSVASSSSEVASSSVASTSEATSSSAVTS |
| | | SSAVSSSTESVSSSSVSSSSAVSSSEAVSSSPVSSVVSSSAGPASSSVAPYNSTIASSSSTAQTSI |
| | | STIAPYNSTTTTTPASSASSVIISTRNGTTVTETDNTLVTKETTVCDYSSTSAVPASTTGYNNS |
| | | TKVSTATICSTCKEGTSTATDFSTLKTTVTVCDSACQAKKSATVVSVQSKTTGIVEQTENGA |
| | | AKAVIGMGAGALAAVAAMLL |
| EndoH-Tir4 fusion | SEQ ID NO: | MRFPSIFTAVLFAASSALAAPVNTTTEDETAQIPAEAVIGYSDLEGDFDVAVLPFSNSTNNG |
| (full ORF, including | 22 | LLFINTTIASIAAKEEGVSLDKREAEAAPAPVKQGPTSVAYVEVNNNSMLNVGKYTLADGG |
| peptides that are | | GNAFDVAVIFAANINYDTGTKTAYLHFNENVQRVLDNAVTQIRPLQQQGIKVLLSVLGNH |
| cleaved off post- | | QGAGFANFPSQQAASAFAKQLSDAVAKYGLDGVDFDDEYAEYGNNGTAQPNDSSFVHLV |
| translationally) | | TALRANMPDKIISLYNIGPAASRLSYGGVDVSDKFDYAWNPYYGTWQVPGIALPKAQLSPA |
| | | AVEIGRTSRSTVADLARRTVDEGYGVYLTYNLDGGDRTADVSAFTRELYGSEAVRTPGSS |
| | | GSSGSSGSSGSSGSSGSSGSSEAAAREAAAREAAAREAAARGGGGGGGGSGGGGSQINEL |
| | | NVVLDDVKTNIADYITLSYTPNSGFSLDQMPAGIMDIAAQLVANPSDDSYTTLYSEVDFSA |
| | | VEHMLTMVPWYSSRLLPELEAMDASLTTSSSAATSSSEVASSSIASSTSSSVAPSSSEVVSSS |
| | | VASSSSEVASSSVASTSEATSSSAVTSSSAVSSSTESVSSSSVSSSSAVSSSEAVSSSPVSSVVS |
| | | SSAGPASSSVAPYNSTIASSSSTAQTSISTIAPYNSTTTTTPASSASSVIISTRNGTTVTETDNTL |
| | | VTKETTVCDYSSTSAVPASTTGYNNSTKVSTATICSTCKEGTSTATDFSTLKTTVTVCDSAC |
| | | QAKKSATVVSVQSKTTGIVEQTENGAAKAVIGMGAGALAAVAAMLL |
| EndoH-Dan1 fusion | SEQ ID NO: | APAPVKQGPTSVAYVEVNNNSMLNVGKYTLADGGGNAFDVAVIFAANINYDTGTKTAYL |
| (partial ORF, without | 23 | HFNENVQRVLDNAVTQIRPLQQQGIKVLLSVLGNHQGAGFANFPSQQAASAFAKQLSDAV |
| peptides that are | | AKYGLDGVDFDDEYAEYGNNGTAQPNDSSFVHLVTALRANMPDKIISLYNIGPAASRLSY |
| cleaved off post- | | GGVDVSDKFDYAWNPYYGTWQVPGIALPKAQLSPAAVEIGRTSRSTVADLARRTVDEGY |
| translationally) | | GVYLTYNLDGGDRTADVSAFTRELYGSEAVRTPGSSGSSGSSGSSGSSGSSGSSGSSEAAAR |
| | | EAAAREAAAREAAARGGGGSGGGGSGGGGSASVTTTLSPYDERVNLIELAVYVSDIGAHL |
| | | SEYYAFQALHKTETYPPELAKAVFAGGDFTTMLTGISGDEVTRMITGVPWYSTRLMGAISE |
| | | ALANEGIATAVPASTTEASSTSTSEASSAATESSSSSESSAETSSNAASTQATVSSESSSAASTI |
| | | ASSAESSVASSVASSVASSASFANTTAPVSSTSSISVTPVVQNGTDSTVTKTQASTVETTITSC |
| | | SNNVCSTVTKPVSSKAQSTATSVTSSASRVIDVTTNGANKFNNGVFGAAAIAGAAALLL |
| EndoH-Dan1 fusion | SEQ ID NO: | MRFPSIFTAVLFAASSALAAPVNTTTEDETAQIPAEAVIGYSDLEGDFDVAVLPFSNSTNNG |
| (full ORF, including | 24 | LLFINTTIASIAAKEEGVSLDKREAEAAPAPVKQGPTSVAYVEVNNNSMLNVGKYTLADGG |
| peptides that are | | GNAFDVAVIFAANINYDTGTKTAYLHFNENVQRVLDNAVTQIRPLQQQGIKVLLSVLGNH |
| cleaved off post- | | QGAGFANFPSQQAASAFAKQLSDAVAKYGLDGVDFDDEYAEYGNNGTAQPNDSSFVHLV |
| translationally) | | TALRANMPDKIISLYNIGPAASRLSYGGVDVSDKFDYAWNPYYGTWQVPGIALPKAQLSPA |
| | | AVEIGRTSRSTVADLARRTVDEGYGVYLTYNLDGGDRTADVSAFTRELYGSEAVRTPGSS |
| | | GSSGSSGSSGSSGSSGSSGSSEAAAREAAAREAAAREAAARGGGGSGGGGSGGGGSASVTT |
| | | TLSPYDERVNLIELAVYVSDIGAHLSEYYAFQALHKTETYPPEIAKAVFAGGDFTTMLTGIS |
| | | GDEVTRMITGVPWYSTRLMGAISEALANEGIATAVPASTTEASSTSTSEASSAATESSSSSES |
| | | SAETSSNAASTQATVSSESSSAASTIASSAESSVASSVASSVASSASFANTTAPVSSTSSISVTP |
| | | VVQNGTDSTVTKTQASTVETTITSCSNNVCSTVTKPVSSKAQSTATSVTSSASRVIDVTING |
| | | ANKFNNGVFGAAAIAGAAALLL |
| EndoH-Sed1 fusion | SEQ ID NO: | APAPVKQGPTSVAYVEVNNNSMLNVGKYTLADGGGNAFDVAVIFAANINYDTGTKTAYL |
| (partial ORF, without | 25 | HFNENVQRVLDNAVTQIRPLQQQGIKVLLSVLGNHQGAGFANFPSQQAASAFAKQLSDAV |
| peptides that are | | AKYGLDGVDFDDEYAEYGNNGTAQPNDSSFVHLVTALRANMPDKIISLYNIGPAASRLSY |
| cleaved off post- | | GGVDVSDKFDYAWNPYYGTWQVPGIALPKAQLSPAAVEIGRTSRSTVADLARRTVDEGY |
| translationally) | | GVYLTYNLDGGDRTADVSAFTRELYGSEAVRTPGSSGSSGSSGSSGSSGSSGSSGSSEAAAR |
| | | EAAAREAAAREAAARGGGGSGGGGSGGGGSQFSNSTSASSTDVTSSSSISTSSGSVTITSSE |
| | | APESDNGTSTAAPTETSTEAPTTAIPTNGTSTEAPTTAIPTNGTSTEAPTDTTTEAPTTALPTN |
| | | GTSTEAPTDTTTEAPTTGLPTNGTTSAFPPTTSLPPSNTTTTPPYNPSTDYTTDYTVVTEYTT |
| | | YCPEPTTFTTNGKTYTVTEPTTLTITDCPCTIEKPTTTSTTEYTVVTEYTTYCPEPTTFTTNGK |
| | | TYTVTEPTTLTITDCPCTIEKSEAPESSVPVTESKGTTTKETGVTTKQTTANPSLTVSTVVPVS |
| | | SSASSHSVVINSNGANVVVPGALGLAGVAMLFL |
| EndoH-Sed1 fusion | SEQ ID NO: | MRFPSIFTAVLFAASSALAAPVNTTTEDETAQIPAEAVIGYSDLEGDFDVAVLPFSNSTNNG |
| (full ORF, including | 26 | LLFINTTIASIAAKEEGVSLDKREAEAAPAPVKQGPTSVAYVEVNNNSMLNVGKYTLADGG |
| peptides that are | | GNAFDVAVIFAANINYDTGTKTAYLHFNENVQRVLDNAVTQIRPLQQQGIKVLLSVLGNH |
| cleaved off post- | | QGAGFANFPSQQAASAFAKQLSDAVAKYGLDGVDFDDEYAEYGNNGTAQPNDSSFVHLV |
| translationally) | | TALRANMPDKIISLYNIGPAASRLSYGGVDVSDKFDYAWNPYYGTWQVPGIALPKAQLSPA |
| | | AVEIGRTSRSTVADLARRTVDEGYGVYLTYNLDGGDRTADVSAFTRELYGSEAVRTPGSS |
| | | GSSGSSGSSGSSGSSGSSGSSEAAAREAAAREAAAREAAARGGGGSGGGGSGGGGSQFSNS |
| | | TSASSTDVTSSSSISTSSGSVTITSSEAPESDNGTSTAAPTETSTEAPTTAIPTNGTSTEAPTTAI |
| | | PTNGTSTEAPTDTTTEAPTTALPTNGTSTEAPTDTTTEAPTTGLPTNGTTSAFPPTTSLPPSNT |
| | | TTTPPYNPSTDYTTDYTVVTEYTTYCPEPTTFTTNGKTYTVTEPTTLTITDCPCTIEKPTTTST |
| | | TEYTVVTEYTTYCPEPTTFTTNGKTYTVTEPTTLTITDCPCTIEKSEAPESSVPVTESKGTTTK |
| | | ETGVTTKQTTANPSLTVSTVVPVSSSASSHSVVINSNGANVVVPGALGLAGVAMLFL |
| N-terminal addition | SEQ ID NO: | EAEA |
| EAEA | 27 | |
| GGGS linker | SEQ ID NO: | GGGGS |
| | 28 | |
| GSS linker | | GSS |
| A rigid linker that | SEQ ID NO: | EAAAREAAAREAAAREAAAR |
| forms 4 turns of an | 30 | |
| alpha helix | | |
| Full linker | SEQ ID NO: | GSSGSSGSSGSSGSSGSSGSSGSSEAAAREAAAREAAAREAAARGGGGGGGGSGGGGS |
| | 31 | |
| AOX1 promoter | SEQ ID NO: | GATCTAACATCCAAAGACGAAAGGTTGAATGAAACCTTTTTGCCATCCGACATCCACA |
| | 32 | GGTCCATTCTCACACATAAGTGCCAAACGCAACAGGAGGGGATACACTAGCAGCAGAC |
| | | CGTTGCAAACGCAGGACCTCCACTCCTCTTCTCCTCAACACCCACTTTTGCCATCGAAA |
| | | AACCAGCCCAGTTATTGGGCTTGATTGGAGCTCGCTCATTCCAATTCCTTCTATTAGGCT |
| | | ACTAACACCATGACTTTATTAGCCTGTCTATCCTGGCCCCCCTGGCGAGGTTCATGTTTG |
| | | TTTATTTCCGAATGCAACAAGCTCCGCATTACACCCGAACATCACTCCAGATGAGGGCT |
| | | TTCTGAGTGTGGGGTCAAATAGTTTCATGTTCCCCAAATGGCCCAAAACTGACAGTTTA |
| | | AACGCTGTCTTGGAACCTAATATGACAAAAGCGTGATCTCATCCAAGATGAACTAAGT |
| | | TTGGTTCGTTGAAATGCTAACGGCCAGTTGGTCAAAAAGAAACTTCCAAAAGTCGGCA |
| | | TACCGTTTGTCTTGTTTGGTATTGATTGACGAATGCTCAAAAATAATCTCATTAATGCTT |
| | | AGCGCAGTCTCTCTATCGCTTCTGAACCCCGGTGCACCTGTGCCGAAACGCAAATGGGG |
| | | AAACACCCGCTTTTTGGATGATTATGCATTGTCTCCACATTGTATGCTTCCAAGATTCTG |
| | | GTGGGAATACTGCTGATAGCCTAACGTTCATGATCAAAATTTAACTGTTCTAACCCCTA |
| | | CTTGACAGCAATATATAAACAGAAGGAAGCTGCCCTGTCTTAAACCTTTTTTTTTATCA |
| | | TCATTATTAGCTTACTTTCATAATTGCGACTGGTTCCAATTGACAAGCTTTTGATTTTAA |
| | | CGACTTTTAACGACAACTTGAGAAGATCAAAAAACAACTAATTATTGGATCCCGA |
| DAK2 promoter | SEQ ID NO: | AAATAAGCATGTTTGTTTCAGATCAAAGATTAGCGTTTCAAAGTTGTGGAAAAGTGACC |
| | 33 | ATGCAACAATATGCAACACATTCGGATTATCTGATAAGTTTCAAAGCTACTAAGTAAGC |
| | | CCGTTTCAAGTCTCCAGACCGACATCTGCCATCCAGTGATTTTCTTAGTCCTGAAAAAT |
| | | ACGATGTGTAAACATAAACCACAAAGATCGGCCTCCGAGGTTGAACCCTTACGAAAGA |
| | | GACATCTGGTAGCGCCAATGCCAAAAAAAAATCACACCAGAAGGACAATTCCCTTCCC |
| | | CCCCAGCCCATTAAAGCTTACCATTTCCTATTCCAATACGTTCCATAGAGGGCATCGCT |
| | | CGGCTCATTTTCGCGTGGGTCATACTAGAGCGGCTAGCTAGTCGGCTGTTTGAGCTCTC |
| | | TAATCGAGGGGTAAGGATGTCTAATATGTCATAATGGCTCACTATATAAAGAACCCGCT |
| | | TGCTCAACCTTCGACTCCTTTCCCGATCCTTTGCTTGTTGCTTCTTCTTTTATAACAGGA |
| | | AACAAAGGAATTTATACACTTTAAGAATT |
| PEX11 promoter | SEQ ID NO: | CTTCCCCATTTCACTGACAGTTTGTAGAAATAGGGCAACAATTGATGCAAATCGATTTT |
| | 34 | CAACGCATTGGTTTTGATAGCATTGATGATCTTGGAGCTGTAAAAGTCCGGCTGGATAA |
| | | GCTCAATGAAATAGGTTGGTTGATCTGGATCTTCTTTTGGGTCATTTTGTTCGCTCTGTA |
| | | TTTCACAAATTGCCAGAATCTCTGCCAACCACAGTGGTAGGTCCAACTTGGTGTTCTGA |
| | | ATCACAGGCTTCCCCGGGTTGTTCTCTAAATAACCGAGGCCCGGCACAGAAATCGTAA |
| | | ACCGACACGGTATCTTTTGTCCGTCCGCCAGTATCTCATCAAGGTCGTAGTAGCCCATG |
| | | ATGAGTATCAAAGGGGATTTGGTTATGCGATGCAACGAGAGATTGTTTATCCCAGATGC |
| | | TGATGTAAAAACCTTAACCAGCGTGACAGTAGAAATAAGACACGTTAAAATTACCCGC |
| | | GCTTCCCTAACAATTGGCTCTGCCTTTCGGCAAGTTTCTAACTGCCCTCCCCTCTCACAT |
| | | GCACCACGAACTTACCGTTCGCTCCTAGCAGAACCACCCCAAAGTTTAATCAGGACCG |
| | | CATTTTAGCCTATTGCTGTAGAACCCCACAACATAACCTGGTCCAGAGCCAGCCCTTTA |
| | | TATATGGTAAATCCCGTTTGAACTTCGAAGTGGAATCGGAATTTTTACATCAAAGAAAC |
| | | TGATACTGAAACTTTTGGCTTCGACTTGGACTTTCTCTTAATC |
| FLD 1 promoter | SEQ ID NO: | AAATCAGCCATTAATCTCACCTCAGTTTTTGAATCAGTAGAATTTTCAATGAAACAAAC |
| | 35 | GGTTGGTATATTATTTGATAGGGTAGCCAAATTTCCAAAAATGAACTTTTCATCAGGTA |
| | | ATATCTTGAATACCGTAATGTAGTGACTATTGGAAGAAACTGCTATCAAATTATATTTC |
| | | GGATAGAAATCCAAACCCCAGACTGATCTCTTGAGTCTCAACTCTAAGTCAGCCGCGA |
| | | CTCTAATTATCTGTGGATTAGGAGTTAGTGTGGACAAAGCATCAGTATAGTATAACTTT |
| | | ACGGTTCCATTATCAGACGCTATTGCAAGAACTTCCTTTCCATTGATCTCTCCAATTCGA |
| | | CAGTAATTGATATCATAAGGTAGGTCTGGAAACACACTGGCGCTTGTATCCCATTCTGC |
| | | AGGAATTTCTGGAACGGTGGTAATGGTAGTTATCCAACGGAGTTGGGGTAGTTGGTAT |
| | | ATCTGGATATGCCGCCTATAGGATAAAAACAGGAGAGAGTGAACCTTGCTTACGGCTA |
| | | CTAGATTGTTCTTGTACTCGGAATTGTCGTTATCGGAAACTAGACTAATCTCATCTGTGT |
| | | GTTGCAGTACTATTGAGTCGTTGTAGTATCTACCAGGAGGGCATTCCATGAACTAGTGA |
| | | GACAAATGAGTTGGATTTTCTCAATAGACATATGCAAGAATGCTACACAACGGATGTC |
| | | GCACTCTTTTTCTTAGTTGATAATATCATCCAATCAGAAGACACGGGCTAGAAGGACTT |
| | | GCTCCCGAAGGATAATCCACTGCTACTATCTCCCTTCCTCACATATAGTCTTGCAGGGC |
| | | TCATGCCCCTTTCTCCTTCGAACTGCCCGATGAGGAAGTCTTTAGCCTATCAAGGAATT |
| | | CGGGACCATCATCAATTTTTAGAGCCTTACCTGATCGCAATCAGGATTTCACTACTCAT |
| | | ATAAATACATCACTCAAACTCCAACTTTGCTTGTTCATACAATTCTTGATATTCACAGG |
| | | ATC |
| FGH1 promoter | SEQ ID NO: | GTGAATTTGTCACGGAATTGACCAAGAGGTCAGACGATCCTGTATCCCATTGAGCCGTT |
| | 36 | ATGCTTTGTGGGGGAAACCCTATTTCTATCGTACTAAGAAAACCAATGGTGAACTCATA |
| | | TTCGGTATCAATGGCGACGATTCCAGCATAGCCTGTAGACAGTAACAACACTAGGGCA |
| | | ACAGCAACTAACATATCTTCATTGATGAAACGTTGTGATCGGTGTGACTTTTATAGTAA |
| | | AAGCTACAACTGTTTGAAATACCAAGATATCATTGTGAATGGCTCAAAAGGGTAATAC |
| | | ATCTGAAAAACCTGAAGTGTGGAAAATTCCGATGGAGCCAACTCATGATAACGCAGAA |
| | | GTCCCATTTTGCCATCTTCTCTTGGTATGAAACGGTAGAAAATGATCCGAGTATGCCAA |
| | | TTGATACTCTTGATTCATGCCCTATAGTTTGCGTAGGGTTTAATTGATCTCCTGGTCTAT |
| | | CGATCTGGGACGCAATGTAGACCCCATTAGTGGAAACACTGAAAGGGATCCAACACTC |
| | | TAGGCGGACCCGCTCACAGTCATTTCAGGACAATCACCACAGGAATCAACTACTTCTCC |
| | | CAGTCTTCCTTGCGTGAAGCTTCAAGCCTACAACATAACACTTCTTACTTAATCTTTGAT |
| | | TCTCGAATTGTTTACCCAATCTTGACAACTTAGCCTAAGCAATACTCTGGGGTTATATAT |
| | | AGCAATTGCTCTTCCTCGCTGTAGCGTTCATTCCATCTTTCTAGAATTCGT |
| DAS2 promoter | SEQ ID NO: | CCTGTTGATAAGACGCATTCTAGAGTTGTTTCATGAAAGGGTTACGGGTGTTGATTGGT |
| | 37 | TTGAGATATGCCAGAGGACAGATCAATCTGTGGTTTGCTAAACTGGAAGTCTGGTAAG |
| | | GACTCTAGCAAGTCCGTTACTCAAAAAGTCATACCAAGTAAGATTACGTAACACCTGG |
| | | GCATGACTTTCTAAGTTAGCAAGTCACCAAGAGGGTCCTATTTAACGTTTGGCGGTATC |
| | | TGAAACACAAGACTTGCCTATCCCATAGTACATCATATTACCTGTCAAGCTATGCTACC |
| | | CCACAGAAATACCCCAAAAGTTGAAGTGAAAAAATGAAAATTACTGGTAACTTCACCC |
| | | CATAACAAACTTAATAATTTCTGTAGCCAATGAAAGTAAACCCCATTCAATGTTCCGAG |
| | | ATTTAGTATACTTGCCCCTATAAGAAACGAAGGATTTCAGCTTCCTTACCCCATGAACA |
| | | GAAATCTTCCATTTACCCCCCACTGGAGAGATCCGCCCAAACGAACAGATAATAGAAA |
| | | AAAGAAATTCGGACAAATAGAACACTTTCTCAGCCAATTAAAGTCATTCCATGCACTCC |
| | | CTTTAGCTGCCGTTCCATCCCTTTGTTGAGCAACACCATCGTTAGCCAGTACGAAAGAG |
| | | GAAACTTAACCGATACCTTGGAGAAATCTAAGGCGCGAATGAGTTTAGCCTAGATATC |
| | | CTTAGTGAAGGGTTGTTCCGATACTTCTCCACATTCAGTCATAGATGGGCAGCTTTGTT |
| | | ATCATGAAGAGACGGAAACGGGCATTAAGGGTTAACCGCCAAATTATATAAAGACAAC |
| | | ATGTCCCCAGTTTAAAGTTTTTCTTTCCTATTCTTGTATCCTGAGTGACCGTTGTGTTTAA |
| | | TATAACAAGTTCGTTTTAACTTAAGACCAAAACCAGTTACAACAAATTATAACCCCTCT |
| | | AAACACTAAAGTTCACTCTTATCAAACTATCAAACATCAAAAGAATTCGCG |
| CAT1 promoter | SEQ ID NO: | TAATCGAACTCCGAATGCGGTTCTCCTGTAACCTTAATTGTAGCATAGATCACTTAAAT |
| | 38 | AAACTCATGGCCTGACATCTGTACACGTTCTTATTGGTCTTTTAGCAATCTTGAAGTCTT |
| | | TCTATTGTTCCGGTCGGCATTACCTAATAAATTCGAATCGAGATTGCTAGTACCTGATA |
| | | TCATATGAAGTAATCATCACATGCAAGTTCCATGATACCCTCTACTAATGGAATTGAAC |
| | | AAAGTTTAAGCTTCTCGCACGAGACCGAATCCATACTATGCACCCCTCAAAGTTGGGAT |
| | | TAGTCAGGAAAGCTGAGCAATTAACTTCCCTCGATTGGCCTGGACTTTTCGCTTAGCCT |
| | | GCCGCAATCGGTAAGTTTCATTATCCCAGCGGGGTGATAGCCTCTGTTGCTCATCAGGC |
| | | CAAAATCATATATAAGCTGTAGACCCAGCACTTCAATTACTTGAAATTCACCATAACAC |
| | | TTGCTCTAGTCAAGACTTACAATTAAA |
| MDH3 promoter | SEQ ID NO: | TAGCTTGGGTAGGACTTGACAAGTACGGCTTCCGTGGTCATACCAAACGCCTTTGTTAC |
| | 39 | CGTTGGCTATACCTAATGACCAAGGCATTTGTGGATTATAACGGTATCGTAGTTGAAAA |
| | | ATATGACGTAACCACTGGTACTAGCCCCCACAAGGTTGATGCTGAATACGGGAATCAA |
| | | GGTGCCGATTTTAAAGGAGTAGCCACTGAAGGGTTTGGCTGGGTCAATGCCTCTTTTAT |
| | | TTTGGGATTAACCTACTTAGATGTCCAAGGCATCCGTGCGATAGGCGCCGTTACGTCCC |
| | | CTGATGTATTTTTCAGGAAGCTCAAACCTTGGGAACGCGCAAGTTATGGCCTAAGGCCA |
| | | TGTAACGAGATAGTCAAGTCAAACTAGAAGTATACGGTTTCCCCGCAGAAATAGCAGA |
| | | AATAGGCGACAAATACATACAACATTTTCATTGTGATAGGGGGCGGCGGTTCCTAGGA |
| | | GGGACAACCCCCAGAAACCTTGTAGACTACGTTTTCACGACGATGGGTTATTACTGTAA |
| | | AGGAAGAATATACTACCCACCAGTTGAATGTTTGAACGGATCAAAGGTCGAAGGGAGT |
| | | ACACGGCCCAACCAACGTAGCTACCGGAGAAAGCAAGACTTTCCCAAACCAAATAGCT |
| | | CCGGGTTTCTTCTCCGGCAACCCGTCAGTTTTTGTGTGGCCGGACAAAAATTCGCACCC |
| | | TCAGTCTAATTGAAAGGTCGGGCTCCGAGCTCTAGGCGTTTGCGCATGTAATATTGCAT |
| | | CCCCTCCCATAGATAATACTGCGCGAACACAGGGTGCAAATTATGATGACCACACATG |
| | | CCAGTGACCAAAACAGTTTTTTAGTCTTTAAAAACCCTCGGAACTTCTGAGTATATAAA |
| | | GGCTTCTCATTTCCTACAAGCAAACAAAGAAGAAACTTCCACTTTCTAACTTTTTATCT |
| | | ATAGACTTTAGAGTTACAACCAACGAACAATAACAAA |
| HAC1 promoter | SEQ ID NO: | TGAAGCTTATCTGCTGAGCAAGTIGTTTGACCAAACTTGAGTCAACAGTGGTTAACTAT |
| | 40 | ATCCTCTATTATTTTAGATGGGAGCACATCAAGTGTACGGGAACAATGCAATCGACAA |
| | | CCTGTAGCCTGACATACATAGCCATCTTGAATTGACAAAACTTAGAATGTCTTGAATGT |
| | | GATAGATATGAGTTCCCAAAAATCTCTTTTACGATTTCCCAGTTGCGGTGTACTATTAC |
| | | ACAGAGGATATCATAGCAGACTTACAATCCTCAGGCATAAAACGAGCTTTCTTATCAA |
| | | AGTGTATTCAAATGGACCATTTGATTGCACCAAGGCATTAGCCCCAAACCATACCACAC |
| | | AGTAACTTGATATTCTCAGCATGCATGGAAATTCCACTCATAACGCGCTATTCACCGCG |
| | | AATACTTATCTATGAAACTGGGTTCTTTAGTATTCTTTGCCAAATTTCACCGATTAGAAA |
| | | TTATTAGGTAATATAATTTCTTTGGGGAACCCCTTCCCGTTACGCCCGCTGCGGCTTTGT |
| | | GGTTCTTTTCCAGTCTTGAGCAAATTACATCTGGTCTAGACAGTTCTTCCGTGCCCCAGT |
| | | ATGCGAGCGCAAACTTTCAATCAAACCTCGTAGCAAATTGGTACTTGAACTTCGTATTT |
| | | AACCGCTATTAAATGTACTGACTCTTACATTATGAAAAATTTTGATAAAGATTTTATATT |
| | | TCATCTCAGTTAATCTCCTAATAATAATAGTCTGCATAACTCAAACGGTACTTCCTTTTC |
| | | GGAACGCGAAGAGTAGTCTCTATGTCATTCTCACACTATCCGCAGCGCAATAGAGAAC |
| | | GAGCATGTTACCCGACTCATCCCTTGTCGATTCGGAAACGATTTATAAATACAATTAGA |
| | | TCGCCACCGATCTTCTTTTGTCAATATTATAAAAATAGTACAGATTTTCCTTAGTCGAAT |
| | | CAGATCGCAGAAA |
| BiP promoter | SEQ ID NO: | AGATCTGAGGGTGTATACGATGTATCGTGCCGAACACATGCACTTGACGGCACAGCAA |
| | 41 | ATGGTATTCAAGAAGACCACTTTAGAATGGGAGTTAATAGGGATGGTTTCATGGAGGT |
| | | TAAAACACTTCAAGGAGGCATCTGAAGCATTCAAGTATGCACTAGGTCTGAGGTTTTCG |
| | | GTCAAGGCATGCAAGAAATTAATTGTATTCTATCTGAACGAACGCTCCAGAATGAACC |
| | | AGCCAGAAACCTCAATTGCCCTCAACAACTTAAATCAATCCACATTATCCATCCAAGAG |
| | | ATTCTCAAGTATCGTTCGTTCCTCGATATCAACCTAATTTCAAACTTGGTCAAACTAGG |
| | | AGTTTGGAATCACCGCTGGTATGCTGAGTTTTCTCCAAAACTCATAGAAAGCCTTGCGG |
| | | TTGTTGTGGAGAACGGAGGGCTTATCAAGGTAGAAAACGAGGTTAAGGCTACCTATTT |
| | | CGATTCACAAGATGGAGTTTACGACTTGATGAACGAGGTATTCAAGTTCATGAAGCATT |
| | | ACGATTATCCTGGGACTGACAACTAAGAGCTCCTAGTGAAGACTTGAGATGGACATGA |
| | | TAAACAATTATAGTGAAAATAGAAACCATAATACAATATTCTAATAGAGGAACCGTTT |
| | | ACCTGTGGTTCCTATTGTGGCCTACTGTTACTAGCTAGTGTAATACACCCTTGCCTCAGC |
| | | TTTGCAAGTTGACAACTCAGCCAAATGATCTTTGAATGCGCGAAACCTCAAGGTCCATC |
| | | GAATTTTCTCGAATTTTCAGTGTTTTCATACAGCGTGTCATCTTCTTTCGCGTACTTATTA |
| | | AAATCGTACCCAGATCCCTTCTTCTTCCTTAATTTCAATTCCAACACTCAAGA |
| RAD30 promoter | SEQ ID NO: | AGATCTTGCAAAATACCTTTCCAGCTTTCCAGCTTCCTAGCACTCATCTTGAAGATATC |
| | 42 | AAATATTCTCCATTCAAACCAACATCAAAAAATAGAATAATTATAATCAGTTTGAAGA |
| | | GCAAGAGTAATTTTAAAGGAAACACATTCATGGTCAGCTAGAAGGTTGACTGAAGAGT |
| | | CGCAAGATATCTGAGAATAAAAAAGAGCATAGCTAACAAGATGAGTAAACACGGCAA |
| | | ACAGATTTAGGAACAGGTGAAGGGTTTCTGGCTCTTCAATGTATATCCTGCTAGCCACC |
| | | CATTCAGAAATAACACAAAGTAGGACCCTACTGAAAAATAAATTTAATACATCTTCAT |
| | | CCTCTCATTAAACCACCGACCACTCAAACCATACCAGCCTTGTCCAATTCCATGCATCG |
| | | TGCTATCCGTCAGAATTTTCAGTGTTAATCGAATCGGTCATTATAGCTCCGTCTGGGGC |
| | | GACAACTTGTCATCACAGAATAGCACAATTATGCGTTGGAATCGTCAAAAAATCACCT |
| | | CCAGGTCTGTATACATACAGAACTGGTTGTAACGACAACCTTGTTTGATTGAGGTGACT |
| | | GGAAGGTGGAAAGAAAGGGAGGAAATAAATATTGCAAGGAAAGAAAAAAAAATTGTT |
| | | CACAGTCACCTCTTCACCTTCGCGATTTCATGTTTCTTTCATGTGCTAACTGATCCCAGG |
| | | GCTTCTCCAGCGCCCTTATCTGTTAG |
| RVS161-2 promoter | SEQ ID NO: | CTGCCCATCTATGACTGAATGTGGAGAAGTATCGGAACAACCCTTCACTAAGGATATCT |
| | 43 | AGGCTAAACTCATTCGCGCCTTAGATTTCTCCAAGGTATCGGTTAAGTTTCCTCTTTCGT |
| | | ACTGGCTAACGATGGTGTTGCTCAACAAAGGGATGGAACGGCAGCTAAAGGGAGTGCA |
| | | TGGAATGACTTTAATTGGCTGAGAAAGTGTTCTATTTGTCCGAATTTCTTTTTTCTATTA |
| | | TCTGTTCGTTTGGGCGGATCTCTCCAGTGGGGGGTAAATGGAAGATTTCTGTTCATGGG |
| | | GTAAGGAAGCTGAAATCCTTCGTTTCTTATAGGGGCAAGTATACTAAATCTCGGAACAT |
| | | TGAATGGGGTTTACTTTCATTGGCTACAGAAATTATTAAGTTTGTTATGGGGTGAAGTT |
| | | ACCAGTAATTTTCATTTTTTCACTTCAACTTTTGGGGTATTTCTGTGGGGTAGCATAGCT |
| | | TGACAGGTAATATGATGTACTATGGGATAGGCAAGTCTTGTGTTTCAGATACCGCCAAA |
| | | CGTTAAATAGGACCCTCTTGGTGACTTGCTAACTTAGAAAGTCATGCCCAGGTGTTACG |
| | | TAATCTTACTTGGTATGACTTTTTGAGTAACGGACTTGCTAGAGTCCTTACCAGACTTCC |
| | | AGTTTAGCAAACCACAGATTGATCTGTCCTCTGGCATATCTCAAACCAATCAACACCCG |
| | | TAACCCTTTCATGAAACAACTCTAGAATGCGTCTTATCAACAGGATTGCCCAAAACAGT |
| | | AATTGGGGCGGTGGAATCTACATGGGAGTTCCATCGTTGTCTCGGTTTTTCTCCCTATA |
| | | AGCTACTCTGGAGACGAAGTAACTAACACCCTCAAATATCATT |
| MPP10 promoter | SEQ ID NO: | TCTGAATCCGACCTCCTCTAATCTACCACTGAAGAGAAGCAGTGTATTGTTCGTCTACG |
| | 44 | TAAATTTGAATGTGTAAATGGCAAACATGGCTTCGGGGATGATTTGGCATATATATTAT |
| | | TGTAGCATCGTCTGTGGCTCTATGAGTTGTGTGGCGGATGATGAAAAGTTTCGTGCTGA |
| | | TCCCACAATGCGGCATTTACCAAATGGGGAAAGACCAGATTTCTTCGCTGCGCCAGCTA |
| | | GGGACAGCATAATGTTCCAAGAAGAAGCGATTACAGGTGGATTACAAAGCGTTCGTCT |
| | | GCAGTTGATGTTCTACGTGATGGGTATGAGTTGTAGTGCTACGCTCCATGAATACTTCT |
| | | AATTTGTCGTTGACAATCCATGAATAATTTAAGTTTGCTTCCCAAGAGTCTATTGCGAA |
| | | GGGTGAGCCGAATCTCTTGGCGTATGCACCCGACTCGTCGGCTTTTGTGCGTTCCTTGC |
| | | AAAGCTCGGTAGCAATCCGTTGGTGGGAGAAATTTGTCTCACGAATTTCAGTTGGGAGT |
| | | AGCTGTTCCTGGTAGCAAGTTCGAGGGGATCTGTGCTCATAAAACGTGCTCACGCCAA |
| | | AAATATTCTTACAAAATCTTCGCGGGGTGTTTGTCTTACATAATCGATTGGATATTTTCT |
| | | TCAAATTTTTTTTTCTTACTGAAGTCCCCTATAGAG |
| THP3 promoter | SEQ ID NO: | TCTTGCCAGTTGTCTCCTAAGATGTCATCGGAGTAGGCTCGGCTAAAGAGTAGTAATGC |
| | 45 | ATCAAGACCAACCAAAACACCTTCCACGAGTTCAGATGAACCTTTTAATAACTTCAGGT |
| | | CACTTTGATGCCGGCACAACTGGGCGAGTTTCGTATAGTTAACTCTGATCTTGCACTCC |
| | | AGAACGGGAATAGGATTGACTTTTTGCTTCCGAGAAACGATTTGCTCTCTCTTCGTCTG |
| | | GCTTTTCACTTTATATCGCACGGAATCAATGGATGGAACTCCTAAAGCTCCTAACTTCG |
| | | ATGATTTGCTAGCCATGACTCTGTGGGACATTTTCTTGCATCTCGTTTGTAACCTGTCTG |
| | | TTCCTACACTAAGTTTATGAGAGGCTACTTTGGATTCTAGCCTCGGTGGTAAAGTGGGA |
| | | GATAACAACGGCATAAGGCAAGAACCAGAAGTACCATAACGGTCTGGTAAAGTTGGTG |
| | | ATAACTTAATTGGAAGAGTGTAAGTAAGACGTGGCTTGTAATAAGGCTTTCCATCAAA |
| | | AAGGTTCTCCGGGTTGGAGTTTGTGAGGCTCACATCTTTGATCAGTCTTTCAATATAAA |
| | | TTGGTAACGTTGATGACAATGCCGGAGGTAATTTCTGTAGTTGTTGATATACGCAGATA |
| | | ACAGATTCAAATCTCCATTGGTTTTCATCATTGTGGCTTAAATTAGATCAGAACATGGT |
| | | AGTATTTAAAAATGGATCTCTTTGCAGATTTACTCAATATAGCGAAAAAAGGAGACATT |
| | | CGTTACAAAATATGAAGATAATTCGCCTCATAACTCGATTAATCAAAACAGACGGTCC |
| | | AGTTCTTCTTTTGGTAGT |
| GBP2 promoter | SEQ ID NO: | ATCTGTACTGGTACTGACAAAGGTTATCCAGAATCCGAGACATTTCAACAACAGAGAT |
| | 46 | TCCAGGCTTCAAAACATCCATTTTATCACCAATATCTAGTAATGCTTGCAACAATTCTG |
| | | GATACTTCTTCTGTGTAACCAAATCTCTTATAAACTGAACAGCTTTCTGTACGTTGTCGT |
| | | CAGTAGTTGGATCAACCTCAGTGGTGACCTGGCCTATCGGTTTTCCAAAAGACTTGTTT |
| | | ATCACGTCCGAAAGCTCCCATTTTTGCAGATGCGCAACTTTAAAAGGCCTGGCTTGAAC |
| | | ATTTGCATCTCTTGTTGTGTGTTCTTTGAGAAAATATTCATCGATCTGGGTGCTTCCAAC |
| | | GACAGAAGATACTCTTCTGAGACCAGAAAGTCCCCAGCCATGCTTCCTAATTACAAAA |
| | | TATTTGTAGGAAGATCCCTGATTAGGACAAAGTTGTCTTCTCATGAGTTCAACTGAAAC |
| | | TGGGGCTCAAACGGATTATGAAAGGGGTGATTAAAGGTTTTCCTAGCCTTACTTTCCAA |
| | | ATGTCGACCGAGACGAACATTTAAAATCCTAACATCAGAAATTTCTATCCTTAATCTCA |
| | | TTGATGGTTAGTACACTTCGCAGAGTCTCCACATTTGCAGACCCTCCTGGATAACCAAA |
| | | GCTTATCTAACAGCGGCATTGGACCTTTGAAAAGACCCTC |
| DAS1 promoter | SEQ ID NO: | AAATCTGAACACGATGAAACCTCCCCGTAGATTCCACCGCCCCGTTACTTTTTTGGGCA |
| | 47 | ATCCCGTTGATAAGATCCATTTTAGAGTTGTTTCTGAAAGGATTACAGGCGTTGAAGGG |
| | | TCAGAGAGATGCCAGAGAACAGACCAATTGGTAGTTTGCTAAAGTGGACGTCTGGCAG |
| | | GTGCTCTATCGTGTTCTTTATTTAGGGCGTTACACTTAGTAGGATTACGTAACAATTTGG |
| | | CTTAACCTTCTAAGTTAGAAAGAAACCAAGAGGGGTCCTCTTTAACGTTCAGCAGTATC |
| | | TAAAACACAAAACCTGCCCTCATAATACATCATTCTATCTGTCAAGCTGTGCTACCCCA |
| | | CAGAAATACCCCCAAGAGTTAAAGTGAAAAGAAAAGCTAAATCTGTTAGACTTCACCC |
| | | CATAACAAACTTGATAGTTCCTGTAGCCAATGAAAGTTAACCCCATTCAATGTTCCGAG |
| | | ATCTAGTATGCTTGCTCCTATAAGGAACGAAGGGTTCCAGCTTCCTTACCCCATCAATG |
| | | GAAATCTCCTATTTACCCCCCACTGGAAAGATCCGTCCGAACGAACGGATAATAGAAA |
| | | AAAGAAATTCGGACAAAATAGAACACTTATTTAGCCAATGAAATCCATTTCCAGCATC |
| | | TCCTTCAACTGCCGTTCCATCCCCTTTGTTGAGCTACACCATCGTCAGCCAGTACCGAAT |
| | | AGGAAACTTAACCGATATCTTGGAGAATTCTAATGCGCGAATGAGTTTAGCCTAGATAT |
| | | CCTTAGTGAAGGGTTGTTCCGATACTTCTCCACATTCAGTCATTTCAGATGGGCAGCAT |
| | | TGTTATCATGAAGAAACGGAAACGGGCAGTAAGGGTTAACCGCCAAATTATATAAAGA |
| | | CAACATGTCCCCAGTTTAAAGTTTTTCTTTCCTATTCTTGTATCCTGAGTGACCGTTGTG |
| | | TTTAAAATAACAAGTTCGTTTTAACTTAAGACCAAAACCAGTTACAACAAATTATTCCC |
| | | CAACTAAACACTAAAGTTCACTCTTATCAAACTATCAAACATCAAAG |
| Methanol inducible | SEQ ID NO: | CTTCCCCATTTCACTGACAGTTTGTAGAAATAGGGCAACAATTGATGCAAATCGATTTT |
| promoter | 48 | CAACGCATTGGTTTTGATAGCATTGATGATCTTGGAGCTGTAAAAGTCCGGCTGGATAA |
| | | GCTCAATGAAATAGGTTGGTTGATCTGGATCTTCTTTTGGGTCATTTTGTTCGCTCTGTA |
| | | TTTCACAAATTGCCAGAATCTCTGCCAACCACAGTGGTAGGTCCAACTTGGTGTTCTGA |
| | | ATCACAGGCTTCCCCGGGTTGTTCTCTAAATAACCGAGGCCCGGCACAGAAATCGTAA |
| | | ACCGACACGGTATCTTTTGTCCGTCCGCCAGTATCTCATCAAGGTCGTAGTAGCCCATG |
| | | ATGAGTATCAAAGGGGATTTGGTTATGCGATGCAACGAGAGATTGTTTATCCCAGATGC |
| | | TGATGTAAAAACCTTAACCAGCGTGACAGTAGAAATAAGACACGTTAAAATTACCCGC |
| | | GCTTCCCTAACAATTGGCTCTGCCTTTCGGCAAGTTTCTAACTGCCCTCCCCTCTCACAT |
| | | GCACCACGAACTTACCGTTCGCTCCTAGCAGAACCACCCCAAAGTTTAATCAGGACCG |
| | | CATTTTAGCCTATTGCTGTAGAACCCCACAACATAACCTGGTCCAGAGCCAGCCCTTTA |
| | | TATATGGTAAATCCCGTTTGAACTTCGAAGTGGAATCGGAATTTTTACATCAAAGAAAC |
| | | TGATACTGAAACTTTTGGCTTCGACTTGGACTTTCTCTTAATCGAATTCGT |
| GCW14 promoter | SEQ ID NO: | CAGGTGAACCCACCTAACTATTTTTAACTGGCATCCAGTGAGCTCGCTGGGTGAAAGCC |
| | 49 | AACCATCTTTTGTTTCGGGGAACCGTGCTCGCCCCGTAAAGTTAATTTTTTTTTCCCGCG |
| | | CAGCTTTAATCTTTCGGCAGAGAAGGCGTTTTCATCGTAGCGTGGGAACAGAATAATCA |
| | | GTTCATGTGCTATACAGGCACATGGCAGCAGTCACTATTTTGCTTTTTAACCTTAAAGTC |
| | | GTTCATCAATCATTAACTGACCAATCAGATTTTTTGCATTTGCCACTTATCTAAAAATAC |
| | | TTTTGTATCTCGCAGATACGTTCAGTGGTTTCCAGGACAACACCCAAAAAAAGGTATCA |
| | | ATGCCACTAGGCAGTCGGTTTTATTTTTGGTCACCCACGCAAAGAAGCACCCACCTCTT |
| | | TTAGGTTTTAAGTTGTGGGAACAGTAACACCGCCTAGAGCTTCAGGAAAAACCAGTAC |
| | | CTGTGACCGCAATTCACCATGATGCAGAATGTTAATTTAAACGAGTGCCAAATCAAGA |
| | | TTTCAACAGACAAATCAATCGATCCATAGTTACCCATTCCAGCCTTTTCGTCGTCGAGC |
| | | CTGCTTCATTCCTGCCTCAGGTGCATAACTTTGCATGAAAAGTCCAGATTAGGGCAGAT |
| | | TTTGAGTTTAAAATAGGAAATATAAACAAATATACCGCGAAAAAGGTTTGTTTATAGCT |
| | | TTTCGCCTGGTGCCGTACGGTATAAATACATACTCTCCTCCCCCCCCTGGTTCTCTTTTT |
| | | CTTTTGTTACTTACATTTTACCGTTCCGT |
| FDH1 promoter | SEQ ID NO: | AAATAAATGGCAGAAGGATCAGCCTGGACGAAGCAACCAGTTCCAACTGCTAAGTAAA |
| | 50 | GAAGATGCTAGACGAAGGAGACTTCAGAGGTGAAAAGTTTGCAAGAAGAGAGCTGCG |
| | | GGAAATAAATTTTCAATTTAAGGACTTGAGTGCGTCCATATTCGTGTACGTGTCCAACT |
| | | GTTTTCCATTACCTAAGAAAAACATAAAGATTAAAAAGATAAACCCAATCGGGAAACT |
| | | TTAGCGTGCCGTTTCGGATTCCGAAAAACTTTTGGAGCGCCAGATGACTATGGAAAGA |
| | | GGAGTGTACCAAAATGGCAAGTCGGGGGCTACTCACCGGATAGCCAATACATTCTCTA |
| | | GGAACCAGGGATGAATCCAGGTTTTTGTTGTCACGGTAGGTCAAGCATTCACTTCTTAG |
| | | GAATATCTCGTTGAAAGCTACTTGAAATCCCATTGGGTGCGGAACCAGCTTCTAATTAA |
| | | ATAGTTCGATGATGTTCTCTAAGTGGGACTCTACGGCTCAAACTTCTACACAGCATCAT |
| | | CTTAGTAGTCCCTTCCCAAAACACCATTCTAGGTTTCGGAACGTAACGAAACAATGTTC |
| | | CTCTCTTCACATTGGGCCGTTACTCTAGCCTTCCGAAGAACCAATAAAAGGGACCGGCT |
| | | GAAACGGGTGTGGAAACTCCTGTCCAGTTTATGGCAAAGGCTACAGAAATCCCAATCT |
| | | TGTCGGGATGTTGCTCCTCCCAAACGCCATATTGTACTGCAGTTGGTGCGCATTTTAGG |
| | | GAAAATTTACCCCAGATGTCCTGATTTTCGAGGGCTACCCCCAACTCCCTGTGCTTATA |
| | | CTTAGTCTAATTCTATTCAGTGTGCTGACCTACACGTAATGATGTCGTAACCCAGTTAA |
| | | ATGGCCGAAAAACTATTTAAGTAAGTTTATTTCTCCTCCAGATGAGACTCTCCTTCTTTT |
| | | CTCCGCTAGTTATCAAACTATAAACCTATTTTACCTCAAATACCTCCAACATCACCCAC |
| | | TTAAACAGAATT |
| FBA1 promoter | SEQ ID NO: | TGCTTAAGTAATTGAAAACAGTGTTGTGATTATATAAGCATGGTATTTGAATAGAACTA |
| | 51 | CTGGGGTTAACTTATCTAGTAGGATGGAAGTTGAGGGAGATCAAGATGCTTAAAGAAA |
| | | AGGATTGGCCAATATGAAAGCCATAATTAGCAATACTTATTTAATCAGATAATTGTGGG |
| | | GCATTGTGACTTGACTTTTACCAGGACTTCAAACCTCAACCATTTAAACAGTTATAGAA |
| | | GACGTACCGTCACTTTTGCTTTTAATGTGATCTAAATGTGATCACATGAACTCAAACTA |
| | | AAATGATATCTTTTACTGGACAAAAATGTTATCCTGCAAACAGAAAGCTTTCTTCTATT |
| | | CTAAGAAGAACATTTACATTGGTGGGAAACCTGAAAACAGAAAATAAATACTCCCCAG |
| | | TGACCCTATGAGCAGGATTTTTGCATCCCTATTGTAGGCCTTTCAAACTCACACCTAAT |
| | | ATTTCCCGCCACTCACACTATCAATGATCACTTCCCAGTTCTCTTCTTCCCCTATTCGTA |
| | | CCATGCAACCCTTACACGCCTTTTCCATTTCGGTTCGGATGCGACTTCCAGTCTGTGGG |
| | | GTACGTAGCCTATTCTCTTAGCCGGTATTTAAACATACAAATTCACCCAAATTCTACCTT |
| | | GATAAGGTAATTGATTAATTTCATAAATGAATTCGCG |
| GAP promoter | SEQ ID NO: | TTTTTGTAGAAATGTCTTGGTGTCCTCGTCCAATCAGGTAGCCATCTCTGAAATATCTGG |
| | 52 | CTCCGTTGCAACTCCGAACGACCTGCTGGCAACGTAAAATTCTCCGGGGTAAAACTTAA |
| | | ATGTGGAGTAATGGAACCAGAAACGTCTCTTCCCTTCTCTCTCCTTCCACCGCCCGTTA |
| | | CCGTCCCTAGGAAATTTTACTCTGCTGGAGAGCTTCTTCTACGGCCCCCTTGCAGCAAT |
| | | GCTCTTCCCAGCATTACGTTGCGGGTAAAACGGAGGTCGTGTACCCGACCTAGCAGCCC |
| | | AGGGATGGAAAAGTCCCGGCCGTCGCTGGCAATAATAGCGGGCGGACGCATGTCATGA |
| | | GATTATTGGAAACCACCAGAATCGAATATAAAAGGCGAACACCTTTCCCAATTTTGGTT |
| | | TCTCCTGACCCAAAGACTTTAAATTTAATTTATTTGTCCCTATTTCAATCAATTGAACAA |
| | | CTAT |
| PGK promoter | SEQ ID NO: | AAATAGCAGTTTGCGGTTTCTTGATTTCATGGGGGGAACAAACAATAGTGTTGCCTTAA |
| | 53 | TTCTAATTGGCATTGTTGCTTGGAATCGAAATTGGGGGATAACGTCATATCTGAAAAGT |
| | | AAACAACTTCGGGAAATCAGGCTGTTTGAATGGCTTGGAAGCGAGATAGAAAGGGGAT |
| | | AGCGAGATAGAGGGGGCGGAGTAGACGAAGGGTGTTAAACTGCTGAAATCTCTCAATC |
| | | TGGAAGAAACGGAATAAATTAACTCCTTGCGATAATAAAATCCGAGTCCGTTATGACC |
| | | CCACACCGTGTTGACCACGGCATACCCCATGGAATCTGGTACAAAGCGTCAGTCTTGA |
| | | AGACACCATCACGTGTAGGAGACTGATTGTCTGACCGTCCAGCAAAAAGGGCATTATA |
| | | AATCTTGCTGTTAAAGGGGTGAGGGGAGATGCAGGTTGTTCTTTTATTCGCCTTGAACT |
| | | TTTTAATTTTCCCGGGGTTGCGGAGCGTGAACAGTTAGCCCGATCTGATAGCTTGCAAG |
| | | ATTCAACAGTTTATCCACTACAGGTCAGAGAGATCGCCGCAGAAGAAATGCTCGTCTC |
| | | GTGTTCCAGCACACATACTGGTGAAGTCGTTATTTTGCCGAAGGGGGGGTAATAAGGTT |
| | | ATGCACCCCCTCTCCACACCCCAGAATCATTTTTTAGCTGGGTTCAAGGCATTAGACTT |
| | | TGCACATTTTTCCCTTAAACACCCTTGAAACGCGGATAAACAGTTGCATGTGCATCCTA |
| | | AAACTAGGTGAGATGCGTACTCCGTGCTCCGATAATAACAGTGGTGTTGGGGTTGCTGC |
| | | TAGCTCACGCACTCCGTTCTTTTTTTTCAACCAGCAAAATTCGATGGGGAGAAACTTGG |
| | | GGTACTTTGCCGACTCCTCCACCATGCTGGTATATAAATAATACTCGCCCACTTTTCGTT |
| | | TGCTGCTTTTATATTTCATAGACTGAAAAAGACTCTTCTTCTACTTTTTCATAATATATC |
| | | TCAGATATCACTACTATAG |
| TEFg_promoter | SEQ ID NO: | GCGATTTAAATTCGCGAAAGAACAGCCTAATAAACTCCGAAGCATGATGGCCTCTATC |
| | 54 | CGGAAAACGTTAAGAGATGTGGCAACAGGAGGGCACATAGAATTTTTAAAGACGCTGA |
| | | AGAATGCTATCATAGTCCGTAAAAATGTGATAGTACTTTGTTTAGTGCGTACGCCACTT |
| | | ATTCGGGGCCAATAGCTAAACCCAGGTTTGCTGGCAGCAAATTCAACTGTAGATTGAA |
| | | TCTCTCTAACAATAATGGTGTTCAATCCCCTGGCTGGTCACGGGGAGGACTATCTTGCG |
| | | TGATCCGCTTGGAAAATGTTGTGTATCCCTTTCTCAATTGCGGAAAGCATCTGCTACTTC |
| | | CCATAGGCACCAGTTACCCAATTGATATTTCCAAAAAAGATTACCATATGTTCATCTAG |
| | | AAGTATAAATACAAGTGGACATTCAATGAATATTTCATTCAATTAGTCATTGACACTTT |
| | | CATCAACTTACTACGTCTTATTCAACAATGAATTCGCG |
| PMP20 promoter | SEQ ID NO: | ACACAGTTATTATTCATTTAAATGTCAAAACAGTAGTGATAAAAGGCTATGAAGGAGG |
| | 55 | TTGTCTAGGGGCTCGCGGAGGAAAGTGATTCAAACAGACCTGCCAAAAAGAGAAAAA |
| | | AGAGGGAATCCCTGTTCTTTCCAATGGAAATGACGTAACTTTAACTTGAAAAATACCCC |
| | | AACCAGAAGGGTTCAAACTCAACAAGGATTGCGTAATTCCTACAAGTAGCTTAGAGCT |
| | | GGGGGAGAGACAACTGAAGGCAGCTTAACGATAACGCGGGGGGATTGGTGCACGACT |
| | | CGAAAGGAGGTATCTTAGTCTTGTAACCTCTTTTTTCCAGAGGCTATTCAAGATTCATA |
| | | GGCGATATCGATGTGGAGAAGGGTGAACAATATAAAAGGCTGGAGAGATGTCAATGA |
| | | AGCAGCTGGATAGATTTCAAATTTTCTAGATTTCAGAGTAATCGCACAAAACGAAGGA |
| | | ATCCCACCAAGACAAAAAAAAAAATTCTAAGGAATTCCGAAACG |
| SHB17 promoter | SEQ ID NO: | AAATTCTTTTTACGTGGTGCGCATACTGGACAGAGGCAGAGTCTCAATTTCTTCTTTTGA |
| | 56 | GACAGGCTACTACAGCCTGTGATTCCTCTTGGTACTTGGATTTGCTTTTATCTGGCTCCG |
| | | TTGGGAACTGTGCCTGGGTTTTGAAGTATCTTGTGGATGTGTTTCTAACACTTTTTCAAT |
| | | CTTCTTGGAGTGAGAATGCAGGACTTTGAACATCGTCTAGCTCGTTGGTAGGTGAACCG |
| | | TTTTACCTTGCATGTGGTTAGGAGTTTTCTGGAGTAACCAAGACCGTCTTATCATCGCCG |
| | | TAAAATCGCTCTTACTGTCGCTAATAATCCCGCTGGAAGAGAAGTTCGAACAGAAGTA |
| | | GCACGCAAAGCTCTTGTCAAATGAGAATTGTTAATCGTTTGACAGGTCACACTCGTGGG |
| | | CTATGTACGATCAACTTGCCGGCTGTTGCTGGAGAGATGACACCAGTTGTGGCATGGCC |
| | | AATTGGTATTCAGCCGTACCACTGTATGGAAAATGAGATTATCTTGTTCTTGATCTAGTT |
| | | TCTTGCCATTTTAGAGTTGCCACATTCGTAGGTTTCAGTACCAATAATGGTAACTTCCAA |
| | | ACTTCCAACGCAGATACCAGAGATCTGCCGATCCTTCCCCAACAATAGGAGCTTACTAC |
| | | GCCATACATATAGCCTATCTATTTTCACTTTCGCGTGGGTGCTTCTATATAAACGGTTCC |
| | | CCATCTTCCGTTTCATACTACTTGAATTTTAAGCACTAAAGAATT |
| PEX8 promoter | SEQ ID NO: | AAATTAACCAGTGTTTTCTTATCTATTTGTCTTTTTACACTAAAGTGAAGTACGAATCCA |
| | 57 | TGCGATTGATTCCTCCTCAGATATCAGCTGAATTCTTGCTTATGTAATACTTGCGCGAAC |
| | | TACATGTGAACTTAGGATTCGATAAGGCTGGGGGGTCAACCAACCCCACTTCAAAGAG |
| | | CCGACCCGTATAAATAGCCTCTGCGTCCTCAGATCAACAAGACGAAGCAATTTTTTTTT |
| | | ACCTATCTTCAGGTGCCTGTTAG |
| PEX4 promoter | SEQ ID NO: | AGGGAGGCAATTAGTTGTCCTTGTGGAATCAAAAGAGCACAAGAAACCTGTGATTGAA |
| | 58 | AGTCTGGGCTGTCTGGGGTTGGCAAGAAAATCATAAAGTTTATATAGTACATTTGTTAG |
| | | TTGCTTCTTTGAATGACACCTTGATCTACATGTTGTTCTTCCCAGTTCCCACCGCGAAGT |
| | | TTCTCTAACTCTCAATCTCTCTTTCCCCACTTGATAATCCAAAGAA |
| TKL3 Promoter | SEQ ID NO: | gtcgaggaaagggtcgtttcggggagttaaatatttttggctatgtagcagacatgtttcgacgctggcgtcgcgtcgatcggaaaatattacccca |
| | 59 | ggaacaagcacttgcttgggttagccaccaccctgcgcaagcctttttgccggctctacacagggccaatgaaatctgggcggaatctgaaacc |
| | | gatgaaacggacgacactggcaacaagctcactgcactattttttttttctagtgaaatagcctatcctcgtctcgctcccctcatacctgtaaaggg |
| | | gtgcaatttagcctcgttccagccattcacgggccactcaacaacacgtcggctaccatggggtgcttgggcaccaaaaggcctataaataggc |
| | | ccccatccgtctgctacacagtcatctctgtcttttcttccc |
| AOX1 terminator | SEQ ID NO: | TCAAGAGGATGTCAGAATGCCATTTGCCTGAGAGATGCAGGCTTCATTTTTGATACTTT |
| | 60 | TTTATTTGTAACCTATATAGTATAGGATTTTTTTTGTCATTTTGTTTCTTCTCGTACGAGC |
| | | TTGCTCCTGATCAGCCTATCTCGCAGCAGATGAATATCTTGTGGTAGGGGTTTGGGAAA |
| | | ATCATTCGAGTTTGATGTTTTTCTTGGTATTTCCCACTCCTCTTCAGAGTACAGAAGATT |
| | | AAGTGAAACCTTCGTTTGTGCG |
| TDH3 terminator | SEQ ID NO: | TCGATTTGTATGTGAAATAGCTGAAATTCGAAAATTTCATTATGGCTGTATCTACTTTAG |
| | 61 | CGTATTAGGCATTTGAGCATTGGCTTGAACAATGCGGGCTGTAGTGTGTCACCAAAGAA |
| | | ACCATTCGGGTTCGGATCTGGAAGTCCTCATCACGTGATGCCGATCTCGTGTATTTTATT |
| | | TTCAGATAACACCTGAAGACTTT |
| RPS25A terminator | SEQ ID NO: | ATTAGTGTACATCTGATAATATAGTACTACCACGTATGATAATGTAGAGAATAGTCTTC |
| | 62 | CTTGTCGAGTGTGTTTGCAGTTTTCTTGAGTTTCAAGGTTTAAATGCTGGTATATTAGTT |
| | | CATCGAAGGTTTCAGCCAATAGCACCTTAAATCAATCAAACTAATTCGACTCTTACGAA |
| | | AGAGCCTACTGTGTTTAGTATCGAAGTCGTTTACCTTTCATGTTGAATAGCTTCCTCTCT |
| | | GACCCTAACATTTCAAGATCCTCCTAAAGTTACCCGGATTGTGAAATTCTAATGATCCA |
| | | CCTGCCCAATGCATTTTTTCTTTATTCAGTTTACCTTTTTTACCTAATATACGAGCTTGTT |
| | | AAAGTAAGTGGCACTGCAATACTAGGCTTATTGTTGATATTATGATGAATCGTTTTCAC |
| | | AAACTTGATTTCCTGTGAACTCACCATGTACTAAGGAAAAAAACATGCATCACCATCTG |
| | | AATATTTGAC |
| RPL2A terminator | SEQ ID NO: | ACTATGTAACTAACGAAACAGCATGTACTAATAGAACCGTATCGAGAATATTTATTTAG |
| | 63 | GTGAGTAGTAGGAGTGAACCAGACAGTCAATTTAGTGAGCTGTCCCAGCTTTTGTGCAT |
| | | TCCAGAATTGCCGGTCAAATTGGTTATGGGTTATGGGGCTTTTCCGATTGAGGTTCAGT |
| | | TTCTGCGGTTATCTCTTTCTTGACCTGGTCTTTTACAGGCTGTTCTTTCTCCCCATGATTA |
| | | TTCTTTAGCTGAAGATACCGCTTAGCCTGATAATGTCGTCGTTTTGTAATCAAAATCTTT |
| | | AGTTGGGCATCGTCTGAGGTTTCCTTTGGCTTCTGGGGTTGTTAGTAGGAACGTAGGAA |
| | | CCATAGTAACTTTTACACATACATTCTTATGATTGCGAAGTAAGCTGAGTCTGCTGCTT |
| | | GGCTCCCGAAGTACTTTCTCTTTCTCTACCGGTTGATTCTCCTTCTGGTGCTCCTAAACG |
| | | ATTGTGTTAGAAGGGATTGAC |
| Signal Peptide | SEQ ID NO: | MFTPVRRRVRTAALALSAAAALVLGSTAASGASATPSPAPAP |
| | 64 | |
| Signal Peptide | SEQ ID NO: | MKLSTVLLSAGLASTTLA |
| | 65 | |
| Signal Peptide | SEQ ID NO: | MRFPSIFTAVLFAASSALA |
| | 66 | |
| Signal Peptide | SEQ ID NO: | MVSLRSIFTSSILAAGLTRAHG |
| | 67 | |
| Signal Peptide | SEQ ID NO: | MKFPVPLLFLLQLFFIIATQG |
| | 68 | |
| Signal Peptide | SEQ ID NO: | MQVKSIVNLLLACSLAVA |
| | 69 | |
| Signal Peptide | SEQ ID NO: | MQFNWNIKTVASILSALTLAQA |
| | 70 | |
| Signal Peptide | SEQ ID NO: | MYRNLIIATALTCGAYSAYVPSEPWSTLTPDASLESALKDYSQTFGIAIKSLDADKIKR |
| | 71 | |
| Signal Peptide | SEQ ID NO: | MNLYLITLLFASLCSAITLPKR |
| | 72 | |
| Signal Peptide | SEQ ID NO: | MFEKSKFVVSFLLLLQLFCVLGVHG |
| | 73 | |
| Signal Peptide | SEQ ID NO: | MQFNSVVISQLLLTLASVSMG |
| | 74 | |
| Signal Peptide | SEQ ID NO: | MKSQLIFMALASLVASAPLEHQQQHHKHEKR |
| | 75 | |
| Signal Peptide | SEQ ID NO: | MKFAISTLLIILQAAAVFA |
| | 76 | |
| Signal Peptide | SEQ ID NO: | MKLLNFLLSFVTLFGLLSGSVFA |
| | 77 | |
| Signal Peptide | SEQ ID NO: | MIFNLKTLAAVAISISQVSA |
| | 78 | |
| Signal Peptide | SEQ ID NO: | MKISALTACAVTLAGLAIAAPAPKPEDCTTTVQKRHQHKR |
| | 79 | |
| Signal Peptide | SEQ ID NO: | MSYLKISALLSVLSVALA |
| | 80 | |
| Signal Peptide | SEQ ID NO: | MLSTILNIFILLLFIQASLQ |
| | 81 | |
| Signal Peptide | SEQ ID NO: | MKLSTNLILAIAAASAVVSAAPVAPAEEAANHLHKR |
| | 82 | |
| Signal Peptide | SEQ ID NO: | MFKSLCMLIGSCLLSSVLA |
| | 83 | |
| Signal Peptide | SEQ ID NO: | MKLAALSTIALTILPVALA |
| | 84 | |
| Signal Peptide | SEQ ID NO: | MSFSSNVPQLFLLLVLLTNIVSG |
| | 85 | |
| Signal Peptide | SEQ ID NO: | MQLQYLAVLCALLLNVQSKNVVDFSRFGDAKISPDDTDLESRERKR |
| | 86 | |
| Signal Peptide | SEQ ID NO: | MKIHSLLLWNLFFIPSILG |
| | 87 | |
| Signal Peptide | SEQ ID NO: | MSTLTLLAVLLSLQNSALA |
| | 88 | |
| Signal Peptide | SEQ ID NO: | MINLNSFLILTVTLLSPALALPKNVLEEQQAKDDLAKR |
| | 89 | |
| Signal Peptide | SEQ ID NO: | MFSLAVGALLLTQAFG |
| | 90 | |
| Signal Peptide | SEQ ID NO: | MKILSALLLLFTLAFA |
| | 91 | |
| Signal Peptide | SEQ ID NO: | MKVSTTKFLAVFLLVRLVCA |
| | 92 | |
| Signal Peptide | SEQ ID NO: | MQFGKVLFAISALAVTALG |
| | 93 | |
| Signal Peptide | SEQ ID NO: | MWSLFISGLLIFYPLVLG |
| | 94 | |
| Signal Peptide | SEQ ID NO: | MRNHLNDLVVLFLLLTVAAQA |
| | 95 | |
| Signal Peptide | SEQ ID NO: | MFLKSLLSFASILTLCKA |
| | 96 | |
| Signal Peptide | SEQ ID NO: | MFVFEPVLLAVLVASTCVTA |
| | 97 | |
| Signal Peptide | SEQ ID NO: | MFSPILSLEIILALATLQSVFA |
| | 98 | |
| Signal Peptide | SEQ ID NO: | MIINHLVLTALSIALA |
| | 99 | |
| Signal Peptide | SEQ ID NO: | MLALVRISTLLLLALTASA |
| | 100 | |
| Signal Peptide | SEQ ID NO: | MRPVLSLLLLLASSVLA |
| | 101 | |
| Signal Peptide | SEQ ID NO: | MVLIQNFLPLFAYTLFFNQRAALA |
| | 102 | |
| Signal Peptide | SEQ ID NO: | MVSLTRLLITGIATALQVNA |
| | 103 | |
| Signal Peptide | SEQ ID NO: | MIFDGTTMSIAIGLLSTLGIGAEA |
| | 104 | |
| Signal Peptide | SEQ ID NO: | MVLVGLLTRLVPLVLLAGTVLLLVFVVLSGG |
| | 105 | |
| Signal Peptide | SEQ ID NO: | MLSILSALTLLGLSCA |
| | 106 | |
| Signal Peptide | SEQ ID NO: | MRLLHISLLSIISVLTKANA |
| | 107 | |
| Signal Peptide | SEQ ID NO: | MRFPSIFTAVLFAASSALAAPVNTTTEDETAQIPAEAVIGYLDLEGDFDVAVLPFSNSTNNG |
| | 108 | LLFINTTIASIAAKEEGVSLDKREAEA |
| Signal Peptide | SEQ ID NO: | MFKSVVYSILAASLANA |
| | 109 | |
| Signal Peptide | SEQ ID NO: | MLLQAFLFLLAGFAAKISA |
| | 110 | |
| Signal Peptide | SEQ ID NO: | MASSNLLSLALFLVLLTHANS |
| | 111 | |
| Signal Peptide | SEQ ID NO: | MNIFYIFLFLLSFVQGLEHTHRRGSLVKR |
| | 112 | |
| Signal Peptide | SEQ ID NO: | MLIIVLLFLATLANSLDCSGDVFFGYTRGDKTDVHKSQALTAVKNIKR |
| | 113 | |
| Signal Peptide | SEQ ID NO: | MESVSSLFNIFSTIMVNYKSLVLALLSVSNLKYARGMPTSERQQGLEER |
| | 114 | |
| Signal Peptide | SEQ ID NO: | MFAFYFLTACISLKGVFG |
| | 115 | |
| Signal Peptide | SEQ ID NO: | MRFSTTLATAATALFFTASQVSA |
| | 116 | |
| Signal Peptide | SEQ ID NO: | MKFAYSLLLPLAGVSASVINYKR |
| | 117 | |
| Signal Peptide | SEQ ID NO: | MKFFAIAALFAAAAVAQPLEDR |
| | 118 | |
| Signal Peptide | SEQ ID NO: | MQFFAVALFATSALA |
| | 119 | |
| Signal Peptide | SEQ ID NO: | MKWVTFISLLFLFSSAYSRGVFRR |
| | 120 | |
| Signal Peptide | SEQ ID NO: | MRSLLILVLCFLPLAALG |
| | 121 | |
| Signal Peptide | SEQ ID NO: | MKVLILACLVALALA |
| | 122 | |
| Signal Peptide | SEQ ID NO: | MFNLKTILISTLASIAVA |
| | 123 | |
| Signal Peptide | SEQ ID NO: | MYRKLAVISAFLATARAQSA |
| | 124 | |
| WT | SEQ ID NO: | MRFPSIFTAVLFAASSALAAPVNTTTEDETAQIPAEAVIGYLDLEGDFDVAVLPFSNSTNNG |
| | 125 | LLFINTTIASIAAKEEGVQLDKR |
| App3 | SEQ ID NO: | MRFPPIFTAALFAASSALAAPANTTTEDETAQIPAEAVIGYLDSEGDSDVAVLPFSNSTNNG |
| | 126 | LSFINTTIASIAAKEEGVQLDKR |
| App8 | SEQ ID NO: | MRFPSIFTAVLFAASSALAAPANTTTEDETAQIPAEAVISYSDLEGDFDAAALPLSNSTNNGL |
| | 127 | SSTNTTIASIAAKEEGVQLDKR |
| App9 | SEQ ID NO: | MRPPSIFTAVLFAASSALAAPANTTTEDETTQIPAEAVATYLDLEGDVDVAVLPFSSSTNNG |
| | 128 | LSFINTTIASIAAKEEGVQLDKR |
| App10 | SEQ ID NO: | MRFPSIFTAALFAASSALAAPANTTTEGETAQTPAEAVIGYRDLEGDFDVAVLPFPNSTNNG |
| | 129 | LLFTNTTTASIAAKEEGVQLDKR |
| appS1 | SEQ ID NO: | MRFPSIFTAVLLAAPSALAAPANATTEDEAAQIPAEAVIGYLDLEGDFDAAVLPFSNSTNNG |
| | 130 | LLSINTTIASIAAKEEGVQLDKR |
| appS4 | SEQ ID NO: | MRFPSIFTAVVFAASSALAAPANTTAEDETAQIPAEAVIGYLGLEGDSDVAALPLSDSTNNG |
| | 131 | SLSTNTTIASIAAKEEGVQLDKR |
| appS6 | SEQ ID NO: | MRLPSIFTAAVFAASSALAAPANTTTEDETAQIPAEAAIGYLDLEGDSDVAVLPLSNSTNNG |
| | 132 | LLFINTTIASIAAKEEGVQLDKR |
| appS8 | SEQ ID NO: | MRFPSIFTAVLFAASSALAAPANTTTEDETAQIPAEAVIGYLDLEGDFDVAVLPFSNSTNDG |
| | 133 | LSFINTTTASIAAKEEGVQLDKR |
| a-Factor | SEQ ID NO: | MRFPSIFTAVLFAASSALAAPVNTTTEDETAQIPA |
| | 134 | |
| PpScw11p | SEQ ID NO: | MLSTILNIFILLLFIQASLQAPIPVVTKYVTEGIAVV |
| | 135 | |
| PpDse4p | SEQ ID NO: | MSFSSNVPQLFLLLVLLTNIVSGAVISVWSTSKVTK |
| | 136 | |
| PpExg1p | SEQ ID NO: | MNLYLITLLFASLCSAITLPKRDIIWDYSSEKIMG |
| | 137 | |
| a-EGFP | SEQ ID NO: | MRFPSIFTAVLFAASSALAAPVNTTTEDETAQIPA |
| | 138 | |
| S-EGFP | SEQ ID NO: | MLSTILNIFILLLFIQASLQEFDYKDDDDKMVSKG |
| | 139 | |
| D-EGFP | SEQ ID NO: | MSFSSNVPQLFLLLVLLTNIVSGEFDYKDDDDKMV |
| | 140 | |
| E-EGFP | SEQ ID NO: | MNLYLITLLFASLCSAEFDYKDDDDKMVSKGEELF |
| | 141 | |
| a-CALB | SEQ ID NO: | MRFPSIFTAVLFAASSALAAPVNTTTEDETAQIPA |
| | 142 | |
| S-CALB | SEQ ID NO: | MLSTILNIFILLLFIQASLQEFLPSGSDPAFSQPK |
| | 143 | |
| D-CALB | SEQ ID NO: | MSFSSNVPQLFLLLVLLTNIVSGEFLPSGSDPAFS |
| | 144 | |
| E-CALB | SEQ ID NO: | MNLYLITLLFASLCSAEFLPSGSDPAFSQPKSVLD |
| | 145 | |
| Amylase (AA) | SEQ ID NO: | MVAWWSLFLYGLQVAAPALAAEVDCSRFPNATDKEGKDVLVCNKDLRPICGTDGVTYTN |
| | 146 | DCLLCAYSIEFGTNISKEHDGECKETVPMNCSSYANTTSEDGKVMVLCNRAFNPVCGTDG |
| | | VTYDNECLLCAHKVEQGASVDKRHDGGCRKELAAVSVDCSEYPKPDCTAEDRPLCGSDN |
| | | KTYGNKCNFCNAVVESNGTLTLSHFGKC |
| Alpha K (AK) | SEQ ID NO: | MRFPSIFTAVLFAASSALAAPVNTTTEDELEGDFDVAVLPFSASIAAKEEGVSLEKRAEVDC |
| | 147 | SRFPNATDKEGKDVLVCNKDLRPICGTDGVTYTNDCLLCAYSIEFGTNISKEHDGECKETVP |
| | | MNCSSYANTTSEDGKVMVLCNRAFNPVCGTDGVTYDNECLLCAHKVEQGASVDKRHDG |
| | | GCRKELAAVSVDCSEYPKPDCTAEDRPLCGSDNKTYGNKCNFCNAVVESNGTLTLSHFGK |
| | | C |
| Alpha T (AT) | SEQ ID NO: | MRFPSIFTAVLFAASSALAAEVDCSRFPNATDKEGKDVLVCNKDLRPICGTDGVTYTNDCL |
| | 148 | LCAYSIEFGTNISKEHDGECKETVPMNCSSYANTTSEDGKVMVLCNRAFNPVCGTDGVTY |
| | | DNECLLCAHKVEQGASVDKRHDGGCRKELAAVSVDCSEYPKPDCTAEDRPLCGSDNKTY |
| | | GNKCNFCNAVVESNGTLTLSHFGKC |
| Lysozyme (LZ) | SEQ ID NO: | MLGKNDPMCLVLVLLGLTALLGICQGAEVDCSRFPNATDKEGKDVLVCNKDLRPICGTDG |
| | 149 | VTYTNDCLLCAYSIEFGTNISKEHDGECKETVPMNCSSYANTTSEDGKVMVLCNRAFNPVC |
| | | GTDGVTYDNECLLCAHKVEQGASVDKRHDGGCRKELAAVSVDCSEYPKPDCTAEDRPLC |
| | | GSDNKTYGNKCNFCNAVVESNGTLTLSHFGKC |
| Killer Protein (KP) | SEQ ID NO: | MTKPTQVLVRSVSILFFITLLHLVVAAEVDCSRFPNATDKEGKDVLVCNKDLRPICGTDGV |
| | 150 | TYTNDCLLCAYSIEFGTNISKEHDGECKETVPMNCSSYANTTSEDGKVMVLCNRAFNPVCG |
| | | TDGVTYDNECLLCAHKVEQGASVDKRHDGGCRKELAAVSVDCSEYPKPDCTAEDRPLCG |
| | | SDNKTYGNKCNFCNAVVESNGTLTLSHFGKC |
| Invertase (IV) | SEQ ID NO: | MLLQAFLFLLAGFAAKISAAEVDCSRFPNATDKEGKDVLVCNKDLRPICGTDGVTYTNDCL |
| | 151 | LCAYSIEFGTNISKEHDGECKETVPMNCSSYANTTSEDGKVMVLCNRAFNPVCGTDGVTY |
| | | DNECLLCAHKVEQGASVDKRHDGGCRKELAAVSVDCSEYPKPDCTAEDRPLCGSDNKTY |
| | | GNKCNFCNAVVESNGTLTLSHFGKC |
| Serum Albumin (SA) | SEQ ID NO: | MKWVTFISLLFLFSSAYSAEVDCSRFPNATDKEGKDVLVCNKDLRPICGTDGVTYTNDCLL |
| | 152 | CAYSIEFGTNISKEHDGECKETVPMNCSSYANTTSEDGKVMVLCNRAFNPVCGTDGVTYD |
| | | NECLLCAHKVEQGASVDKRHDGGCRKELAAVSVDCSEYPKPDCTAEDRPLCGSDNKTYG |
| | | NKCNFCNAVVESNGTLTLSHFGKC |
| Glucoamyl (GA) | SEQ ID NO: | MSFRSLLALSGLVCSGLAAEVDCSRFPNATDKEGKDVLVCNKDLRPICGTDGVTYTNDCLL |
| | 153 | CAYSIEFGTNISKEHDGECKETVPMNCSSYANTTSEDGKVMVLCNRAFNPVCGTDGVTYD |
| | | NECLLCAHKVEQGASVDKRHDGGCRKELAAVSVDCSEYPKPDCTAEDRPLCGSDNKTYG |
| | | NKCNFCNAVVESNGTLTLSHFGKC |
| Inulase (IN)-IC | SEQ ID NO: | MKLAYSLLLPLAGVSAAEVDCSRFPNATDKEGKDVLVCNKDLRPICGTDGVTYTNDCLLC |
| | 154 | AYSIEFGTNISKEHDGECKETVPMNCSSYANTTSEDGKVMVLCNRAFNPVCGTDGVTYDN |
| | | ECLLCAHKVEQGASVDKRHDGGCRKELAAVSVDCSEYPKPDCTAEDRPLCGSDNKTYGN |
| | | KCNFCNAVVESNGTLTLSHFGKC |
| Alpha KS (AKS) | SEQ ID NO: | MRFPSIFTAVLFAASSALAAPVNTTTEDELEGDFDVAVLPFSASIAAKEEGVSLEKREAEAA |
| | 155 | EVDCSRFPNATDKEGKDVLVCNKDLRPICGTDGVTYTNDCLLCAYSIEFGTNISKEHDGEC |
| | | KETVPMNCSSYANTTSEDGKVMVLCNRAFNPVCGTDGVTYDNECLLCAHKVEQGASVDK |
| | | RHDGGCRKELAAVSVDCSEYPKPDCTAEDRPLCGSDNKTYGNKCNFCNAVVESNGTLTLS |
| | | HFGKC |
| Ovomucoid signal | SEQ ID NO: | MAMAGVFVLFSFVLCGFLPDAAFG |
| peptide | 156 | |
| Lysozyme signal | SEQ ID NO: | MRSLLILVLCFLPLAALG |
| peptide | 157 | |
| Ovalbumin Signal | SEQ ID NO: | MRFPSIFTAVLFAASSALAAPVNTTTEDETAQIPAEAVIGYSDLEGDFDVAVLPFSNSTNNG |
| Peptide | 158 | LLFINTTIASIAAKEEGVSLDKREAEA |
| Ovotransferrin Signal | SEQ ID NO: | MKLILCTVLSLGIAAVCFA |
| Peptide | 159 | |
| Bovine Lactoferrin | SEQ ID NO: | MKLFVPALLSLGALGLCLA |
| Signal Peptide | 160 | |
| Porcine Lactoferrin | SEQ ID NO: | MKLFIPALLFLGTLGLCLA |
| Signal Peptide | 161 | |
| Kid Lipase Signal | SEQ ID NO: | MESKALLLLALSVWLQSLTVSHG |
| Peptide | 162 | |
| Porcine Lipase | SEQ ID NO: | MLLIWTLSLLLGAVLG |
| Signal Peptide | 163 | |
| Ovomucoid | SEQ ID NO: | AEVDCSRFPNATDKEGKDVLVCNKDLRPICGTDGVTYTNDCLLCAYSIEFGTNISKEHDGE |
| (canonical) | 164 | CKETVPMNCSSYANTTSEDGKVMVLCNRAFNPVCGTDGVTYDNECLLCAHKVEQGASVD |
| | | KRHDGGCRKELAAVSVDCSEYPKPDCTAEDRPLCGSDNKTYGNKCNFCNAVVESNGTLTL |
| | | SHFGKC* |
| Ovomucoid | SEQ ID NO: | AEVDCSRFPNATDMEGKDVLVCNKDLRPICGTDGVTYTNDCLLCAYSVEFGTNISKEHDG |
| | 165 | ECKETVPMNCSSYANTTSEDGKVMVLCNRAFNPVCGTDGVTYDNECLLCAHKVEQGASV |
| | | DKRHDGGCRKELAAVSVDCSEYPKPDCTAEDRPLCGSDNKTYGNKCNFCNAVVESNGTL |
| | | TLSHFGKC* |
| Ovomucoid | SEQ ID NO: | AEVDCSRFPNATDMEGKDVLVCNKDLRPICGTDGVTYTNDCLLCAYSVEFGTNISKEHDG |
| G162M F167A | 166 | ECKETVPMNCSSYANTTSEDGKVMVLCNRAFNPVCGTDGVTYDNECLLCAHKVEQGASV |
| | | DKRHDGGCRKELAAVSVDCSEYPKPDCTAEDRPLCGSDNKTYMNKCNACNAVVESNGTL |
| | | TLSHFGKC* |
| Ovomucoid isoform | SEQ ID NO: | MAMAGVFVLFSFVLCGFLPDAAFGAEVDCSRFPNATDKEGKDVLVCNKDLRPICGTDGVT |
| 1 precursor full | 167 | YTNDCLLCAYSIEFGTNISKEHDGECKETVPMNCSSYANTTSEDGKVMVLCNRAFNPVCGT |
| length | | DGVTYDNECLLCAHKVEQGASVDKRHDGGCRKELAAVSVDCSEYPKPDCTAEDRPLCGS |
| | | DNKTYGNKCNFCNAVVESNGTLTLSHFGKC |
| Ovomucoid [Gallus | SEQ ID NO: | MAMAGVFVLFSFVLCGFLPDAVFGAEVDCSRFPNATDMEGKDVLVCNKDLRPICGTDGVT |
| gallus] | 168 | YTNDCLLCAYSVEFGTNISKEHDGECKETVPMNCSSYANTTSEDGKVMVLCNRAFNPVCG |
| | | TDGVTYDNECLLCAHKVEQGASVDKRHDGGCRKELAAVSVDCSEYPKPDCTAEDRPLCG |
| | | SDNKTYGNKCNFCNAVVESNGTLTLSHFGKC |
| Ovomucoid isoform | SEQ ID NO: | MAMAGVFVLFSFVLCGFLPDAAFGAEVDCSRFPNATDKEGKDVLVCNKDLRPICGTDGVT |
| 2 precursor [Gallus | 169 | YTNDCLLCAYSIEFGTNISKEHDGECKETVPMNCSSYANTTSEDGKVMVLCNRAFNPVCGT |
| gallus] | | DGVTYDNECLLCAHKVEQGASVDKRHDGGCRKELAAVDCSEYPKPDCTAEDRPLCGSDN |
| | | KTYGNKCNFCNAVVESNGTLTLSHFGKC |
| Ovomucoid [Gallus | SEQ ID NO: | AEVDCSRFPNATDKEGKDVLVCNKDLRPICGTDGVTYNNECLLCAYSIEFGTNISKEHDGE |
| gallus] | 170 | CKETVPMNCSSYANTTSEDGKVMVLCNRAFNPVCGTDGVTYDNECLLCAHKVEQGASVD |
| | | KRHDGECRKELAAVSVDCSEYPKPDCTAEDRPLCGSDNKTYGNKCNFCNAVVESNGTLTL |
| | | SHFGKC |
| Ovomucoid [Numida | SEQ ID NO: | MAMAGVFVLFSFALCGFLPDAAFGVEVDCSRFPNATNEEGKDVLVCTEDLRPICGTDGVT |
| meleagris] | 171 | YSNDCLLCAYNIEYGTNISKEHDGECREAVPVDCSRYPNMTSEEGKVLILCNKAFNPVCGT |
| | | DGVTYDNECLLCAHNVEQGTSVGKKHDGECRKELAAVDCSEYPKPACTMEYRPLCGSDN |
| | | KTYDNKCNFCNAVVESNGTLTLSHFGKC |
| PREDICTED: | SEQ ID NO: | MQTITWRQPQGDHLRSRAPAATCRAGQYLTMAMAGIFVLFSFALCGFLPDAAFGVEVDCS |
| Ovomucoid isoform | 172 | RFPNTTNEEGKDVLVCTEDLRPICGTDGVTHSECLLCAYNIEYGTNISKEHDGECREAVPM |
| X1 [Meleagris | | DCSRYPNTTNEEGKVMILCNKALNPVCGTDGVTYDNECVLCAHNLEQGTSVGKKHDGGC |
| gallopavo] | | RKELAAVSVDCSEYPKPACTLEYRPLCGSDNKTYGNKCNFCNAVVESNGTLTLSHFGKC |
| Ovomucoid | SEQ ID NO: | VEVDCSRFPNTTNEEGKDVLVCTEDLRPICGTDGVTHSECLLCAYNIEYGTNISKEHDGECR |
| [Meleagris | 173 | EAVPMDCSRYPNTTSEEGKVMILCNKALNPVCGTDGVTYDNECVLCAHNLEQGTSVGKK |
| gallopavo] | | HDGECRKELAAVSVDCSEYPKPACTLEYRPLCGSDNKTYGNKCNFCNAVVESNGTLTLSH |
| | | FGKC |
| PREDICTED: | SEQ ID NO: | MQTITWRQPQGDHLRSRAPAATCRAGQYLTMAMAGIFVLFSFALCGFLPDAAFGVEVDCS |
| Ovomucoid isoform | 174 | RFPNTTNEEGKDVLVCTEDLRPICGTDGVTHSECLLCAYNIEYGTNISKEHDGECREAVPM |
| X2 [Meleagris | | DCSRYPNTTNEEGKVMILCNKALNPVCGTDGVTYDNECVLCAHNLEQGTSVGKKHDGGC |
| gallopavo] | | RKELAAVDCSEYPKPACTLEYRPLCGSDNKTYGNKCNFCNAVVESNGTLTLSHFGKC |
| Ovomucoid | SEQ ID NO: | EYGTNISIKHNGECKETVPMDCSRYANMTNEEGKVMMPCDRTYNPVCGTDGVTYDNECQ |
| [Bambusicola | 175 | LCAHNVEQGTSVDKKHDGVCGKELAAVSVDCSEYPKPECTAEERPICGSDNKTYGNKCNF |
| thoracicus] | | CNAVVYVQP |
| Ovomucoid | SEQ ID NO: | VDCSRFPNTTNEEGKDVLACTKELHPICGTDGVTYSNECLLCYYNIEYGTNISKEHDGECTE |
| [Callipepla | 176 | AVPVDCSRYPNTTSEEGKVLIPCNRDFNPVCGSDGVTYENECLLCAHNVEQGTSVGKKHD |
| squamata] | | GGCRKEFAAVSVDCSEYPKPDCTLEYRPLCGSDNKTYASKCNFCNAVVIWEQEKNTRHHA |
| | | SHSVFFISARLVC |
| Ovomucoid [Colinus | SEQ ID NO: | MLPLGLREYGTNTSKEHDGECTEAVPVDCSRYPNTTSEEGKVRILCKKDINPVCGTDGVTY |
| virginianus] | 177 | DNECLLCSHSVGQGASIDKKHDGGCRKEFAAVSVDCSEYPKPACMSEYRPLCGSDNKTYV |
| | | NKCNFCNAVVYVQPWLHSRCRLPPTGTSFLGSEGRETSLLTSRATDLQVAGCTAISAMEAT |
| | | RAAALLGLVLLSSFCELSHLCFSQASCDVYRLSGSRNLACPRIFQPVCGTDNVTYPNECSLC |
| | | RQMLRSRAVYKKHDGRCVKVDCTGYMRATGGLGTACSQQYSPLYATNGVIYSNKCTFCS |
| | | AVANGEDIDLLAVKYPEEESWISVSPTPWRMLSAGA |
| Ovomucoid-like | SEQ ID NO: | MSWWGIKPALERPSQEQSTSGQPVDSGSTSTTTMAGIFVLLSLVLCCFPDAAFGVEVDCSRF |
| isoform X2 [Anser | 178 | PNTTNEEGKEVLLCTKDLSPICGTDGVTYSNECLLCAYNIEYGTNISKDHDGECKEAVPVD |
| cygnoides | | CSTYPNMTNEEGKVMLVCNKMFSPVCGTDGVTYDNECMLCAHNVEQGTSVGKKYDGKC |
| domesticus] | | KKEVATVDCSDYPKPACTVEYMPLCGSDNKTYDNKCNFCNAVVDSNGTLTLSHFGKC |
| Ovomucoid-like | SEQ ID NO: | MSSQNQLHRRRRPLPGGQDLNKYYWPHCTSDRFSWLLHVTAEQFRHCVCIYLQPALERPS |
| isoform X1 [Anser | 179 | QEQSTSGQPVDSGSTSTTTMAGIFVLLSLVLCCFPDAAFGVEVDCSRFPNTTNEEGKEVLLC |
| cygnoides | | TKDLSPICGTDGVTYSNECLLCAYNIEYGTNISKDHDGECKEAVPVDCSTYPNMTNEEGKV |
| domesticus] | | MLVCNKMFSPVCGTDGVTYDNECMLCAHNVEQGTSVGKKYDGKCKKEVATVDCSDYPK |
| | | PACTVEYMPLCGSDNKTYDNKCNFCNAVVDSNGTLTLSHFGKC |
| Ovomucoid | SEQ ID NO: | VEVDCSRFPNTTNEEGKDEVVCPDELRLICGTDGVTYNHECMLCFYNKEYGTNISKEQDGE |
| [Coturnix japonica] | 180 | CGETVPMDCSRYPNTTSEDGKVTILCTKDFSFVCGTDGVTYDNECMLCAHNVVQGTSVGK |
| | | KHDGECRKELAAVSVDCSEYPKPACPKDYRPVCGSDNKTYSNKCNFCNAVVESNGTLTLN |
| | | HFGKC |
| Ovomucoid | SEQ ID NO: | MAMAGVFLLFSFALCGFLPDAAFGVEVDCSRFPNTTNEEGKDEVVCPDELRLICGTDGVTY |
| [Coturnix japonica] | 181 | NHECMLCFYNKEYGTNISKEQDGECGETVPMDCSRYPNTTSEDGKVTILCTKDFSFVCGTD |
| | | GVTYDNECMLCAHNIVQGTSVGKKHDGECRKELAAVSVDCSEYPKPACPKDYRPVCGSD |
| | | NKTYSNKCNFCNAVVESNGTLTLNHFGKC |
| Ovomucoid [Anas | SEQ ID NO: | MAGVFVLLSLVLCCFPDAAFGVEVDCSRFPNTTNEEGKDVLLCTKELSPVCGTDGVTYSNE |
| platyrhynchos] | 182 | CLLCAYNIEYGTNISKDHDGECKEAVPADCSMYPNMTNEEGKMTLLCNKMFSPVCGTDG |
| | | VTYDNECMLCAHNVEQGTSVGKKYDGKCKKEVATVDCSGYPKPACTMEYMPLCGSDNK |
| | | TYGNKCNFCNAVVDSNGTLTLSHFGEC |
| Ovomucoid, partial | SEQ ID NO: | QVDCSRFPNTTNEEGKEVLLCTKELSPVCGTDGVTYSNECLLCAYNIEYGTNISKDHDGEC |
| [Anas platyrhynchos] | 183 | KEAVPADCSMYPNMTNEEGKMTLLCNKMFSPVCGTDGVTYDNECMLCAHNVEQGTSVG |
| | | KKYDGKCKKEVATVSVDCSGYPKPACTMEYMPLCGSDNKTYGNKCNFCNAVV |
| Ovomucoid-like | SEQ ID NO: | MTMPGAFVVLSFVLCCFPDATFGVEVDCSTYPNTTNEEGKEVLVCSKILSPICGTDGVTYSN |
| [Tyto alba] | 184 | ECLLCANNIEYGTNISKYHDGECKEFVPVNCSRYPNTTNEEGKVMLICNKDLSPVCGTDGV |
| | | TYDNECLLCAHNLEPGTSVGKKYDGECKKEIATVDCSDYPKPVCSLESMPLCGSDNKTYS |
| | | NKCNFCNAVVDSNETLTLSHFGKC |
| Ovomucoid | SEQ ID NO: | MTMAGVFVLLSFALCCFPDAAFGVEVDCSTYPNTTNEEGKEVLVCTKILSPICGTDGVTYS |
| [Balearica regulorum | 185 | NECLLCAYNIEYGTNVSKDHDGECKEVVPVDCSRYPNSTNEEGKVVMLCSKDLNPVCGTD |
| gibbericeps] | | GVTYDNECVLCAHNVESGTSVGKKYDGECKKETATVDCSDYPKPACTLEYMPFCGSDSKT |
| | | YSNKCNFCNAVVDSNGTLTLSHFGKC |
| Turkey vulture | SEQ ID NO: | MTTAGVFVLLSFALCSFPDAAFGVEVDCSTYPNTTNEEGKEVLVCTKILSPICGTDGVTYSN |
| [Cathartes aura] | 186 | ECLLCAYNIEYGTNVSKDHDGECKEFVPVDCSRYPNTTNEDGKVVLLCNKDLSPICGTDGV |
| OVD (native | | TYDNECLLCARNLEPGTSVGKKYDGECKKEIATVDCSDYPKPVCSLEYMPLCGSDSKTYSN |
| sequence) | | KCNFCNAVVDSNGTLTLSHFGKC |
| bolded is native | | |
| signal sequence | | |
| Ovomucoid-like | SEQ ID NO: | MTTAGVFVLLSFTLCSFPDAAFGVEVDCSPYPNTTNEEGKEVLVCNKILSPICGTDGVTYSN |
| [Cuculus canorus] | 187 | ECLLCAYNLEYGTNISKDYDGECKEVAPVDCSRHPNTTNEEGKVELLCNKDLNPICGTNGV |
| | | TYDNECLLCARNLESGTSIGKKYDGECKKEIATVDCSDYPKPVCTLEEMPLCGSDNKTYGN |
| | | KCNFCNAVVDSNGTLTLSHFGKC |
| Ovomucoid | SEQ ID NO: | MTTAVVFVLLSFALCCFPDAAFGVEVDCSTYPNSTNEEGKDVLVCPKILGPICGTDGVTYS |
| [Antrostomus | 188 | NECLLCAYNIQYGTNVSKDHDGECKEIVPVDCSRYPNTTNEEGKVVFLCNKNFDPVCGTD |
| carolinensis] | | GDTYDNECMLCARSLEPGTTVGKKHDGECKREIATVDCSDYPKPTCSAEDMPLCGSDSKT |
| | | YSNKCNFCNAVVDSNGTLTLSRFGKC |
| Ovomucoid [Cariama | SEQ ID NO: | MTMTGVFVLLSFAICCFPDAAFGVEVDCSTYPNTTNEEGKEVLVCTKILSPICGTDGVTYSN |
| cristata] | 189 | ECLLCAYNIEYGTNVSKDHDGECKEVVPVDCSKYPNTTNEEGKVVLLCSKDLSPVCGTDG |
| | | VTYDNECLLCARNLEPGSSVGKKYDGECKKEIATIDCSDYPKPVCSLEYMPLCGSDSKTYD |
| | | NKCNFCNAVVDSNGTLTLSHFGKC |
| Ovomucoid-like | SEQ ID NO: | MTTAGVFVLLSFVLCCFPDAVFGVEVDCSTYPNTTNEEGKEVLVCTKILSPICGTDGVTYSN |
| isoform X2 | 190 | ECLLCAYNIEYGTNVSKDHDGECKEVVPVNCSRYPNTTNEEGKVVLRCSKDLSPVCGTDG |
| [Pygoscelis adeliae] | | VTYDNECLMCARNLEPGAVVGKNYDGECKKEIATVDCSDYPKPVCSLEYMPLCGSDSKTY |
| | | SNKCNFCNAVVDSNGTLTLSHFGKC |
| Ovomucoid-like | SEQ ID NO: | MTTAGVFVLLSIALCCFPDAAFGVEVDCSAYSNTTSEEGKEVLSCTKILSPICGTDGVTYSN |
| [Nipponia nippon] | 191 | ECLLCAYNIEYGTNISKDHDGECKEVVSVDCSRYPNTTNEEGKAVLLCNKDLSPVCGTDGV |
| | | TYDNECLLCAHNLEPGTSVGKKYDGACKKEIATVDCSDYPKPVCTLEYLPLCGSDSKTYSN |
| | | KCDFCNAVVDSNGTLTLSHFGKC |
| Ovomucoid-like | SEQ ID NO: | MTTAGVFVLLSFALCCFPDAAFGVEVDCSTYPNTTNEEGKEVLVCTKILSPICGTDGTTYSN |
| [Phaethon lepturus] | 192 | ECLLCAYNIEYGTNVSKDHDGECKVVPVDCSKYPNTTNEDGKVVLLCNKALSPICGTDRV |
| | | TYDNECLMCAHNLEPGTSVGKKHDGECQKEVATVDCSDYPKPVCSLEYMPLCGSDGKTY |
| | | SNKCNFCNAVVNSNGTLTLSHFEKC |
| Ovomucoid-like | SEQ ID NO: | MTTAGVFVLLSFVLCCFFPDAAFGVEVDCSTYPNTTNEEGKEVLVCAKILSPVCGTDGVTY |
| isoform X1 | 193 | SNECLLCAHNIENGTNVGKDHDGKCKEAVPVDCSRYPNTTDEEGKVVLLCNKDVSPVCGT |
| [Melopsittacus | | DGVTYDNECLLCAHNLEAGTSVDKKNDSECKTEDTTLAAVSVDCSDYPKPVCTLEYLPLC |
| undulatus] | | GSDNKTYSNKCRFCNAVVDSNGTLTLSRFGKC |
| Ovomucoid | SEQ ID NO: | MTTAGVFVLLSFALCCSPDAAFGVEVDCSTYPNTTNEEGKEVLACTKILSPICGTDGVTYSN |
| [Podiceps cristatus] | 194 | ECLLCAYNMEYGTNVSKDHDGKCKEVVPVDCSRYPNTTNEEGKVVLLCNKDLSPVCGTD |
| | | GVTYDNECLLCARNLEPGASVGKKYDGECKKEIATVDCSDYPKPVCSLEHMPLCGSDSKT |
| | | YSNKCTFCNAVVDSNGTLTLSHFGKC |
| Ovomucoid-like | SEQ ID NO: | MTTAGVFVLLSFALCCFPDAAFGVEVDCSTYPNTTNEEGREVLVCTKILSPICGTDGVTYSN |
| [Fulmarus glacialis] | 195 | ECLLCAYNIEYGTNVSKDHDGECKEVAPVGCSRYPNTTNEEGKVVLLCNKDLSPVCGTDG |
| | | VTYDNECLLCARHLEPGTSVGKKYDGECKKEIATVDCSDYPKPVCSLEYMPLCGSDSKTYS |
| | | NKCNFCNAVLDSNGTLTLSHFGKC |
| Ovomucoid | SEQ ID NO: | MTTAGVFVLLSFALCCFPDAVFGVEVDCSTYPNTTNEEGKEVLVCTKILSPICGTDGVTYSN |
| [Aptenodytes | 196 | ECLLCAYNIEYGTNVSKDHDGECKEVVPVDCSRYPNTTNEEGKVVLRCNKDLSPVCGTDG |
| forsteri] | | VTYDNECLMCARNLEPGAIVGKKYDGECKKEIATVDCSDYPKPVCSLEYMPLCGSDSKTY |
| | | SNKCNFCNAVVDSNGTLILSHFGKC |
| Ovomucoid-like | SEQ ID NO: | MTTAGVFVLLSFVLCCFPDAVFGVEVDCSTYPNTTNEEGKEVLVCTKILSPICGTDGVTYSN |
| isoform X1 | 197 | ECLLCAYNIEYGTNVSKDHDGECKEVVPVDCSRYPNTTNEEGKVVLRCSKDLSPVCGTDG |
| [Pygoscelis adeliae] | | VTYDNECLMCARNLEPGAVVGKNYDGECKKEIATVDCSDYPKPVCSLEYMPLCGSDSKTY |
| | | SNKCNFCNAVVDSNGTLTLSHFGKC |
| Ovomucoid isoform | SEQ ID NO: | MSSQNQLPSRCRPLPGSQDLNKYYQPHCTGDRFCWLFYVTVEQFRHCICIYLQLALERPSH |
| X1 [Aptenodytes | 198 | EQSGQPADSRNTSTMTTAGVFVLLSFALCCFPDAVFGVEVDCSTYPNTTNEEGKEVLVCTK |
| forsteri] | | ILSPICGTDGVTYSNECLLCAYNIEYGTNVSKDHDGECKEVVPVDCSRYPNTTNEEGKVVL |
| | | RCNKDLSPVCGTDGVTYDNECLMCARNLEPGAIVGKKYDGECKKEIATVDCSDYPKPVCS |
| | | LEYMPLCGSDSKTYSNKCNFCNAVVDSNGTLILSHFGKC |
| Ovomucoid, partial | SEQ ID NO: | MTTAVVFVLLSFALCCFPDAAFGVEVDCSTYPNSTNEEGKDVLVCPKILGPICGTDGVTYS |
| [Antrostomus | 199 | NECLLCAYNIQYGTNVSKDHDGECKEIVPVDCSRYPNTTNEEGKVVFLCNKNFDPVCGTD |
| carolinensis] | | GDTYDNECMLCARSLEPGTTVGKKHDGECKREIATVDCSDYPKPTCSAEDMPLCGSDSKT |
| | | YSNKCNFCNAVV |
| rOVD as expressed | SEQ ID NO: | EAEAAEVDCSRFPNATDKEGKDVLVCNKDLRPICGTDGVTYTNDCLLCAYSIEFGTNISKE |
| in pichia secreted | 200 | HDGECKETVPMNCSSYANTTSEDGKVMVLCNRAFNPVCGTDGVTYDNECLLCAHKVEQG |
| form 1 | | ASVDKRHDGGCRKELAAVSVDCSEYPKPDCTAEDRPLCGSDNKTYGNKCNFCNAVVESN |
| | | GTLTLSHFGKC |
| rOVD as expressed | SEQ ID NO: | EEGVSLEKREAEAAEVDCSRFPNATDKEGKDVLVCNKDLRPICGTDGVTYTNDCLLCAYSI |
| in pichia secreted | 201 | EFGTNISKEHDGECKETVPMNCSSYANTTSEDGKVMVLCNRAFNPVCGTDGVTYDNECLL |
| form 2 | | CAHKVEQGASVDKRHDGGCRKELAAVSVDCSEYPKPDCTAEDRPLCGSDNKTYGNKCNF |
| | | CNAVVESNGTLTLSHFGKC |
| rOVD [gallus] | SEQ ID NO: | MRFPSIFTAVLFAASSALAAPVNTTTEDETAQIPAEAVIGYSDLEGDFDVAVLPFSNSTNNG |
| coding sequence | 202 | LLFINTTIASIAAKEEGVSLEKREAEAAEVDCSRFPNATDKEGKDVLVCNKDLRPICGTDGV |
| containing an alpha | | TYTNDCLLCAYSIEFGTNISKEHDGECKETVPMNCSSYANTTSEDGKVMVLCNRAFNPVCG |
| mating factor signal | | TDGVTYDNECLLCAHKVEQGASVDKRHDGGCRKELAAVSVDCSEYPKPDCTAEDRPLCG |
| sequence (bolded) as | | SDNKTYGNKCNFCNAVVESNGTLTLSHFGKC |
| expressed in pichia | | |
| Turkey vulture OVD | SEQ ID NO: | MRFPSIFTAVLFAASSALAAPVNTTTEDETAQIPAEAVIGYSDLEGDFDVAVLPFSNSTNNG |
| coding sequence | 203 | LLFINTTIASIAAKEEGVSLEKREAEAVEVDCSTYPNTTNEEGKEVLVCTKILSPICGTDGVT |
| containing secretion | | YSNECLLCAYNIEYGTNVSKDHDGECKEFVPVDCSRYPNTTNEDGKVVLLCNKDLSPICGT |
| signals as expressed | | DGVTYDNECLLCARNLEPGTSVGKKYDGECKKEIATVDCSDYPKPVCSLEYMPLCGSDSK |
| in pichia | | TYSNKCNFCNAVVDSNGTLTLSHFGKC |
| bolded is an alpha | | |
| mating factor signal | | |
| sequence | | |
| Turkey vulture OVD | SEQ ID NO: | EAEAVEVDCSTYPNTTNEEGKEVLVCTKILSPICGTDGVTYSNECLLCAYNIEYGTNVSKDH |
| in secreted form | 204 | DGECKEFVPVDCSRYPNTTNEDGKVVLLCNKDLSPICGTDGVTYDNECLLCARNLEPGTSV |
| expressed in Pichia | | GKKYDGECKKEIATVDCSDYPKPVCSLEYMPLCGSDSKTYSNKCNFCNAVVDSNGTLTLS |
| | | HFGKC |
| Humming bird | SEQ ID NO: | MTMAGVFVLLSFILCCFPDTAFGVEVDCSIYPNTTSEEGKEVLVCTETLSPICGSDGVTYNN |
| OVD (native | 205 | ECQLCAYNVEYGTNVSKDHDGECKEIVPVDCSRYPNTTEEGRVVMLCNKALSPVCGTDGV |
| sequence) | | TYDNECLLCARNLESGTSVGKKFDGECKKEIATVDCTDYPKPVCSLDYMPLCGSDSKTYSN |
| bolded is the native | | KCNFCNAVMDSNGTLTLNHFGKC |
| signal sequence | | |
| Humming bird OVD | SEQ ID NO: | MRFPSIFTAVLFAASSALAAPVNTTTEDETAQIPAEAVIGYSDLEGDFDVAVLPFSNSTNNG |
| coding sequence as | 206 | LLFINTTIASIAAKEEGVSLDKREAEAVEVDCSIYPNTTSEEGKEVLVCTETLSPICGSDGVTY |
| expressed in Pichia | | NNECQLCAYNVEYGTNVSKDHDGECKEIVPVDCSRYPNTTEEGRVVMLCNKALSPVCGTD |
| bolded is an alpha | | GVTYDNECLLCARNLESGTSVGKKFDGECKKEIATVDCTDYPKPVCSLDYMPLCGSDSKT |
| mating factor signal | | YSNKCNFCNAVMDSNGTLTLNHFGKC |
| sequence | | |
| Humming bird OVD | SEQ ID NO: | EAEAVEVDCSIYPNTTSEEGKEVLVCTETLSPICGSDGVTYNNECQLCAYNVEYGTNVSKD |
| in secreted form from | 207 | HDGECKEIVPVDCSRYPNTTEEGRVVMLCNKALSPVCGTDGVTYDNECLLCARNLESGTS |
| Pichia | | VGKKFDGECKKEIATVDCTDYPKPVCSLDYMPLCGSDSKTYSNKCNFCNAVMDSNGTLTL |
| | | NHFGKC |
| Ovalbumin related | SEQ ID NO: | MFFYNTDFRMGSISAANAEFCFDVFNELKVQHTNENILYSPLSIIVALAMVYMGARGNTEY |
| protein X | 208 | QMEKALHFDSIAGLGGSTQTKVQKPKCGKSVNIHLLFKELLSDITASKANYSLRIANRLYAE |
| | | KSRPILPIYLKCVKKLYRAGLETVNFKTASDQARQLINSWVEKQTEGQIKDLLVSSSTDLDT |
| | | TLVLVNAIYFKGMWKTAFNAEDTREMPFHVTKEESKPVQMMCMNNSFNVATLPAEKMKI |
| | | LELPFASGDLSMLVLLPDEVSGLERIEKTINFEKLTEWTNPNTMEKRRVKVYLPQMKIEEKY |
| | | NLTSVLMALGMTDLFIPSANLTGISSAESLKISQAVHGAFMELSEDGIEMAGSTGVIEDIKHS |
| | | PELEQFRADHPFLFLIKHNPTNTIVYFGRYWSP* |
| Ovalbumin related | SEQ ID NO: | MDSISVTNAKFCFDVFNEMKVHHVNENILYCPLSILTALAMVYLGARGNTESQMKKVLHF |
| protein Y | 209 | DSITGAGSTTDSQCGSSEYVHNLFKELLSEITRPNATYSLEIADKLYVDKTFSVLPEYLSCAR |
| | | KFYTGGVEEVNFKTAAEEARQLINSWVEKETNGQIKDLLVSSSIDFGTTMVFINTIYFKGIW |
| | | KIAFNTEDTREMPFSMTKEESKPVQMMCMNNSFNVATLPAEKMKILELPYASGDLSMLVL |
| | | LPDEVSGLERIEKTINFDKLREWTSTNAMAKKSMKVYLPRMKIEEKYNLTSILMALGMTDL |
| | | FSRSANLTGISSVDNLMISDAVHGVFMEVNEEGTEATGSTGAIGNIKHSLELEEFRADHPFLF |
| | | FIRYNPTNAILFFGRYWSP* |
| Ovalbumin | SEQ ID NO: | MGSIGAASMEFCFDVFKELKVHHANENIFYCPIAIMSALAMVYLGAKDSTRTQINKVVRFD |
| | 210 | KLPGFGDSIEAQCGTSVNVHSSLRDILNQITKPNDVYSFSLASRLYAEERYPILPEYLQCVKE |
| | | LYRGGLEPINFQTAADQARELINSWVESQTNGIIRNVLQPSSVDSQTAMVLVNAIVFKGLW |
| | | EKAFKDEDTQAMPFRVTEQESKPVQMMYQIGLFRVASMASEKMKILELPFASGTMSMLVL |
| | | LPDEVSGLEQLESIINFEKLTEWTSSNVMEERKIKVYLPRMKMEEKYNLTSVLMAMGITDV |
| | | FSSSANLSGISSAESLKISQAVHAAHAEINEAGREVVGSAEAGVDAASVSEEFRADHPFLFCI |
| | | KHIATNAVLFFGRCVSP* |
| Chicken Ovalbumin | SEQ ID NO: | MRFPSIFTAVLFAASSALAAPVNTTTEDETAQIPAEAVIGYSDLEGDFDVAVLPFSNSTNNG |
| with bolded signal | 211 | LLFINTTIASIAAKEEGVSLDKREAEAGSIGAASMEFCFDVFKELKVHHANENIFYCPIAIMS |
| sequence | | ALAMVYLGAKDSTRTQINKVVRFDKLPGFGDSIEAQCGTSVNVHSSLRDILNQITKPNDVY |
| | | SFSLASRLYAEERYPILPEYLQCVKELYRGGLEPINFQTAADQARELINSWVESQINGIIRNV |
| | | LQPSSVDSQTAMVLVNAIVFKGLWEKAFKDEDTQAMPFRVTEQESKPVQMMYQIGLFRV |
| | | ASMASEKMKILELPFASGTMSMLVLLPDEVSGLEQLESIINFEKLTEWTSSNVMEERKIKVY |
| | | LPRMKMEEKYNLTSVLMAMGITDVFSSSANLSGISSAESLKISQAVHAAHAEINEAGREVV |
| | | GSAEAGVDAASVSEEFRADHPFLFCIKHIATNAVLFFGRCVSP |
| Chicken OVA | SEQ ID NO: | EAEAGSIGAASMEFCFDVFKELKVHHANENIFYCPIAIMSALAMVYLGAKDSTRTQINKVV |
| sequence as secreted | 212 | RFDKLPGFGDSIEAQCGTSVNVHSSLRDILNQITKPNDVYSFSLASRLYAEERYPILPEYLQC |
| from pichia | | VKELYRGGLEPINFQTAADQARELINSWVESQINGIIRNVLQPSSVDSQTAMVLVNAIVFKG |
| | | LWEKAFKDEDTQAMPFRVTEQESKPVQMMYQIGLFRVASMASEKMKILELPFASGTMSM |
| | | LVLLPDEVSGLEQLESIINFEKLTEWTSSNVMEERKIKVYLPRMKMEEKYNLTSVLMAMGI |
| | | TDVFSSSANLSGISSAESLKISQAVHAAHAEINEAGREVVGSAEAGVDAASVSEEFRADHPF |
| | | LFCIKHIATNAVLFFGRCVSP |
| Predicted Ovalbumin | SEQ ID NO: | MRVPAQLLGLLLLWLPGARCGSIGAASMEFCFDVFKELKVHHANENIFYCPIAIMSALAMV |
| [Achromobacter | 213 | YLGAKDSTRTQINKVVRFDKLPGFGDSIEAQCGTSVNVHSSLRDILNQITKPNDVYSFSLAS |
| denitrificans] | | RLYAEERYPILPEYLQCVKELYRGGLEPINFQTAADQARELINSWVESQINGIIRNVLQPSSV |
| | | DSQTAMVLVNAIVFKGLWEKAFKDEDTQAMPFRVTEQESKPVQMMYQIGLFRVASMASE |
| | | KMKILELPFASGTMSMLVLLPDEVSGLEQLESIINFEKLTEWTSSNVMEERKIKVYLPRMK |
| | | MEEKYNLTSVLMAMGITDVFSSSANLSGISSAESLKISQAVHAAHAEINEAGREVVGSAEA |
| | | GVDAASVSEEFRADHPFLFCIKHIATNAVLFFGRCVSPLEIKRAAAHHHHHH |
| OLLAS epitope- | SEQ ID NO: | MTSGFANELGPRLMGKLTMGSIGAASMEFCFDVFKELKVHHANENIFYCPIAIMSALAMV |
| tagged ovalbumin | 214 | YLGAKDSTRTQINKVVRFDKLPGFGDSIEAQCGTSVNVHSSLRDILNQITKPNDVYSFSLAS |
| | | RLYAEERYPILPEYLQCVKELYRGGLEPINFQTAADQARELINSWVESQTNGIIRNVLQPSSV |
| | | DSQTAMVLVNAIVFKGLWEKTFKDEDTQAMPFRVTEQESKPVQMMYQIGLFRVASMASE |
| | | KMKILELPFASGTMSMLVLLPDEVSGLEQLESIINFEKLTEWTSSNVMEERKIKVYLPRMK |
| | | MEEKYNLTSVLMAMGITDVFSSSANLSGISSAESLKISQAVHAAHAEINEAGREVVGSAEA |
| | | GVDAASVSEEFRADHPFLFCIKHIATNAVLFFGRCVSPSR |
| Serpin family protein | SEQ ID NO: | MGGRRVRWEVYISRAGYVNRQIAWRRHHRSLTMRVPAQLLGLLLLWLPGARCGSIGAAS |
| [Achromobacter | 215 | MEFCFDVFKELKVHHANENIFYCPIAIMSALAMVYLGAKDSTRTQINKVVRFDKLPGFGDS |
| denitrificans] | | IEAQCGTSVNVHSSLRDILNQITKPNDVYSFSLASRLYAEERYPILPEYLQCVKELYRGGLEP |
| | | INFQTAADQARELINSWVESQINGIIRNVLQPSSVDSQTAMVLVNAIVFKGLWEKAFKDED |
| | | TQAMPFRVTEQESKPVQMMYQIGLFRVASMASEKMKILELPFASGTMSMLVLLPDEVSGL |
| | | EQLESIINFEKLTEWTSSNVMEERKIKVYLPRMKMEEKYNLTSVLMAMGITDVFSSSANLS |
| | | GISSAESLKISQAVHAAHAEINEAGREVVGSAEAGVDAASVSEEFRADHPFLFCIKHIATNA |
| | | VLFFGRCVSPLEIKRAAAHHHHHH |
| PREDICTED: | SEQ ID NO: | MGSIGAVSMEFCFDVFKELKVHHANENIFYSPFTIISALAMVYLGAKDSTRTQINKVVRFDK |
| ovalbumin isoform | 216 | LPGFGDSVEAQCGTSVNVHSSLRDILNQITKPNDVYSFSLASRLYAEETYPILPEYLQCVKEL |
| X1 [Meleagris | | YRGGLESINFQTAADQARGLINSWVESQTNGMIKNVLQPSSVDSQTAMVLVNAIVFKGLW |
| gallopavo] | | EKAFKDEDTQAIPFRVTEQESKPVQMMYQIGLFKVASMASEKMKILELPFASGTMSMWVL |
| | | LPDEVSGLEQLETTISFEKMTEWISSNIMEERRIKVYLPRMKMEEKYNLTSVLMAMGITDLF |
| | | SSSANLSGISSAGSLKISQAVHAAYAEIYEAGREVIGSAEAGADATSVSEEFRVDHPFLYCIK |
| | | HNLTNSILFFGRCISP |
| Ovalbumin precursor | SEQ ID NO: | MGSIGAVSMEFCFDVFKELKVHHANENIFYSPFTIISALAMVYLGAKDSTRTQINKVVRFDK |
| [Meleagris | 217 | LPGFGDSVEAQCGTSVNVHSSLRDILNQITKPNDVYSFSLASRLYAEETYPILPEYLQCVKEL |
| gallopavo] | | YRGGLESINFQTAADQARGLINSWVESQTNGMIKNVLQPSSVDSQTAMVLVNAIVFKGLW |
| | | EKAFKDEDTQAIPFRVTEQESKPVQMMYQIGLFKVASMASEKMKILELPFASGTMSMWVL |
| | | LPDEVSGLEQLETTISFEKMTEWISSNIMEERRIKVYLPRMKMEEKYNLTSVLMAMGITDLF |
| | | SSSANLSGISSAGSLKISQAAHAAYAEIYEAGREVIGSAEAGADATSVSEEFRVDHPFLYCIK |
| | | HNLTNSILFFGRCISP |
| Hypothetical protein | SEQ ID NO: | YYRVPCMVLCTAFHPYIFIVLLFALDNSEFTMGSIGAVSMEFCFDVFKELRVHHPNENIFFCP |
| [Bambusicola | 218 | FAIMSAMAMVYLGAKDSTRTQINKVIRFDKLPGFGDSTEAQCGKSANVHSSLKDILNQITK |
| thoracicus] | | PNDVYSFSLASRLYADETYSIQSEYLQCVNELYRGGLESINFQTAADQARELINSWVESQTN |
| | | GIIRNVLQPSSVDSQTAMVLVNAIVFRGLWEKAFKDEDTQTMPFRVTEQESKPVQMMYQI |
| | | GSFKVASMASEKMKILELPLASGTMSMLVLLPDEVSGLEQLETTISFEKLTEWTSSNVMEE |
| | | RKIKVYLPRMKMEEKYNLTSVLMAMGITDLFRSSANLSGISLAGNLKISQAVHAAHAEINE |
| | | AGRKAVSSAEAGVDATSVSEEFRADRPFLFCIKHIATKVVFFFGRYTSP |
| Egg albumin | SEQ ID NO: | MGSIGAASMEFCFDVFKELKVHHANDNMLYSPFAILSTLAMVFLGAKDSTRTQINKVVHF |
| | 219 | DKLPGFGDSIEAQCGTSVNVHSSLRDILNQITKQNDAYSFSLASRLYAQETYTVVPEYLQCV |
| | | KELYRGGLESVNFQTAADQARGLINAWVESQINGIIRNILQPSSVDSQTAMVLVNAIAFKG |
| | | LWEKAFKAEDTQTIPFRVTEQESKPVQMMYQIGSFKVASMASEKMKILELPFASGTMSML |
| | | VLLPDDVSGLEQLESIISFEKLTEWTSSSIMEERKVKVYLPRMKMEEKYNLTSLLMAMGITD |
| | | LFSSSANLSGISSVGSLKISQAVHAAHAEINEAGRDVVGSAEAGVDATEEFRADHPFLFCVK |
| | | HIETNAILLFGRCVSP |
| Ovalbumin isoform | SEQ ID NO: | MASIGAVSTEFCVDVYKELRVHHANENIFYSPFTIISTLAMVYLGAKDSTRTQINKVVRFDK |
| X2 [Numida | 220 | LPGFGDSIEAQCGTSVNVHSSLRDILNQITKPNDVYSFSLASRLYAEETYPILPEYLQCVKEL |
| meleagris] | | YRGGLESINFQTAADQARELINSWVESQTSGIIKNVLQPSSVNSQTAMVLVNAIYFKGLWE |
| | | RAFKDEDTQAIPFRVTEQESKPVQMMSQIGSFKVASVASEKVKILELPFVSGTMSMLVLLPD |
| | | EVSGLEQLESTISTEKLTEWTSSSIMEERKIKVFLPRMRMEEKYNLTSVLMAMGMTDLFSSS |
| | | ANLSGISSAESLKISQAVHAAYAEIYEAGREVVSSAEAGVDATSVSEEFRVDHPFLLCIKHN |
| | | PTNSILFFGRCISP |
| Ovalbumin isoform | SEQ ID NO: | MALCKAFHPYIFIVLLFDVDNSAFTMASIGAVSTEFCVDVYKELRVHHANENIFYSPFTIIST |
| X1 [Numida | 221 | LAMVYLGAKDSTRTQINKVVRFDKLPGFGDSIEAQCGTSVNVHSSLRDILNQITKPNDVYSF |
| meleagris] | | SLASRLYAEETYPILPEYLQCVKELYRGGLESINFQTAADQARELINSWVESQTSGIIKNVLQ |
| | | PSSVNSQTAMVLVNAIYFKGLWERAFKDEDTQAIPFRVTEQESKPVQMMSQIGSFKVASVA |
| | | SEKVKILELPFVSGTMSMLVLLPDEVSGLEQLESTISTEKLTEWTSSSIMEERKIKVFLPRMR |
| | | MEEKYNLTSVLMAMGMTDLFSSSANLSGISSAESLKISQAVHAAYAEIYEAGREVVSSAEA |
| | | GVDATSVSEEFRVDHPFLLCIKHNPTNSILFFGRCISP |
| PREDICTED: | SEQ ID NO: | MGSIGAASMEFCFDVFKELKVHHANDNMLYSPFAILSTLAMVFLGAKDSTRTQINKVVHF |
| Ovalbumin isoform | 222 | DKLPGFGDSIEAQCGTSANVHSSLRDILNQITKQNDAYSFSLASRLYAQETYTVVPEYLQCV |
| X2 [Coturnix | | KELYRGGLESVNFQTAADQARGLINAWVESQINGIIRNILQPSSVDSQTAMVLVNAIAFKG |
| japonica] | | LWEKAFKAEDTQTIPFRVTEQESKPVQMMHQIGSFKVASMASEKMKILELPFASGTMSML |
| | | VLLPDDVSGLEQLESTISFEKLTEWTSSSIMEERKVKVYLPRMKMEEKYNLTSLLMAMGIT |
| | | DLFSSSANLSGISSVGSLKISQAVHAAYAEINEAGRDVVGSAEAGVDATEEFRADHPFLFCV |
| | | KHIETNAILLFGRCVSP |
| PREDICTED: | SEQ ID NO: | MGLCTAFHPYIFIVLLFALDNSEFTMGSIGAASMEFCFDVFKELKVHHANDNMLYSPFAILS |
| ovalbumin isoform | 223 | TLAMVFLGAKDSTRTQINKVVHFDKLPGFGDSIEAQCGTSANVHSSLRDILNQITKQNDAY |
| X1 [Coturnix | | SFSLASRLYAQETYTVVPEYLQCVKELYRGGLESVNFQTAADQARGLINAWVESQINGIIR |
| japonica] | | NILQPSSVDSQTAMVLVNAIAFKGLWEKAFKAEDTQTIPFRVTEQESKPVQMMHQIGSFKV |
| | | ASMASEKMKILELPFASGTMSMLVLLPDDVSGLEQLESTISFEKLTEWTSSSIMEERKVKVY |
| | | LPRMKMEEKYNLTSLLMAMGITDLFSSSANLSGISSVGSLKISQAVHAAYAEINEAGRDVV |
| | | GSAEAGVDATEEFRADHPFLFCVKHIETNAILLFGRCVSP |
| Egg albumin | SEQ ID NO: | MGSIGAASMEFCFDVFKELKVHHANDNMLYSPFAILSTLAMVFLGAKDSTRTQINKVVHF |
| | 224 | DKLPGFGDSIEAQCGTSANVHSSLRDILNQITKQNDAYSFSLASRLYAQETYTVVPEYLQCV |
| | | KELYRGGLESVNFQTAADQARGLINAWVESQTNGIIRNILQPSSVDSQTAMVLVNAIAFKG |
| | | LWEKAFKAEDTQTIPFRVTEQESKPVQMMHQIGSFKVASMASEKMKILELPFASGTMSML |
| | | VLLPDDVSGLEQLESTISFEKLTEWTSSSIMEERKVKVYLPRMKMEEKYNLTSLLMAMGIT |
| | | DLFSSSANLSGISSVGSLKIPQAVHAAYAEINEAGRDVVGSAEAGVDATEEFRADHPFLFCV |
| | | KHIETNAILLFGRCVSP |
| ovalbumin [Anas | SEQ ID NO: | MGSIGAASTEFCFDVFRELRVQHVNENIFYSPFSIISALAMVYLGARDNTRTQIDKVVHFDK |
| platyrhynchos] | 225 | LPGFGESMEAQCGTSVSVHSSLRDILTQITKPSDNFSLSFASRLYAEETYAILPEYLQCVKEL |
| | | YKGGLESISFQTAADQARELINSWVESQTNGIIKNILQPSSVDSQTTMVLVNAIYFKGMWEK |
| | | AFKDEDTQAMPFRMTEQESKPVQMMYQVGSFKVAMVTSEKMKILELPFASGMMSMFVLL |
| | | PDEVSGLEQLESTISFEKLTEWTSSTMMEERRMKVYLPRMKMEEKYNLTSVFMALGMTDL |
| | | FSSSANMSGISSTVSLKMSEAVHAACVEIFEAGRDVVGSAEAGMDVTSVSEEFRADHPFLFF |
| | | IKHNPTNSILFFGRWMSP |
| PREDICTED: | SEQ ID NO: | MGSIGAASTEFCFDVFRELKVQHVNENIFYSPLSIISALAMVYLGARDNTRTQIDQVVHFDK |
| ovalbumin-like | 226 | IPGFGESMEAQCGTSVSVHSSLRDILTEITKPSDNFSLSFASRLYAEETYTILPEYLQCVKELY |
| [Anser cygnoides | | KGGLESISFQTAADQARELINSWVESQINGIIKNILQPSSVDSQTTMVLVNAIYFKGMWEKA |
| domesticus] | | FKDEDTQTMPFRMTEQESKPVQMMYQVGSFKLATVTSEKVKILELPFASGMMSMCVLLPD |
| | | EVSGLEQLETTISFEKLTEWTSSTMMEERRMKVYLPRMKMEEKYNLTSVFMALGMTDLFS |
| | | SSANMSGISSTVSLKMSEAVHAACVEIFEAGRDVVGSAEAGMDVTSVSEEFRADHPFLFFIK |
| | | HNPSNSILFFGRWISP |
| PREDICTED: | SEQ ID NO: | MGSIGAASTEFCFDVFKELKVQHVNENIFYSPLTIISALSMVYLGARENTRAQIDKVLHFDK |
| Ovalbumin-like | 227 | MPGFGDTIESQCGTSVSIHTSLKDMFTQITKPSDNYSLSFASRLYAEETYPILPEYLQCVKEL |
| [Aquila chrysaetos | | YKGGLETISFQTAAEQARELINSWVESQTNGMIKNILQPSSVDPQTKMVLVNAIYFKGVWE |
| canadensis] | | KAFKDEDTQEVPFRVTEQESKPVQMMYQIGSFKVAVMASEKMKILELPYASGQLSMLVLL |
| | | PDDVSGLEQLESAITFEKLMAWTSSTTMEERKMKVYLPRMKIEEKYNLTSVLMALGVTDL |
| | | FSSSANLSGISSAESLKISKAVHEAFVEIYEAGSEVVGSTEAGMEVTSVSEEFRADHPFLFLIK |
| | | HNPTNSILFFGRCFSP |
| PREDICTED: | SEQ ID NO: | MGSIGAASTEFCFDVFKELKVQHVNENIFYSPLTIISALSMVYLGARENTRTQIDKVLHFDK |
| Ovalbumin-like | 228 | MTGFGDTVESQCGTSVSIHTSLKDIFTQITKPSDNYSLSLASRLYAEETYPILPEYLQCVKEL |
| [Haliaeetus albicilla] | | YKGGLETVSFQTAAEQARELINSWVESQTNGMIKNILQPSSVDPQTKMVLVNAIYFKGVW |
| | | EKAFKDEDTQEVPFRVTEQESKPVQMMYQIGSFKVAVMASEKMKILELPYASGQLSMLVL |
| | | LPDDVSGLEQLESAITSEKLMEWTSSTTMEERKMKVYLPRMKIEEKYNLTSVLMALGVTD |
| | | LFSSSADLSGISSAESLKISKAVHEAFVEIYEAGSEVVGSTEGGMEVTSVSEEFRADHPFLFLI |
| | | KHKPTNSILFFGRCFSP |
| PREDICTED: | SEQ ID NO: | MGSIGAASTEFCFDVFKELKVQHVNENIFYSPLTIISALSMVYLGARENTRTQIDKVLHFDK |
| Ovalbumin-like | 229 | MTGFGDTVESQCGTSVSIHTSLKDIFTQITKPSDNYSLSLASRLYAEETYPILPEYLQCVKEL |
| [Haliaeetus | | YKGGLETVSFQTAAEQARELINSWVESQTNGMIKNILQPSSVDPQTKMVLVNAIYFKGVW |
| leucocephalus] | | EKAFKDEDTQEVPFRVTEQESKPVQMMYQIGSFKVAVMASEKMKILELPYASGQLSMLVL |
| | | LPDDVSGLEQLESAITSEKLMEWTSSTTMEERKMKVYLPRMKIEEKYNLTSVLMALGVTD |
| | | LFSSSADLSGISSAESLKISKAVHEAFVEIYEAGSEVVGSTEGGMEVTSFSEEFRADHPFLFLI |
| | | KHKPTNSILFFGRCFSP |
| PREDICTED: | SEQ ID NO: | MGSIGAASTEFCFDVFKELKVQHVNENIFYSPLSIISALSMVYLGARENTRAQIDKVVHFDKI |
| Ovalbumin | 230 | TGFGETIESQCGTSVSVHTSLKDMFTQITKPSDNYSLSFASRLYAEETYPILPEYLQCVKELY |
| [Fulmarus glacialis] | | KGGLETTSFQTAADQARELINSWVESQTNGMIKNILQPGSVDPQTEMVLVNAIYFKGMWE |
| | | KAFKDEDTQAVPFRMTEQESKTVQMMYQIGSFKVAVMASEKMKILELPYASGELSMLVM |
| | | LPDDVSGLEQLETAITFEKLMEWTSSNMMEERKMKVYLPRMKMEEKYNLTSVLMALGVT |
| | | DLFSSSANLSGISSAESLKMSEAVHEAFVEIYEAGSEVVGSTGAGMEVTSVSEEFRADHPFL |
| | | FLIKHNPTNSILFFGRCFSP |
| PREDICTED: | SEQ ID NO: | MGSIGAASTEFCFDVFKELRVQHVNENVCYSPLIIISALSLVYLGARENTRAQIDKVVHFDKI |
| Ovalbumin-like | 231 | TGFGESIESQCGTSVSVHTSLKDMFNQITKPSDNYSLSVASRLYAEERYPILPEYLQCVKELY |
| [Chlamydotis | | KGGLESISFQTAADQAREAINSWVESQTNGMIKNILQPSSVDPQTEMVLVNAIYFKGMWQK |
| macqueenii] | | AFKDEDTQAVPFRISEQESKPVQMMYQIGSFKVAVMAAEKMKILELPYASGELSMLVLLPD |
| | | EVSGLEQLENAITVEKLMEWTSSSPMEERIMKVYLPRMKIEEKYNLTSVLMALGITDLFSSS |
| | | ANLSGISAEESLKMSEAVHQAFAEISEAGSEVVGSSEAGIDATSVSEEFRADHPFLFLIKHNA |
| | | TNSILFFGRCFSP |
| PREDICTED: | SEQ ID NO: | MGSISAASTEFCFDVFKELKVQHVNENIFYSPLSIISALSMVYLGARENTRAQIEKVVHFDKI |
| Ovalbumin like | 232 | TGFGESIESQCSTSVSVHTSLKDMFTQITKPSDNYSLSFASRFYAEETYPILPEYLQCVKELY |
| [Nipponia nippon] | | KGGLETINFRTAADQARELINSWVESQTNGMIKNILQPGSVDPQTDMVLVNAIYFKGMWE |
| | | KAFKDEDTQALPFRVTEQESKPVQMMYQIGSFKVAVLASEKVKILELPYASGQLSMLVLLP |
| | | DDVSGLEQLETAITVEKLMEWTSSNNMEERKIKVYLPRIKIEEKYNLTSVLMALGITDLFSS |
| | | SANLSGISSAESLKVSEAIHEAFVEIYEAGSEVAGSTEAGIEVTSVSEEFRADHPFLFLIKHNA |
| | | TNSILFFGRCFSP |
| PREDICTED: | SEQ ID NO: | MVSIGAASTEFCFDVFKELKVQHVNENIFYSPLSIISALSMVYLGARENTRAQIDKVVHFDKI |
| Ovalbumin-like | 233 | TGFEETIESQCSTSVSVHTSLKDMFTQITKPSDNYSLSFASRLYAEETYPILPEYLQCVKELY |
| isoform X2 [Gavia | | KGGLETISFQTAADQARELINSWVESQTDGMIKNILQPGSVDPQTEMVLVNAIYFKGMWEK |
| stellata] | | AFKDEDTQAVPFRMTEQESKPVQMMYQIGSFKVAVMASEKMKILELPYASGGMSMLVML |
| | | PDDVSGLEQLETAITFEKLMEWTSSNMMEERKMKVYLPRMKMEEKYNLTSVLMALGMT |
| | | DLFSSSANLSGISSAESLKMSEAVHEAFVEIYEAGSEAVGSTGAGMEVTSVSEEFRADHPFL |
| | | FLIKHNPTNSILFFGRCFSP |
| PREDICTED: | SEQ ID NO: | MGSIGAASTEFCFDVFKELKVQHVNENIFYSPLSIISALSMVYLGARENTRAQIDKVVHFDKI |
| Ovalbumin | 234 | TGFGEPIESQCGISVSVHTSLKDMITQITKPSDNYSLSFASRLYAEETYPILPEYLQCVKELYK |
| [Pelecanus crispus] | | GGLETISFQTAADQARELINSWVENQTNGMIKNILQPGSVDPQTEMVLVNAVYFKGMWEK |
| | | AFKDEDTQAVPFRMTEQESKPVQMMYQIGSFKVAVMASEKIKILELPYASGELSMLVLLPD |
| | | DVSGLEQLETAITLDKLTEWTSSNAMEERKMKVYLPRMKIEKKYNLTSVLIALGMTDLFSS |
| | | SANLSGISSAESLKMSEAIHEAFLEIYEAGSEVVGSTEAGMEVTSVSEEFRADHPFLFLIKHN |
| | | PTNSILFFGRCLSP |
| PREDICTED: | SEQ ID NO: | MGSIGAASTEFCFDVFKELKVQHVNENIFYSPLTIISALSMVYLGARENTRAQIDKVVHFDK |
| Ovalbumin-like | 235 | IPGFGDTTESQCGTSVSVHTSLKDMFTQITKPSDNYSVSFASRLYAEETYPILPEFLECVKEL |
| [Charadrius | | YKGGLESISFQTAADQARELINSWVESQTNGMIKNILQPGSVDSQTEMVLVNAIYFKGMWE |
| vociferus] | | KAFKDEDTQTVPFRMTEQETKPVQMMYQIGTFKVAVMPSEKMKILELPYASGELCMLVM |
| | | LPDDVSGLEELESSITVEKLMEWTSSNMMEERKMKVFLPRMKIEEKYNLTSVLMALGMTD |
| | | LFSSSANLSGISSAEPLKMSEAVHEAFIEIYEAGSEVVGSTGAGMEITSVSEEFRADHPFLFLI |
| | | KHNPTNSILFFGRCVSP |
| PREDICTED: | SEQ ID NO: | MGSIGAVSTEFCFDVFKELKVQHVNENIFYSPLSIISALSMVYLGARENTRAQIDKVVHFDKI |
| Ovalbumin-like | 236 | TGSGETIEAQCGTSVSVHTSLKDMFTQITKPSENYSVGFASRLYADETYPIIPEYLQCVKELY |
| [Eurypyga helias] | | KGGLEMISFQTAADQARELINSWVESQTNGMIKNILQPGSVDPQTEMILVNAIYFKGVWEK |
| | | AFKDEDTQAVPFRMTEQESKPVQMMYQFGSFKVAAMAAEKMKILELPYASGALSMLVLL |
| | | PDDVSGLEQLESAITFEKLMEWTSSNMMEEKKIKVYLPRMKMEEKYNFTSVLMALGMTD |
| | | LFSSSANLSGISSADSLKMSEVVHEAFVEIYEAGSEVVGSTGSGMEAASVSEEFRADHPFLF |
| | | LIKHNPTNSILFFGRCFSP |
| PREDICTED: | SEQ ID NO: | MVSIGAASTEFCFDVFKELKVQHVNENIFYSPLSIISALSMVYLGARENTRAQIDKVVHFDKI |
| Ovalbumin-like | 237 | TGFEETIESQVQKKQCSTSVSVHTSLKDMFTQITKPSDNYSLSFASRLYAEETYPILPEYLQC |
| isoform X1 [Gavia | | VKELYKGGLETISFQTAADQARELINSWVESQTDGMIKNILQPGSVDPQTEMVLVNAIYFK |
| stellata] | | GMWEKAFKDEDTQAVPFRMTEQESKPVQMMYQIGSFKVAVMASEKMKILELPYASGGMS |
| | | MLVMLPDDVSGLEQLETAITFEKLMEWTSSNMMEERKMKVYLPRMKMEEKYNLTSVLM |
| | | ALGMTDLFSSSANLSGISSAESLKMSEAVHEAFVEIYEAGSEAVGSTGAGMEVTSVSEEFRA |
| | | DHPFLFLIKHNPTNSILFFGRCFSP |
| PREDICTED: | SEQ ID NO: | MGSIGAASGEFCFDVFKELKVQHVNENIFYSPLSIISALSMVYLGARENTRAQIDKVVHFDK |
| Ovalbumin-like | 238 | IIGFGESIESQCGTSVSVHTSLKDMFAQITKPSDNYSLSFASRLYAEETFPILPEYLQCVKELY |
| [Egretta garzetta] | | KGGLETLSFQTAADQARELINSWVESQTNGMIKDILQPGSVDPQTEMVLVNAIYFKGVWE |
| | | KAFKDEDTQTVPFRMTEQESKPVQMMYQIGSFKVAVVAAEKIKILELPYASGALSMLVLLP |
| | | DDVSSLEQLETAITFEKLTEWTSSNIMEERKIKVYLPRMKIEEKYNLTSVLMDLGITDLFSSS |
| | | ANLSGISSAESLKVSEAIHEAIVDIYEAGSEVVGSSGAGLEGTSVSEEFRADHPFLFLIKHNPT |
| | | SSILFFGRCFSP |
| PREDICTED: | SEQ ID NO: | MGSIGAASTEFCFDVFKELKVQHVNENIFYSPLSIISALSMVYLGARENTRAQIDKVVHFDKI |
| Ovalbumin-like | 239 | TGSGEAIESQCGTSVSVHISLKDMFTQITKPSDNYSLSFASRLYAEETYPILPEYLQCVKELY |
| [Balearica regulorum | | KEGLATISFQTAADQAREFINSWVESQTNGMIKNILQPGSVDPQTQMVLVNAIYFKGVWEK |
| gibbericeps] | | AFKDEDTQAVPFRMTKQESKPVQMMYQIGSFKVAVMASEKMKILELPYASGQLSMLVML |
| | | PDDVSGLEQIENAITFEKLMEWTNPNMMEERKMKVYLPRMKMEEKYNLTSVLMALGMT |
| | | DLFSSSANLSGISSAESLKMSEAVHEAFVEIYEAGSEVVGSTGAGIEVTSVSEEFRADHPFLF |
| | | LIKHNPTNSILFFGRCFSP |
| PREDICTED: | SEQ ID NO: | MGSIGEASTEFCIDVFRELKVQHVNENIFYSPLSIISALSMVYLGARENTRAQIDQVVHFDKI |
| Ovalbumin-like | 240 | TGFGDTVESQCGSSLSVHSSLKDIFAQITQPKDNYSLNFASRLYAEETYPILPEYLQCVKELY |
| [Nestor notabilis] | | KGGLETISFQTAADQARELINSWVESQTNGMIKNILQPSSVDPQTEMVLVNAIYFKGVWEK |
| | | AFKDEETQAVPFRITEQENRPVQIMYQFGSFKVAVVASEKIKILELPYASGQLSMLVLLPDE |
| | | VSGLEQLENAITFEKLTEWTSSDIMEEKKIKVFLPRMKIEEKYNLTSVLVALGIADLFSSSAN |
| | | LSGISSAESLKMSEAVHEAFVEIYEAGSEVVGSSGAGIEAASDSEEFRADHPFLFLIKHKPTN |
| | | SILFFGRCFSP |
| PREDICTED: | SEQ ID NO: | MGSIGAASTEFCFDIFNELKVQHVNENIFYSPLSIISALSMVYLGARENTKAQIDKVVHFDKI |
| Ovalbumin-like | 241 | TGFGESIESQCSTSASVHTSFKDMFTQITKPSDNYSLSFASRLYAEETYPILPEYSQCVKELY |
| [Pygoscelis adeliae] | | KGGLESISFQTAADQARELINSWVESQTNGMIKNILQPGSVDPQTELVLVNAIYFKGTWEK |
| | | AFKDKDTQAVPFRVTEQESKPVQMMYQIGSYKVAVIASEKMKILELPYASGELSMLVLLPD |
| | | DVSGLEQLETAITFEKLMEWTSSNMMEERKVKVYLPRMKIEEKYNLTSVLMALGMTDLFS |
| | | PSANLSGISSAESLKMSEAIHEAFVEIYEAGSEVVGSTEAGMEVTSVSEEFRADHPFLFLIKC |
| | | NLTNSILFFGRCFSP |
| Ovalbumin-like | SEQ ID NO: | MGSISTASTEFCFDVFKELKVQHVNENIFYSPLSIISALSMVYLGARENTRAQIEKVVHFDKI |
| [Athene cunicularia] | 242 | TGFGESIESQCGTSVSVHTSLKDMLIQISKPSDNYSLSFASKLYAEETYPILPEYLQCVKELY |
| | | KGGLESINFQTAADQARQLINSWVESQTNGMIKDILQPSSVDPQTEMVLVNAIYFKGIWEK |
| | | AFKDEDTQEVPFRITEQESKPVQMMYQIGSFKVAVIASEKIKILELPYASGELSMLIVLPDDV |
| | | SGLEQLETAITFEKLIEWTSPSIMEERKTKVYLPRMKIEEKYNLTSVLMALGMTDLFSPSAN |
| | | LSGISSAESLKMSEAIHEAFVEIYEAGSEVVGSAEAGMEATSVSEFRVDHPFLFLIKHNPANII |
| | | LFFGRCVSP |
| PREDICTED: | SEQ ID NO: | MGSIGAASTEFCFDVFKELKVQHVNENIFYSPLTIISALSLVYLGARENTRAQIDKVFHFDKI |
| Ovalbumin-like | 243 | SGFGETTESQCGTSVSVHTSLKEMFTQITKPSDNYSVSFASRLYAEDTYPILPEYLQCVKELY |
| [Calidris pugnax] | | KGGLETISFQTAADQAREVINSWVESQTNGMIKNILQPGSVDSQTEMVLVNAIYFKGMWE |
| | | KAFKDEDTQTMPFRITEQERKPVQMMYQAGSFKVAVMASEKMKILELPYASGEFCMLIML |
| | | PDDVSGLEQLENSFSFEKLMEWTTSNMMEERKMKVYIPRMKMEEKYNLTSVLMALGMTD |
| | | LFSSSANLSGISSAETLKMSEAVHEAFMEIYEAGSEVVGSTGSGAEVTGVYEEFRADHPFLF |
| | | LVKHKPTNSILFFGRCVSP |
| PREDICTED: | SEQ ID NO: | MGSIGAASTEFCFDIFNELKVQHVNENIFYSPLSIISALSMVYLGARENTKAQIDKVVHFDKI |
| Ovalbumin | 244 | TGFGETIESQCSTSVSVHTSLKDTFTQITKPSDNYSLSFASRLYAEETYPILPEYSQCVKELYK |
| [Aptenodytes | | GGLETISFQTAADQARELINSWVESQTNGMIKNILQPGSVDPQTELVLVNAIYFKGTWEKAF |
| forsteri] | | KDKDTQAVPFRVTEQESKPVQMMYQIGSYKVAVIASEKMKILELPYASRELSMLVLLPDD |
| | | VSGLEQLETAITFEKLMEWTSSNMMEERKVKVYLPRMKIEEKYNLTSVLMALGMTDLFSP |
| | | SANLSGISSAESLKMSEAVHEAFVEIYEAGSEVVGSTGAGMEVTSVSEEFRADHPFLFLIKC |
| | | NPTNSILFFGRCFSP |
| PREDICTED: | SEQ ID NO: | MGSISAASAEFCLDVFKELKVQHVNENIFYSPLSIISALSMVYLGARENTRAQIDKVVHFDKI |
| Ovalbumin-like | 245 | TGSGETIEFQCGTSANIHPSLKDMFTQITRLSDNYSLSFASRLYAEERYPILPEYLQCVKELY |
| [Pterocles gutturalis] | | KGGLETISFQTAADQARELINSWVESQTNGMIKNILQPGSVNPQTEMVLVNAIYFKGLWEK |
| | | AFKDEDTQTVPFRMTEQESKPVQMMYQVGSFKVAVMASDKIKILELPYASGELSMLVLLP |
| | | DDVTGLEQLETSITFEKLMEWTSSNVMEERTMKVYLPHMRMEEKYNLTSVLMALGVTDL |
| | | FSSSANLSGISSAESLKMSEAVHEAFVEIYESGSQVVGSTGAGTEVTSVSEEFRVDHPFLFLI |
| | | KHNPTNSILFFGRCFSP |
| Ovalbumin-like | SEQ ID NO: | MGSIGAASVEFCFDVFKELKVQHVNENIFYSPLSIISALSMVYLGARENTKAQIDKVVHFDK |
| [Falco peregrinus] | 246 | IAGFGEAIESQCVTSASIHSLKDMFTQITKPSDNYSLSFASRLYAEEAYSILPEYLQCVKELY |
| | | KGGLETISFQTAADQARDLINSWVESQTNGMIKNILQPGAVDLETEMVLVNAIYFKGMWE |
| | | KAFKDEDTQTVPFRMTEQESKPVQMMYQVGSFKVAVMASDKIKILELPYASGQLSMVVV |
| | | LPDDVSGLEQLEASITSEKLMEWTSSSIMEEKKIKVYFPHMKIEEKYNLTSVLMALGMTDLF |
| | | SSSANLSGISSAEKLKVSEAVHEAFVEISEAGSEVVGSTEAGTEVTSVSEEFKADHPFLFLIK |
| | | HNPTNSILFFGRCFSP |
| PREDICTED: | SEQ ID NO: | MGSIGAASSEFCFDIFKELKVQHVNENIFYSPLSIISALSMVYLGARENTRAQIDKVVPFDKIT |
| Ovalbumin -like | 247 | ASGESIESQCSTSVSVHTSLKDIFTQITKSSDNHSLSFASRLYAEETYPILPEYLQCVKELYEG |
| isoform X2 | | GLETISFQTAADQARELINSWIESQTNGRIKNILQPGSVDPQTEMVLVNAIYFKGMWEKAFK |
| [Phalacrocorax | | DEDTQAVPFRMTEQESKPVQVMHQIGSFKVAVLASEKIKILELPYASGELSMLVLLPDDVS |
| carbo] | | GLEQLETAITFEKLMEWTSPNIMEERKIKVFLPRMKIEEKYNLTSVLMALGITDLFSPLANLS |
| | | GISSAESLKMSEAIHEAFVEISEAGSEVIGSTEAEVEVINDPEEFRADHPFLFLIKHNPTNSILF |
| | | FGRCFSP |
| PREDICTED: | SEQ ID NO: | MGSIGAASTEFCFDVFKELKAQYVNENIFYSPMTIITALSMVYLGSKENTRAQIAKVAHFDK |
| Ovalbumin-like | 248 | ITGFGESIESQCGASASIQFSLKDLFTQITKPSGNHSLSVASRIYAEETYPILPEYLECMKELYK |
| [Merops nubicus] | | GGLETINFQTAANQARELINSWVERQTSGMIKNILQPSSVDSQTEMVLVNAIYFRGLWEKA |
| | | FKVEDTQATPFRITEQESKPVQMMHQIGSFKVAVVASEKIKILELPYASGRLTMLVVLPDDV |
| | | SGLKQLETTITFEKLMEWTTSNIMEERKIKVYLPRMKIEEKYNLTSVLMALGLTDLFSSSAN |
| | | LSGISSAESLKMSEAVHEAFVEIYEAGSEVVASAEAGMDATSVSEEFRADHPFLFLIKDNTS |
| | | NSILFFGRCFSP |
| PREDICTED: | SEQ ID NO: | MGSIGAASTEFCFDVFKELKGQHVNENIFFCPLSIVSALSMVYLGARENTRAQIVKVAHFDK |
| Ovalbumin-like | 249 | IAGFAESIESQCGTSVSIHTSLKDMFTQITKPSDNYSLNFASRLYAEETYPIIPEYLQCVKELY |
| [Tauraco | | KGGLETISFQTAADQAREIINSWVESQTNGMIKNILRPSSVHPQTELVLVNAVYFKGTWEK |
| erythrolophus] | | AFKDEDTQAVPFRITEQESKPVQMMYQIGSFKVAAVTSEKMKILEVPYASGELSMLVLLPD |
| | | DVSGLEQLETAITAEKLIEWTSSTVMEERKLKVYLPRMKIEEKYNLTTVLTALGVTDLFSSS |
| | | ANLSGISSAQGLKMSNAVHEAFVEIYEAGSEVVGSKGEGTEVSSVSDEFKADHPFLFLIKHN |
| | | PTNSIVFFGRCFSP |
| PREDICTED: | SEQ ID NO: | MGSIGAASTEFCFDVFKELKVHHVNENILYSPLAIISALSMVYLGAKENTRDQIDKVVHFDK |
| Ovalbumin-like | 250 | ITGIGESIESQCSTAVSVHTSLKDVFDQITRPSDNYSLAFASRLYAEKTYPILPEYLQCVKELY |
| [Cuculus canorus] | | KGGLETIDFQTAADQARQLINSWVEDETNGMIKNILRPSSVNPQTKIILVNAIYFKGMWEKA |
| | | FKDEDTQEVPFRITEQETKSVQMMYQIGSFKVAEVVSDKMKILELPYASGKLSMLVLLPDD |
| | | VYGLEQLETVITVEKLKEWTSSIVMEERITKVYLPRMKIMEKYNLTSVLTAFGITDLFSPSA |
| | | NLSGISSTESLKVSEAVHEAFVEIHEAGSEVVGSAGAGIEATSVSEEFKADHPFLFLIKHNPT |
| | | NSILFFGRCFSP |
| Ovalbumin | SEQ ID NO: | MGSIGAASTEFCLDVFKELKVQHVNENIFYSPLSIISALSMVYLGARENTRAQIDKVVHFDK |
| [Antrostomus | 251 | ITGFEDSIESQCGTSVSVHTSLKDMFTQITKPSDNYSVGFASRLYAAETYQILPEYSQCVKEL |
| carolinensis] | | YKGGLETINFQKAADQATELINSWVESQTNGMIKNILQPSSVDPQTQIFLVNAIYFKGMWQ |
| | | RAFKEEDTQAVPFRISEKESKPVQMMYQIGSFKVAVIPSEKIKILELPYASGLLSMLVILPDD |
| | | VSGLEQLENAITLEKLMQWTSSNMMEERKIKVYLPRMRMEEKYNLTSVFMALGITDLFSSS |
| | | ANLSGISSAESLKMSDAVHEASVEIHEAGSEVVGSTGSGTEASSVSEEFRADHPYLFLIKHNP |
| | | TDSIVFFGRCFSP |
| PREDICTED: | SEQ ID NO: | MGSIGAASTEFCFDVFKELKFQHVDENIFYSPLTIISALSMVYLGARENTRAQIDKVVHFDKI |
| Ovalbumin-like | 252 | AGFEETVESQCGTSVSVHTSLKDMFAQITKPSDNYSLSFASRLYAEETYPILPEYLQCVKEL |
| [Opisthocomus | | YKGGLETISFQTAADQARDLINSWVESQTNGMIKNILQPSSVGPQTELILVNAIYFKGMWQ |
| hoazin] | | KAFKDEDTQEVPFRMTEQQSKPVQMMYQTGSFKVAVVASEKMKILALPYASGQLSLLVM |
| | | LPDDVSGLKQLESAITSEKLIEWTSPSMMEERKIKVYLPRMKIEEKYNLTSVLMALGITDLF |
| | | SPSANLSGISSAESLKMSQAVHEAFVEIYEAGSEVVGSTGAGMEDSSDSEEFRVDHPFLFFIK |
| | | HNPTNSILFFGRCFSP |
| PREDICTED: | SEQ ID NO: | MGSIGPLSVEFCCDVFKELRIQHPRENIFYSPVTIISALSMVYLGARDNTKAQIEKAVHFDKI |
| Ovalbumin-like | 253 | PGFGESIESQCGTSLSIHTSLKDIFTQITKPSDNYTVGIASRLYAEEKYPILPEYLQCIKELYKG |
| [Lepidothrix | | GLEPINFQTAAEQARELINSWVESQTNGMIKNILQPSSVNPETDMVLVNAIYFKGLWEKAF |
| coronata] | | KDEDIQTVPFRITEQESKPVQMMFQIGSFRVAEITSEKIRILELPYASGQLSLWVLLPDDISGL |
| | | EQLETAITFENLKEWTSSTKMEERKIKVYLPRMKIEEKYNLTSVLTSLGITDLFSSSANLSGIS |
| | | SAESLKVSSAFHEASVEIYEAGSKVVGSTGAEVEDTSVSEEFRADHPFLFLIKHNPSNSIFFFG |
| | | RCFSP |
| PREDICTED: | SEQ ID NO: | MGSIGTASAEFCFDVFKELKVHHVNENIFYSPLSIISALSMVYLGARENTKTQMEKVIHFDKI |
| Ovalbumin [Struthio | 254 | TGLGESMESQCGTGVSIHTALKDMLSEITKPSDNYSLSLASRLYAEQTYAILPEYLQCIKELY |
| camelus australis] | | KESLETVSFQTAADQARELINSWIESQTNGVIKNFLQPGSVDSQTELVLVNAIYFKGMWEK |
| | | AFKDEDTQEVPFRITEQESRPVQMMYQAGSFKVATVAAEKIKILELPYASGELSMLVLLPD |
| | | DISGLEQLETTISFEKLTEWTSSNMMEDRNMKVYLPRMKIEEKYNLTSVLIALGMTDLFSPA |
| | | ANLSGISAAESLKMSEAIHAAYVEIYEADSEIVSSAGVQVEVTSDSEEFRVDHPFLFLIKHNP |
| | | TNSVLFFGRCISP |
| PREDICTED: | SEQ ID NO: | MGSIGAVSTEFSCDVFKELRIHHVQENIFYSPVTIISALSMIYLGARDSTKAQIEKAVHFDKIP |
| Ovalbumin-like | 255 | GFGESIESQCGTSLSIHTSIKDMFTKITKASDNYSIGIASRLYAEEKYPILPEYLQCVKELYKG |
| [Acanthisitta chloris] | | GLESISFQTAAEQAREIINSWVESQTNGMIKNILQPSSVDPQTDIVLVNAIYFKGLWEKAFRD |
| | | EDTQTVPFKITEQESKPVQMMYQIGSFKVAEITSEKIKILEVPYASGQLSLWVLLPDDISGLE |
| | | KLETAITFENLKEWTSSTKMEERKIKVYLPRMKIEEKYNLTSVLTALGITDLFSSSANLSGIS |
| | | SAESLKVSEAFHEAIVEISEAGSKVVGSVGAGVDDTSVSEEFRADHPFLFLIKHNPTSSIFFFG |
| | | RCFSP |
| PREDICTED: | SEQ ID NO: | MGSIGAASTEFCFDVFKELKVQHVNENIFYSPLSIISALSMVYLGARENTRAQIDKVVHFDKI |
| Ovalbumin-like | 256 | AGFGESTESQCGTSVSAHTSLKDMSNQITKLSDNYSLSFASRLYAEETYPILPEYSQCVKEL |
| [Tyto alba] | | YKGGLESISFQTAAYQARELINAWVESQTNGMIKDILQPGSVDSQTKMVLVNAIYFKGIWE |
| | | KAFKDEDTQEVPFRMTEQETKPVQMMYQIGSFKVAVIAAEKIKILELPYASGQLSMLVILPD |
| | | DVSGLEQLETAITFEKLTEWTSASVMEERKIKVYLPRMSIEEKYNLTSVLIALGVTDLFSSSA |
| | | NLSGISSAESLRMSEAIHEAFVETYEAGSTESGTEVTSASEEFRVDHPFLFLIKHKPTNSILFF |
| | | GRCFSP |
| PREDICTED: | SEQ ID NO: | MGSIGAASSEFCFDIFKELKVQHVNENIFYSPLSIISALSMVYLGARENTRAQIDKVVPFDKIT |
| Ovalbumin -like | 257 | ASGESIESQVQKIQCSTSVSVHTSLKDIFTQITKSSDNHSLSFASRLYAEETYPILPEYLQCVK |
| isoform X1 | | ELYEGGLETISFQTAADQARELINSWIESQTNGRIKNILQPGSVDPQTEMVLVNAIYFKGMW |
| [Phalacrocorax | | EKAFKDEDTQAVPFRMTEQESKPVQVMHQIGSFKVAVLASEKIKILELPYASGELSMLVLL |
| carbo] | | PDDVSGLEQLETAITFEKLMEWTSPNIMEERKIKVFLPRMKIEEKYNLTSVLMALGITDLFSP |
| | | LANLSGISSAESLKMSEAIHEAFVEISEAGSEVIGSTEAEVEVINDPEEFRADHPFLFLIKHNP |
| | | TNSILFFGRCFSP |
| Ovalbumin-like | SEQ ID NO: | MGSIGPLSVEFCCDVFKELRIQHARENIFYSPVTIISALSMVYLGARDNTKAQIEKAVHFDKI |
| [Pipra filicauda] | 258 | PGFGESIESQCGTSLSIHTSLKDIFTQITKPSDNYTVGIASRLYAEEKYPILPEYLQCIKELYKG |
| | | GLEPISFQTAAEQARELINSWVESQTNGIIKNILQPSSVNPETDMVLVNAIYFKGLWEKAFK |
| | | DEGTQTVPFRITEQESKPVQMMFQIGSFRVAEIASEKIRILELPYASGQLSLWVLLPDDISGLE |
| | | QLETAITFENLKEWTSSTKMEERKIKVYLPRMKIEEKYNLTSVLTSLGITDLFSSSANLSGISS |
| | | AERLKVSSAFHEASMEINEAGSKVVGAGVDDTSVSEEFRVDRPFLFLIKHNPSNSIFFFGRCF |
| | | SP |
| Ovalbumin | SEQ ID NO: | MGSIGAASTEFCFDMFKELKVHHVNENIIYSPLSIISILSMVFLGARENTKTQMEKVIHFDKIT |
| [Dromaius | 259 | GFGESLESQCGTSVSVHASLKDILSEITKPSDNYSLSLASKLYAEETYPVLPEYLQCIKELYK |
| novaehollandiae] | | GSLETVSFQTAADQARELINSWVETQTNGVIKNFLQPGSVDPQTEMVLVDAIYFKGTWEK |
| | | AFKDEDTQEVPFRITEQESKPVQMMYQAGSFKVATVAAEKMKILELPYASGELSMFVLLPD |
| | | DISGLEQLETTISIEKLSEWTSSNMMEDRKMKVYLPHMKIEEKYNLTSVLVALGMTDLFSPS |
| | | ANLSGISTAQTLKMSEAIHGAYVEIYEAGSEMATSTGVLVEAASVSEEFRVDHPFLFLIKHN |
| | | PSNSILFFGRCIFP |
| Chain A, Ovalbumin | SEQ ID NO: | MGSIGAASTEFCFDMFKELKVHHVNENIIYSPLSIISILSMVFLGARENTKTQMEKVIHFDKIT |
| | 260 | GFGESLESQCGTSVSVHASLKDILSEITKPSDNYSLSLASKLYAEETYPVLPEYLQCIKELYK |
| | | GSLETVSFQTAADQARELINSWVETQTNGVIKNFLQPGSVDPQTEMVLVDAIYFKGTWEK |
| | | AFKDEDTQEVPFRITEQESKPVQMMYQAGSFKVATVAAEKMKILELPYASGELSMFVLLPD |
| | | DISGLEQLETTISIEKLSEWTSSNMMEDRKMKVYLPHMKIEEKYNLTSVLVALGMTDLFSPS |
| | | ANLSGISTAQTLKMSEAIHGAYVEIYEAGSEMATSTGVLVEAASVSEEFRVDHPFLFLIKHN |
| | | PSNSILFFGRCIFPHHHHHH |
| Ovalbumin-like | SEQ ID NO: | MGSIGPLSVEFCCDVFKELRIQHARENIFYSPVTIISALSMVYLGARDNTKAQIEKAVHFDKI |
| [Corapipo altera] | 261 | PGFGESIESQCGTSLSIHTSLKDIFTQITKPSDNYTVGIASRLYAEEKYPILPEYLQCIKELYKG |
| | | GLEPISFQTAAEQARELINSWVESQTNGMIKNILQPSAVNPETDMVLVNAIYFKGLWEKAF |
| | | KDEGTQTVPFRITEQESKPVQMMFQIGSFRVAEITSEKIRILELPYASGQLSLWVLLPDDISGL |
| | | EQLETAITFENLKEWTSSTKMEERKIKVYLPRMKIEEKYNLTSVLTSLGITDLFSSSANLSGIS |
| | | SAERLKVSSAFHEASMEIYEAGSKVVGSTGAGVDDTSVSEEFRVDRPFLFLIKHNPSNSIFFF |
| | | GRCFSP |
| Ovalbumin-like | SEQ ID NO: | MEDQRGNTGFTMGSIGAASTEFCIDVFRELRVQHVNENIFYSPLTIISALSMVYLGARENTR |
| protein [Amazona | 262 | AQIDQVVHFDKIAGFGDTVESQCGSSPSVHNSLKTVXAQITQPRDNYSLNLASRLYAEESYP |
| aestiva] | | ILPEYLQCVKELYNGGLETVSFQTAADQARELINSWVESQINGIIKNILQPSSVDPQTEMVL |
| | | VNAIYFKGLWEKAFKDEETQAVPFRITEQENRPVQMMYQFGSFKVAXVASEKIKILELPYA |
| | | SGQLSMLVLLPDEVSGLEQNAITFEKLTEWTSSDLMEERKIKVFFPRVKIEEKYNLTAVLVS |
| | | LGITDLFSSSANLSGISSAENLKMSEAVHEAXVEIYEAGSEVAGSSGAGIEVASDSEEFRVDH |
| | | PFLFLIXHNPTNSILFFGRCFSP |
| PREDICTED: | SEQ ID NO: | MGSIGAASTEFCIDVFRELRVQHVNENIFYSPLSIISALSMVYLGARENTRAQIDEVFHFDKI |
| Ovalbumin-like | 263 | AGFGDTVDPQCGASLSVHKSLQNVFAQITQPKDNYSLNLASRLYAEESYPILPEYLQCVKE |
| [Melopsittacus | | LYNEGLETVSFQTGADQARELINSWVENQTNGVIKNILQPSSVDPQTEMVLVNAIYFKGLW |
| undulatus] | | QKAFKDEETQAVPFRITEQENRPVQMMYQFGSFKVAVVASEKVKILELPYASGQLSMWVL |
| | | LPDEVSGLEQLENAITFEKLTEWTSSDLTEERKIKVFLPRVKIEEKYNLTAVLMALGVTDLF |
| | | SSSANFSGISAAENLKMSEAVHEAFVEIYEAGSEVVGSSGAGIEAPSDSEEFRADHPFLFLIK |
| | | HNPTNSILFFGRCFSP |
| Ovalbumin-like | SEQ ID NO: | MGSIGPLSVEFCCDVFKELRIQHARDNIFYSPVTIISALSMVYLGARDNTKAQIEKAVHFDKI |
| [Neopelma | 264 | PGFGESIESQCGTSLSVHTSLKDIFTQITKPRENYTVGIASRLYAEEKYPILPEYLQCIKELYK |
| chrysocephalum] | | GGLEPISFQTAAEQARELINSWVESQTNGMIKNILQPSSVNPETDMVLVNAIYFKGLWKKA |
| | | FKDEGTQTVPFRITEQESKPVQMMFQIGSFRVAEITSEKIRILELPYASGQLSLWVLLPDDISG |
| | | LEQLESAITFENLKEWTSSTKMEERKIKVYLPRMKIEEKYNLTSVLTSLGITDLFSSSANLSGI |
| | | SSAEKLKVSSAFHEASMEIYEAGNKVVGSTGAGVDDTSVSEEFRVDRPFLFLIKHNPSNSIFF |
| | | FGRCFSP |
| PREDICTED: | SEQ ID NO: | MGSIGAASAEFCVDVFKELKDQHVNNIVFSPLMIISALSMVNIGAREDTRAQIDKVVHFDKI |
| Ovalbumin-like | 265 | TGYGESIESQCGTSIGIYFSLKDAFTQITKPSDNYSLSFASKLYAEETYPILPEYLKCVKELYK |
| [Buceros rhinoceros | | GGLETISFQTAADQARELINSWVESQTNGMIKNILQPSSVDPQTEMVLVNAIYFKGLWEKA |
| silvestris] | | FKDEDTQAVPFRITEQESKPVQMMYQIGSFKVAVIASEKIKILELPYASGQLSLLVLLPDDVS |
| | | GLEQLESAITSEKLLEWTNPNIMEERKTKVYLPRMKIEEKYNLTSVLVALGITDLFSSSANLS |
| | | GISSAEGLKLSDAVHEAFVEIYEAGREVVGSSEAGVEDSSVSEEFKADRPFIFLIKHNPTNGI |
| | | LYFGRYISP |
| PREDICTED: | SEQ ID NO: | MGSIGAANTDFCFDVFKELKVHHANENIFYSPLSIVSALAMVYLGARENTRAQIDKALHFD |
| Ovalbumin-like | 266 | KILGFGETVESQCDTSVSVHTSLKDMLIQITKPSDNYSFSFASKIYTEETYPILPEYLQCVKEL |
| [Cariama cristata] | | YKGGVETISFQTAADQAREVINSWVESHTNGMIKNILQPGSVDPQTKMVLVNAVYFKGIW |
| | | EKAFKEEDTQEMPFRINEQESKPVQMMYQIGSFKLTVAASENLKILEFPYASGQLSMMVILP |
| | | DEVSGLKQLETSITSEKLIKWTSSNTMEERKIRVYLPRMKIEEKYNLKSVLMALGITDLFSSS |
| | | ANLSGISSAESLKMSEAVHEAFVEIYEAGSEVTSSTGTEMEAENVSEEFKADHPFLFLIKHNP |
| | | TDSIVFFGRCMSP |
| Ovalbumin [Manacus | SEQ ID NO: | MGSIGPLSVEFCCDVFKELRIQHARENIFYSPVTIISALSMVYLGARDNTKAQIEKAVHFDKI |
| vitellinus] | 267 | PGFGESIESQCGTSLSIHTSLKDIFTQITKPSDNYTVGIASRLYAEEKYPILPEYLQCIKELYKG |
| | | GLEPISFQTAAEQARELINSWVESQTNGMIKNILQPSSVNPETDMVLVNAIYFKGLWEKAFK |
| | | DESTQTVPFRITEQESKPVQMMFQIGSFRVAEIASEKIRILELPYASGQLSLWVLLPDDISGLE |
| | | QLETAITFENLKEWTSSTKMEERKIKVYLPRMKIEEKYNLTSVLTSLGITDLFSSSANLSGISS |
| | | AERLKVSSAFHEASMEIYEAGSRVVEAGVDDTSVSEEFRVDRPFLFLIKHNPSNSIFFFGRCF |
| | | SP |
| Ovalbumin-like | SEQ ID NO: | MGSIGPVSTEFCCDIFKELRIQHARENIIYSPVTIISALSMVYLGARDNTKAQIEKAVHFDKIP |
| [Empidonax traillii] | 268 | GFGESIESQCGTSLSIHTSLKDILTQITKPSDNYTVGIASRLYAEEKYPILSEYLQCIKELYKGG |
| | | LEPISFQTAAEQARELINSWVESQTNGMIKNILQPSSVNPETDMVLVNAIYFKGLWEKAFKD |
| | | EGTQTVPFRITEQESKPVQMMFQIGSFKVAEITSEKIRILELPYASGKLSLWVLLPDDISGLEQ |
| | | LETAITFENLKEWTSSTRMEERKIKVYLPRMKIEEKYNLTSVLTSLGITDLFSSSANLSGISSA |
| | | ERLKVSSAFHEVFVEIYEAGSKVEGSTGAGVDDTSVSEEFRADHPFLFLVKHNPSNSIIFFGR |
| | | CYLP |
| PREDICTED: | SEQ ID NO: | MGSTGAASMEFCFALFRELKVQHVNENIFFSPVTIISALSMVYLGARENTRAQLDKVAPFD |
| Ovalbumin-like | 269 | KITGFGETIGSQCSTSASSHTSLKDVFTQITKASDNYSLSFASRLYAEETYPILPEYLQCVKEL |
| [Leptosomus | | YKGGLESISFQTAADQARELINSWVESQTNGMIKDILRPSSVDPQTKIILITAIYFKGMWEKA |
| discolor] | | FKEEDTQAVPFRMTEQESKPVQMMYQIGSFKVAVIPSEKLKILELPYASGQLSMLVILPDDV |
| | | SGLEQLETAITTEKLKEWTSPSMMKERKMKVYFPRMRIEEKYNLTSVLMALGITDLFSPSA |
| | | NLSGISSAESLKVSEAVHEASVDIDEAGSEVIGSTGVGTEVTSVSEEIRADHPFLFLIKHKPTN |
| | | SILFFGRCFSP |
| Hypothetical protein | SEQ ID NO: | MEHAQLTQLVNSNMTSNTCHEADEFENIDFRMDSISVTNTKFCFDVFNEMKVHHVNENIL |
| H355 008077 | 270 | YSPLSILTALAMVYLGARGNTESQMKKALHFDSITGAGSTTDSQCGSSEYIHNLFKEFLTEIT |
| [Colinus virginianus] | | RTNATYSLEIADKLYVDKTFTVLPEYINCARKFYTGGVEEVNFKTAAEEARQLINSWVEKE |
| | | TNGQIKDLLVPSSVDFGTMMVFINTIYFKGIWKTAFNTEDTREMPFSMTKQESKPVQMMCL |
| | | NDTFNMATLPAEKMRILELPYASGELSMLVLLPDEVSGLEQIEKAINFEKLREWTSTNAME |
| | | KKSMKVYLPRMKIEEKYNLTSTLMALGMTDLFSRSANLTGISSVENLMISDAVHGAFMEV |
| | | NEEGTEAAGSTGAIGNIKHSVEFEEFRADHPFLFLIRYNPTNVILFFDNSEFTMGSIGAVSTEF |
| | | CFDVFKELRVHHANENIFYSPFTVISALAMVYLGAKDSTRTQINKVVRFDKLPGFGDSIEAQ |
| | | CGTSANVHSSLRDILNQITKPNDIYSFSLASRLYADETYTILPEYLQCVKELYRGGLESINFQ |
| | | TAADQARELINSWVESQTSGIIRNVLQPSSVDSQTAMVLVNAIYFKGLWEKGFKDEDTQA |
| | | MPFRVTEQENKSVQMMYQIGTFKVASVASEKMKILELPFASGTMSMWVLLPDEVSGLEQL |
| | | ETTISIEKLTEWTSSSVMEERKIKVFLPRMKMEEKYNLTSVLMAMGMTDLFSSSANLSGISS |
| | | TLQKKGFRSQELGDKYAKPMLESPALTPQVTAWDNSWIVAHPAAIEPDLCYQIMEQKWKP |
| | | FDWPDFRLPMRVSCRFRTMEALNKANTSFALDFFKHECQEDDDENILFSPFSISSALATVYL |
| | | GAKGNTADQMAKTEIGKSGNIHAGFKALDLEINQPTKNYLLNSVNQLYGEKSLPFSKEYLQ |
| | | LAKKYYSAEPQSVDFLGKANEIRREINSRVEHQTEGKIKNLLPPGSIDSLTRLVLVNALYFK |
| | | GNWATKFEAEDTRHRPFRINMHTTKQVPMMYLRDKFNWTYVESVQTDVLELPYVNNDLS |
| | | MFILLPRDITGLQKLINELTFEKLSAWTSPELMEKMKMEVYLPRFTVEKKYDMKSTLSKMG |
| | | IEDAFTKVDSCGVTNVDEITTHIVSSKCLELKHIQINKKLKCNKAVAMEQVSASIGNFTIDLF |
| | | NKLNETSRDKNIFFSPWSVSSALALTSLAAKGNTAREMAEDPENEQAENIHSGFKELMTAL |
| | | NKPRNTYSLKSANRIYVEKNYPLLPTYIQLSKKYYKAEPYKVNFKTAPEQSRKEINNWVEK |
| | | QTERKIKNFLSSDDVKNSTKSILVNAIYFKAEWEEKFQAGNTDMQPFRMSKNKSKLVKMM |
| | | YMRHTFPVLIMEKLNFKMIELPYVKRELSMFILLPDDIKDSTTGLEQLERELTYEKLSEWAD |
| | | SKKMSVTLVDLHLPKFSMEDRYDLKDALKSMGMASAFNSNADFSGMTGFQAVPMESLSA |
| | | STNSFTLDLYKKLDETSKGQNIFFASWSIATALAMVHLGAKGDTATQVAKGPEYEETENIH |
| | | SGFKELLSAINKPRNTYLMKSANRLFGDKTYPLLPKFLELVARYYQAKPQAVNFKTDAEQ |
| | | ARAQINSWVENETESKIQNLLPAGSIDSHTVLVLVNAIYFKGNWEKRFLEKDTSKMPFRLS |
| | | KTETKPVQMMFLKDTFLIHHERTMKFKIIELPYVGNELSAFVLLPDDISDNTTGLELVEREL |
| | | TYEKLAEWSNSASMMKAKVELYLPKLKMEENYDLKSVLSDMGIRSAFDPAQADFTRMSE |
| | | KKDLFISKVIHKAFVEVNEEDRIVQLASGRLTGRCRTLANKELSEKNRTKNLFFSPFSISSAL |
| | | SMILLGSKGNTEAQIAKVLSLSKAEDAHNGYQSLLSEINNPDTKYILRTANRLYGEKTFEFL |
| | | SSFIDSSQKFYHAGLEQTDFKNASEDSRKQINGWVEEKTEGKIQKLLSEGIINSMTKLVLVN |
| | | AIYFKGNWQEKFDKETTKEMPFKINKNETKPVQMMFRKGKYNMTYIGDLETTVLEIPYVD |
| | | NELSMIILLPDSIQDESTGLEKLERELTYEKLMDWINPNMMDSTEVRVSLPRFKLEENYELK |
| | | PTLSTMGMPDAFDLRTADFSGISSGNELVLSEVVHKSFVEVNEEGTEAAAATAGIMLLRCA |
| | | MIVANFTADHPFLFFIRHNKTNSILFCGRFCSP |
| PREDICTED: | SEQ ID NO: | MGSIGTASTEFCFDMFKEMKVQHANQNIIFSPLTIISALSMVYLGARDNTKAQMEKVIHFDK |
| Ovalbumin isoform | 271 | ITGFGESVESQCGTSVSIHTSLKDMLSEITKPSDNYSLSLASRLYAEETYPILPEYLQCMKEL |
| X2 [Apteryx australis | | YKGGLETVSFQTAADQARELINSWVESQTNGVIKNFLQPGSVDPQTEMVLVNAIYFKGMW |
| mantelli] | | EKAFKDEDTQEVPFRITEQESKPVQMMYQVGSFKVATVAAEKMKILEIPYTHRELSMFVLL |
| | | PDDISGLEQLETTISFEKLTEWTSSNMMEERKVKVYLPHMKIEEKYNLTSVLMALGMTDLF |
| | | SPSANLSGISTAQTLMMSEAIHGAYVEIYEAGREMASSTGVQVEVTSVLEEVRADKPFLFFI |
| | | RHNPTNSMVVFGRYMSP |
| Hypothetical protein | SEQ ID NO: | MTSNTCHEADEFENIDFRMDSISVTNTKFCFDVFNEMKVHHVNENILYSPLSILTALAMVYL |
| ASZ78_006007 | 272 | GARGNTESQMKKALHFDSITGGGSTTDSQCGSSEYIHNLFKEFLTEITRTNATYSLEIADKL |
| [Callipepla | | YVDKTFTVLPEYINCARKFYTGGVEEVNFKTAAEEARQLMNSWVEKETNGQIKDLLVPSS |
| squamata] | | VDFGTMMVFINTIYFKGIWKTAFNTEDTREMPFSMTKQESKPVQMMCLNDTFNMVTLPAE |
| | | KMRILELPYASGELSMLVLLPDEVSGLERIEKAINFEKLREWTSTNAMEKKSMKVYLPRMK |
| | | JEEKYNLTSTLMALGMTDLFSRSANLTGISSVDNLMISDAVHGAFMEVNEEGTEAAGSTGA |
| | | IGNIKHSVEFEEFRADHPFLFLIRYNPTNVILFFDNSEFTMGSIGAVSTEFCFDVFKELRVHHA |
| | | NENIFYSPFTIISALAMVYLGAKDSTRTQINKVVRFDKLPGFGDSIEAQCGTSANVHSSLRDI |
| | | LNQITKPNDIYSFSLASRLYADETYTILPEYLQCVKELYRGGLESINFQTAADQARELINSWV |
| | | ESQTSGIIRNVLQPSSVDSQTAMVLVNAIYFKGLWEKGFKDEDTQAIPFRVTEQENKSVQM |
| | | MYQIGTFKVASVASEKMKILELPFASGTMSMWVLLPDEVSGLEQLETTISIEKLTEWTSSSV |
| | | MEERKIKVFLPRMKMEEKYNLTSVLMAMGMTDLFSSSANLSGISSTLQKKGFRSQELGDK |
| | | YAKPMLESPALTPQATAWDNSWIVAHPPAIEPDLYYQIMEQKWKPFDWPDFRLPMRVSCR |
| | | FRTMEALNKANTSFALDFFKHECQEDDSENILFSPFSISSALATVYLGAKGNTADQMAKVL |
| | | HFNEAEGARNVTTTIRMQVYSRTDQQRLNRRACFQKTEIGKSGNIHAGFKGLNLEINQPTK |
| | | NYLLNSVNQLYGEKSLPFSKEYLQLAKKYYSAEPQSVDFVGTANEIRREINSRVEHQTEGKI |
| | | KNLLPPGSIDSLTRLVLVNALYFKGNWATKFEAEDTRHRPFRINTHTTKQVPMMYLSDKFN |
| | | WTYVESVQTDVLELPYVNNDLSMFILLPRDITGLQKLINELTFEKLSAWTSPELMEKMKME |
| | | VYLPRFTVEKKYDMKSTLSKMGIEDAFTKVDNCGVTNVDEITIHVVPSKCLELKHIQINKEL |
| | | KCNKAVAMEQVSASIGNFTIDLFNKLNETSRDKNIFFSPWSVSSALALTSLAAKGNTAREM |
| | | AEDPENEQAENIHSGFNELLTALNKPRNTYSLKSANRIYVEKNYPLLPTYIQLSKKYYKAEP |
| | | HKVNFKTAPEQSRKEINNWVEKQTERKIKNFLSSDDVKNSTKLILVNAIYFKAEWEEKFQA |
| | | GNTDMQPFRMSKNKSKLVKMMYMRHTFPVLIMEKLNFKMIELPYVKRELSMFILLPDDIK |
| | | DSTTGLEQLERELTYEKLSEWADSKKMSVTLVDLHLPKFSMEDRYDLKDALRSMGMASA |
| | | FNSNADFSGMTGERDLVISKVCHQSFVAVDEKGTEAAAATAVIAEAVPMESLSASTNSFTL |
| | | DLYKKLDETSKGQNIFFASWSIATALTMVHLGAKGDTATQVAKGPEYEETENIHSGFKELL |
| | | SALNKPRNTYSMKSANRLFGDKTYPLLPTKTKPVQMMFLKDTFLIHHERTMKFKIIELPYM |
| | | GNELSAFVLLPDDISDNTTGLELVERELTYEKLAEWSNSASMMKVKVELYLPKLKMEENY |
| | | DLKSALSDMGIRSAFDPAQADFTRMSEKKDLFISKVIHKAFVEVNEEDRIVQLASGRLTGNT |
| | | EAQIAKVLSLSKAEDAHNGYQSLLSEINNPDTKYILRTANRLYGEKTFEFLSSFIDSSQKFYH |
| | | AGLEQTDFKNASEDSRKQINGWVEEKTEGKIQKLLSEGIINSMTKLVLVNAIYFKGNWQEK |
| | | FDKETTKEMPFKINKNETKPVQMMFRKGKYNMTYIGDLETTVLEIPYVDNELSMIILLPDSI |
| | | QDESTGLEKLERELTYEKLMDWINPNMMDSTEVRVSLPRFKLEENYELKPTLSTMGMPDA |
| | | FDLRTADFSGISSGNELVLSEVVHKSFVEVNEEGTEAAAATAGIMLLRCAMIVANFTADHP |
| | | FLFFIRHNKTNSILFCGRFCSP |
| PREDICTED: | SEQ ID NO: | MASIGAASTEFCFDVFKELKTQHVKENIFYSPMAIISALSMVYIGARENTRAEIDKVVHFDKI |
| Ovalbumin-like | 273 | TGFGNAVESQCGPSVSVHSSLKDLITQISKRSDNYSLSYASRIYAEETYPILPEYLQCVKEVY |
| [Mesitornis unicolor] | | KGGLESISFQTAADQARENINAWVESQTNGMIKNILQPSSVNPQTEMVLVNAIYLKGMWE |
| | | KAFKDEDTQTMPFRVTQQESKPVQMMYQIGSFKVAVIASEKMKILELPYTSGQLSMLVLLP |
| | | DDVSGLEQVESAITAEKLMEWTSPSIMEERTMKVYLPRMKMVEKYNLTSVLMALGMTDL |
| | | FTSVANLSGISSAQGLKMSQAIHEAFVEIYEAGSEAVGSTGVGMEITSVSEEFKADLSFLFLI |
| | | RHNPTNSIIFFGRCISP |
| Ovalbumin, partial | SEQ ID NO: | MGSIGAASTEFCFDVFRELRVQHVNENIFYSPFSIISALAMVYLGARDNTRTQIDKISQFQAL |
| [Anas platyrhynchos] | 274 | SDEHLVLCIQQLGEFFVCTNRERREVTRYSEQTEDKTQDQNTGQIHKIVDTCMLRQDILTQI |
| | | TKPSDNFSLSFASRLYAEETYAILPEYLQCVKELYKGGLESISFQTAADQARELINSWVESQT |
| | | NGIIKNILQPSSVDSQTTMVLVNAIYFKGMWEKAFKDEDTQAMPFRMTEQESKPVQMMYQ |
| | | VGSFKVAMVTSEKMKILELPFASGMMSMFVLLPDEVSGLEQLESTISFEKLTEWTSSTMME |
| | | ERRMKVYLPRMKMEEKYNLTSVFMALGMTDLFSSSANMSGISSTVSLKMSEAVHAACVEI |
| | | FEAGRDVVGSAEAGMDVTSVSEEFRADHPFLFFIKHNPTNSILFFGRWMSP |
| PREDICTED: | SEQ ID NO: | MGSIGAASAEFCLDIFKELKVQHVNENIIFSPMTIISALSLVYLGAKEDTRAQIEKVVPFDKIP |
| Ovalbumin-like | 275 | GFGEIVESQCPKSASVHSSIQDIFNQIIKRSDNYSLSLASRLYAEESYPIRPEYLQCVKELDKE |
| [Chaetura pelagica] | | GLETISFQTAADQARQLINSWVESQTNGMIKNILQPSSVNSQTEMVLVNAIYFRGLWQKAF |
| | | KDEDTQAVPFRITEQESKPVQMMQQIGSFKVAEIASEKMKILELPYASGQLSMLVLLPDDV |
| | | SGLEKLESSITVEKLIEWTSSNLTEERNVKVYLPRLKIEEKYNLTSVLAALGITDLFSSSANLS |
| | | GISTAESLKLSRAVHESFVEIQEAGHEVEGPKEAGIEVTSALDEFRVDRPFLFVTKHNPTNSI |
| | | LFLGRCLSP |
| PREDICTED: | SEQ ID NO: | MGSISAASGEFCLDIFKELKVQHVNENIFYSPMVIVSALSLVYLGARENTRAQIDKVIPFDKI |
| Ovalbumin-like | 276 | TGSSEAVESQCGTPVGAHISLKDVFAQIAKRSDNYSLSFVNRLYAEETYPILPEYLQCVKEL |
| [Apaloderma | | YKGGLETISFQTAADQAREIINSWVESQTDGKIKNILQPSSVDPQTKMVLVSAIYFKGLWEK |
| vittatum] | | SFKDEDTQAVPFRVTEQESKPVQMMYQIGSFKVAAIAAEKIKILELPYASEQLSMLVLLPDD |
| | | VSGLEQLEKKISYEKLTEWTSSSVMEEKKIKVYLPRMKIEEKYNLTSILMSLGITDLFSSSAN |
| | | LSGISSTKSLKMSEAVHEASVEIYEAGSEASGITGDGMEATSVFGEFKVDHPFLFMIKHKPT |
| | | NSILFFGRCISP |
| Ovalbumin-like | SEQ ID NO: | MGSIGPVSTEVCCDIFRELRSQSVQENVCYSPLLIISTLSMVYIGAKDNTKAQIEKAIHFDKIP |
| [Corvus cornix | 277 | GFGESTESQCGTSVSIHTSLKDIFTQITKPSDNYSISIARRLYAEEKYPILPEYIQCVKELYKGG |
| cornix] | | LESISFQTAAEKSRELINSWVESQTNGTIKNILQPSSVSSQTDMVLVSAIYFKGLWEKAFKEE |
| | | DTQTIPFRITEQESKPVQMMSQIGTFKVAEIPSEKCRILELPYASGRLSLWVLLPDDISGLEQL |
| | | ETAITFENLKEWTSSSKMEERKIRVYLPRMKIEEKYNLTSVLKSLGITDLFSSSANLSGISSAE |
| | | SLKVSAAFHEASVEIYEAGSKGVGSSEAGVDGTSVSEEIRADHPFLFLIKHNPSDSILFFGRC |
| | | FSP |
| PREDICTED: | SEQ ID NO: | MGSIGAASTEFCFDVFKELKVQHVNENIIISPLSIISALSMVYLGAREDTRAQIDKVVHFDKIT |
| Ovalbumin-like | 278 | GFGEAIESQCPTSESVHASLKETFSQLTKPSDNYSLAFASRLYAEETYPILPEYLQCVKELYK |
| [Calypte anna] | | GGLETINFQTAAEQARQVINSWVESQTDGMIKSLLQPSSVDPQTEMILVNAIYFRGLWERAF |
| | | KDEDTQELPFRITEQESKPVQMMSQIGSFKVAVVASEKVKILELPYASGQLSMLVLLPDDVS |
| | | GLEQLESSITVEKLIEWISSNTKEERNIKVYLPRMKIEEKYNLTSVLVALGITDLFSSSANLSG |
| | | ISSAESLKISEAVHEAFVEIQEAGSEVVGSPGPEVEVTSVSEEWKADRPFLFLIKHNPTNSILF |
| | | FGRYISP |
| PREDICTED: | SEQ ID NO: | MGSIGPVSTEVCCDIFRELRSQSVQENVCYSPLLIISTLSMVYIGAKDNTKAQIEKAIHFDKIP |
| Ovalbumin [Corvus | 279 | GFGESTESQCGTSVSIHTSLKDIFTQITKPSDNYSISIARRLYAEEKYPILQEYIQCVKELYKG |
| brachyrhynchos] | | GLESISFQTAAEKSRELINSWVESQTNGTIKNILQPSSVSSQTDMVLVSAIYFKGLWEKAFKE |
| | | EDTQTIPFRITEQESKPVQMMSQIGTFKVAEIPSEKCRILELPYASGRLSLWVLLPDDISGLEQ |
| | | LETSITFENLKEWTSSSKMEERKIRVYLPRMKIEEKYNLTSVLKSLGITDLFSSSANLSGISSA |
| | | ESLKVSAVFHEASVEIYEAGSKGVGSSEAGVDGTSVSEEIRADHPFLFLIKHNPSDSILFFGR |
| | | CFSP |
| Hypothetical protein | SEQ ID NO: | MLNLMHPKQFCCTMGSIGPVSTEVCCDIFRELRSQSVQENVCYSPLLIISTLSMVYIGAKDN |
| DUI87 08270 | 280 | TKAQIEKAIHFDKIPGFGESTESQCGTSVSIHTSLKDIFTQITKPSDNYSISIASRLYAEEKYPIL |
| [Hirundo rustica | | PEYIQCVKELYKGGLESISFQTAAEKSRELINSWVESQTNGTIKNILQPSSVSSQTDMVLVSA |
| rustica] | | IYFKGLWEKAFKEEDTQTVPFRITEQESKPVQMMSQIGTFKVAEIPSEKCRILELPYASGRLS |
| | | LWVLLPDDISGLEQLETAITSENLKEWTSSSKMEERKIKVYLPRMKIEEKYNLTSVLKSLGIT |
| | | DLFSSSANLSGISSAESLKVSGAFHEAFVEIYEAGSKAVGSSGAGVEDTSVSEEIRADHPFLF |
| | | FIKHNPSDSILFFGRCFSP |
| Ostrich OVA | SEQ ID NO: | EAEAGSIGTASAEFCFDVFKELKVHHVNENIFYSPLSIISALSMVYLGARENTKTQMEKVIHF |
| sequence as secreted | 281 | DKITGLGESMESQCGTGVSIHTALKDMLSEITKPSDNYSLSLASRLYAEQTYAILPEYLQCIK |
| from pichia | | ELYKESLETVSFQTAADQARELINSWIESQTNGVIKNFLQPGSVDSQTELVLVNAIYFKGM |
| | | WEKAFKDEDTQEVPFRITEQESRPVQMMYQAGSFKVATVAAEKIKILELPYASGELSMLVL |
| | | LPDDISGLEQLETTISFEKLTEWTSSNMMEDRNMKVYLPRMKIEEKYNLTSVLIALGMTDLF |
| | | SPAANLSGISAAESLKMSEAIHAAYVEIYEADSEIVSSAGVQVEVTSDSEEFRVDHPFLFLIK |
| | | HNPTNSVLFFGRCISP |
| Ostrich construct | SEQ ID NO: | MRFPSIFTAVLFAASSALAAPVNTTTEDETAQIPAEAVIGYSDLEGDFDVAVLPFSNSTNNG |
| (secretion signal + | 282 | LLFINTTIASIAAKEEGVSLEKREAEAGSIGTASAEFCFDVFKELKVHHVNENIFYSPLSIISAL |
| mature protein) | | SMVYLGARENTKTQMEKVIHFDKITGLGESMESQCGTGVSIHTALKDMLSEITKPSDNYSL |
| | | SLASRLYAEQTYAILPEYLQCIKELYKESLETVSFQTAADQARELINSWIESQTNGVIKNFLQ |
| | | PGSVDSQTELVLVNAIYFKGMWEKAFKDEDTQEVPFRITEQESRPVQMMYQAGSFKVATV |
| | | AAEKIKILELPYASGELSMLVLLPDDISGLEQLETTISFEKLTEWTSSNMMEDRNMKVYLPR |
| | | MKIEEKYNLTSVLIALGMTDLFSPAANLSGISAAESLKMSEAIHAAYVEIYEADSEIVSSAGV |
| | | QVEVTSDSEEFRVDHPFLFLIKHNPTNSVLFFGRCISP |
| Duck OVA sequence | SEQ ID NO: | EAEAGSIGAASTEFCFDVFRELRVQHVNENIFYSPFSIISALAMVYLGARDNTRTQIDKVVHF |
| as secreted from | 283 | DKLPGFGESMEAQCGTSVSVHSSLRDILTQITKPSDNFSLSFASRLYAEETYAILPEYLQCVK |
| pichia | | ELYKGGLESISFQTAADQARELINSWVESQINGIIKNILQPSSVDSQTTMVLVNAIYFKGMW |
| | | EKAFKDEDTQAMPFRMTEQESKPVQMMYQVGSFKVAMVTSEKMKILELPFASGMMSMF |
| | | VLLPDEVSGLEQLESTISFEKLTEWTSSTMMEERRMKVYLPRMKMEEKYNLTSVFMALGM |
| | | TDLFSSSANMSGISSTVSLKMSEAVHAACVEIFEAGRDVVGSAEAGMDVTSVSEEFRADHP |
| | | FLFFIKHNPTNSILFFGRWMSP |
| Duck construct | SEQ ID NO: | MRFPSIFTAVLFAASSALAAPVNTTTEDETAQIPAEAVIGYSDLEGDFDVAVLPFSNSTNNG |
| (secretion signal + | 284 | LLFINTTIASIAAKEEGVSLEKREAEAGSIGAASTEFCFDVFRELRVQHVNENIFYSPFSIISAL |
| mature protein) | | AMVYLGARDNTRTQIDKVVHFDKLPGFGESMEAQCGTSVSVHSSLRDILTQITKPSDNFSLS |
| | | FASRLYAEETYAILPEYLQCVKELYKGGLESISFQTAADQARELINSWVESQINGIIKNILQP |
| | | SSVDSQTTMVLVNAIYFKGMWEKAFKDEDTQAMPFRMTEQESKPVQMMYQVGSFKVAM |
| | | VTSEKMKILELPFASGMMSMFVLLPDEVSGLEQLESTISFEKLTEWTSSTMMEERRMKVYL |
| | | PRMKMEEKYNLTSVFMALGMTDLFSSSANMSGISSTVSLKMSEAVHAACVEIFEAGRDVV |
| | | GSAEAGMDVTSVSEEFRADHPFLFFIKHNPTNSILFFGRWMSP |
| Ovoglobulin G2 | SEQ ID NO: | TRAPDCGGILTPLGLSYLAEVSKPHAEVVLRQDLMAQRASDLFLGSMEPSRNRITSVKVAD |
| | 285 | LWLSVIPEAGLRLGIEVELRIAPLHAVPMPVRISIRADLHVDMGPDGNLQLLTSACRPTVQA |
| | | QSTREAESKSSRSILDKVVDVDKLCLDVSKLLLFPNEQLMSLTALFPVTPNCQLQYLPLAAP |
| | | VFSKQGIALSLQTTFQVAGAVVPVPVSPVPFSMPELASTSTSHLILALSEHFYTSLYFTLERA |
| | | GAFNMTIPSMLTTATLAQKITQVGSLYHEDLPITLSAALRSSPRVVLEEGRAALKLFLTVHIG |
| | | AGSPDFQSFLSVSADVTAGLQLSVSDTRMMISTAVIEDAELSLAASNVGLVRAALLEELFLA |
| | | PVCQQVPAWMDDVLREGVHLPHLSHFTYTDVNVVVHKDYVLVPCKLKLRSTMA* |
| Ovoglobulin G3 | SEQ ID NO: | MDSISVTNAKFCFDVFNEMKVHHVNENILYCPLSILTALAMVYLGARGNTESQMKKVLHF |
| | 286 | DSITGAGSTTDSQCGSSEYVHNLFKELLSEITRPNATYSLEIADKLYVDKTFSVLPEYLSCAR |
| | | KFYTGGVEEVNFKTAAEEARQLINSWVEKETNGQIKDLLVSSSIDFGTTMVFINTIYFKGIW |
| | | KIAFNTEDTREMPFSMTKEESKPVQMMCMNNSFNVATLPAEKMKILELPYASGDLSMLVL |
| | | LPDEVSGLERIEKTINFDKLREWTSTNAMAKKSMKVYLPRMKIEEKYNLTSILMALGMTDL |
| | | FSRSANLTGISSVDNLMISDAVHGVFMEVNEEGTEATGSTGAIGNIKHSLELEEFRADHPFLF |
| | | FIRYNPTNAILFFGRYWSP* |
| B-ovomucin | SEQ ID NO: | CSTWGGGHFSTFDKYQYDFTGTCNYIFATVCDESSPDFNIQFRRGLDKKIARIIIELGPSVIIV |
| | 287 | EKDSISVRSVGVIKLPYASNGIQIAPYGRSVRLVAKLMEMELVVMWNNEDYLMVLTEKKY |
| | | MGKTCGMCGNYDGYELNDFVSEGKLLDTYKFAALQKMDDPSEICLSEEISIPAIPHKKYAV |
| | | ICSQLLNLVSPTCSVPKDGFVTRCQLDMQDCSEPGQKNCTCSTLSEYSRQCAMSHQVVFN |
| | | WRTENFCSVGKCSANQIYEECGSPCIKTCSNPEYSCSSHCTYGCFCPEGTVLDDISKNRTCV |
| | | HLEQCPCTLNGETYAPGDTMKAACRTCKCTMGQWNCKELPCPGRCSLEGGSFVTTFDSRS |
| | | YRFHGVCTYILMKSSSLPHNGTLMAIYEKSGYSHSETSLSAIIYLSTKDKIVISQNELLTDDD |
| | | ELKRLPYKSGDITIFKQSSMFIQMHTEFGLELVVQTSPVFQAYVKVSAQFQGRTLGLCGNY |
| | | NGDTTDDFMTSMDITEGTASLFVDSWRAGNCLPAMERETDPCALSQLNKISAETHCSILTK |
| | | KGTVFETCHAVVNPTPFYKRCVYQACNYEETFPYICSALGSYARTCSSMGLILENWRNSMD |
| | | NCTITCTGNQTFSYNTQACERTCLSLSNPTLECHPTDIPIEGCNCPKGMYLNHKNECVRKSH |
| | | CPCYLEDRKYILPDQSTMTGGITCYCVNGRLSCTGKLQNPAESCKAPKKYISCSDSLENKY |
| | | GATCAPTCQMLATGIECIPTKCESGCVCADGLYENLDGRCVPPEECPCEYGGLSYGKGEQI |
| | | QTECEICTCRKGKWKCVQKSRCSSTCNLYGEGHITTFDGQRFVFDGNCEYILAMDGCNVN |
| | | RPLSSFKIVTENVICGKSGVTCSRSISIYLGNLTIILRDETYSISGKNLQVKYNVKKNALHLMF |
| | | DIIIPGKYNMTLIWNKHMNFFIKISRETQETICGLCGNYNGNMKDDFETRSKYVASNELEFV |
| | | NSWKENPLCGDVYFVVDPCSKNPYRKAWAEKTCSIINSQVFSACHNKVNRMPYYEACVR |
| | | DSCGCDIGGDCECMCDAIAVYAMACLDKGICIDWRTPEFCPVYCEYYNSHRKTGSGGAYS |
| | | YGSSVNCTWHYRPCNCPNQYYKYVNIEGCYNCSHDEYFDYEKEKCMPCAMQPTSVTLPT |
| | | ATQPTSPSTSSASTVLTETTNPPV* |
| Lysozyme | SEQ ID NO: | KVFGRCELAAAMKRHGLDNYRGYSLGNWVCAAKFESNFNTQATNRNTDGSTDYGILQIN |
| | 288 | SRWWCNDGRTPGSRNLCNIPCSALLSSDITASVNCAKKIVSDGNGMNAWVAWRNRCKGT |
| | | DVQAWIRGCRL* |
| Lysozyme | SEQ ID NO: | KVFGRCELAAAMKRHGLDNYRGYSLGNWVCVAKFESNFNTQATNRNTDGSTDYGILQIN |
| | 289 | SRWWCNDGRTPGSRNLCNIPCSALLSSDITASVNCAKKIVSDGNGMSAWVAWRNRCKGT |
| | | DVQAWIRGCRL* |
| Lysozyme C | SEQ ID NO: | KVFERCELARTLKRLGMDGYRGISLANWMCLAKWESGYNTRATNYNAGDRSTDYGIFQI |
| (Human) | 290 | NSRYWCNDGKTPGAVNACHLSCSALLQDNIADAVACAKRVVRDPQGIRAWVAWRNRCQ |
| | | NRDVRQYVQGCGV* |
| Lysozyme C (Bos | SEQ ID NO: | KVFERCELARTLKKLGLDGYKGVSLANWLCLTKWESSYNTKATNYNPSSESTDYGIFQINS |
| taurus) | 291 | KWWCNDGKTPNAVDGCHVSCRELMENDIAKAVACAKHIVSEQGITAWVAWKSHCRDHD |
| | | VSSYVEGCTL* |
| Ovoinhibitor | SEQ ID NO: | IEVNCSLYASGIGKDGTSWVACPRNLKPVCGTDGSTYSNECGICLYNREHGANVEKEYDGE |
| | 292 | CRPKHVMIDCSPYLQVVRDGNTMVACPRILKPVCGSDSFTYDNECGICAYNAEHHTNISKL |
| | | HDGECKLEIGSVDCSKYPSTVSKDGRTLVACPRILSPVCGTDGFTYDNECGICAHNAEQRT |
| | | HVSKKHDGKCRQEIPEIDCDQYPTRKTTGGKLLVRCPRILLPVCGTDGFTYDNECGICAHN |
| | | AQHGTEVKKSHDGRCKERSTPLDCTQYLSNTQNGEAITACPFILQEVCGTDGVTYSNDCSL |
| | | CAHNIELGTSVAKKHDGRCREEVPELDCSKYKTSTLKDGRQVVACTMIYDPVCATNGVTY |
| | | ASECTLCAHNLEQRTNLGKRKNGRCEEDITKEHCREFQKVSPICTMEYVPHCGSDGVTYSN |
| | | RCFFCNAYVQSNRTLNLVSMAAC* |
| Cystatin | SEQ ID NO: | MAGARGCVVLLAAALMLVGAVLGSEDRSRLLGAPVPVDENDEGLQRALQFAMAEYNRA |
| | 293 | SNDKYSSRVVRVISAKRQLVSGIKYILQVEIGRTTCPKSSGDLQSCEFHDEPEMAKYTTCTF |
| | | VVYSIPWLNQIKLLESKCQ* |
| Porcine Lipase | SEQ ID NO: | SEVCFPRLGCFSDDAPWAGIVQRPLKILPWSPKDVDTRFLLYTNQNQNNYQELVADPSTIT |
| | 294 | NSNFRMDRKTRFIIHGFIDKGEEDWLSNICKNLFKVESVNCICVDWKGGSRTGYTQASQNIR |
| | | IVGAEVAYFVEVLKSSLGYSPSNVHVIGHSLGSHAAGEAGRRTNGTIERITGLDPAEPCFQG |
| | | TPELVRLDPSDAKFVDVIHTDAAPIIPNLGFGMSQTVGHLDFFPNGGKQMPGCQKNILSQIV |
| | | DIDGIWEGTRDFVACNHLRSYKYYADSILNPDGFAGFPCDSYNVFTANKCFPCPSEGCPQM |
| | | GHYADRFPGKTNGVSQVFYLNTGDASNFARWRYKVSVTLSGKKVTGHILVSLFGNEGNSR |
| | | QYEIYKGTLQPDNTHSDEFDSDVEVGDLQKVKFIWYNNNVINPTLPRVGASKITVERNDGK |
| | | VYDFCSQETVREEVLLTLNPC* |
| Kid Lipase | SEQ ID NO: | GLVAADRITGGKDFRDIESKFALRTPEDTAEDTCHLIPGVTESVANCHFNHSSKTFVVIHGW |
| | 295 | TVTGMYESWVPKLVAALYKREPDSNVIVVDWLSRAQQHYPVSAGYTKLVGQDVAKFMN |
| | | WMADEFNYPLGNVHLLGYSLGAHAAGIAGSLTSKKVNRITGLDPAGPNFEYAEAPSRLSPD |
| | | DADFVDVLHTFTRGSPGRSIGIQKPVGHVDIYPNGGTFQPGCNIGEALRVIAERGLGDVDQL |
| | | VKCSHERSVHLFIDSLLNEENPSKAYRCNSKEAFEKGLCLSCRKNRCNNMGYEINKVRAKR |
| | | SSKMYLKTRSQMPYKVFHYQVKIHFSGTESNTYTNQAFEISLYGTVAESENIPFTLPEVSTN |
| | | KTYSFLLYTEVDIGELLMLKLKWISDSYFSWSNWWSSPGFDIGKIRVKAGETQKKVIFCSRE |
| | | KMSYLQKGKSPVIFVKCHDKSLNRKSG* |
| Porcine Lactoferrin | SEQ ID NO: | APKKGVRWCVISTAEYSKCRQWQSKIRRTNPMFCIRRASPTDCIRAIAAKRADAVTLDGGL |
| | 296 | VFEADQYKLRPVAAEIYGTEENPQTYYYAVAVVKKGFNFQLNQLQGRKSCHTGLGRSAG |
| | | WNIPIGLLRRFLDWAGPPEPLQKAVAKFFSQSCVPCADGNAYPNLCQLCIGKGKDKCACSS |
| | | QEPYFGYSGAFNCLHKGIGDVAFVKESTVFENLPQKADRDKYELLCPDNTRKPVEAFRECH |
| | | LARVPSHAVVARSVNGKENSIWELLYQSQKKFGKSNPQEFQLFGSPGQQKDLLFRDATIGF |
| | | LKIPSKIDSKLYLGLPYLTAIQGLRETAAEVEARQAKVVWCAVGPEELRKCRQWSSQSSQN |
| | | LNCSLASTTEDCIVQVLKGEADAMSLDGGFIYTAGKCGLVPVLAENQKSRQSSSSDCVHRP |
| | | TQGYFAVAVVRKANGGITWNSVRGTKSCHTAVDRTAGWNIPMGLLVNQTGSCKFDEFFS |
| | | QSCAPGSQPGSNLCALCVGNDQGVDKCVPNSNERYYGYTGAFRCLAENAGDVAFVKDVT |
| | | VLDNTNGQNTEEWARELRSDDFELLCLDGTRKPVTEAQNCHLAVAPSHAVVSRKEKAAQ |
| | | VEQVLLTEQAQFGRYGKDCPDKFCLFRSETKNLLFNDNTEVLAQLQGKTTYEKYLGSEYV |
| | | TAIANLKQCSVSPLLEACAFMMR* |
| Bovine Lactoferrin | SEQ ID NO: | APRKNVRWCTISQPEWFKCRRWQWRMKKLGAPSITCVRRAFALECIRALAEKKADAVTLD |
| | 297 | GGMVFEAGRDPYKLRPVAAEIYGTKESPQTHYYAVAVVKKGSNFQLDQLQGRKSCHTGL |
| | | GRSAGWIIPMGILRPYLSWTESLEPLQGAVAKFFSASCVPCIDRQAYPNLCQLCKGEGENQC |
| | | ACSSREPYFGYSGAFKCLQDGAGDVAFVKETTVFENLPEKADRDQYELLCLNNSRAPVDA |
| | | FKECHLAQVPSHAVVARSVDGKEDLIWKLLSKAQEKFGKNKSRSFQLFGSPPGQRDLLFKD |
| | | SALGFLRIPSKVDSALYLGSRYLTTLKNLRETAEEVKARYTRVVWCAVGPEEQKKCQQWS |
| | | QQSGQNVTCATASTTDDCIVLVLKGEADALNLDGGYIYTAGKCGLVPVLAENRKSSKHSS |
| | | LDCVLRPTEGYLAVAVVKKANEGLTWNSLKDKKSCHTAVDRTAGWNIPMGLIVNQTGSC |
| | | AFDEFFSQSCAPGADPKSRLCALCAGDDQGLDKCVPNSKEKYYGYTGAFRCLAEDVGDVA |
| | | FVKNDTVWENTNGESTADWAKNLNREDFRLLCLDGTRKPVTEAQSCHLAVAPNHAVVSR |
| | | SDRAAHVKQVLLHQQALFGKNGKNCPDKFCLFKSETKNLLFNDNTECLAKLGGRPTYEEY |
| | | LGTEYVTALIANLKKCSTSPLLEACAFLTR* |
| Saccharomyces | SEQ ID NO: | APVNTTTEDETAQIPAEAVIGYSDLEGDFDVAVLPFSNSTNNGLLFINTTIASIAAKEEGVSL |
| cerevisiae α-mating | 298 | DKR |
| factor signal peptide | | |
| and secretion signal | | |
| Saccharomyces | SEQ ID NO: | APVNTTTEDETAQIPAEAVIGYSDLEGDFDVAVLPFSNSTNNGLLFINTTIASIAAKEEGVSL |
| cerevisiae α-mating | 299 | DKREAEA |
| factor signal peptide | | |
| and secretion signal | | |
| ending with EAEA | | |
| EndoH- | SEQ ID NO: | MTIAHHCIFLVILAFLALINVASGAPAPVKQGPTSVAYVEVNNNSMLNVGKYTLADGGGN |
| Saccharomyces | 300 | AFDVAVIFAANINYDTGTKTAYLHFNENVQRVLDNAVTQIRPLQQQGIKVLLSVLGNHQG |
| cerevisiae Flo5 | | AGFANFPSQQAASAFAKQLSDAVAKYGLDGVDFDDEYAEYGNNGTAQPNDSSFVHLVTA |
| fusion (full ORF, | | LRANMPDKIISLYNIGPAASRLSYGGVDVSDKFDYAWNPYYGTWQVPGIALPKAQLSPAA |
| including peptides | | VEIGRTSRSTVADLARRTVDEGYGVYLTYNLDGGDRTADVSAFTRELYGSEAVRTPGSSGS |
| that are cleaved off | | SGSSGSSGSSGSSGSSGSSEAAAREAAAREAAAREAAARGGGGSGGGGSGGGGSATEACLP |
| post-translationally) | | AGQRKSGMNINFYQYSLKDSSTYSNAAYMAYGYASKTKLGSVGGQTDISIDYNIPCVSSSG |
| | | TFPCPQEDSYGNWGCKGMGACSNSQGIAYWSTDLFGFYTTPTNVTLEMTGYFLPPQTGSY |
| | | TFSFATVDDSAILSVGGSIAFECCAQEQPPITSTNFTINGIKPWDGSLPDNITGTVYMYAGYY |
| | | YPLKVVYSNAVSWGTLPISVELPDGTTVSDNFEGYVYSFDDDLSQSNCTIPDPSIHTTSTITT |
| | | TTEPWTGTFTSTSTEMTTITDTNGQLTDETVIVIRTPTTASTITTTTEPWTGTFTSTSTEMTTV |
| | | TGTNGQPTDETVIVIRTPTSEGLITTTTEPWTGTFTSTSTEMTTVTGTNGQPTDETVIVIRTPT |
| | | SEGLITTTTEPWTGTFTSTSTEVTTITGTNGQPTDETVIVIRTPTSEGLITTTTEPWTGTFTSTS |
| | | TEMTTVTGTNGQPTDETVIVIRTPTSEGLISTTTEPWTGTFTSTSTEVTTITGTNGQPTDETVI |
| | | VIRTPTSEGLITTTTEPWTGTFTSTSTEMTTVTGTNGQPTDETVIVIRTPTSEGLITRTTEPWT |
| | | GTFTSTSTEVTTITGTNGQPTDETVIVIRTPTTAISSSLSSSSGQITSSITSSRPIITPFYPSNGTSV |
| | | ISSSVISSSVTSSLVTSSSFISSSVISSSTTTSTSIFSESSTSSVIPTSSSTSGSSESKTSSASSSSSSSS |
| | | ISSESPKSPTNSSSSLPPVTSATTGQETASSLPPATTTKTSEQTTLVTVTSCESHVCTESISSAIV |
| | | STATVTVSGVTTEYTTWCPISTTETTKQTKGTTEQTKGTTEQTTETTKQTTVVTISSCESDIC |
| | | SKTASPAIVSTSTATINGVTTEYTTWCPISTTESKQQTTLVTVTSCESGVCSETTSPAIVSTAT |
| | | ATVNDVVTVYPTWRPQTTNEQSVSSKMNSATSETTTNTGAAETKTAVTSSLSRFNHAETQ |
| | | TASATDVIGHSSSVVSVSETGNTMSLTSSGLSTMSQQPRSTPASSMVGSSTASLEISTYAGSA |
| | | NSLLAGSGLSVFIASLLLAII |
| A flexible GS linker | SEQ ID NO: | GSSGSSGSSGSSGSSGSSGSSGSS |
| with higher S content | 301 | |
| A flexible GS linker | SEQ ID NO: | GGGGGGGGSGGGGS |
| with much higher G | 302 | |
| content | | |
| EndoH-Tir4 | SEQ ID NO: | tgtcaaaacagtagtgataaaaggctatgaaggaggttgtctaggggctcgcggaggaaagtgattcaaacagacctgccaaaaagagaaaaa |
| construct: | 303 | agagggaatccctgttctttccaatggaaatgacgtaactttaacttgaaaaataccccaaccagaagggttcaaactcaacaaggattgcgtaatt |
| | | cctacaagtagcttagagctgggggagagacaactgaaggcagcttaacgataacgcggggggattggtgcacgactcgaaaggaggtatctt |
| | | agtcttgtaacctcttttttccagaggctattcaagattcataggcgatatcgatgtggagaagggtgaacaatataaaaggctggagagatgtcaat |
| | | gaagcagctggatagatttcaaattttctagatttcagagtaatcgcacaaaacgaaggaatcccaccaagacaaaaaaaaaaattctaagGAA |
| | | TTCCGAAACGATGAGATTCCCTTCTATTTTCACTGCTGTTTTGTTCGCTGCTTCTTCTGCT |
| | | TTGGCTGCTCCAGTTAACACTACTACCGAAGACGAAACTGCTCAAATTCCTGCTGAAGC |
| | | TGTTATTGGTTACTCTGACTTGGAAGGTGACTTCGACGTTGCTGTTTTGCCATTCTCTAA |
| | | CTCTACTAACAACGGTTTGTTGTTCATTAACACTACTATTGCTTCTATTGCTGCTAAGGA |
| | | AGAAGGTGTTTCTTTGGACAAGAGAGAGGCCGAAGCTGCTCCAGCCCCCGTAAAGCAG |
| | | GGACCAACCTCTGTAGCTTACGTAGAGGTAAATAACAACTCCATGTTAAACGTAGGAA |
| | | AATACACACTGGCCGACGGAGGAGGCAACGCATTTGACGTAGCTGTGATATTCGCCGC |
| | | TAATATTAACTATGACACGGGCACCAAGACAGCATATCTACATTTCAACGAGAACGTA |
| | | CAAAGAGTGCTTGACAACGCCGTAACCCAAATCCGTCCTCTTCAGCAACAAGGAATTA |
| | | AGGTCTTGTTGTCTGTTTTAGGAAATCATCAGGGCGCTGGATTCGCTAACTTTCCCTCTC |
| | | AGCAAGCAGCCTCCGCATTTGCTAAACAATTGTCAGACGCCGTCGCCAAGTACGGCTT |
| | | AGACGGAGTTGACTTCGATGACGAGTATGCTGAATACGGAAACAATGGCACCGCACAG |
| | | CCAAACGACTCCTCTTTTGTTCATCTTGTCACCGCTCTACGTGCCAATATGCCAGATAA |
| | | AATTATTTCCCTTTATAATATAGGTCCTGCTGCTTCTCGTTTGTCCTACGGCGGTGTAGA |
| | | TGTCTCTGACAAGTTTGATTACGCCTGGAATCCTTATTACGGCACGTGGCAGGTCCCCG |
| | | GAATCGCACTTCCTAAGGCCCAGCTATCTCCTGCAGCAGTTGAAATCGGCAGAACGTCT |
| | | CGTTCCACCGTGGCAGATTTAGCAAGGAGAACAGTCGATGAAGGATATGGAGTGTACT |
| | | TAACGTATAATCTTGATGGCGGCGAtAGGACCGCAGACGTATCTGCCTTTACTAGAGAG |
| | | TTGTATGGAAGTGAGGCCGTTCGTACTCCCGGTTCATCAGGGTCCTCAGGATCATCCGG |
| | | TAGTAGTGGTTCATCCGGTTCATCCGGATCAAGTGGCTCCTCTGAAGCTGCAGCAAGGG |
| | | AGGCTGCAGCCCGTGAGGCAGCCGCTAGAGAAGCCGCCGCTAGGGGTGGTGGCGGCTC |
| | | TGGCGGAGGCGGTTCCGGTGGCGGAGGCTCTCAAATCAACGAATTGAACGTTGTTTTA |
| | | GATGATGTTAAGACCAACATTGCCGACTACATCACCCTATCCTACACTCCAAATTCAGG |
| | | TTTTTCCTTGGACCAAATGCCAGCTGGTATTATGGATATTGCTGCGCAATTGGTTGCAA |
| | | ATCCAAGTGATGACTCCTACACCACTTTGTACTCTGAAGTGGACTTTTCTGCTGTTGAG |
| | | CATATGTTGACTATGGTCCCATGGTACTCTTCTAGACTGCTTCCAGAATTAGAAGCAAT |
| | | GGATGCTTCTCTAACTACCTCAAGTTCTGCTGCCACATCTTCAAGTGAAGTTGCTAGCT |
| | | CTTCTATTGCTTCATCCACTAGCTCTTCTGTTGCACCATCCTCAAGTGAAGTTGTCAGCT |
| | | CTTCCGTTGCTTCATCCTCAAGTGAAGTTGCCAGCTCCTCTGTTGCGTCTACAAGCGAA |
| | | GCTACTAGTTCTTCTGCTGTCACATCTTCCTCCGCTGTTTCCTCTTCGACCGAGTCTGTT |
| | | AGCTCTTCCTCTGTCAGTTCTTCCTCAGCCGTTTCCTCTTCTGAAGCTGTCAGTTCCTCTC |
| | | CAGTTTCCTCAGTTGTTTCATCTTCGGCCGGACCTGCTAGCTCAAGCGTTGCTCCTTACA |
| | | ACTCAACCATTGCTAGCTCTTCTTCCACTGCCCAGACTTCTATCTCGACCATTGCTCCTT |
| | | ACAACTCCACAACCACCACCACCCCAGCTAGTTCTGCTTCCAGCGTTATTATCTCAACC |
| | | AGAAACGGTACCACTGTTACTGAAACTGACAACACTCTTGTCACCAAAGAAACCACTG |
| | | TCTGTGACTACTCTTCAACATCTGCCGTTCCAGCTTCCACCACCGGTTACAACAATTCTA |
| | | CTAAGGTTTCAACCGCTACTATCTGCAGTACATGCAAAGAAGGTACCTCTACTGCAACT |
| | | GACTTCTCTACACTAAAGACTACAGTTACCGTATGTGACTCCGCCTGTCAAGCTAAGAA |
| | | GTCTGCTACCGTTGTTAGCGTTCAATCTAAAACTACCGGTATCGTTGAACAAACCGAAA |
| | | ACGGTGCTGCCAAGGCTGTTATCGGTATGGGTGCCGGTGCTTTAGCTGCTGTTGCCGCC |
| | | ATGCTACTATGAGCGGCCGCtcgatttgtatgtgaaatagctgaaattcgaaaatttcattatggctgtatctactttagcgtattag |
| | | gcatttgagcattggcttgaacaatgcgggctgtagtgtgtcaccaaagaaaccattcgggttcggatctggaagtcctcatcacgtgatgccgat |
| | | ctcgtgtattttattttcagataacacctgaagacttt |
| EndoH-Dan1 | SEQ ID NO: | tgtcaaaacagtagtgataaaaggctatgaaggaggttgtctaggggctcgcggaggaaagtgattcaaacagacctgccaaaaagagaaaaa |
| construct: | 304 | agagggaatccctgttctttccaatggaaatgacgtaactttaacttgaaaaataccccaaccagaagggttcaaactcaacaaggattgcgtaatt |
| | | cctacaagtagcttagagctgggggagagacaactgaaggcagcttaacgataacgcggggggattggtgcacgactcgaaaggaggtatctt |
| | | agtcttgtaacctcttttttccagaggctattcaagattcataggcgatatcgatgtggagaagggtgaacaatataaaaggctggagagatgtcaat |
| | | gaagcagctggatagatttcaaattttctagatttcagagtaatcgcacaaaacgaaggaatcccaccaagacaaaaaaaaaaattctaagGAA |
| | | TTCCGAAACGATGAGATTCCCTTCTATTTTCACTGCTGTTTTGTTCGCTGCTTCTTCTGCT |
| | | TTGGCTGCTCCAGTTAACACTACTACCGAAGACGAAACTGCTCAAATTCCTGCTGAAGC |
| | | TGTTATTGGTTACTCTGACTTGGAAGGTGACTTCGACGTTGCTGTTTTGCCATTCTCTAA |
| | | CTCTACTAACAACGGTTTGTTGTTCATTAACACTACTATTGCTTCTATTGCTGCTAAGGA |
| | | AGAAGGTGTTTCTTTGGACAAGAGAGAGGCCGAAGCTGCTCCAGCCCCCGTAAAGCAG |
| | | GGACCAACCTCTGTAGCTTACGTAGAGGTAAATAACAACTCCATGTTAAACGTAGGAA |
| | | AATACACACTGGCCGACGGAGGAGGCAACGCATTTGACGTAGCTGTGATATTCGCCGC |
| | | TAATATTAACTATGACACGGGCACCAAGACAGCATATCTACATTTCAACGAGAACGTA |
| | | CAAAGAGTGCTTGACAACGCCGTAACCCAAATCCGTCCTCTTCAGCAACAAGGAATTA |
| | | AGGTCTTGTTGTCTGTTTTAGGAAATCATCAGGGCGCTGGATTCGCTAACTTTCCCTCTC |
| | | AGCAAGCAGCCTCCGCATTTGCTAAACAATTGTCAGACGCCGTCGCCAAGTACGGCTT |
| | | AGACGGAGTTGACTTCGATGACGAGTATGCTGAATACGGAAACAATGGCACCGCACAG |
| | | CCAAACGACTCCTCTTTTGTTCATCTTGTCACCGCTCTACGTGCCAATATGCCAGATAA |
| | | AATTATTTCCCTTTATAATATAGGTCCTGCTGCTTCTCGTTTGTCCTACGGCGGTGTAGA |
| | | TGTCTCTGACAAGTTTGATTACGCCTGGAATCCTTATTACGGCACGTGGCAGGTCCCCG |
| | | GAATCGCACTTCCTAAGGCCCAGCTATCTCCTGCAGCAGTTGAAATCGGCAGAACGTCT |
| | | CGTTCCACCGTGGCAGATTTAGCAAGGAGAACAGTCGATGAAGGATATGGAGTGTACT |
| | | TAACGTATAATCTTGATGGCGGCGAtAGGACCGCAGACGTATCTGCCTTTACTAGAGAG |
| | | TTGTATGGAAGTGAGGCCGTTCGTACTCCCGGTTCATCAGGGTCCTCAGGATCATCCGG |
| | | TAGTAGTGGTTCATCCGGTTCATCCGGATCAAGTGGCTCCTCTGAAGCTGCAGCAAGGG |
| | | AGGCTGCAGCCCGTGAGGCAGCCGCTAGAGAAGCCGCCGCTAGGGGTGGTGGCGGCTC |
| | | TGGCGGAGGCGGTTCCGGTGGCGGAGGCTCTGCTTCTGTGACGACAACGCTGTCTCCCT |
| | | ACGATGAGAGAGTCAACCTAATAGAGTTAGCAGTATATGTATCAGACATTGGAGCACA |
| | | CTTAAGTGAGTATTACGCCTTCCAAGCCTTGCATAAAACAGAAACATATCCACCAGAG |
| | | ATTGCAAAAGCCGTTTTTGCTGGAGGAGACTTTACCACAATGCTTACAGGTATAAGTGG |
| | | TGATGAGGTAACCCGTATGATTACTGGAGTTCCCTGGTACTCCACAAGGCTTATGGGAG |
| | | CCATATCAGAAGCTTTGGCAAACGAAGGAATAGCCACGGCTGTACCCGCCTCTACCAC |
| | | GGAGGCTAGTTCTACCAGTACGTCCGAAGCTTCATCTGCCGCCACCGAATCCTCCTCAT |
| | | CCAGTGAGTCTAGTGCAGAAACGTCTTCTAATGCAGCATCTACACAAGCTACTGTGTCT |
| | | TCCGAATCATCCAGTGCAGCCTCTACCATAGCATCATCCGCCGAATCTTCTGTTGCATC |
| | | AAGTGTAGCATCTTCAGTTGCATCTAGTGCTTCTTTTGCAAACACCACTGCTCCTGTTTC |
| | | TTCTACCTCATCCATCAGTGTGACCCCAGTGGTCCAAAACGGCACTGATTCCACCGTGA |
| | | CAAAGACACAAGCCTCAACAGTAGAGACTACGATAACGTCTTGCTCCAACAACGTGTG |
| | | CTCAACAGTCACGAAGCCCGTAAGTAGTAAGGCACAGAGTACAGCCACTTCAGTCACT |
| | | TCATCAGCTAGTAGAGTTATAGATGTTACTACAAATGGCGCAAATAAGTTTAATAATGG |
| | | CGTGTTCGGCGCAGCCGCTATTGCTGGTGCCGCAGCATTATTACTATGAGCGGCCGCtcg |
| | | atttgtatgtgaaatagctgaaattcgaaaatttcattatggctgtatctactttagcgtattaggcatttgagcattggcttgaacaatgcgggctgtag |
| | | tgtgtcaccaaagaaaccattcgggttcggatctggaagtcctcatcacgtgatgccgatctcgtgtattttattttcagataacacctgaagacttt |
| EndoH-Sed1 | SEQ ID NO: | tgtcaaaacagtagtgataaaaggctatgaaggaggttgtctaggggctcgcggaggaaagtgattcaaacagacctgccaaaaagagaaaaa |
| construct: | 305 | agagggaatccctgttctttccaatggaaatgacgtaactttaacttgaaaaataccccaaccagaagggttcaaactcaacaaggattgcgtaatt |
| | | cctacaagtagcttagagctgggggagagacaactgaaggcagcttaacgataacgcggggggattggtgcacgactcgaaaggaggtatctt |
| | | agtcttgtaacctcttttttccagaggctattcaagattcataggcgatatcgatgtggagaagggtgaacaatataaaaggctggagagatgtcaat |
| | | gaagcagctggatagatttcaaattttctagatttcagagtaatcgcacaaaacgaaggaatcccaccaagacaaaaaaaaaaattctaagGAA |
| | | TTCCGAAACGATGAGATTCCCTTCTATTTTCACTGCTGTTTTGTTCGCTGCTTCTTCTGCT |
| | | TTGGCTGCTCCAGTTAACACTACTACCGAAGACGAAACTGCTCAAATTCCTGCTGAAGC |
| | | TGTTATTGGTTACTCTGACTTGGAAGGTGACTTCGACGTTGCTGTTTTGCCATTCTCTAA |
| | | CTCTACTAACAACGGTTTGTTGTTCATTAACACTACTATTGCTTCTATTGCTGCTAAGGA |
| | | AGAAGGTGTTTCTTTGGACAAGAGAGAGGCCGAAGCTGCTCCAGCCCCCGTAAAGCAG |
| | | GGACCAACCTCTGTAGCTTACGTAGAGGTAAATAACAACTCCATGTTAAACGTAGGAA |
| | | AATACACACTGGCCGACGGAGGAGGCAACGCATTTGACGTAGCTGTGATATTCGCCGC |
| | | TAATATTAACTATGACACGGGCACCAAGACAGCATATCTACATTTCAACGAGAACGTA |
| | | CAAAGAGTGCTTGACAACGCCGTAACCCAAATCCGTCCTCTTCAGCAACAAGGAATTA |
| | | AGGTCTTGTTGTCTGTTTTAGGAAATCATCAGGGCGCTGGATTCGCTAACTTTCCCTCTC |
| | | AGCAAGCAGCCTCCGCATTTGCTAAACAATTGTCAGACGCCGTCGCCAAGTACGGCTT |
| | | AGACGGAGTTGACTTCGATGACGAGTATGCTGAATACGGAAACAATGGCACCGCACAG |
| | | CCAAACGACTCCTCTTTTGTTCATCTTGTCACCGCTCTACGTGCCAATATGCCAGATAA |
| | | AATTATTTCCCTTTATAATATAGGTCCTGCTGCTTCTCGTTTGTCCTACGGCGGTGTAGA |
| | | TGTCTCTGACAAGTTTGATTACGCCTGGAATCCTTATTACGGCACGTGGCAGGTCCCCG |
| | | GAATCGCACTTCCTAAGGCCCAGCTATCTCCTGCAGCAGTTGAAATCGGCAGAACGTCT |
| | | CGTTCCACCGTGGCAGATTTAGCAAGGAGAACAGTCGATGAAGGATATGGAGTGTACT |
| | | TAACGTATAATCTTGATGGCGGCGAtAGGACCGCAGACGTATCTGCCTTTACTAGAGAG |
| | | TTGTATGGAAGTGAGGCCGTTCGTACTCCCGGTTCATCAGGGTCCTCAGGATCATCCGG |
| | | TAGTAGTGGTTCATCCGGTTCATCCGGATCAAGTGGCTCCTCTGAAGCTGCAGCAAGGG |
| | | AGGCTGCAGCCCGTGAGGCAGCCGCTAGAGAAGCCGCCGCTAGGGGTGGTGGCGGCTC |
| | | TGGCGGAGGCGGTTCCGGTGGCGGAGGCTCTCAATTCTCAAATTCAACATCAGCCTCCT |
| | | CCACTGATGTTACTTCAAGTTCCTCTATCAGTACATCATCTGGTTCAGTGACAATCACCT |
| | | CTAGTGAAGCCCCAGAATCAGATAACGGAACAAGTACGGCTGCACCCACAGAAACCTC |
| | | TACGGAGGCACCAACAACTGCCATCCCTACTAACGGCACCTCTACTGAAGCACCTACG |
| | | ACTGCAATCCCAACAAATGGTACGTCCACTGAGGCTCCTACCGATACCACCACTGAAG |
| | | CTCCAACCACTGCTTTGCCTACAAACGGCACTTCCACAGAGGCTCCTACTGATACAACT |
| | | ACTGAAGCTCCAACCACAGGCCTTCCTACTAATGGCACTACATCCGCATTCCCCCCTAC |
| | | GACCTCCCTGCCACCTAGTAACACTACTACGACTCCCCCTTACAATCCTAGTACCGACT |
| | | ACACTACGGATTACACGGTTGTTACTGAGTACACAACCTACTGCCCCGAGCCCACTACA |
| | | TTCACAACGAACGGCAAAACGTACACTGTAACGGAACCTACGACGCTGACAATTACCG |
| | | ATTGTCCTTGTACAATAGAAAAACCCACAACCACGAGTACAACAGAATATACCGTCGT |
| | | CACAGAATATACTACTTATTGTCCAGAACCAACTACCTTCACCACGAACGGTAAAACTT |
| | | ATACCGTTACTGAGCCCACGACTCTTACAATAACTGATTGTCCCTGCACTATCGAAAAA |
| | | TCTGAAGCACCAGAGTCATCTGTCCCTGTGACTGAATCTAAGGGAACGACCACTAAGG |
| | | AAACCGGAGTAACAACAAAGCAGACTACGGCCAATCCCTCACTGACTGTCTCCACGGT |
| | | CGTACCCGTGTCATCATCCGCCAGTTCTCATAGTGTCGTTATCAACTCAAACGGAGCTA |
| | | ATGTGGTTGTACCTGGCGCATTAGGACTTGCTGGTGTGGCCATGTTGTTCCTGTAAGCG |
| | | GCCGCtcgatttgtatgtgaaatagctgaaattcgaaaatttcattatggctgtatctactttagcgtattaggcatttgagcattggcttgaacaat |
| | | gcgggctgtagtgtgtcaccaaagaaaccattcgggttcggatctggaagtcctcatcacgtgatgccgatctcgtgtattttattttcagataacac |
| | | ctgaagacttt |
| MFalpha(EAEA)- | SEQ ID NO: | ATGAGATTCCCTTCTATTTTCACTGCTGTTTTGTTCGCTGCTTCTTCTGCTTTGGCTGCTCCAGTTAACACTACT |
| BT2623(mannosidase)- | 350 | ACCGAAGACGAAACTGCTCAAATTCCTGCTGAAGCTGTTATTGGTTACTCTGACTTGGAAGGTGACTTCGAC |
| linker- | | GTTGCTGTTTTGCCATTCTCTAACTCTACTAACAACGGTTTGTTGTTCATTAACACTACTATTGCTTCTATTGCT |
| ScSED1(anchor | | GCTAAGGAAGAAGGTGTTTCTTTGGACAAGAGAGAGGCCGAAGCTATGAAGAAAGTAATTAAGAAATATT |
| protein) fusion | | TTTTCCTAGCCTTGGCAATCATTATGTACTCATGTAACGAAGACGAGAAATATGACATTCTTGAACGTTATAC |
| protein used in P1 | | CCCTGAAACTATAACCTCTGACGAGATCGCACCTGTACTAAACCTTCAAGCCCAGTACATGGATTCAAACAG |
| producing strains | | TGAAATAGTTCTTGTGACTTGGATGAACCCAGAGGATGATTTTCTGAGTAAAGTTGAGATtTCTTGCTGCAG |
| | | TGCTAACGATAACTTACTGGGTGAGCCCGTCCTTCTTGATGCCGTCTCAACCAAGGTCGGCTCCTACCAGAC |
| | | GTCCCTTTCTGTCGAAGAACGTGGATATGTTAAGATCGTAGCTATAAATGAAAAGGGAGTTAGGTCTGAGG |
| | | CTAGGACGGCTGAGATTTTGTCATCTCAACAAGACTTCGTCTATCGTGCAGACTGCCTTATGTCTAGTGTGAT |
| | | TGAACTGTTCTTTGGAGGAAGGTACAATGCATGGAACGAAAATTACCCCAATGCAACCGGCCCTTACTGGG |
| | | ATGGAATCGCCGCTGTGTGGGGTCAGGGTGCAGCCTATTCTGGTTTCGTAACTATGTACAAAGTTACCAAA |
| | | GAAACAAATAACGAAAAACTAAGGGCTAAGTATGCAGAAAAGGAGGAAACATTCCTGAACTCTATAGACAT |
| | | CTTTTTAAATAATGGCTCTGGCAGAAAGTCATTTGCCTACGGCACGTACATCGGTCCTAACGACGAGCGTTA |
| | | TTACGATGATAATGTGTGGATAGGTATAGAAATGGCAAACTTATATGAGCTGACAGGAAACGAGGTGTACC |
| | | TACAACATGCCAATACCGTGTGGAATTTCATATTAGAAGGCATTGATGATGTAACGGGAGGTGGCGTATAC |
| | | TGGAAGGAGGGTGCAGTTTCCAAACACACGTGCTCAACCGCCCCCGCAGCTGTAATGGCTTTGAAACTTTAC |
| | | CAGTTGTCCAAGAATGAATCCTACTTAGAGATCGCCAAATCCTTGTATTCCTACTGCAAAGATGTCTTGCAAG |
| | | ATCCAAACGATTATCTTTTTTACGACAACGTGAGGCTAAGTGACCCTTCAGATAAGAACAGTGAACTAAAAG |
| | | TATCAAAAGACAAGTTCACTTACAACAGTGGTCAGCCCATGCTTGCAGCAGCCATGCTGTATCGTATAACCA |
| | | AAGAAGAGCAGTTTCTGAAAGACGCCCAAAACATTGCCCAATCAATATACAAGAAATGGTTCAAAAATTAC |
| | | CATTCATCAATCTTAGATAGGGATATAATGATTTTGTCTGATCCAAACACCTGGTTTAACGCAGTCATGTTTA |
| | | GGGGTTTTGTCGAGCTGTATAAAATCGACAAAAATGATGTTTATGTTAAGGCAGTTAAGAACACAATGGAG |
| | | CATGCTTGGCAATCAAACTGCCGTAACAGACTTACCAATCTTATGTCTGACGACTATGCCGGAGACAAGAAG |
| | | GAGGGTAAGTGGAACATTAAGACCCAAGGAGCTTTTGTTGAAATTTTTTCTTTGATTGGCGAGTTAGAACAG |
| | | TTAGGCTGTTTCCAGGAAGGTTCATCAGGGTCCTCAGGATCATCCGGTAGTAGTGGTTCATCCGGTTCATCC |
| | | GGATCAAGTGGCTCCTCTGAAGCTGCAGCAAGGGAGGCTGCAGCCCGTGAGGCAGCCGCTAGAGAAGCCG |
| | | CCGCTAGGGGTGGTGGCGGCTCTGGCGGAGGCGGTTCCGGTGGCGGAGGCTCTCAATTCTCAAATTCAAC |
| | | ATCAGCCTCCTCCACTGATGTTACTTCAAGTTCCTCTATCAGTACATCATCTGGTTCAGTGACAATCACCTCTA |
| | | GTGAAGCCCCAGAATCAGATAACGGAACAAGTACGGCTGCACCCACAGAAACCTCTACGGAGGCACCAAC |
| | | AACTGCCATCCCTACTAACGGCACCTCTACTGAAGCACCTACGACTGCAATCCCAACAAATGGTACGTCCACT |
| | | GAGGCTCCTACCGATACCACCACTGAAGCTCCAACCACTGCTTTGCCTACAAACGGCACTTCCACAGAGGCT |
| | | CCTACTGATACAACTACTGAAGCTCCAACCACAGGCCTTCCTACTAATGGCACTACATCCGCATTCCCCCCTA |
| | | CGACCTCCCTGCCACCTAGTAACACTACTACGACTCCCCCTTACAATCCTAGTACCGACTACACTACGGATTA |
| | | CACGGTTGTTACTGAGTACACAACCTACTGCCCCGAGCCCACTACATTCACAACGAACGGCAAAACGTACAC |
| | | TGTAACGGAACCTACGACGCTGACAATTACCGATTGTCCTTGTACAATAGAAAAACCCACAACCACGAGTAC |
| | | AACAGAATATACCGTCGTCACAGAATATACTACTTATTGTCCAGAACCAACTACCTTCACCACGAACGGTAA |
| | | AACTTATACCGTTACTGAGCCCACGACTCTTACAATAACTGATTGTCCCTGCACTATCGAAAAATCTGAAGCA |
| | | CCAGAGTCATCTGTCCCTGTGACTGAATCTAAGGGAACGACCACTAAGGAAACCGGAGTAACAACAAAGCA |
| | | GACTACGGCCAATCCCTCACTGACTGTCTCCACGGTCGTACCCGTGTCATCATCCGCCAGTTCTCATAGTGTC |
| | | GTTATCAACTCAAACGGAGCTAATGTGGTTGTACCTGGCGCATTAGGACTTGCTGGTGTGGCCATGTTGTTC |
| | | CTGTAA |
| CCW12 homolog | SEQ ID NO: | MLTKVISLAILTASAFADSGEFTLWNLSPGDPYDSTFWGVSEGLIVPVEPGVTFVITDDLQL |
| (GQ68_04433) | 306 | KTTDDQFVTVGEDSALGLGAEGSVEFSIINEDGITSLYYNGELVTAYICEGAEPQIYLTGSEE |
| (PAS_chr4_0151) | | DPECVSYTVAVIGVDGEAPPTFPEEDDETTTTDDPTDEPTDEPTDEPTDEPTDEPTDEPTDEP |
| | | TDEPTDEPTDEPTDEPTDEPTDEPTDEPTDEPTDEPTEEPTEEPTEEPTDEPTPPPPHWGNETV |
| | | TATKTEYETTKVTITSCEETKCYETTSDAWVSTCTTEIGGKVTKIVTWCPIPSTPGPKPPKPT |
| | | KPTETKPTTVPAPTTKKPETPTTKKPETPAPEKPEKTTTVIPPPTTEKPSTLSTSSVTGSVTIPTI |
| | | TATGGAGSNFNLGGLTVGVAGIAMALFV |
| CCW12 homolog | SEQ ID NO: | MFEKSKFVVSFLLLLQLFCVLGVHGQESGNGTTSDTAYACDIGATPFDGFNATIYQYQASD |
| GQ68_01574 (chr1) | 307 | DNSIQDPVFMSTGYLQRNQLHSTTGVTNPGFNIFTAGVATTTLYGIPNVNYQNMLLELKGY |
| | | FRADASGNYGLSLRNIDDSAILFFGRETAFECCNENLIPLDEAPTDYSLFTIKEGEASTNPDS |
| | | YTYTQYLEAGRYYPVRTFFANIRTRAVFNFTMTLPDGSELTDFQNYIFQFGALNQQQCQAE |
| | | IVTRENYTTTTEPWTGTFEATTTVIPSGTEPGTVIVQTPYSTIDSTSTWTGTFTTFTTDADGST |
| | | LAVVPSSTIDDHFASTETVLTDTAISTTVITVTSCGTSKCTKTTALTGVTQRTLTIDDRTTVVT |
| | | TYCPLPTDVATIKTASVSGSEVVQTIYTAKHSQAVSYVHPSTVTITREVCDAQTCTQATIVT |
| | | GEILQTTVVDSGSTTVVPKYVPVETHEPTFELSTL |
| CCW14 homolog | SEQ ID NO: | MQFTFASTSVVVSLIAALAKPAVATPPACLLACAAEVVKESSDCDALNNIQCICENEGSAIH |
| GQ68_01658 | 308 | ACLESTCPDGLSSTALQSFEDVCESVGTEANLDESSSSQSSSSSSSSESSSSSVSSSSSSASSSS |
| (PAS_chr1-4_0510) | | ETSSSVTSSSVTSSSTAVSSSTESSSSVEPSTSHSSSHSSSEVSSTVAPTTSVAPTTSSITTSSTSL |
| | | TSATTSSVTISIEPTSDAADKVIIPGLAGLVGALAVGLI |
| CCW22 homologs | SEQ ID NO: | MQYRSLFLGSALLAAANAAVYNTTVTDVVSELETTVLTITSCAEDKCITSKSTGLITTSTLT |
| GQ68_02511 (chr 1) | 309 | KHGVVTVVTTVCDLPSTTKSYVPPAKTTTIPPPEKTTTTVPPPAKTTTTVPPPAKTTSTVPPP |
| | | AKTSSHHESTITVTVPSSTSTKKIETESTTYHFVTQTTTARNITPPAITTQSHGAAGMNAANF |
| | | VGLGAAAVAAAALVL |
| CCW22 homolog | SEQ ID NO: | MSLLLFLVLGAFLLSSVKAADIGAFRLRVYTPGRFTNGALNFNNWGYQYLDASSSNGQLF |
| GQ68_03003 (chr 3) | 310 | AGYATVTSVTTFLAPDDEGFVWGSSLGGYPGFLGIGAGATAFHLTGIPGDALSWYIEDNIL |
| | | KTSSPTYVCSRNDGDVVVGIEANTRWLAMHDTSQLPPNYYCFQADYEIVALWYIPDTTST |
| | | WTGTETSTTTDDDGSVIELVPTPLPDTTSTWTGTFTTFTTDDDGSVIELVPTPLPDSTSTWTG |
| | | TYTTFTTDEDGSTIAVVPSSTIDSTSTWTGTYTTFTTDEDGSTIAVVPSSTIDSTSTWTGTYTT |
| | | FTTDEDGSTIAVYHHLLSTPHPPGLVLTPRSLPMRMEVLLLWYHHLLSTLHPPGLVLTPRSL |
| | | PMRMEVLLLWYHRLLSTPHPGLVLTPRSLPMRMEVLLLYHHLLSTPHPPGLVLTPRSLPMR |
| | | MEVLLLWY |
| FLO5 homolog | SEQ ID NO: | MKLQLQSFVFFLLSAVNVLADDSYGCSIATSPRSTGFVANLYEFPNMAISNAELKTYVRYR |
| GQ68_04296 (chr 4) | 311 | YKEGRLYDTISNIISPYFYYQGQGANSAYGTLYGRPNVYLYNFSMELKGYFRPPITGQYTID |
| | | FNGANVDDAAMVFFGKAGAFDCCNSDYILPEQSAEYSLYSVYPHTATDQILSATIYLEAGK |
| | | YYPLRVTYTNIGNIGSLDLRVVLPSGASITSLGAFVYQFPNNLSPGTCTPDVEYFTTTTQAW |
| | | TGTYETTYTVPPSGTQPGTVIIETPESYVTTTQPWTGTYETTYTVPPTGTEPGTVIIETPESYV |
| | | TTTQPWTGTYETTYTVPPSGTEPGTVIIETPESYVTTTQPWTGTYETTYTVPPSGTEPGTVIIE |
| | | TPESYVTTTQPWTGTYETTYTVPPSGTEPGIVIIETPESYVTTTQPWTGTYETTYTVPPSGTEP |
| | | GTVVIETPEITDCEAVCCGAVPTSDPLRRRDVCDCETFCCPGDTNCETYVTTTQPWTGTYET |
| | | TYTVPPSGTEPGTVIIETPESYVTTTQPWTGTYETTYTVPPSGTEPGIVIIETPESYVTTTQPWT |
| | | GTYETTYTVPPTGTEPGTVIIETPESYVTTTQPWTGTYETTYTVPPSGTEPGIVIIETPESYVTT |
| | | TQPWTGTYETTYTVPPSGTEPGTVIIETPESYVTTTQPWTGTYETTYTVPPTGTEPGTVIIETP |
| | | ESYVTTTQPWTGTYETTYTVPPSGTEPGIVIIETPESYVTTTQPWTGTYETTYTVPPTGTEPG |
| | | TVIIETPESYVTTTQPWTGTYETTYTVPPTGTEPGTVIIETPESYVTTTQPWTGTYETTYTVPP |
| | | SGTEPGTVIIETPESYVTTTQPWTGTYETTYTVPPSGTEPGTVVIETPEITDCEAVCCGAVPTS |
| | | DPLRRRDVCDCETFCCPGDTNCETYVTTTQPWTGTYETTYTVPPSGTEPGTVIIETPESYVT |
| | | TTQPWTGTYETTYTVPPTGTEPGTVIIETPESYVTTTQPWTGTYETTYTVPPSGTQPGTVIIET |
| | | PESYVTTTQPWTGTYETTYTVPPTGTEPGTVIIETPESYVTTTQPWTGTYETTYTVPPSGTEP |
| | | GTVIIETPESYVTTTQPWTGTYETTYTVPPSGTQPGTVIIETPESYVTTTQPWTGTYETTYTVP |
| | | PTGTEPGTVIIETPESYVTTTQPWTGTYETTYTVPPSGTEPGIVIIETPESYVTTTQPWTGTYE |
| | | TTYTVPPTGTEPGTVIIETPESYVTTTQPWTGTYETTYTVPPTGTEPGTVIIETPESYVTTTQP |
| | | WTGTYETTYTVPPSGTEPGTVIIETPESYVTTTQPWTGTYETTYTVPPSGTEPGTVVIETPEIT |
| | | DCEAVCCGAVPTSDPLRRRDVCDCETFCCPGDTNCETYVTTTQPWTGTYETTYTVPPSGTE |
| | | PGTVIIETPESYVTTTQPWTGTYETTYTVPPTGTEPGTVIIETPESYVTTTQPWTGTYETTYTV |
| | | PPSGTQPGTVIIETPESYVTTTQPWTGTYETTYTVPPTGTEPGTVIIETPESYVTTTQPWTGTY |
| | | ETTYTVPPSGTEPGTVIIETPESYVTTTQPWTGTYETTYTVPPSGTQPGTVIIETPESYVTTTQ |
| | | PWTGTYETTYTVPPTGTEPGTVIIETPESYVTTTQPWTGTYETTYTVPPSGTEPGIVIIETPESY |
| | | VTTTQPWTGTYETTYTVPPTGTEPGTVIIETPESYVTTTQPWTGTYETTYTVPPSGTEPGTVII |
| | | ETPESYVTTTQPWTGTYETTYTVPPSGTQPGTVIIETPESYVTTTQPWTGTYETTYTVPPSGT |
| | | EPGTVIVETPDVPGSYVTTTQPWTGTYETTHTVPPTGTEPGTVVVETPDVPGSYVTTTQPW |
| | | TGTYETTHTVPPTGTEPGTVVVETPDVPGSYVTTTQPWTGTYETTYTVPPSGTEPGTVIVET |
| | | PDVPGSYVTTTQPWTGTYETTHTVPPTGTEPGTVVVETPDVPGSYVTTTQPWTGVYKTTYT |
| | | VPPSGTIPGTVIIETPFGYFNTSSISTKTDKRTITSVVPCSQCSESKTQYITPTGPGDVTVIISQPP |
| | | SKITLSSPEDKTKTDFITSTGSIGGGSPPSHPNDKPGIITTPTQPIGGGNPSDIPSAISSVSSGGNS |
| | | RASVPSFSTSSAISVQVSSLYDENSGSTFEVSLLFSVVSGFFLTLMV |
| FLO5 homolog | SEQ ID NO: | MKFPVPLLFLLQLFFIIATQGDESGNGDESDTAYGCDITSNAFDGFDATIYEYNANDLKLIRD |
| GQ68_03011 | 312 | PVFMSTGYLGRNVLNKISGVTVPGFNIWNPRSRTATVYGVQNVNYYNMVLELKGYFKAA |
| (PAS_chr3_1145) | | VSGDYKLTLSNIDDSSMLFFGKNTAFQCCDTGSIPVDQAPTDYSLFTIKPSNQVNSEVISSTQ |
| | | YLEAGKYYPVRIVFVNALERALFNFKLTIPSGTVLDDFQDYIYQFGALDENSCYETTVSKIT |
| | | EWTTYTTPWTGTFETTRTITPTGTEGTVVIETPESYVTTTQPWTGTYETTYTVPPTGTEPGTV |
| | | IIETPEIIDCEAVCCGPFLTAFSFRKREECQCENICCPGDTNCETYVTTTQPWTGTYETTYTVP |
| | | PTGTEPGTVIIETPESYVTTTQPWTGTYETTYTVPPTGTEPGTVIIETPESYVTTTQPWTGTYE |
| | | TTYTVPPSGTEPGTVVIETPEIVDCEAYCCASVAIKKRELCQCENFCCSWDQSCQTYVTTTQ |
| | | PWTGTYETTYTVPPTGTEPGTVIIETPESYVTTTQPWTGTYETTYTVPPTGTEPGTVIIETPES |
| | | YVTTTQPWTGTYETTYTVPPTGTEPGTVIIETPEIIDCEAVCCGPFLTAFSFRKREECQCENIC |
| | | CPGDTNCETYVTTTQPWTGTYETTYTVPPTGTEPGTVIIETPESYVTTTQPWTGTYETTYTV |
| | | PPTGTEPGTVIIETPESYVTTTQPWTGTYETTYTVPPTGTEPGTVIIETPEIINCEAVCCGPFLT |
| | | AFSFRKREECQCENICCPGDTNCETYVTTTQPWTGTYETTYTVPPTGTEPGTVIIETPESYVT |
| | | TTQPWTGTYETTYTVPSTGTEPGTVIIETPESYVTTTQPWTGTYETTFTVPPTGTEPGTVVIE |
| | | TPESYVTTTQPWTGTYETTYSVPPSGTEPGTVVIETPEASTARTKFTTVTSSWTGVFTTTKTL |
| | | PASGTEPATIVIQTPTGYFNTSSLVSTRTKTNVDTVTRVIPCPICTAPKTITVVPEEPNESVSVII |
| | | SQPQSSSTDTTLSKPDSVRVISQPETASQMDTSLSKTDSAVISTETAGNNIIPLAGSHSYNTIV |
| | | TTVTDSPQVAQSTTATSSSNVHLTISTQTTTPSLVYSSSLSTVHQVSPSNGGFRSSITVHPLLS |
| | | VIGAIFGALFM |
| FLO5 homolog | SEQ ID NO: | MTKFTILLLVLLKFYSILAIEVDGSANGQPLAHPIVVEVHEATKWITHTSPWTGTPEAIRTVT |
| GQ68_03079 (chr 3) | 313 | GETPYEQKIARYDEFNPRLANREIIDCVAFCCGDATSSPSITEPESTATELPESYVTINRPWSL |
| | | SWIPDVPPGSPYWSTSTIPPSGTEPGTVIIYFYLYDDARKRREINFGSTQPYHGRPKLLGSIEK |
| | | RELCQCDAVCCLGDLSCEVYVTTTQPWTGTYETTYTITPTGSEPGTVIIETPELYVTTTQPW |
| | | TGTYETTYTITPTGSEPGTVIIETPESYVTTTQPWTGTYETTYTITPTGSEPGTVIIETPESYVTT |
| | | TQPWTGTYETTYTITPTGSEPGTVIIETPESYVTTTQPWTGTYETTYTVPPSGTEPGAVIIETP |
| | | ELYVTTTQPWTGTYETTYTITPTGSEPGTVIIETPESYVTTTQPWTGTYETTYTVPPSGTEPG |
| | | TVIIETPELYVTTTQPWTGTYETTYTITPTGSEPGTVIVEIPVSYVNSTQISTSTYDTTDTVLSS |
| | | GVEPGTIAIETPIVYLNTSVSAFSRPWTKIDTVTQFSSCAVCSKPETITVTPENPIDTVTIIISQP |
| | | QSTSQSNTPTSFKANSTSAFSRFDEDSIPVFGSYSYEITVNIDVNTEDDTTTNLNADTTIIIGSL |
| | | SAIRTVAGSSSNYHASNISPTINSQKTASSVVVHSDSSATVYQFSPSNGAPWLSVQISTLLSV |
| | | VGTLLAAVLL |
| FLO5 homolog | SEQ ID NO: | MNFRYLLILPIYASIVLGQVGDFQLLLNAKEPIRNSPSLLSSNYGNLTLPAMANGALESHFD |
| GQ68_04277 (chr 4) | 314 | YGNAYVGDDQITVVYHLPDEHGQINAYRQDTDEYIGYLGLVIDDYGEYTYLSVIMPGVQY |
| | | DQTTSVNWYIENEELKSTSINVQPLLGCYYKNPPQYSWYWASIDEPGNIASSNFVCEPCKV |
| | | YVDFVPTPSADITSMWTGSETSWTTDADGTVIELVPTPSADATSVWTGDHTTWTTDDDGN |
| | | VIEQIPTPSADITSMWTGSETSWTTDADGTVIELVPTPSADITSMWTGSETSWTTDADGTVIE |
| | | LVPTPSADATSVWTGDHTTWTTDDDGNVIEQIPTPSADITSMWTGSETSWTTDADGTVIEL |
| | | VPTPSADATSVWTGDHTTWTTDDDGNVIEQIPTPSADITSMWTGSETSWTTDADGTVIELV |
| | | PTPSADITSMWTGSETSWTTDADGTVIELVPTPSADATSVWTGDHTTWTTDDDGNVIEQIP |
| | | TPSADITSMWTGSETSWTTDADGTVIELVPTPSADITSMWTGSETSWTTDADGTVIELVPTP |
| | | SADITSMWTGSETSWTTDADGTVIELVPTPSADATSVWTGDHTTWTTDDDGNVIEQIPTPS |
| | | ADITSMWTGSETSWTTDADGTVIELVPTPSADITSMWTGSETSWTTDADGTVIELVPTPSAD |
| | | ITSMWTGSETSWTTDADGTVIELVPTPSADITSMWTGSETSWTTDADGTVIELVPTPSADIT |
| | | SMWTGSETSWTTDADGTVIELVPTPSADITSMWTGSETSWTTDADGTVIELVPTPSADATS |
| | | VWTGDHTTWTTDDDGNVIEQIPTPSADITSMWTGSETSWTTDADGTVIELVPTPSADITSM |
| | | WTGSETSWTTDADGTVIELVPTPSADATSVWTGDHTTWTTDDDGNVIEQIPTPSADITSMW |
| | | TGSETSWTTDADGTVIELVPTPSADATSVWTGDHTTWTTDDDGNVIEQIPTPSADITSMWT |
| | | GSETSWTTDADGTVIELVPTPSADITSMWTGSETSWTTDADGTVIELVPTPSADITSMWTGS |
| | | ETSWTTDADGTVIELVPTPSADATSVWTGDHTTWTTDDDGNVIEQIPTPSADITSMWTGSE |
| | | TSWTTDADGTVIELVPTPSADITSMWTGSETSWTTDADGTVIELVPTPSADATSVWTGDHT |
| | | TWTTDDDGNVIEQIPTPSADITSMWTGSETSWTTDADGTVIELVPTPSADITSMWTGSETSW |
| | | TTDADGTVIELVPTPSADITSMWTGSETSWTTDADGTVIELVPTPSADATSVWTGDHTTWT |
| | | TDDDGNVIEQIPTPSADITSMWTGSETSWTTDADGTVIELVPTPSADITSMWTGSETSWTTD |
| | | ADGTVIELVPTPSADITSMWTGSETSWTTDADGTVIELVPTPSADATSVWTGDHTTWTTDD |
| | | DGNVIEQIPTPSADITSMWTGSETSWTTDADGTVIELVPTPSADITSMWTGSETSWTTDADG |
| | | TVIELVPTPSADATSVWTGDHTTWTTDDDGNVIEQIPTPSADITSMWTGSETSWTTDADGT |
| | | VIELVPTPSADITSMWTGSETSWTTDADGTVIELVPTPSADITSMWTGSETSWTTDADGTVI |
| | | ELVPTPSADATSVWTGDHTTWTTDDDGNVIEQIPTPSADITSMWTGSETSWTTDADGTVIE |
| | | LVPTPSADITSMWTGSETSWTTDADGTVIELVPTPSADITSMWTGSETSWTTDADGTVIELV |
| | | PTPSADATSVWTGDHTTWTTDDDGNVIEQIPTPSADITSMWTGSETSWTTDADGTVIELVP |
| | | TPSADITSMWTGSETSWTTDADGTVIELVPTPSADITSMWTGSETSWTTDADGTVIELVPTP |
| | | SADTTSVWTGSYTTWTTDEDGTVIEQVPTPSADTPSADTTSVWTGSYTTWTTDEDGTVIEQ |
| | | VPTPSADTTSVWTGSYTTWTTDEDGTVIEQVPTPSADTPSADTTSVWTGSYTTWTTEVGDG |
| | | GSSTVVELVPTESSTSTNVMQTPVPSSGVSDGVSVFNGFNVEVFHYPADNYELANEISFLSY |
| | | GYENLGLVTTVTGVSDINFDTDSNWPYYIDRDALGNTGSYVNATIEYEGFFRAPVDGEYVF |
| | | SFSSTDYNSILFVGSPAAADQALQKREVQFLKPETSPDYVLLFNNTRDLGKTVSTTQYLLAD |
| | | QYYPLRVVIAAISQHALLDFQIKLPNGASLTQYQGYVYNFALEGSESTTVIGDKTSTWTGSY |
| | | TTWTTDSDGSTIVVVPPATITADKTSTWTGSYTTWTTDSDGSTVVICPSITSDHNDKPSESTL |
| | | TDSSISTTVVTVTSCDIEKCTKTTALTGVRETTLTTGGTTTVVTTYCPLPTDIVTVKTTSIDGS |
| | | EVLQTIYTAKPNHVVPDVQTSTVTITREVCDAFTCTHATIVTGEILKTTTLADTHYTTVVPV |
| | | YVPLETYQPAVELSTLETVLKSSDLASGPVVTAGSVQPSYQSGGVAESSLTVSEFEAHSTSD |
| | | TVSQPSTISLQTGEANALKWSSFFGAALVPLVNVFFV |
| FLO5 homolog | SEQ ID NO: | MQNTNDKLIIRTFYSISTIHGLLSINIFSDTRVYKFAIYSTDAVSLEPRTKNNMSLVTVLACFII |
| GQ68_01371 (chr 1) | 315 | FAAHAFGQDTFYMLKVRTLTPNGYPLADSLSNPMQYWDLYYVPGGPRRLESSFVNWQPT |
| | | TAAPINQFYCRLGTDGHMTGYNRVTGSVIGKLSFGTNAATALAFGSYDGDPSYPPQAFSISS |
| | | SVSGTMTYLNVHYVNARSITWYSTTTATGETNVYINVASTGYTGDRTTYQAELWVEPFVP |
| | | NIPVDTTTSIWTGSQTSYTTEVGENGGSTVIELIPTPPADATSTWTGTYTTRTTDADGSVIEQI |
| | | PTPSADTTSVWTGTYTTWTTDADGSVIEQIPTPSADTTSVWTGTYTTWTTDADGSVIEQIPT |
| | | PSADTTATWTGTETSYTTDVGEDGSSTVIELVPTPSADTTATWTGTETSYTTDVGEDGSSTV |
| | | VELVPTPSADTTATWTGTETSYTTDVGEDGSSTVIELVPTPSADTTATWTGTETSYTTDVGE |
| | | DGSSTVIELVPTPSADTTATWTGTETSYTTDVGEDGSSTVVELVPTPSADTTATWTGTETSY |
| | | TTDVGEDGSSTVIELVPTPSADTTATWTGTETSYTTDVGEDGSSTVIELVPTPSADTTATWT |
| | | GTETSYTTDVGEDGSSTVIELVPTPSADTTATWTGTETSYTTDVGEDGSSTVIELVPTPSADT |
| | | TATWTGTETSYTTDVGEDGSSTVIELVPTPSADTTATWTGTETSYTTDVGEDGSSTVIELVP |
| | | TPSADTTATWTGTETSYTTDVGEDGSSTVIELVPTPSADTTATWTGTETSYTTDVGEDGSST |
| | | VIELVPTPSADTTATWTGTETSYTTDVGEDGSSTVIELVPTPSADTTATWTGTETSYTTDVG |
| | | EDGSSTVIELVPTPSADTTATWTGTETSYTTDVGEDGSSTVIELVPTPSADTTATWTGTETSY |
| | | TTDVGEDGSSTVIELVPTPTPSADTTATWTGTETSYTTDVGEDGSSTVIELVPTPSADTTAT |
| | | WTGTETSYTTDVGEDGSSTVIELVPTPSADTTATWTGTETSYTTDVGEDGSSTVIELVPTPS |
| | | ADTTATWTGTETSYTTDVGEDGSSTVIELVPTPTPSADTTATWTGTETSYTTDVGEDGSSTV |
| | | IELVPTPSADTTATWTGTETSYTTDVGEDGSSTVIELVPTPSADTTATWTGTETSYTTDVGE |
| | | DGSSTVVELVPTPTPSADTTATWTGTETSYTTDVGEDGSSTVVELVPTPSADTTATWTGTE |
| | | TSYTTDVGEDGSSTVIELVPTPSADTTATWTGTETSYTTDVGEDGSSTVVELVPTPSADTTA |
| | | TWTGTETSYTTDVGEDGSSTVVELVPTPTADTTATWTGTETSYTTDVGEDGSSTVIELVPTP |
| | | SADTTATWTGTETSYTTDVGEDGSSTVVELVPTPSADTTATWTGTETSYTTDVGEDGSSTV |
| | | VELVPTPSADTTATWTGTETSYTTDVGEDGSSTVIELVPTPSADTTATWTGTETSYTTDVGE |
| | | DGSSTVVELVPTPSADTTATWTGTETSYTTDVGEDGSSTVVELVPTPTADTTATWTGTETS |
| | | YTTDVGEDGSSTVIELVPTPSADTTATWTGTETSYTTDVGEDGSSTVVELVPTPSADTTAT |
| | | WTGTETSYTTDVGEDGSSTVIELVPTPSADTTATWTGTETSYTTDVGEDGSSTVIELVPTPS |
| | | ADTTATWTGTETSYTTDVGEDGSSTVIELVPTPTPSADTTATWTGTETSYTTDVGEDGSSTV |
| | | IELVPTPSADTTATWTGTETSYTTDVGEDGSSTVIELVPTPTPSADTTATWTGTETSYTTDV |
| | | GEDGSSTVVELVPTPSADTTATWTGTETSYTTDVGEDGSSTVIELVPTPSADTTATWTGTET |
| | | SYTTDVGEDGSSTVVELVPTPSADTTATWTGTETSYTTDVGEDGSSTVIELVPTPSADTTAT |
| | | WTGTETSYTTDVGEDGSSTVIELVPTPSADTTATWTGTETSYTTDVGEDGSSTVIELVPTPS |
| | | ADTTATWTGTETSYTTDVGEDGSSTVIELVPTPSADTTATWTGTETSYTTDVGEDGSSTVIE |
| | | LVPTPSADTTATWTGTETSYTTDVGEDGSSTVIELVPTPSADTTATWTGTETSYTTDVGEDG |
| | | SSTVIELVPTPSADTTATWTGTETSYTTDVGEDGSSTVIELVPTPSADTTATWTGTETSYTTD |
| | | VGEDGSSTVIELVPTPSADTTATWTGTETSYTTDVGEDGSSTVIELVPTPSADTTATWTGTE |
| | | TSYTTDVGEDGSSTVVELVPTPTPSADTTATWTGTETSYTTDVGEDGSSTVIELVPSDTETA |
| | | TNIVETPVPSSGVSDGVSVFDGFNVEVFHYPADNYELANEIGFLSYGYENLGLVTNATGVS |
| | | DINFDTDSNWPYYIDRDALGNTGSYVNATIEYEGFFRAPVDGEYVFSFSNTDYNSILFVGSP |
| | | AAAGQALQKRRVQFLKPETSPDHVLLFNNTRDLGQTISTTQYLLADQYYPLRVVIAAISQH |
| | | ALLDFQIKLPNGALLTQYQGYVYNFALEGSESTTVIGDKTSTWTGSYTTWTTDSDGSTVVV |
| | | VPSATITADKTSTWTGSYTTWTTDSDGSTIVICPSITSDHNDKPSESTLTDGSISTTVVTVTSC |
| | | DIEKCTKTTALTGVTETTLTTGGTTTVVTTYCPLPTDIVTVKTTSISGSEVLQTIYTAKPSHV |
| | | VPNVHTLTVTITREVCDAFTCTQATIVTGEILKTTTLADTHSTTVVPVYVPLESYQSAVELST |
| | | LETVLKSSDFASGSAVTAGSAQPSYQSGGVAESSLTGSELEAHSTSDTVSQPSTISPQTGEAN |
| | | ALRWSSFFGAALVPLVNVFFV |
| FLO5 homolog | SEQ ID NO: | MTKLTILLSVLLQLFSVLAEVPKKTEWSSHTTYWTSTLEALRTVTPTGTERAVIGEAPYEYK |
| GQ68_04678 | 316 | LIGNDQFDPGLNAKREIIDCEAVCCGAVPTSDPLKRRDVCECENVCCPGDDCETYVTTTQP |
| (PAS_chr4_0363) | | WTGTYETTYTVPPSGTEPGTVVIETPEITDCEAVCCGAVPTSDPLRRRDVCECENVCCPGDD |
| | | CETYVTTTQPWTGTYETTYTVPPSGTEPGTVVIETPEITDCEAVCCGAVPTSDPLRRRDVCE |
| | | CENVCCPGDDCETYVTTTQPWTGTYETTYTVPPTGTEPGTVVIETPVTYVTTTQPWTGTYE |
| | | TTYTVPPTGTEPGTVVIETPEITDCEAVCCGAVPTSDPLRRRDVCECENVCCPGDDCETYVT |
| | | TTQPWTGTYETTYTVPPTGTEPGTVVIETPVTYVTTTQPWTGTYETTYTVPPTGTEPGTVVI |
| | | ETPVTYVTTTQPWTGTYETTYTVPPTGTEPGTVVIETPVTYVTTTQPWTGTYETTYTIPPTG |
| | | TEPGTVVIETPEITDCEAVCCGAVPTSDPLRRRDVCECENVCCPGDDCETYVTTTQPWTGT |
| | | YETTYTVPPTGTEPGTVVIETPVTYVTTTQPWTGTYETTYTVPPTGTEPGTVVIETPVTYVTT |
| | | TQPWTGTYETTYTVPPTGTEPGTVVIETPVTYVTTTKPWTGTYETTHTVPASGTEPGTVIIET |
| | | PIKYLNTSISASTSTWTKINTVTQFISCPVCTIPKTITVTPKISNETVTIIISQPHGTSSRTTTVVK |
| | | TDGASVSSHSYKTALTTDVKPEEKTSTKLGTVTTVSGSHSAIDTVTGSLSDYHASSIPHTVK |
| | | SEEKASSTVTHTISSSTVYQVSPSNGASWLSVRLNTALSIIGTLFAAVFI |
| FLO5 homolog | SEQ ID NO: | MSKTKNGGSEFVHIAYVFHIEASTPSDYINMIQIVLFPHQAQITKRMNLVTLLVCNLLCVSL |
| GQ68_04282 (chr 4) | 317 | TLGQGVYRLKFPALVVTGRESVGTTVVNYDFLVGNTGQYGDLGEFFYDGEPYYCWNSTD |
| | | SQPLSCSSSSSLLISTQNVTISHPDEDGTVYAYAERDGGLLGRFTVGSVSADWPQWAVIVYS |
| | | TSSSAHPSSWYVDDNKLKLTSGLGPNNSTTLQACYFTQSSGRDRYAISLEGSPAYTGQVSC |
| | | QATEFDLEFIPPSADTTSIWDGSYTTWTTDSNGIVVEQIPTPSADTTSIWTGSETSWTTDSDG |
| | | TVIELVPTPSADATSIWTGDHTTWTTDSEGNVIEQIPTPSADTTSIWTGSETSWTTDSDGTVI |
| | | ELVPTPSADATSIWTGDHTTWTTDSEGNVIEQIPTPSADATSIWTGDHTTWTTDREGNVIEQI |
| | | PTPSADTTSIWTGSETSWTTDSDGTVIELVPTPSADATSIWTGDHTTWTTDSEGNVIEQIPTP |
| | | SADATSIWTGSETSWTTDSDGTVIELVPTPSADATSIWTGDHTTWTTDSEGNVIEQIPTPSAD |
| | | ATSIWTGDHTTWTTDSEGNVIEQIPTPSADTTSIWTGSETSWTTDSDGTVIELVPTPSADATSI |
| | | WTGDHTTWTTDSEGNVIEQIPTPSADATSIWTGDHTTWTTDSEGNVIEQIPTPSADTTSIWT |
| | | GSETSWTTDSDGTVIELVPTPSADATSIWTGDHTTWTTDSEGNVIEQIPTPSADATSIWTGD |
| | | HTTWTTDSEGNVIEQIPTPSADTTSIWTGSETSWTTDSDGTVIELVPTPSADATSIWTGDHTT |
| | | WTTDSEGNVIEQIPTPSADATSIWTGSETSWTTDSDGTVIELVPTPSADATSIWTGDHTTWT |
| | | TDSEGNVIEQIPTPSADATSIWTGDHTTWTTDSEGNVIEQIPTPSADTTSIWTGDHTTWTTEV |
| | | GGDGSSIVVELVPSETGTATNVVQTPVPSSGISDGVSALDGFNVEVFHYPADNYELANEISF |
| | | LSYGYENLGLVTTATGVSDINFDTDSNWPSYIDRNALGNTGSYVNATIKYEGFFRAPVDGD |
| | | YEFSFSNIDYNSILFVGSAAADQALRKREAQFLKPETSPNHILFFNNSRDVGQTISTTQYLSA |
| | | DSYYPLRVVIAAVSQHALLDFQIKLPNGVSLTQFQGYVYNFALEGAESTTVIGDKTSTWTG |
| | | TYTTWTTDSEGSTIVLCPSIISDHNGKPADTTLTDGSISTTVVTVTSCDIKKCTKTTALTGVT |
| | | QKTLTVKGTTTVVTAYCPLPTDVATVKTISVGGSEVLQTVYTAKPSHIVPDVQTLTVTITRE |
| | | VCDALTCIPATIVTGEILKTTTLADTHSTTVIPVYVPLETHQPALDLITLETVLKSSDFANGPA |
| | | ITSVSVESLSHQSGVVVSEFDSDSTSGAVSQPSSAVSLQTGKASALKWSPFLGAAVISLFNVF |
| | | FV |
| FLO5 homolog | SEQ ID NO: | MNLFTILAWGFLYVPLVLGEGYYSLNFDARVPIALGILGSSYQKYTIMADRSLLGGSNIDLD |
| GQ68_03013 | 318 | VTFSGIIELLTNRVHIVVSLPDADGRVSVYDMYSGTSLGYLSFVCSLTTCEVHAVSSSSGAT |
| (PAS_chr3_0015) | | TWTLDGNQLIPTSPSTVYACYRSLVGLLAQYTLNDRTSITAQCEQTNLYVELAIPAFPETTA |
| | | VWTGTYTTWTTDESGSVIEQMPTPSADTTTTWTGTYTTWTTDADGSVIEQIPTPPADTTSV |
| | | WTGTYTTRTTDADGSVIEQIPTPSADTTSIWTGTYTTWTTDADGSVIEQIPTPSADTTSVWT |
| | | GTYTTWTTDADGSVIEQIPTPSADTTSVWTGTYTTWTTDADGSVIEQIPTPSADTTSVWTGT |
| | | YTTWTTDADGSVIEQIPTPSTDTTLAPSADTTSIWTGTYTTWTTDADGSVIEQIPTPSADTTSI |
| | | WTGTYTTWTTDADGSVIEQIPTPSADTTSVWTGTYTTWTTDADGSVIEQIPTPSTDTTLAPS |
| | | ADTTSIWTGTYTTWTTDADGSVIEQIPTPSADTTSVWTGTYTTWTTDADGSVIEQIPTPSAD |
| | | TTSVWTGTYTTWTTDADGSVIEQIPTPSADTTSVWTGTYTTWTTDADGSVIEQIPTPSADTT |
| | | SVWTGTYTTWTTDADGSVIEQIPTPSADTTSVWTGTYTTWTTDADGSVIEQIPTPSADTTSI |
| | | WTGTYTTWTTDADGSVIEQIPTPSADTTSVWTGTYTTWTTDADGSVIEQIPTPSADTTLAPS |
| | | ADTTSIWTGTYTTWTTDADGSVIEQIPTPSADTTSIWTGTYTTWTTDADGSVIEQIPTPSADT |
| | | TSVWTGTYTTWTTDADGSVIEQIPTPSADTTSVWTGTYTTWTTDADGSVIEQIPTPSADTTS |
| | | VWTGTYTTWTTDADGSVIEQIPTPSADTTSVWTGTYTTWTTDADGSVIEQIPTPSADTTSV |
| | | WTGTYTTWTTDADGSVIEQIPTPSADTTSVWTGTYTTWTTDADGSVIEQIPTPSTDTTLAPS |
| | | ADTTSIWTGTYTTWTTDADGSVIEQIPTPSADTTSVWTGTYTTWTTDADGSVIEQIPTPSAD |
| | | TTSVWTGTYTTWTTDADGSVIEQIPTPSADTTSVWTGTYTTWTTDADGSVIEQIPTPSTDTT |
| | | LAPSADTTSIWTGTYTTWTTDADGSVIEQIPTPSADTTSIWTGTYTTWTTDADGSVIEQIPTP |
| | | SADTTSVWTGTYTTWTTDADGSVIEQIPTPSADTTSVWTGTYTTWTTDADGSVIEQIPTPSA |
| | | DTTSVWTGTYTTWTTDADGSVIEQIPTPSADTTSVWTGTYTTWTTDADGSVIEQSPTPSAY |
| | | TTSVWTGTYTTWTTDADGSVIEQIPTPSADTTSVWTGTYTTWTTDADGSVIEQIPTPSADTT |
| | | SVWTGTYTTWTTDADGSVIEQIPTPSADTTSVWTGTYTTWTTDADGSVIEQIPTPSADTTSV |
| | | WTGTYTTWTTDADGSVIEQIPTPSADTTSVWTGTYTTWTTDADGSVIEQIPTPSADTTSVW |
| | | TGTYTTWTTDADGSVIEQSPTPSAYTTSVWTGTYTTWTTDADGSVIEQIPTPSADTTLAPSA |
| | | DTTSIWTGTYTTWTTDADGSVIEQIPTPSADTTSIWTGTYTTWTTDADGSVIEQIPTPSADTT |
| | | SVWTGTYTTWTTDADGSVIEQIPTPSADTTSVWTGTYTTWTTDADGSVIEQIPTPSADTTSV |
| | | WTGTYTTWTTDADGSVIEQIPTPSTDTTLAPSADTTSIWTGTYTTWTTDADGSVIEQIPTPSA |
| | | DTTSVWTGTYTTWTTDADGSVIEQIPTPSTDTTLAPSADTTSIWTGTYTTWTTDADGSVIEQ |
| | | IPTPSADTTSVWTGTYTTWTTDADGSVIEQIPTPSADTTSVWTGTYTTWTTDADGSVIEQIPT |
| | | PSADTTSVWTGTYTTWTTDADGSVIEQIPTPSADTTLAPSADTTSIWTGTYTTWTTDADGS |
| | | VIEQIPTPSADTTSVWTGTYTTWTTDAAGTVIEVIPSGTSISSDVIPTPLPTSGVDIDTIPYDAF |
| | | NVAVYHYPADNYELANNLGFLTSGYEGLGQVTTATSVGNINFDTSSGWPYYIESNALGNT |
| | | GSYVNATIEYVGFFQAPANGNYELSFSNIDYNAILFLGSPATDSSLAKREVQFLKPETSSEYV |
| | | LFFDHGKDAGQTVSTTQYLSAGLYYPLRIVLAAVSERAQLDFQITLPDGRVLDQYQGYVY |
| | | NFAHEGIESATSSAHETSWSRFTNSTIYSHSSTIGIITSSTDAPHSVINPTAIETTSTDTSISTVA |
| | | VTTSICDTKDCVKTTVITPNSPLPTQTVSLTTTTIDRSEVVQTAHSAVPSQFAPDAHPSAVTIT |
| | | REQCDAYSCSQATIVSGKVLQTTTVSDSTTVVPLDTPQLSVEASTLETRLKSTQSSRAPTVT |
| | | VQTSQSSRHSEDVTESSVHVSEFDAQSTSATSASALQAPSSISLQTGGANTLRLSAFLGTALL |
| | | PMLNVLFI |
| SED 1 homolog | SEQ ID NO: | MQFSIVATLALAGSALAAYSNVTYTYETTITDVVTELTTYCPEPTTFVHKNKTITVTAPTTL |
| (GQ68_01572) | 319 | TITDCPCTISKTTKITTDVPPTTHSTPHTTTTHVPSTSTPAPTHSVSTISHGGAAKAGVAGLAG |
| | | VAAAAAYFL |
| Erp1 | SEQ ID NO: | MLLTSLLQVFACCLVLPAQVTAFYYYTSGAERKCFHKELSKGTLFQATYKAQIYDDQLQN |
| | 320 | YRDAGAQDFGVLIDIEETFDDNHLVVHQKGSASGDLTFLASDSGEHKICIQPEAGGWLIKA |
| | | KTKIDVEFQVGSDEKLDSKGKATIDILHAKVNVLNSKIGEIRREQKLMRDREATFRDASEA |
| | | VNSRAMWWIVIQLIVLAVTCGWQMKHLGKFFVKQKIL |
| Erp2 | SEQ ID NO: | MIKSTIALPSFFIVLILALVNSVAASSSYAPVAISLPAFSKECLYYDMVTEDDSLAVGYQVLT |
| | 321 | GGNFEIDFDITAPDGSVITSEKQKKYSDFLLKSFGVGKYTFCFSNNYGTALKKVEITLEKEKT |
| | | LTDEHEADVNNDDIIANNAVEEIDRNLNKITKTLNYLRAREWRNMSTVNSTESRLTWLSILI |
| | | IIIIAVISIAQVLLIQFLFTGRQKNYV |
| Emp24 | SEQ ID NO: | MASFATKFVIACFLFFSASAHNVLLPAYGRRCFFEDLSKGDELSISFQFGDRNPQSSSQLTGD |
| | 322 | FIIYGPERHEVLKTVRDTSHGEITLSAPYKGHFQYCFLNENTGIETKDVTFNIHGVVYVDLD |
| | | DPNTNTLDSAVRKLSKLTREVKDEQSYIVIRERTHRNTAESTNDRVKWWSIFQLGVVIANS |
| | | LFQIYYLRRFFEVTSLV |
| Erv25 | SEQ ID NO: | MQVLQLWLTTLISLVVAVQGLHFDIAASTDPEQVCIRDFVTEGQLVVADIHSDGSVGDGQK |
| | 323 | LNLFVRDSVGNEYRRKRDFAGDVRVAFTAPSSTAFDVCFENQAQYRGRSLSRAIELDIESG |
| | | AEARDWNKISANEKLKPIEVELRRVEEITDEIVDELTYLKNREERLRDTNESTNRRVRNFSIL |
| | | VIIVLSSLGVWQVNYLKNYFKTKHII |
| Erp3 | SEQ ID NO: | MSNLCVLFFQFFFLAQFFAEASPLTFELNKGRKECLYTLTPEIDCTISYYFAVQQGESNDFDV |
| | 324 | NYEIFAPDDKNKPIIERSGERQGEWSFIGQHKGEYAICFYGGKAHDKIVDLDFKYNCERQD |
| | | DIRNERRKARKAQRNLRDSKTDPLQDSVENSIDTIERQLHVLERNIQYYKSRNTRNHHTVC |
| | | STEHRIVMFSIYGILLIIGMSCAQIAILEFIFRESRKHNV* |
| Erp5 | SEQ ID NO: | MKYNIVHGICLLFAITQAVGAVHFYAKSGETKCFYEHLSRGNLLIGDLDLYVEKDGLFEED |
| | 325 | PESSLTITVDETFDNDHRVLNQKNSHTGDVTFTALDTGEHRFCFTPFYSKKSATLRVFIELEI |
| | | GNVEALDSKKKEDMNSLKGRVGQLTQRLSSIRKEQDAIREKEAEFRNQSESANSKIMTWSV |
| | | FQLLILLGTCAFQLRYLKNFFVKQKVV |
| Flo5-2 from | SEQ ID NO: | DESGNGDESDTAYGCDITSNAFDGFDATIYEYNANDLKLIRDPVFMSTGYLGRNVLNKISG |
| Komagataella phaffii | 326 | VTVPGFNIWNPRSRTATVYGVQNVNYYNMVLELKGYFKAAVSGDYKLTLSNIDDSSMLFF |
| | | GKNTAFQCCDTGSIPVDQAPTDYSLFTIKPSNQVNSEVISSTQYLEAGKYYPVRIVFVNALE |
| | | RALFNFKLTIPSGTVLDDFQDYIYQFGALDENSCYETTVSKITEWTTYTTPWTGTFETTRTIT |
| | | PTGTEGTVVIETPESYVTTTQPWTGTYETTYTVPPTGTEPGTVIIETPEIIDCEAVCCGPFLTA |
| | | FSFRKREECQCENICCPGDTNCETYVTTTQPWTGTYETTYTVPPTGTEPGTVIIETPESYVTT |
| | | TQPWTGTYETTYTVPPTGTEPGTVIIETPESYVTTTQPWTGTYETTYTVPPSGTEPGTVVIET |
| | | PEIVDCEAYCCASVAIKKRELCQCENFCCSWDQSCQTYVTTTQPWTGTYETTYTVPPTGTE |
| | | PGTVIIETPESYVTTTQPWTGTYETTYTVPPTGTEPGTVIIETPESYVTTTQPWTGTYETTYTV |
| | | PPTGTEPGTVIIETPEIIDCEAVCCGPFLTAFSFRKREECQCENICCPGDTNCETYVTTTQPWT |
| | | GTYETTYTVPPTGTEPGTVIIETPESYVTTTQPWTGTYETTYTVPPTGTEPGTVIIETPESYVT |
| | | TTQPWTGTYETTYTVPPTGTEPGTVIIETPEIINCEAVCCGPFLTAFSFRKREECQCENICCPG |
| | | DTNCETYVTTTQPWTGTYETTYTVPPTGTEPGTVIIETPESYVTTTQPWTGTYETTYTVPST |
| | | GTEPGTVIIETPESYVTTTQPWTGTYETTFTVPPTGTEPGTVVIETPESYVTTTQPWTGTYET |
| | | TYSVPPSGTEPGTVVIETPESYVTTTQPWTGTYETTYSVPPSGTEPGTVVIETPEASTARTKF |
| | | TTVTSSWTGVFTTTKTLPASGTEPATIVIQTPTGYFNTSSLVSTRTKTNVDTVTRVIPCPICTA |
| | | PKTITVVPEEPNESVSVIISQPQSSSTDTTLSKPDSVRVISQPETASQMDTSLSKTDSAVISTET |
| | | AGNNIIPLAGSHSYNTIVTTVTDSPQVAQSTTATSSSNVHLTISTQTTTPSLVYSSSLSTVHQV |
| | | SPSNGGFRSSITVHPLLSVIGAIFGALFM |
| Flo5-2 from | SEQ ID NO: | MKFPVPLLFLLQLFFIIATQGDESGNGDESDTAYGCDITSNAFDGFDATIYEYNANDLKLIRD |
| Komagataella phaffii | 327 | PVFMSTGYLGRNVLNKISGVTVPGFNIWNPRSRTATVYGVQNVNYYNMVLELKGYFKAA |
| (underlined is signal | | VSGDYKLTLSNIDDSSMLFFGKNTAFQCCDTGSIPVDQAPTDYSLFTIKPSNQVNSEVISSTQ |
| peptide, used in some | | YLEAGKYYPVRIVFVNALERALFNFKLTIPSGTVLDDFQDYIYQFGALDENSCYETTVSKIT |
| versions and not | | EWTTYTTPWTGTFETTRTITPTGTEGTVVIETPESYVTTTQPWTGTYETTYTVPPTGTEPGTV |
| others) | | IIETPEIIDCEAVCCGPFLTAFSFRKREECQCENICCPGDTNCETYVTTTQPWTGTYETTYTVP |
| | | PTGTEPGTVIIETPESYVTTTQPWTGTYETTYTVPPTGTEPGTVIIETPESYVTTTQPWTGTYE |
| | | TTYTVPPSGTEPGTVVIETPEIVDCEAYCCASVAIKKRELCQCENFCCSWDQSCQTYVTTTQ |
| | | PWTGTYETTYTVPPTGTEPGTVIIETPESYVTTTQPWTGTYETTYTVPPTGTEPGTVIIETPES |
| | | YVTTTQPWTGTYETTYTVPPTGTEPGTVIIETPEIIDCEAVCCGPFLTAFSFRKREECQCENIC |
| | | CPGDTNCETYVTTTQPWTGTYETTYTVPPTGTEPGTVIIETPESYVTTTQPWTGTYETTYTV |
| | | PPTGTEPGTVIIETPESYVTTTQPWTGTYETTYTVPPTGTEPGTVIIETPEIINCEAVCCGPFLT |
| | | AFSFRKREECQCENICCPGDTNCETYVTTTQPWTGTYETTYTVPPTGTEPGTVIIETPESYVT |
| | | TTQPWTGTYETTYTVPSTGTEPGTVIIETPESYVTTTQPWTGTYETTFTVPPTGTEPGTVVIE |
| | | TPESYVTTTQPWTGTYETTYSVPPSGTEPGTVVIETPESYVTTTQPWTGTYETTYSVPPSGTE |
| | | PGTVVIETPEASTARTKFTTVTSSWTGVFTTTKTLPASGTEPATIVIQTPTGYFNTSSLVSTRT |
| | | KTNVDTVTRVIPCPICTAPKTITVVPEEPNESVSVIISQPQSSSTDTTLSKPDSVRVISQPETAS |
| | | QMDTSLSKTDSAVISTETAGNNIIPLAGSHSYNTIVTTVTDSPQVAQSTTATSSSNVHLTISTQ |
| | | TTTPSLVYSSSLSTVHQVSPSNGGFRSSITVHPLLSVIGAIFGALFM |
| Flo11 from | SEQ ID NO: | SSGKTCPTSEVSPACYANQWETTFPPSDIKITGATWVQDNIYDVTLSYEAESLELENLTELKI |
| Komagataella phaffii | 328 | IGLNSPTGGTKLVWSLNSKVYDIDNPAKWTTTLRVYTKSSADDCYVEMYPFQIQVDWCEA |
| (no signal sequence) | | GASTDGCSAWKWPKSYDYDIGCDNMQDGVSRKHHPVYKWPKKCSSNCGVEPTTSDEPEE |
| | | PTTSEEPEEPTTSEEPEEPTSSDEEPTTSEEPEEPTTSDEPEEPTTSEEPEEPTTSEEPEEPTTSEE |
| | | PTTSEEPEEPTSSDEEPTTSDEPEEPTTSDEPEEPTTSEEPTTSEEPEEPTTSSEEPTPSEEPEGPT |
| | | CPTSEVSPACYADQWETTFPPSDIKITGATWVEDNIYDVTLSYEAESLELENLTELKIIGLNS |
| | | PTGGTKVVWSLNSGIYDIDNPAKWTTTLRVYTKSSADDCYVEMYPFQIQVDWCEAGASTD |
| | | GCSAWKWPKSYDYDIGCDNMQDGVSRKHHPVYKWPKKCSSDCGVEPTTSDEPEEPTTSEE |
| | | PVEPTSSDEEPTTSEEPTTSEEPEEPTTSDEPEEPTTSEEPEEPTTSEEPEEPTTSEEPTTSEEPEE |
| | | PTSSDEEPTTSDEPEEPTTSEEPEEPTTSEEPEEPTTSEEPEEPTTSDEPEEPTTSEEPEEPTTSEE |
| | | PEEPTSSDEEPTTSEEPEEPTTSEEPEEPTTSEEPEEPTTSEEPEEPTSSDEEPTTSEEPEEPTTSD |
| | | EPEEPTTSEEPEEPTTSEEPEEPTSSDEEPTTSEEPEEPTTSDEPEEPTTSEEPEEPTTSEEPEEPT |
| | | TSEEPEEPTTSEEPEEPTSSDEEPTTSEEPEEPTTSDEPEEPTTSEEPEEPTTSEEPEEPTTSEEPE |
| | | EPTTSDEEPGTTEEPLVPTTKTETDVSTTLLTVTDCGTKTCTKSLVITGVTKETVTTHGKTTV |
| | | ITTYCPLPTETVTPTPVTVTSTIYADESVTKTTVYTTGAVEKTVTVGGSSTVVVVHTPLTTA |
| | | VVQSQSTDEIKTVVTARPSTTTIVRDVCYNSVCSVATIVTGVTEKTITFSTGSITVVPTYVPL |
| | | VESEEHQRTASTSETRATSVVVPTVVGQSSSASATSSIFPSVTIHEGVANTVKNSMISGAVAL |
| | | LFNALFL |
| Flo11 from | SEQ ID NO: | MVSLRSIFTSSILAAGLTRAHGSSGKTCPTSEVSPACYANQWETTFPPSDIKITGATWVQDNI |
| Komagataella phaffii | 329 | YDVTLSYEAESLELENLTELKIIGLNSPTGGTKLVWSLNSKVYDIDNPAKWTTTLRVYTKSS |
| (with signal | | ADDCYVEMYPFQIQVDWCEAGASTDGCSAWKWPKSYDYDIGCDNMQDGVSRKHHPVYK |
| sequence) | | WPKKCSSNCGVEPTTSDEPEEPTTSEEPEEPTTSEEPEEPTSSDEEPTTSEEPEEPTTSDEPEEP |
| | | TTSEEPEEPTTSEEPEEPTTSEEPTTSEEPEEPTSSDEEPTTSDEPEEPTTSDEPEEPTTSEEPTTS |
| | | EEPEEPTTSSEEPTPSEEPEGPTCPTSEVSPACYADQWETTFPPSDIKITGATWVEDNIYDVTL |
| | | SYEAESLELENLTELKIIGLNSPTGGTKVVWSLNSGIYDIDNPAKWTTTLRVYTKSSADDCY |
| | | VEMYPFQIQVDWCEAGASTDGCSAWKWPKSYDYDIGCDNMQDGVSRKHHPVYKWPKKC |
| | | SSDCGVEPTTSDEPEEPTTSEEPVEPTSSDEEPTTSEEPTTSEEPEEPTTSDEPEEPTTSEEPEEP |
| | | TTSEEPEEPTTSEEPTTSEEPEEPTSSDEEPTTSDEPEEPTTSEEPEEPTTSEEPEEPTTSEEPEEP |
| | | TTSDEPEEPTTSEEPEEPTTSEEPEEPTSSDEEPTTSEEPEEPTTSEEPEEPTTSEEPEEPTTSEEP |
| | | EEPTSSDEEPTTSEEPEEPTTSDEPEEPTTSEEPEEPTTSEEPEEPTSSDEEPTTSEEPEEPTTSDE |
| | | PEEPTTSEEPEEPTTSEEPEEPTTSEEPEEPTTSEEPEEPTSSDEEPTTSEEPEEPTTSDEPEEPTT |
| | | SEEPEEPTTSEEPEEPTTSEEPEEPTTSDEEPGTTEEPLVPTTKTETDVSTTLLTVTDCGTKTCT |
| | | KSLVITGVTKETVTTHGKTTVITTYCPLPTETVTPTPVTVTSTIYADESVTKTTVYTTGAVEK |
| | | TVTVGGSSTVVVVHTPLTTAVVQSQSTDEIKTVVTARPSTTTIVRDVCYNSVCSVATIVTGV |
| | | TEKTITFSTGSITVVPTYVPLVESEEHQRTASTSETRATSVVVPTVVGQSSSASATSSIFPSVTI |
| | | HEGVANTVKNSMISGAVALLFNALFL |
| Adhesin domain only | SEQ ID NO: | DESGNGDESDTAYGCDITSNAFDGFDATIYEYNANDLKLIRDPVFMSTGYLGRNVLNKISG |
| of Flo5-2 from | 330 | VTVPGFNIWNPRSRTATVYGVQNVNYYNMVLELKGYFKAAVSGDYKLTLSNIDDSSMLFF |
| Komagataella phaffii | | GKNTAFQCCDTGSIPVDQAPTDYSLFTIKPSNQVNSEVISSTQYLEAGKYYPVRIVFVNALE |
| (without signal | | RALFNFKLTIPSGTVLDDFQDYIYQFGALDENSC |
| peptide or | | |
| extension + anchor | | |
| domains) | | |
| Flo5-2 displayed | SEQ ID NO: | EAEADESGNGDESDTAYGCDITSNAFDGFDATIYEYNANDLKLIRDPVFMSTGYLGRNVLN |
| EndoH, single | 331 | KISGVTVPGFNIWNPRSRTATVYGVQNVNYYNMVLELKGYFKAAVSGDYKLTLSNIDDSS |
| NO SS or end. | | MLFFGKNTAFQCCDTGSIPVDQAPTDYSLFTIKPSNQVNSEVISSTQYLEAGKYYPVRIVFV |
| | | NALERALFNFKLTIPSGTVLDDFQDYIYQFGALDENSCGSSGSSGSSGSSGSSGSSGSSGSSE |
| | | AAAREAAAREAAAREAAARGGGGSGGGGSGGGGSAPAPVKQGPTSVAYVEVNNNSMLN |
| | | VGKYTLADGGGNAFDVAVIFAANINYDTGTKTAYLHFNENVQRVLDNAVTQIRPLQQQGI |
| | | KVLLSVLGNHQGAGFANFPSQQAASAFAKQLSDAVAKYGLDGVDFDDEYAEYGNNGTAQ |
| | | PNDSSFVHLVTALRANMPDKIISLYNIGPAASRLSYGGVDVSDKFDYAWNPYYGTWQVPGI |
| | | ALPKAQLSPAAVEIGRTSRSTVADLARRTVDEGYGVYLTYNLDGGDRTADVSAFTRELYG |
| | | SEAVRTP |
| Flo5-2 displayed | SEQ ID NO: | MRFPSIFTAVLFAASSALAAPVNTTTEDETAQIPAEAVIGYSDLEGDFDVAVLPFSNSTNNG |
| EndoH, single | 332 | LLFINTTIASIAAKEEGVSLDKREAEADESGNGDESDTAYGCDITSNAFDGFDATIYEYNAN |
| | | DLKLIRDPVFMSTGYLGRNVLNKISGVTVPGFNIWNPRSRTATVYGVQNVNYYNMVLELK |
| | | GYFKAAVSGDYKLTLSNIDDSSMLFFGKNTAFQCCDTGSIPVDQAPTDYSLFTIKPSNQVNS |
| | | EVISSTQYLEAGKYYPVRIVFVNALERALFNFKLTIPSGTVLDDFQDYIYQFGALDENSCGSS |
| | | GSSGSSGSSGSSGSSGSSGSSEAAAREAAAREAAAREAAARGGGGSGGGGSGGGGSAPAPV |
| | | KQGPTSVAYVEVNNNSMLNVGKYTLADGGGNAFDVAVIFAANINYDTGTKTAYLHFNEN |
| | | VQRVLDNAVTQIRPLQQQGIKVLLSVLGNHQGAGFANFPSQQAASAFAKQLSDAVAKYGL |
| | | DGVDFDDEYAEYGNNGTAQPNDSSFVHLVTALRANMPDKIISLYNIGPAASRLSYGGVDVS |
| | | DKFDYAWNPYYGTWQVPGIALPKAQLSPAAVEIGRTSRSTVADLARRTVDEGYGVYLTYN |
| | | LDGGDRTADVSAFTRELYGSEAVRTP |
| Flo5-2 displayed | SEQ ID NO: | EAEADESGNGDESDTAYGCDITSNAFDGFDATIYEYNANDLKLIRDPVFMSTGYLGRNVLN |
| EndoH, double | 333 | KISGVTVPGFNIWNPRSRTATVYGVQNVNYYNMVLELKGYFKAAVSGDYKLTLSNIDDSS |
| No SS plus the other | | MLFFGKNTAFQCCDTGSIPVDQAPTDYSLFTIKPSNQVNSEVISSTQYLEAGKYYPVRIVFV |
| stuff | | NALERALFNFKLTIPSGTVLDDFQDYIYQFGALDENSCGSSGSSGSSGSSGSSGSSGSSGSSE |
| | | AAAREAAAREAAAREAAARGGGGSGGGGSGGGGSAPAPVKQGPTSVAYVEVNNNSMLN |
| | | VGKYTLADGGGNAFDVAVIFAANINYDTGTKTAYLHFNENVQRVLDNAVTQIRPLQQQGI |
| | | KVLLSVLGNHQGAGFANFPSQQAASAFAKQLSDAVAKYGLDGVDFDDEYAEYGNNGTAQ |
| | | PNDSSFVHLVTALRANMPDKIISLYNIGPAASRLSYGGVDVSDKFDYAWNPYYGTWQVPGI |
| | | ALPKAQLSPAAVEIGRTSRSTVADLARRTVDEGYGVYLTYNLDGGDRTADVSAFTRELYG |
| | | SEAVRTPGSSGSSGSSGSSGSSGSSGSSGSSEAAAREAAAREAAAREAAARGGGGSGGGGS |
| | | GGGGSDESGNGDESDTAYGCDITSNAFDGFDATIYEYNANDLKLIRDPVFMSTGYLGRNVL |
| | | NKISGVTVPGFNIWNPRSRTATVYGVQNVNYYNMVLELKGYFKAAVSGDYKLTLSNIDDS |
| | | SMLFFGKNTAFQCCDTGSIPVDQAPTDYSLFTIKPSNQVNSEVISSTQYLEAGKYYPVRIVFV |
| | | NALERALFNFKLTIPSGTVLDDFQDYIYQFGALDENSCGS |
| Flo5-2 displayed | SEQ ID NO: | MRFPSIFTAVLFAASSALAAPVNTTTEDETAQIPAEAVIGYSDLEGDFDVAVLPFSNSTNNG |
| EndoH, double | 334 | LLFINTTIASIAAKEEGVSLDKREAEADESGNGDESDTAYGCDITSNAFDGFDATIYEYNAN |
| With SS | | DLKLIRDPVFMSTGYLGRNVLNKISGVTVPGFNIWNPRSRTATVYGVQNVNYYNMVLELK |
| | | GYFKAAVSGDYKLTLSNIDDSSMLFFGKNTAFQCCDTGSIPVDQAPTDYSLFTIKPSNQVNS |
| | | EVISSTQYLEAGKYYPVRIVFVNALERALFNFKLTIPSGTVLDDFQDYIYQFGALDENSCGSS |
| | | GSSGSSGSSGSSGSSGSSGSSEAAAREAAAREAAAREAAARGGGGSGGGGSGGGGSAPAPV |
| | | KQGPTSVAYVEVNNNSMLNVGKYTLADGGGNAFDVAVIFAANINYDTGTKTAYLHFNEN |
| | | VQRVLDNAVTQIRPLQQQGIKVLLSVLGNHQGAGFANFPSQQAASAFAKQLSDAVAKYGL |
| | | DGVDFDDEYAEYGNNGTAQPNDSSFVHLVTALRANMPDKIISLYNIGPAASRLSYGGVDVS |
| | | DKFDYAWNPYYGTWQVPGIALPKAQLSPAAVEIGRTSRSTVADLARRTVDEGYGVYLTYN |
| | | LDGGDRTADVSAFTRELYGSEAVRTPGSSGSSGSSGSSGSSGSSGSSGSSEAAAREAAAREA |
| | | AAREAAARGGGGSGGGGSGGGGSDESGNGDESDTAYGCDITSNAFDGFDATIYEYNANDL |
| | | KLIRDPVFMSTGYLGRNVLNKISGVTVPGFNIWNPRSRTATVYGVQNVNYYNMVLELKGY |
| | | FKAAVSGDYKLTLSNIDDSSMLFFGKNTAFQCCDTGSIPVDQAPTDYSLFTIKPSNQVNSEVI |
| | | SSTQYLEAGKYYPVRIVFVNALERALFNFKLTIPSGTVLDDFQDYIYQFGALDENSCGS |
| FLO5 | SEQ ID NO: | MTIAHHCIFLVILAFLALINVASGATEACLPAGQRKSGMNINFYQYSLKDSSTYSNAAYMA |
| Saccharomyces | 335 | YGYASKTKLGSVGGQTDISIDYNIPCVSSSGTFPCPQEDSYGNWGCKGMGACSNSQGIAYW |
| cerevisiae | | STDLFGFYTTPTNVTLEMTGYFLPPQTGSYTFSFATVDDSAILSVGGSIAFECCAQEQPPITST |
| | | NFTINGIKPWDGSLPDNITGTVYMYAGYYYPLKVVYSNAVSWGTLPISVELPDGTTVSDNF |
| | | EGYVYSFDDDLSQSNCTIPDPSIHTTSTITTTTEPWTGTFTSTSTEMTTITDTNGQLTDETVIVI |
| | | RTPTTASTITTTTEPWTGTFTSTSTEMTTVTGTNGQPTDETVIVIRTPTSEGLITTTTEPWTGT |
| | | FTSTSTEMTTVTGTNGQPTDETVIVIRTPTSEGLITTTTEPWTGTFTSTSTEVTTITGTNGQPT |
| | | DETVIVIRTPTSEGLITTTTEPWTGTFTSTSTEMTTVTGTNGQPTDETVIVIRTPTSEGLISTTT |
| | | EPWTGTFTSTSTEVTTITGTNGQPTDETVIVIRTPTSEGLITTTTEPWTGTFTSTSTEMTTVTG |
| | | TNGQPTDETVIVIRTPTSEGLITRTTEPWTGIFTSTSTEVTTITGTNGQPTDETVIVIRTPTTAIS |
| | | SSLSSSSGQITSSITSSRPIITPFYPSNGTSVISSSVISSSVTSSLVTSSSFISSSVISSSTTTSTSIFSE |
| | | SSTSSVIPTSSSTSGSSESKTSSASSSSSSSSISSESPKSPTNSSSSLPPVTSATTGQETASSLPPAT |
| | | TTKTSEQTTLVTVTSCESHVCTESISSAIVSTATVTVSGVTTEYTTWCPISTTETTKQTKGTTE |
| | | QTKGTTEQTTETTKQTTVVTISSCESDICSKTASPAIVSTSTATINGVTTEYTTWCPISTTESK |
| | | QQTTLVTVTSCESGVCSETTSPAIVSTATATVNDVVTVYPTWRPQTTNEQSVSSKMNSATS |
| | | ETTTNTGAAETKTAVTSSLSRFNHAETQTASATDVIGHSSSVVSVSETGNTMSLTSSGLSTM |
| | | SQQPRSTPASSMVGSSTASLEISTYAGSANSLLAGSGLSVFIASLLLAII |
| EndoH-Sed1 fusion | SEQ ID NO: | EAEAAPAPVKQGPTSVAYVEVNNNSMLNVGKYTLADGGGNAFDVAVIFAANINYDTGTK |
| (partial ORF, without | 336 | TAYLHFNENVQRVLDNAVTQIRPLQQQGIKVLLSVLGNHQGAGFANFPSQQAASAFAKQL |
| peptides that are | | SDAVAKYGLDGVDFDDEYAEYGNNGTAQPNDSSFVHLVTALRANMPDKIISLYNIGPAAS |
| cleaved off post- | | RLSYGGVDVSDKFDYAWNPYYGTWQVPGIALPKAQLSPAAVEIGRTSRSTVADLARRTVD |
| translationally) | | EGYGVYLTYNLDGGDRTADVSAFTRELYGSEAVRTPGSSGSSGSSGSSGSSGSSGSSGSSEA |
| | | AAREAAAREAAAREAAARGGGGSGGGGSGGGGSQFSNSTSASSTDVTSSSSISTSSGSVTIT |
| | | SSEAPESDNGTSTAAPTETSTEAPTTAIPTNGTSTEAPTTAIPTNGTSTEAPTDTTTEAPTTAL |
| | | PTNGTSTEAPTDTTTEAPTTGLPTNGTTSAFPPTTSLPPSNTTTTPPYNPSTDYTTDYTVVTE |
| | | YTTYCPEPTTFTTNGKTYTVTEPTTLTITDCPCTIEKPTTTSTTEYTVVTEYTTYCPEPTTFTT |
| | | NGKTYTVTEPTTLTITDCPCTIEKSEAPESSVPVTESKGTTTKETGVTTKQTTANPSLTVSTV |
| | | VPVSSSASSHSVVINSN |
| EndoH-Sed1 fusion | SEQ ID NO: | MRFPSIFTAVLFAASSALAAPVNTTTEDETAQIPAEAVIGYSDLEGDFDVAVLPFSNSTNNG |
| (full ORF, including | 337 | LLFINTTIASIAAKEEGVSLDKREAEAAPAPVKQGPTSVAYVEVNNNSMLNVGKYTLADGG |
| peptides that are | | GNAFDVAVIFAANINYDTGTKTAYLHFNENVQRVLDNAVTQIRPLQQQGIKVLLSVLGNH |
| cleaved off post- | | QGAGFANFPSQQAASAFAKQLSDAVAKYGLDGVDFDDEYAEYGNNGTAQPNDSSFVHLV |
| translationally) | | TALRANMPDKIISLYNIGPAASRLSYGGVDVSDKFDYAWNPYYGTWQVPGIALPKAQLSPA |
| | | AVEIGRTSRSTVADLARRTVDEGYGVYLTYNLDGGDRTADVSAFTRELYGSEAVRTPGSS |
| | | GSSGSSGSSGSSGSSGSSGSSEAAAREAAAREAAAREAAARGGGGSGGGGSGGGGSQFSNS |
| | | TSASSTDVTSSSSISTSSGSVTITSSEAPESDNGTSTAAPTETSTEAPTTAIPTNGTSTEAPTTAI |
| | | PTNGTSTEAPTDTTTEAPTTALPTNGTSTEAPTDTTTEAPTTGLPTNGTTSAFPPTTSLPPSNT |
| | | TTTPPYNPSTDYTTDYTVVTEYTTYCPEPTTFTTNGKTYTVTEPTTLTITDCPCTIEKPTTTST |
| | | TEYTVVTEYTTYCPEPTTFTTNGKTYTVTEPTTLTITDCPCTIEKSEAPESSVPVTESKGTTTK |
| | | ETGVTTKQTTANPSLTVSTVVPVSSSASSHSVVINSNGANVVVPGALGLAGVAMLFL |
| EndoH-Flo5-2 fusion | SEQ ID NO: | APAPVKQGPTSVAYVEVNNNSMLNVGKYTLADGGGNAFDVAVIFAANINYDTGTKTAYL |
| (partial ORF, without | 338 | HFNENVQRVLDNAVTQIRPLQQQGIKVLLSVLGNHQGAGFANFPSQQAASAFAKQLSDAV |
| signal peptide that is | | AKYGLDGVDFDDEYAEYGNNGTAQPNDSSFVHLVTALRANMPDKIISLYNIGPAASRLSY |
| cleaved off post- | | GGVDVSDKFDYAWNPYYGTWQVPGIALPKAQLSPAAVEIGRTSRSTVADLARRTVDEGY |
| translationally) | | GVYLTYNLDGGDRTADVSAFTRELYGSEAVRTPGSSGSSGSSGSSGSSGSSGSSGSSEAAAR |
| | | EAAAREAAAREAAARGGGGSGGGGSGGGGSDESGNGDESDTAYGCDITSNAFDGFDATIY |
| | | EYNANDLKLIRDPVFMSTGYLGRNVLNKISGVTVPGFNIWNPRSRTATVYGVQNVNYYNM |
| | | VLELKGYFKAAVSGDYKLTLSNIDDSSMLFFGKNTAFQCCDTGSIPVDQAPTDYSLFTIKPS |
| | | NQVNSEVISSTQYLEAGKYYPVRIVFVNALERALFNFKLTIPSGTVLDDFQDYIYQFGALDE |
| | | NSCYETTVSKITEWTTYTTPWTGTFETTRTITPTGTEGTVVIETPESYVTTTQPWTGTYETTY |
| | | TVPPTGTEPGTVIIETPEIIDCEAVCCGPFLTAFSFRKREECQCENICCPGDTNCETYVTTTQP |
| | | WTGTYETTYTVPPTGTEPGTVIIETPESYVTTTQPWTGTYETTYTVPPTGTEPGTVIIETPESY |
| | | VTTTQPWTGTYETTYTVPPSGTEPGTVVIETPEIVDCEAYCCASVAIKKRELCQCENFCCSW |
| | | DQSCQTYVTTTQPWTGTYETTYTVPPTGTEPGTVIIETPESYVTTTQPWTGTYETTYTVPPT |
| | | GTEPGTVIIETPESYVTTTQPWTGTYETTYTVPPTGTEPGTVIIETPEIIDCEAVCCGPFLTAFS |
| | | FRKREECQCENICCPGDTNCETYVTTTQPWTGTYETTYTVPPTGTEPGTVIIETPESYVTTTQ |
| | | PWTGTYETTYTVPPTGTEPGTVIIETPESYVTTTQPWTGTYETTYTVPPTGTEPGTVIIETPEII |
| | | NCEAVCCGPFLTAFSFRKREECQCENICCPGDTNCETYVTTTQPWTGTYETTYTVPPTGTEP |
| | | GTVIIETPESYVTTTQPWTGTYETTYTVPSTGTEPGTVIIETPESYVTTTQPWTGTYETTFTVP |
| | | PTGTEPGTVVIETPESYVTTTQPWTGTYETTYSVPPSGTEPGTVVIETPESYVTTTQPWTGTY |
| | | ETTYSVPPSGTEPGTVVIETPEASTARTKFTTVTSSWTGVFTTTKTLPASGTEPATIVIQTPTG |
| | | YFNTSSLVSTRTKTNVDTVTRVIPCPICTAPKTITVVPEEPNESVSVIISQPQSSSTDTTLSKPD |
| | | SVRVISQPETASQMDTSLSKTDSAVISTETAGNNIIPLAGSHSYNTIVTTVTDSPQVAQSTTA |
| | | TSSSNVHLTISTQTTTPSLVYSSSLSTVHQVSPSNGGFRSSITVHPLLSVIGAIFGALFM |
| EndoH-Flo5-2 fusion | SEQ ID NO: | MKFPVPLLFLLQLFFIIATQGAPAPVKQGPTSVAYVEVNNNSMLNVGKYTLADGGGNAFD |
| (full ORF, including | 339 | VAVIFAANINYDTGTKTAYLHFNENVQRVLDNAVTQIRPLQQQGIKVLLSVLGNHQGAGF |
| signal peptide that is | | ANFPSQQAASAFAKQLSDAVAKYGLDGVDFDDEYAEYGNNGTAQPNDSSFVHLVTALRA |
| cleaved off post- | | NMPDKIISLYNIGPAASRLSYGGVDVSDKFDYAWNPYYGTWQVPGIALPKAQLSPAAVEIG |
| translationally) | | RTSRSTVADLARRTVDEGYGVYLTYNLDGGDRTADVSAFTRELYGSEAVRTPGSSGSSGSS |
| | | GSSGSSGSSGSSGSSEAAAREAAAREAAAREAAARGGGGSGGGGSGGGGSDESGNGDESD |
| | | TAYGCDITSNAFDGFDATIYEYNANDLKLIRDPVFMSTGYLGRNVLNKISGVTVPGFNIWN |
| | | PRSRTATVYGVQNVNYYNMVLELKGYFKAAVSGDYKLTLSNIDDSSMLFFGKNTAFQCC |
| | | DTGSIPVDQAPTDYSLFTIKPSNQVNSEVISSTQYLEAGKYYPVRIVFVNALERALFNFKLTIP |
| | | SGTVLDDFQDYIYQFGALDENSCYETTVSKITEWTTYTTPWTGTFETTRTITPTGTEGTVVIE |
| | | TPESYVTTTQPWTGTYETTYTVPPTGTEPGTVIIETPEIIDCEAVCCGPFLTAFSFRKREECQC |
| | | ENICCPGDTNCETYVTTTQPWTGTYETTYTVPPTGTEPGTVIIETPESYVTTTQPWTGTYETT |
| | | YTVPPTGTEPGTVIIETPESYVTTTQPWTGTYETTYTVPPSGTEPGTVVIETPEIVDCEAYCCA |
| | | SVAIKKRELCQCENFCCSWDQSCQTYVTTTQPWTGTYETTYTVPPTGTEPGTVIIETPESYV |
| | | TTTQPWTGTYETTYTVPPTGTEPGTVIIETPESYVTTTQPWTGTYETTYTVPPTGTEPGTVIIE |
| | | TPEIIDCEAVCCGPFLTAFSFRKREECQCENICCPGDTNCETYVTTTQPWTGTYETTYTVPPT |
| | | GTEPGTVIIETPESYVTTTQPWTGTYETTYTVPPTGTEPGTVIIETPESYVTTTQPWTGTYETT |
| | | YTVPPTGTEPGTVIIETPEIINCEAVCCGPFLTAFSFRKREECQCENICCPGDTNCETYVTTTQ |
| | | PWTGTYETTYTVPPTGTEPGTVIIETPESYVTTTQPWTGTYETTYTVPSTGTEPGTVIIETPES |
| | | YVTTTQPWTGTYETTFTVPPTGTEPGTVVIETPESYVTTTQPWTGTYETTYSVPPSGTEPGT |
| | | VVIETPESYVTTTQPWTGTYETTYSVPPSGTEPGTVVIETPEASTARTKFTTVTSSWTGVFTT |
| | | TKTLPASGTEPATIVIQTPTGYFNTSSLVSTRIKTNVDTVTRVIPCPICTAPKTITVVPEEPNES |
| | | VSVIISQPQSSSTDTTLSKPDSVRVISQPETASQMDTSLSKTDSAVISTETAGNNIIPLAGSHSY |
| | | NTIVTTVTDSPQVAQSTTATSSSNVHLTISTQTTTPSLVYSSSLSTVHQVSPSNGGFRSSITVH |
| | | PLLSVIGAIFGALFM |
| EndoH-Flo11 fusion | SEQ ID NO: | APAPVKQGPTSVAYVEVNNNSMLNVGKYTLADGGGNAFDVAVIFAANINYDTGTKTAYL |
| (partial ORF, without | 340 | HFNENVQRVLDNAVTQIRPLQQQGIKVLLSVLGNHQGAGFANFPSQQAASAFAKQLSDAV |
| signal peptide that is | | AKYGLDGVDFDDEYAEYGNNGTAQPNDSSFVHLVTALRANMPDKIISLYNIGPAASRLSY |
| cleaved off post- | | GGVDVSDKFDYAWNPYYGTWQVPGIALPKAQLSPAAVEIGRTSRSTVADLARRTVDEGY |
| translationally) | | GVYLTYNLDGGDRTADVSAFTRELYGSEAVRTPGSSGSSGSSGSSGSSGSSGSSGSSEAAAR |
| | | EAAAREAAAREAAARGGGGGGGGSGGGGSSSGKTCPTSEVSPACYANQWETTFPPSDIKI |
| | | TGATWVQDNIYDVTLSYEAESLELENLTELKIIGLNSPTGGTKLVWSLNSKVYDIDNPAKW |
| | | TTTLRVYTKSSADDCYVEMYPFQIQVDWCEAGASTDGCSAWKWPKSYDYDIGCDNMQD |
| | | GVSRKHHPVYKWPKKCSSNCGVEPTTSDEPEEPTTSEEPEEPTTSEEPEEPTSSDEEPTTSEEP |
| | | EEPTTSDEPEEPTTSEEPEEPTTSEEPEEPTTSEEPTTSEEPEEPTSSDEEPTTSDEPEEPTTSDEP |
| | | EEPTTSEEPTTSEEPEEPTTSSEEPTPSEEPEGPTCPTSEVSPACYADQWETTFPPSDIKITGAT |
| | | WVEDNIYDVTLSYEAESLELENLTELKIIGLNSPTGGTKVVWSLNSGIYDIDNPAKWTTTLR |
| | | VYTKSSADDCYVEMYPFQIQVDWCEAGASTDGCSAWKWPKSYDYDIGCDNMQDGVSRK |
| | | HHPVYKWPKKCSSDCGVEPTTSDEPEEPTTSEEPVEPTSSDEEPTTSEEPTTSEEPEEPTTSDE |
| | | PEEPTTSEEPEEPTTSEEPEEPTTSEEPTTSEEPEEPTSSDEEPTTSDEPEEPTTSEEPEEPTTSEE |
| | | PEEPTTSEEPEEPTTSDEPEEPTTSEEPEEPTTSEEPEEPTSSDEEPTTSEEPEEPTTSEEPEEPTT |
| | | SEEPEEPTTSEEPEEPTSSDEEPTTSEEPEEPTTSDEPEEPTTSEEPEEPTTSEEPEEPTSSDEEPT |
| | | TSEEPEEPTTSDEPEEPTTSEEPEEPTTSEEPEEPTTSEEPEEPTTSEEPEEPTSSDEEPTTSEEPE |
| | | EPTTSDEPEEPTTSEEPEEPTTSEEPEEPTTSEEPEEPTTSDEEPGTTEEPLVPTTKTETDVSTTL |
| | | LTVTDCGTKTCTKSLVITGVTKETVTTHGKTTVITTYCPLPTETVTPTPVTVTSTIYADESVT |
| | | KTTVYTTGAVEKTVTVGGSSTVVVVHTPLTTAVVQSQSTDEIKTVVTARPSTTTIVRDVCY |
| | | NSVCSVATIVTGVTEKTITFSTGSITVVPTYVPLVESEEHQRTASTSETRATSVVVPTVVGQS |
| | | SSASATSSIFPSVTIHEGVANTVKNSMISGAVALLFNALFL |
| EndoH-Flo11 fusion | SEQ ID NO: | MVSLRSIFTSSILAAGLTRAHGAPAPVKQGPTSVAYVEVNNNSMLNVGKYTLADGGGNAF |
| (full ORF, including | 341 | DVAVIFAANINYDTGTKTAYLHFNENVQRVLDNAVTQIRPLQQQGIKVLLSVLGNHQGAG |
| signal peptide that is | | FANFPSQQAASAFAKQLSDAVAKYGLDGVDFDDEYAEYGNNGTAQPNDSSFVHLVTALR |
| cleaved off post- | | ANMPDKIISLYNIGPAASRLSYGGVDVSDKFDYAWNPYYGTWQVPGIALPKAQLSPAAVEI |
| translationally) | | GRTSRSTVADLARRTVDEGYGVYLTYNLDGGDRTADVSAFTRELYGSEAVRTPGSSGSSG |
| | | SSGSSGSSGSSGSSGSSEAAAREAAAREAAAREAAARGGGGSGGGGSGGGGSSSGKTCPTS |
| | | EVSPACYANQWETTFPPSDIKITGATWVQDNIYDVTLSYEAESLELENLTELKIIGLNSPTGG |
| | | TKLVWSLNSKVYDIDNPAKWTTTLRVYTKSSADDCYVEMYPFQIQVDWCEAGASTDGCS |
| | | AWKWPKSYDYDIGCDNMQDGVSRKHHPVYKWPKKCSSNCGVEPTTSDEPEEPTTSEEPEE |
| | | PTTSEEPEEPTSSDEEPTTSEEPEEPTTSDEPEEPTTSEEPEEPTTSEEPEEPTTSEEPTTSEEPEE |
| | | PTSSDEEPTTSDEPEEPTTSDEPEEPTTSEEPTTSEEPEEPTTSSEEPTPSEEPEGPTCPTSEVSPA |
| | | CYADQWETTFPPSDIKITGATWVEDNIYDVTLSYEAESLELENLTELKIIGLNSPTGGTKVV |
| | | WSLNSGIYDIDNPAKWTTTLRVYTKSSADDCYVEMYPFQIQVDWCEAGASTDGCSAWKW |
| | | PKSYDYDIGCDNMQDGVSRKHHPVYKWPKKCSSDCGVEPTTSDEPEEPTTSEEPVEPTSSD |
| | | EEPTTSEEPTTSEEPEEPTTSDEPEEPTTSEEPEEPTTSEEPEEPTTSEEPTTSEEPEEPTSSDEEP |
| | | TTSDEPEEPTTSEEPEEPTTSEEPEEPTTSEEPEEPTTSDEPEEPTTSEEPEEPTTSEEPEEPTSSD |
| | | EEPTTSEEPEEPTTSEEPEEPTTSEEPEEPTTSEEPEEPTSSDEEPTTSEEPEEPTTSDEPEEPTTS |
| | | EEPEEPTTSEEPEEPTSSDEEPTTSEEPEEPTTSDEPEEPTTSEEPEEPTTSEEPEEPTTSEEPEEP |
| | | TTSEEPEEPTSSDEEPTTSEEPEEPTTSDEPEEPTTSEEPEEPTTSEEPEEPTTSEEPEEPTTSDEE |
| | | PGTTEEPLVPTTKTETDVSTTLLTVTDCGTKTCTKSLVITGVTKETVTTHGKTTVITTYCPLP |
| Suc2-Tir4p fusion | SEQ ID NO: | TETVTPTPVTVTSTIYADESVTKTTVYTTGAVEKTVTVGGSSTVVVVHTPLTTAVVQSQST |
| protein | 342 | DEIKTVVTARPSTTTIVRDVCYNSVCSVATIVTGVTEKTITFSTGSITVVPTYVPLVESEEHQR |
| | | TASTSETRATSVVVPTVVGQSSSASATSSIFPSVTIHEGVANTVKNSMISGAVALLFNALFL |
| | | MRFPSIFTAVLFAASSALAAPVQTTTEDELEGDFDVAVLPFSASIAAKEEGVSLEKREAEAS |
| | | MTNETSDRPLVHFTPNKGWMNDPNGLWYDEKDAKWHLYFQYNPNDTVWGTPLFWGHA |
| | | TSDDLTNWEDQPIAIAPKRNDSGAFSGSMVVDYNNTSGFFNDTIDPRQRCVAIWTYNTPES |
| | | EEQYISYSLDGGYTFTEYQKNPVLAANSTQFRDPKVFWYEPSQKWIMTAAKSQDYKIEIYS |
| | | SDDLKSWKLESAFANEGFLGYQYECPGLIEVPTEQDPSKSYWVMFISINPGAPAGGSFNQYF |
| | | VGSFNGTHFEAFDNQSRVVDFGKDYYALQTFFNTDPTYGSALGIAWASNWEYSAFVPTNP |
| | | WRSSMSLVRKFSLNTEYQANPETELINLKAEPILNISNAGPWSRFATNTTLTKANSYNVDLS |
| | | NSTGTLEFELVYAVNTTQTISKSVFADLSLWFKGLEDPEEYLRMGFEVSASSFFLDRGNSKV |
| | | KFVKENPYFTNRMSVNNQPFKSENDLSYYKVYGLLDQNILELYFNDGDVVSTNTYFMTTG |
| | | NALGSVNMTTGVDNLFYIDKFQVREVKGSSGSSGSSGSSGSSGSSGSSGSSEAAAREAAAR |
| | | EAAAREAAARGGGGSGGGGSGGGGSQINELNVVLDDVKTNIADYITLSYTPNSGFSLDQM |
| | | PAGIMDIAAQLVANPSDDSYTTLYSEVDFSAVEHMLTMVPWYSSRLLPELEAMDASLTTSS |
| | | SAATSSSEVASSSIASSTSSSVAPSSSEVVSSSVASSSSEVASSSVASTSEATSSSAVTSSSAVS |
| | | SSTESVSSSSVSSSSAVSSSEAVSSSPVSSVVSSSAGPASSSVAPYNSTIASSSSTAQTSISTIAP |
| | | YNSTTTTTPASSASSVIISTRNGTTVTETDNTLVTKETTVCDYSSTSAVPASTTGYNNSTKVS |
| | | TATICSTCKEGTSTATDFSTLKTTVTVCDSACQAKKSATVVSVQSKTTGIVEQTENGAAKA |
| | | VIGMGAGALAAVAAMLL |
| BMT1 | SEQ ID NO: | MVDLFQWLKFYSMRRLGQVAITLVLLNLFVFLGYKFTPSTVIGSPSWEPAVVPTVFNESYL |
| (XP_002493883/ | 343 | DSLQFTDINVDSFLSDTNGRISVTCDSLAYKGLVKTSKKKELDCDMAYIRRKIFSSEEYGVL |
| GQ68_04782T0) | | ADLEAQDITEEQRIKKHWFTFYGSSVYLPEHEVHYLVRRVLFSKVGRADTPVISLLVAQLY |
| | | DKDWNELTPHTLEIVNPATGNVTPQTFPQLIHVPIEWSVDDKWKGTEDPRVFLKPSKTGVS |
| | | EPIVLFNLQSSLCDGKRGMFVTSPFRSDKVNLLDIEDKERPNSEKNWSPFFLDDVEVSKYST |
| | | GYVHFVYSFNPLKVIKCSLDTGACRMIYESPEEGRFGSELRGATPMVKLPVHLSLPKGKEV |
| | | WVAFPRTRLRDCGCSRTTYRPVLTLFVKEGNKFYTELISSSIDFHIDVLSYDAKGESCSGSIS |
| | | VLIPNGIDSWDVSKKQGGKSDILTLTLSEADRNTVVVHVKGLLDYLLVLNGEGPIHDSHSF |
| | | KNVLSTNHFKSDTTLLNSVKAAECAIFSSRDYCKKYGETRGEPARYAKQMENERKEKEKK |
| | | EKEAKEKLEAEKAEMEEAVRKAQEAIAQKEREKEEAEQEKKAQQEAKEKEAEEKAAKEK |
| | | EAKENEAKKKIIVEKLAKEQEEAEKLEAKKKLYQLQEEERS |
| BMT2 | SEQ ID NO: | MRTRLNFLLLCIASVLSVIWIGVLLTWNDNNLGGISLNGGKDSAYDDLLSLGSENDMEVDS |
| (XP_002493882/ | 344 | YVTNIYDNAPVLGCTDLSYHGLLKVTPKHDLACDLEFIRAQILDIDVYSAIKDLEDKALTVK |
| GQ68_04781T0) | | QKVEKHWFTFYGSSVFLPEHDVHYLVRRVIFSAEGKANSPVTSIIVAQIYDKNWNELNGHF |
| | | LDILNPNTGKVQHNTFPQVLPIATNFVKGKKFRGAEDPRVVLRKGRFGPDPLVMFNSLTQD |
| | | NKRRRIFTISPFDQFKTVMYDIKDYEMPRYEKNWVPFFLKDNQEAVHFVYSFNPLRVLKCS |
| | | LDDGSCDIVFEIPKVDSMSSELRGATPMINLPQAIPMAKDKEIWVSFPRTRIANCGCSRTTYR |
| | | PMLMLFVREGSNFFVELLSTSLDFGLEVLPYSGNGLPCSADHSVLIPNSIDNWEVVDSNGDD |
| | | ILTLSFSEADKSTSVIHIRGLYNYLSELDGYQGPEAEDEHNFQRILSDLHFDNKTTVNNFIKV |
| | | QSCALDAAKGYCKEYGLTRGEAERRRRVAEERKKKEKEEEEKKKKKEKEEEEKKRIEEEK |
| | | KKIEEKERKEKEKEEAERKKLQEMKKKLEEITEKLEKGQRNKEIDPKEKQREEEERKERVR |
| | | KIAEKQRKEAEKKEAEKKANDKKDLKIRQ |
| MNN2 | SEQ ID NO: | MFGKRRQVRKLLIWVVLLLIVYFFGLQFRAKNSAHQSSIRSFYADNKEFFDRQYSRYDEYD |
| (XP_002492593/ | 345 | IIDNMNSHNELLQEQFRNGKLAAGLRGVAEEPNSDEVTDDTAIEEDEQAAMINFPKRSPQR |
| GQ68_03403T0) | | EKSLVELRKFYKNVLSIIINNKPAMPIENPRDPTPNENALKRKFGKSGIINIALHDTDPSLPILS |
| | | EAYLRDSLQLSPSFIASLSKSHSAVVKAFPPSFPANAYNGTGIVFIGGQKFSWLSLLSIENLRK |
| | | TGSKVPVELIIPFAHEYEPQLCEEILPKLNATCVLLQETVGIDLLKSGHLKGYQFKSLALLAS |
| | | SFEQVLLVDSDNIIVENPDPIFDSEVFQRTGLVLWPDFWRRVTHPDYYKIAGIKLGSERVRH |
| | | VVDSYTDPSLYTSSSEDPFTDIPLHDREGAIPDGSTESGQILISKTKHCQTILLSLYYNFFGPD |
| | | YYYPLFTQGASGEGDKETFLAAANYYKLPFYNIKKGVDVIGYWKPDQSAYQGCGMLQYD |
| | | PIVDYQNLQTFLKTHKGSRVNKLEQSELDKPGLLSRLIPKFFFRKTFDEHQLQSHFTKDRSKI |
| | | MFIHSNFPKLDPFGLKLHNYLFVDQDTHKPRIRMYADQTGLSFDFELRQWIIIHEYFCEYPD |
| | | FNLKYLENANVKPQDLCMFIKEELNFLQNNPIQLT |
| MNN2/5 homolog 1- | SEQ ID NO: | MLFGLIRHSRRQLLFLGALVTVIVLIFTLPNTSPIEANGVKSEEGSITPIIPVLESPANSLEKIVD |
| MNNF1 | 346 | TASEERIGGATLEEGHENNKEEQALENAERAKEKEKTEAIAAEEEKLKAAELLRQQETTRE |
| (XP_002490149/ | | KEAAKEDDSKKPNQELVEQDTYLDDIPDDVEDNIIISEQDRKKIILPSYTPKTDPAYSKRATA |
| GQ68_02166T0) | | LKIFYNDFFIKVADSGPNTAPITKKTRKKGKSKLKGDVSSGDKYEGPVLTEDFLRFMEIYSD |
| | | EFIDAVSESHSKIVNLMPESFPKGMYQGDGIVIIGGGVYSWYGLLAIRNLRDGGNTLPVELM |
| | | LPSDNEYEPQLCEQILPSLNAKCIMLSDIVDQDVLKKLDFKGYQFKALSLLASSFENVLSLD |
| | | SDNIPVANVSHLFDHEPFSETGLVSWPDFWRRTTNPRYYEAAGIKIGEYQVRNCLDGFVPES |
| | | DFVHIGLKDIPLHDRNGTIPDASTESGQLLVNKNKHAKTLMLMFYYNFYGPGYYYPLLSQG |
| | | MAGEGDKETFLAAANFFGLPFYQVKAGPGILGHHDSTGAFTGVAIVQYDPIADYELTKENF |
| | | VGEKRKGIEAPKAFYGNNNKSPLFHHCNFPKLDPVKLIKEKKLIDNKTHKFNRMYGPNTKL |
| | | KYDFEERQWKYTKEYLCEKKYNLLYFTEQYKNYGQGYSQERICKFSDRFLKFLSDNPIRIE |
| | | G |
| MNN2/5 homolog 2- | SEQ ID NO: | MFNSLAPMRLKKLLKVFCASVVLLAATSVVLFFHFGGQIIIPIPERTVTLSTPPANDTWQFQ |
| MNNF2 | 347 | QFFNGYLDALLENNLSYPIPERWNHEVTNVRFFNRIGELLSESRLQELIHFSPEFIEDTSDKFD |
| (XP_002493020/ | | NIVEQIPAKWPYENMYRGDGYVIVGGGRHTFLALLNINALRRAGNKLPVEVVLPTYDDYE |
| GQ68_03863TO) | | EDFCENHFPLLNARCVILEERFGDQVYPRLQLGGYQFKIFAIAASSFKNCFLLDSDNIPLRKM |
| | | DKIFSSELYKNKTMITWPDFWLRSTSPHYYHNITKTPIGDKRVRYFNDFYTNPNEYYYGDE |
| | | DPRSEIPFHDREGTIPDWTTESGQLVINKEVHFPAILLGLFYNFNGPMGFYPLLSQGGAGEG |
| | | DKDTFVAASHYYNLPYYQVYKNCEMLYGWVDHANSGRIEHSAIVQYNPIVDYENLQSVK |
| | | AKAEIILKNHEPDSRKKSSKPKSYSKTRLSTHVKGSIYSYRRLFRDSFNKANSDEMFLHCHT |
| | | PKIEPYRIMEDDLTLGRNKEAKQRWYGGRKNRVRFGYDVELYIWELIDQYICDKNIQYKIF |
| | | EGKDRDALCGSFMREQLGFLRSTGD |

1. An engineered eukaryotic cell that expresses a surface-displayed fusion protein comprising a catalytic domain of an enzyme and an anchoring domain of a glycosylphosphatidylinositol (GPI)-anchored protein, wherein the anchoring domain comprises at least about 200 amino acids, and at least about 30% of the residues in the anchoring domain are serines or threonines.

2. The engineered eukaryotic cell claim 1, wherein the anchoring domain comprises at least about 225 amino acids, at least about 250 amino acids, at least about 275 amino acids, at least about 300 amino acids, at least about 325 amino acids, at least about 350 amino acids, at least about 375 amino acids, or at least about 400 amino acids.

3. The engineered eukaryotic cell of claim 1, wherein at least about 35% of the residues in the anchoring domain are serines or threonines, at least about 40% of the residues in the anchoring domain are serines or threonines, at least about 45% of the residues in the anchoring domain are serines or threonines, or at least about 50% of the residues in the anchoring domain are serines or threonines.

4. The engineered eukaryotic cell of claim 1, wherein the serines or threonines in the anchoring domain are capable of being O-mannosylated.

5. The engineered eukaryotic cell of claim 1, wherein the fusion protein having an anchoring domain comprising at least about 325 amino acids provides greater enzymatic activity relative to a fusion protein having an anchoring domain comprising less than about 300 amino acids.

6. The engineered eukaryotic cell of claim 1, wherein the fusion protein having an anchoring domain comprising at least about 300 amino acids provides greater enzymatic activity relative to a fusion protein having an anchoring domain comprising less than about 250 amino acids.

7. The engineered eukaryotic cell of claim 1, wherein the fusion protein comprises the anchoring domain of the GPI anchored protein.

8. The engineered eukaryotic cell of claim 1, wherein the fusion protein comprises the GPI anchored protein without its native signal peptide.

9. The engineered eukaryotic cell of claim 1, wherein the GPI anchored protein is not native to the engineered eukaryotic cell.

10. The engineered eukaryotic cell of claim 1, wherein the GPI anchored protein is naturally expressed by a S. cerevisiae cell and the engineered eukaryotic cell is not a S. cerevisiae cell.

11. The engineered eukaryotic cell of claim 1, wherein the GPI anchored protein is selected from Tir4, Dan1, Dan4, Sag1, FIG. 2, or Sed1.

12. The engineered eukaryotic cell of claim 1, wherein the anchoring domain of the GPI anchored protein comprises an amino acid sequence that is at least 70% identical, at least 75% identical, at least 80% identical, at least 85% identical, at least 90% identical, or at least 95% identical, to one of SEQ ID NO: 1 to SEQ ID NO: 14.

13. The engineered eukaryotic cell of claim 1, wherein the anchoring domain of the GPI anchored protein comprises an amino acid sequence of one of SEQ ID NO: 1 to SEQ ID NO: 14.

14. The engineered eukaryotic cell of claim 1, wherein the engineered eukaryotic cell is a yeast cell.

15. The engineered eukaryotic cell of claim 1, wherein the engineered eukaryotic cell is a Pichia species.

16. The engineered eukaryotic cell of claim 15, wherein the Pichia species is Pichia pastoris.

17. The engineered eukaryotic cell of claim 1, wherein the engineered eukaryotic cell comprises a genomic modification that expresses the fusion protein and/or comprises an extrachromosomal modification that expresses the fusion protein.

18. The engineered eukaryotic cell of claim 1, wherein the fusion protein comprises a portion of the enzyme in addition to its catalytic domain.

19. The engineered eukaryotic cell of claim 1, wherein the fusion protein comprises substantially the entire amino acid sequence of the enzyme.

20. The engineered eukaryotic cell of claim 1, wherein the enzyme catalyzes a post-translational modification of a protein secreted by the engineered eukaryotic cell, the enzyme catalyzes a reaction which removes impurities secreted by the engineered eukaryotic cell, and/or the enzyme catalyzes a reaction which allows the engineered eukaryotic cell to rely on alternate carbon sources.

21. The engineered eukaryotic cell of claim 20, wherein the catalyzed post-translational modification comprises deglycosylation, acetylation, adenylation, alkylation, amidation, glycosylation, hydroxylation, methylation, proteolysis, or phosphorylation.

22. The engineered eukaryotic cell of claim 20, wherein the enzyme catalyzing a post-translational modification is endoglycosidase H.

23. The engineered eukaryotic cell of claim 20, wherein the enzyme that catalyzes a reaction that removes impurities comprises a hydrolase, a decarboxylase, an esterase, a lipase, a phosphatase, a glycosidase, a peptidase, a protease, or a nucleosidase.

24. The engineered eukaryotic cell of claim 20, wherein the enzyme that catalyzes a reaction that removes impurities is a mannosidase.

25. The engineered eukaryotic cell of claim 20, wherein the enzyme that catalyzes a reaction which allows the engineered eukaryotic cell to rely on alternate carbon sources comprises a sucrase, an invertase, an amylase, a cellulase, an isomaltase, a lactase, a maltase, or a sugar isomerase.

26. The engineered eukaryotic cell of claim 25, wherein the enzyme that catalyzes a reaction which allows the engineered eukaryotic cell to rely on alternate carbon sources is a sucrase or an invertase.

27. The engineered eukaryotic cell of claim 1, wherein the enzyme comprises an amino acid sequence that is at least 70% identical, at least 75% identical, at least 80% identical, at least 85% identical, at least 90% identical, or at least 95% identical, to one of SEQ ID NO: 15 to SEQ ID NO: 20.

28. The engineered eukaryotic cell of claim 1, wherein the enzyme comprises an amino acid sequence of one of SEQ ID NO: 15 to SEQ ID NO: 20.

29. The engineered eukaryotic cell of claim 1, wherein the fusion protein comprises an amino acid sequence that is at least 70% identical, at least 75% identical, at least 80% identical, at least 85% identical, at least 90% identical, or at least 95% identical, to an amino acid sequence selected from SEQ ID NO: 21 to SEQ ID NO: 26.

30. The engineered eukaryotic cell of claim 1, wherein the fusion protein comprises an amino acid sequence selected from: SEQ ID NO: 24 to SEQ ID NO: 26.

31. The engineered eukaryotic cell of claim 1, wherein in the fusion protein, the catalytic domain is N-terminal to the anchoring domain.

32. The engineered eukaryotic cell of claim 1, wherein the fusion protein comprises a linker between the catalytic domain and the anchoring domain.

33. The engineered eukaryotic cell of claim 1, wherein the fusion protein comprises a linker having an amino acid sequence that is at least 95% identical to SEQ ID NO: 31.

34. The engineered eukaryotic cell of claim 1, wherein, upon translation, the fusion protein comprises a signal peptide and/or a secretory signal.

35. The engineered eukaryotic cell of claim 1, wherein the engineered eukaryotic cell comprises two or more fusion proteins, three or more fusion proteins, or four fusion proteins.

36. The engineered eukaryotic cell of claim 35, wherein the two or more fusion proteins comprise different enzyme types.

37. The engineered eukaryotic cell of claim 35, wherein the two or more fusion proteins comprise the same enzyme type.

38. The engineered eukaryotic cell of claim 35, wherein two of the three or more fusion proteins or two of the four or more fusion proteins comprise different enzyme types.

39. The engineered eukaryotic cell of claim 35, wherein two of the three or more fusion proteins or two of the four or more fusion proteins comprise the same enzyme type.

40. The engineered eukaryotic cell of claim 35, wherein three of the three or more fusion proteins or three of the four or more fusion proteins comprise different enzyme types.

41. The engineered eukaryotic cell of claim 35, wherein three of the three or more fusion proteins or three of the four or more fusion proteins comprise the same enzyme type.

42. The engineered eukaryotic cell of claim 35, wherein each of the two or more, three or more, or four fusion proteins comprise different enzyme types.

43. The engineered eukaryotic cell of claim 35, wherein each of the two or more, three or more, or four fusion proteins comprise the same enzyme type.

44. The engineered eukaryotic cell of claim 35, wherein the enzyme types are selected from an enzyme that catalyzes a post-translational modification of a protein secreted by the engineered eukaryotic cell, an enzyme that catalyzes a reaction which removes impurities secreted by the engineered eukaryotic cell, and/or an enzyme that catalyzes a reaction which allows the engineered eukaryotic cell to rely on alternate carbon sources.

45. The engineered eukaryotic cell of claim 1, wherein the engineered eukaryotic cell comprises a mutation in its AOX1 gene and/or its AOX2 gene.

46. The engineered eukaryotic cell of claim 1, wherein the engineered eukaryotic cell comprises a genomic modification that overexpresses a secreted recombinant protein and/or comprises an extrachromosomal modification that overexpresses a secreted recombinant protein.

47. The engineered eukaryotic cell of claim 46, wherein the secreted recombinant protein is an animal protein.

48. The engineered eukaryotic cell of claim 47, wherein the animal protein is an egg protein.

49. The engineered eukaryotic cell of claim 48, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

50. The engineered eukaryotic cell of claim 46, wherein the genomic modification and/or the extrachromosomal modification that overexpresses the secreted recombinant protein comprises an inducible promoter.

51. The engineered eukaryotic cell of claim 50, wherein the inducible promoter is an AOX1, DAK2, PEX11, FLD1, FGH1, DAS1, DAS2, CAT1, MDH3, HAC1, BiP, RAD30, RVS161-2, MPP10, THP3, TLR, GBP2, PMP20, SHB17, PEX8, PEX4, or TKL3 promoter.

52. The engineered eukaryotic cell of claim 46, wherein the genomic modification and/or the extrachromosomal modification that overexpresses a secreted recombinant protein comprises an AOX1, TDH3, MOX, RPS25A, or RPL2A terminator.

53. The engineered eukaryotic cell of claim 46, wherein the genomic modification and/or the extrachromosomal modification that overexpresses a secreted recombinant protein encodes a signal peptide and/or a secretory signal.

54. The engineered eukaryotic cell of claim 46, wherein the genomic modification and/or the extrachromosomal modification that overexpresses a secreted recombinant protein comprises codons that are optimized for the species of the engineered eukaryotic cell.

55. The engineered eukaryotic cell of claim 46, wherein the secreted recombinant protein is designed to be secreted from the cell and/or is capable of being secreted from the cell.

56. The engineered eukaryotic cell of claim 1, wherein the engineered eukaryotic cell comprises an additional genomic modification comprising a knockout of a coding sequence for a cell wall protein or an additional genomic modification that overexpresses a cell wall protein.

57. The engineered eukaryotic cell of claim 56, wherein the engineered eukaryotic cell comprises an additional genomic modification comprising a knockout of the coding sequences for more than one cell wall proteins or an additional genomic modification that overexpresses more than one a cell wall proteins.

58. The engineered eukaryotic cell of claim 56, wherein the cell wall protein is a mannoprotein.

59. The engineered eukaryotic cell of claim 56, wherein the cell wall protein is one or more of a CCW12 homolog, a CCW14 homolog, a CCW22 homolog, a FLO5 homolog, or a SED1 homolog.

60. The engineered eukaryotic cell of claim 56, wherein the cell wall protein comprises the amino acid sequence of any one of SEQ ID NO: 306 to SEQ ID NO: 319.

61. The engineered eukaryotic cell of claim 56, wherein the additional genomic modification reduces the number of native cell wall proteins expressed by the engineered eukaryotic cell, thereby allowing additional space for localization of the surface-displayed fusion protein.

62. The engineered eukaryotic cell of claim 1, wherein the engineered eukaryotic cell comprises a further genomic modification that overexpresses a protein related to the p24 complex.

63. The engineered eukaryotic cell of claim 62, wherein the engineered eukaryotic cell comprises a further genomic modification comprising that overexpresses more than one protein related to the p24 complex.

64. The engineered eukaryotic cell of claim 62, wherein the protein related to the p24 complex is selected from Erp1, Erp2, Erp3, Erp5, Emp24, and Erv25.

65. The engineered eukaryotic cell of claim 62, wherein the protein related to the p24 complex comprises the amino acid sequence of any one of SEQ ID NO: 320 to SEQ ID NO: 325.

66. The engineered eukaryotic cell of claim 62, wherein the further genomic modification promotes trafficking of the surface-displayed fusion protein through the secretory pathway.

67. The engineered eukaryotic cell of claim 1, wherein the engineered eukaryotic cell further encodes one or more additional fusion proteins comprising a catalytic domain of an enzyme and an adhesion or anchoring domain from a cell surface protein selected from Sed1p, Flo5-2, Flo11, Saccharomyces cerevisiae Flo5, CWP, and PIR with the adhesion or anchoring domain having the ability to capture exopolysaccharides and retain the additional fusion protein at the extracellular surface.

68. A method for expressing a surface-displayed fusion protein comprising a catalytic domain of an enzyme and an anchoring domain of glycosylphosphatidylinositol (GPI)-anchored protein, the method comprising obtaining the engineered eukaryotic cell of claim 1 and culturing the engineered eukaryotic cell under conditions that promote expression of the fusion protein.

69. The method of claim 68, wherein when the engineered eukaryotic cell comprises a genomic modification and/or an extrachromosomal modification that overexpresses a secreted recombinant protein comprises an inducible promoter, the method comprises culturing the engineered eukaryotic cell under conditions that promote expression of the fusion protein by contacting the engineered eukaryotic with an agent that activates the inducible promoter.

70. The method of claim 69, wherein the inducible promoter is an AOX1, DAK2, PEX11, FLD1, FGH1, DAS1, DAS2, CAT1, MDH3, HAC1, BiP, RAD30, RVS161-2, MPP10, THP3, TLR, GBP2, PMP20, SHB17, PEX8, PEX4, or TKL3 promoter.

71. The method of claim 70, wherein when the inducible promoter is an AOX1, DAK2, PEX11, FLD1, FGH1, DAS2, CAT1, PMP20, SHB17, PEX8, PEX4, TKL3 or DAS1 promoter and the agent that activates the inducible promoter is methanol.

72. The method of claim 68, wherein the secreted recombinant protein is designed to be secreted from the cell and/or is capable of being secreted from the cell.

73. A population of engineered eukaryotic cells of claim 1.

74. A bioreactor comprising the population of engineered eukaryotic cells of claim 73.

75. A composition comprising an engineered eukaryotic cell of claim 1 and a secreted recombinant protein.

76. The composition of claim 75, wherein the secreted recombinant protein is an animal protein.

77. The composition of claim 76, wherein the animal protein is an egg protein.

78. The composition of claim 77, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

79. A composition comprising an engineered eukaryotic cell of claim 1, a secreted recombinant protein that has been deglycosylated, and one or more oligosaccharides cleaved from the secreted recombinant protein.

80. The composition of claim 79, wherein the secreted recombinant protein is an animal protein.

81. The composition of claim 80, wherein the animal protein is an egg protein.

82. The composition of claim 81, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

83. A method for post-translationally modifying a secreted recombinant protein, the method comprising contacting a secreted recombinant protein with a fusion protein anchored to the engineered eukaryotic cell of claim 1, wherein the fusion protein comprises a catalytic enzyme that deglycosylates, acetylates, adenylates, alkylates, amidates, glycosylates, hydroxylates, methylates, or phosphorylates.

84. A method for removing impurities secreted by an engineered eukaryotic cell, the method comprising culturing the engineered eukaryotic cell of claim 1 under conditions that an impurity is secreted by the engineered eukaryotic cell and contacting the impurity with a fusion protein anchored to the engineered eukaryotic cell, wherein the fusion protein comprises a catalytic enzyme that cleaves the impurity, denatures the impurity, modifies the impurity, and/or detoxifies the impurity.

85. A method for allowing an engineered eukaryotic cell to rely on alternate carbon sources, the method comprising contacting an alternate carbon source with a fusion protein anchored to the engineered eukaryotic cell of claim 1, wherein the fusion protein comprises a catalytic enzyme that cleaves the alternate carbon source into a carbon source that can be taken in by the cell and used as a carbon source by the cell.

86. The method of claim 85, wherein when the fusion protein comprises an invertase, the engineered eukaryotic cell is capable of growing on sucrose as its primary carbon source.

87. The method of claim 86, wherein when the fusion protein comprises the anchoring domain is from Tir4, the engineered eukaryotic cell has increased growth when grown on sucrose as its primary carbon source relative to a eukaryotic cell that is not engineered to rely on sucrose as an alternate carbon source.

88. A surface-displayed fusion protein comprising a catalytic domain of an enzyme and an anchoring domain of a glycosylphosphatidylinositol (GPI)-anchored protein, wherein the anchoring domain comprises at least about 200 amino acids and/or at least about 30% of the residues in the anchoring domain are serines or threonines.

89. A polynucleotide encoding the surface-displayed fusion protein of claim 88.

90. A vector comprising a polynucleotide encoding a surface-displayed fusion protein of claim 88.

91. A host cell comprising the vector of claim 90.


SEQUENCE LISTING (347 sequences)

SEQ ID NO: 1 - protein, 368 aa, Saccharomyces cerevisiae
QINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVASSSSEVASSSVASTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 2 - protein, 390 aa, Saccharomyces cerevisiae
MAYSKITLLAALAAIAYAQTQAQINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVASSSSEVASSSVASTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 3 - protein, 465 aa, Saccharomyces cerevisiae
QINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVAPSSSEVVSSSVAPSSSEVVSSSVASSSSEVASSSVAPSSSEVVSSSVASSSSEVASSSVAPSSSEVVSSSVAPSSSEVVSSSVASSSSEVASSSVAPSSSEVVSSSVASSTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 4 - protein, 487 aa, Saccharomyces cerevisiae
MAYSKITLLAALAAIAYAQTQAQINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVAPSSSEVVSSSVAPSSSEVVSSSVASSSSEVASSSVAPSSSEVVSSSVASSSSEVASSSVAPSSSEVVSSSVAPSSSEVVSSSVASSSSEVASSSVAPSSSEVVSSSVASSTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 5 - protein, 279 aa, Saccharomyces cerevisiae
ASVTTTLSPYDERVNLIELAVYVSDIGAHLSEYYAFQALHKTETYPPEIAKAVFAGGDFTTMLTGISGDEVTRMITGVPWYSTRLMGAISEALANEGIATAVPASTTEASSTSTSEASSAATESSSSSESSAETSSNAASTQATVSSESSSAASTIASSAESSVASSVASSVASSASFANTTAPVSSTSSISVTPVVQNGTDSTVTKTQASTVETTITSCSNNVCSTVTKPVSSKAQSTATSVTSSASRVIDVTTNGANKFNNGVFGAAAIAGAAALLL

SEQ ID NO: 6 - protein, 298 aa, Saccharomyces cerevisiae
MSRISILAVAAALVASATAASVTTTLSPYDERVNLIELAVYVSDIGAHLSEYYAFQALHKTETYPPEIAKAVFAGGDFTTMLTGISGDEVTRMITGVPWYSTRLMGAISEALANEGIATAVPASTTEASSTSTSEASSAATESSSSSESSAETSSNAASTQATVSSESSSAASTIASSAESSVASSVASSVASSASFANTTAPVSSTSSISVTPVVQNGTDSTVTKTQASTVETTITSCSNNVCSTVTKPVSSKAQSTATSVTSSASRVIDVTTNGANKFNNGVFGAAAIAGAAALLL

SEQ ID NO: 7 - protein, 1142 aa, Saccharomyces cerevisiae
ITATTTLSPYDERVNLIELAVYVSDIRAHIFQYYSFRNHHKTETYPSEIAAAVFDYGDFTTRLTGISGDEVTRMITGVPWYSTRLKPAISSALSKDGIYTAIPTSTSTTTTKSSTSTTPTTTITSTTSTTSTTPTTSTTSTTPTTSTTSTTPTTSTTSTTPTTSTTSTTPTTSTTSTTPTTSTTSTTPTTSTTSTTPTTSTTSTTPTTSTTPTTSTTSTTSQTSTKSTTPTTSSTSTTPTTSTTPTTSTTSTAPTTSTTSTTSTTSTISTAPTTSTTSSTFSTSSASASSVISTTATTSTTFASLTTPATSTASTDHTTSSVSTTNAFTTSATTTTTSDTYISSSSPSQVTSSAEPTTVSEVTSSVEPTRSSQVTSSAEPTTVSEFTSSVEPTRSSQVTSSAEPTTVSEFTSSVEPTRSSQVTSSAEPTTVSEFTSSVEPTRSSQVTSSAEPTTVSEFTSSVEPTRSSQVTSSAEPTTVSEFTSSVEPIRSSQVTSSAEPTTVSEVTSSVEPIRSSQVTTTEPVSSFGSTFSEITSSAEPLSFSKATTSAESISSNQITISSELIVSSVITSSSEIPSSIEVLTSSGISSSVEPTSLVGPSSDESISSTESLSATSTFTSAVVSSSKAADFFTRSTVSAKSDVSGNSSTQSTTFFATPSTPLAVSSTVVTSSTDSVSPNIPFSEISSSPESSTAITSTSTSFIAERTSSLYLSSSNMSSFTLSTFTVSQSIVSSFSMEPTSSVASFASSSPLLVSSRSNCSDARSSNTISSGLFSTIENVRNATSTFTNLSTDEIVITSCKSSCTNEDSVLTKTQVSTVETTITSCSGGICTTLMSPVTTINAKANTLTTTETSTVETTITTCPGGVCSTLTVPVTTITSEATTTATISCEDNEEDITSTETELLTLETTITSCSGGICTTLMSPVTTINAKANTLTTTETSTVETTITTCSGGVCSTLTVPVTTITSEATTTATISCEDNEEDVASTKTELLTMETTITSCSGGICTTLMSPVSSFNSKATTSNNAESTIPKAIKVSCSAGACTTLTTVDAGISMFTRTGLSITQTTVTNCSGGTCTMLTAPIATATSKVISPIPKASSATSIAHSSASYTVSINTNGAYNFDKDNIFGTAIVAVVALLLL

SEQ ID NO: 8 - protein, 1161 aa, Saccharomyces cerevisiae
MVNISIVAGIVALATSAAAITATTTLSPYDERVNLIELAVYVSDIRAHIFQYYSFRNHHKTETYPSEIAAAVFDYGDFTTRLTGISGDEVTRMITGVPWYSTRLKPAISSALSKDGIYTAIPTSTSTTTTKSSTSTTPTTTITSTTSTTSTTPTTSTTSTTPTTSTTSTTPTTSTTSTTPTTSTTSTTPTTSTTSTTPTTSTTSTTPTTSTTSTTPTTSTTSTTPTTSTTPTTSTTSTTSQTSTKSTTPTTSSTSTTPTTSTTPTTSTTSTAPTTSTTSTTSTTSTISTAPTTSTTSSTFSTSSASASSVISTTATTSTTFASLTTPATSTASTDHTTSSVSTTNAFTTSATTTTTSDTYISSSSPSQVTSSAEPTTVSEVTSSVEPTRSSQVTSSAEPTTVSEFTSSVEPTRSSQVTSSAEPTTVSEFTSSVEPTRSSQVTSSAEPTTVSEFTSSVEPTRSSQVTSSAEPTTVSEFTSSVEPTRSSQVTSSAEPTTVSEFTSSVEPIRSSQVTSSAEPTTVSEVTSSVEPIRSSQVTTTEPVSSFGSTFSEITSSAEPLSFSKATTSAESISSNQITISSELIVSSVITSSSEIPSSIEVLTSSGISSSVEPTSLVGPSSDESISSTESLSATSTFTSAVVSSSKAADFFTRSTVSAKSDVSGNSSTQSTTFFATPSTPLAVSSTVVTSSTDSVSPNIPFSEISSSPESSTAITSTSTSFIAERTSSLYLSSSNMSSFTLSTFTVSQSIVSSFSMEPTSSVASFASSSPLLVSSRSNCSDARSSNTISSGLFSTIENVRNATSTFTNLSTDEIVITSCKSSCTNEDSVLTKTQVSTVETTITSCSGGICTTLMSPVTTINAKANTLTTTETSTVETTITTCPGGVCSTLTVPVTTITSEATTTATISCEDNEEDITSTETELLTLETTITSCSGGICTTLMSPVTTINAKANTLTTTETSTVETTITTCSGGVCSTLTVPVTTITSEATTTATISCEDNEEDVASTKTELLTMETTITSCSGGICTTLMSPVSSFNSKATTSNNAESTIPKAIKVSCSAGACTTLTTVDAGISMFTRTGLSITQTTVTNCSGGTCTMLTAPIATATSKVISPIPKASSATSIAHSSASYTVSINTNGAYNFDKDNIFGTAIVAVVALLLL

SEQ ID NO: 9 - protein, 631 aa, Saccharomyces cerevisiae
ININDITFSNLEITPLTANKQPDQGWTATFDFSIADASSIREGDEFTLSMPHVYRIKLLNSSQTATISLADGTEAFKCYVSQQAAYLYENTTFTCTAQNDLSSYNTIDGSITFSLNFSDGGSSYEYELENAKFFKSGPMLVKLGNQMSDVVNFDPAAFTENVFHSGRSTGYGSFESYHLGMYCPNGYFLGGTEKIDYDSSNNNVDLDCSSVQVYSSNDFNDWWFPQSYNDTNADVTCFGSNLWITLDEKLYDGEMLWVNALQSLPANVNTIDHALEFQYTCLDTIANTTYATQFSTTREFIVYQGRNLGTASAKSSFISTTTTDLTSINTSAYSTGSISTVETGNRTTSEVISHVVTTSTKLSPTATTSLTIAQTSIYSTDSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 10 - protein, 650 aa, Saccharomyces cerevisiae
MFTFLKIILWLFSLALASAININDITFSNLEITPLTANKQPDQGWTATFDFSIADASSIREGDEFTLSMPHVYRIKLLNSSQTATISLADGTEAFKCYVSQQAAYLYENTTFTCTAQNDLSSYNTIDGSITFSLNFSDGGSSYEYELENAKFFKSGPMLVKLGNQMSDVVNFDPAAFTENVFHSGRSTGYGSFESYHLGMYCPNGYFLGGTEKIDYDSSNNNVDLDCSSVQVYSSNDFNDWWFPQSYNDTNADVTCFGSNLWITLDEKLYDGEMLWVNALQSLPANVNTIDHALEFQYTCLDTIANTTYATQFSTTREFIVYQGRNLGTASAKSSFISTTTTDLTSINTSAYSTGSISTVETGNRTTSEVISHVVTTSTKLSPTATTSLTIAQTSIYSTDSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 11 - protein, 1587 aa, Saccharomyces cerevisiae
QIVFYQNSSTSLPVPTLVSTSIADFHESSSTGEVQYSSSYSYVQPSIDSFTSSSFLTSFEAPTETSSSYAVSSSLITSDTFSSYSDIFDEETSSLISTSAASSEKASSTLSSTAQPHRTSHSSSSFELPVTAPSSSSLPSSTSLTFTSVNPSQSWTSFNSEKSSALSSTIDFTSSEISGSTSPKSLESFDTTGTITSSYSPSPSSKNSNQTSLLSPLEPLSSSSGDLILSSTIQATTNDQTSKTIPTLVDATSSLPPTLRSSSMAPTSGSDSISHNFTSPPSKTSGNYDVLTSNSIDPSLFTTTSEYSSTQLSSLNRASKSETVNFTASIASTPFGTDSATSLIDPISSVGSTASSFVGISTANFSTQGNSNYVPESTASGSSQYQDWSSSSLPLSQTTWVVINTTNTQGSVTSTTSPAYVSTATKTVDGVITEYVTWCPLTQTKSQAIGVSSSISSVPQASSFSGSSILSSNSSTLAASNNVPESTASGSSQYQDWSSSSLPLSQTTWVVINTTNTQGSVTSTTSPAYVSTATKTVDGVITEYVTWCPLTQTKSQAIGISSSTISATQTSKPSSILTLGISTLQLSDATFKGTETINTHLMTESTSITEPTYFSGTSDSFYLCTSEVNLASSLSSYPNFSSSEGSTATITNSTVTFGSTSKYPSTSVSNPTEASQHVSSSVNSLTDFTSNSTETIAVISNIHKTSSNKDYSLTTTQLKTSGMQTLVLSTVTTTVNGAATEYTTWCPASSIAYTTSISYKTLVLTTEVCSHSECTPTVITSVTATSSTIPLLSTSSSTVLSSTVSEGAKNPAASEVTINTQVSATSEATSTSTQVSATSATATASESSTTSQVSTASETISTLGTQNFTTTGSLLFPALSTEMINTTVVSRKTLIISTEVCSHSKCVPTVITEVVTSKGTPSNGHSSQTLQTEAVEVTLSSHQTVTMSTEVCSNSICTPTVITSVQMRSTPFPYLTSSTSSSSLASTKKSSLEASSEMSTFSVSTQSLPLAFTSSEKRSTTSVSQWSNTVLTNTIMSSSSNVISTNEKPSSTTSPYNFSSGYSLPSSSTPSQYSLSTATTTINGIKTVYTTWCPLAEKSTVAASSQSSRSVDRFVSSSKPSSSLSQTSIQYTLSTATTTISGLKTVYTTWCPLTSKSTLGATTQTSSTAKVRITSASSATSTSISLSTSTESESSSGYLSKGVCSGTECTQDVPTQSSSPASTLAYSPSVSTSSSSSFSTTTASTLTSTHTSVPLLPSSSSISASSPSSTSLLSTSLPSPAFTSSTLPTATAVSSSTFIASSLPLSSKSSLSLSPVSSSILMSQFSSSSSSSSSLASLPSLSISPTVDTVSVLQPTTSIATLTCTDSQCQQEVSTICNGSNCDDVTSTATTPPSTVTDTMTCTGSECQKTTSSSCDGYSCKVSETYKSSATISACSGEGCQASATSELNSQYVTMTSVITPSAITTTSVEVHSTESTISITTVKPVTYTSSDTNGELITITSSSQTVIPSVTTIITRTKVAITSAPKPTTTTYVEQRLSSSGIATSFVAAASSTWITTPIVSTYAGSASKFLCSKFFMIMVMVINFI

SEQ ID NO: 12 - protein, 1609 aa, Saccharomyces cerevisiae
MNSFASLGLIYSVVNLLTRVEAQIVFYQNSSTSLPVPTLVSTSIADFHESSSTGEVQYSSSYSYVQPSIDSFTSSSFLTSFEAPTETSSSYAVSSSLITSDTFSSYSDIFDEETSSLISTSAASSEKASSTLSSTAQPHRTSHSSSSFELPVTAPSSSSLPSSTSLTFTSVNPSQSWTSFNSEKSSALSSTIDFTSSEISGSTSPKSLESFDTTGTITSSYSPSPSSKNSNQTSLLSPLEPLSSSSGDLILSSTIQATTNDQTSKTIPTLVDATSSLPPTLRSSSMAPTSGSDSISHNFTSPPSKTSGNYDVLTSNSIDPSLFTTTSEYSSTQLSSLNRASKSETVNFTASIASTPFGTDSATSLIDPISSVGSTASSFVGISTANFSTQGNSNYVPESTASGSSQYQDWSSSSLPLSQTTWVVINTTNTQGSVTSTTSPAYVSTATKTVDGVITEYVTWCPLTQTKSQAIGVSSSISSVPQASSFSGSSILSSNSSTLAASNNVPESTASGSSQYQDWSSSSLPLSQTTWVVINTTNTQGSVTSTTSPAYVSTATKTVDGVITEYVTWCPLTQTKSQAIGISSSTISATQTSKPSSILTLGISTLQLSDATFKGTETINTHLMTESTSITEPTYFSGTSDSFYLCTSEVNLASSLSSYPNFSSSEGSTATITNSTVTFGSTSKYPSTSVSNPTEASQHVSSSVNSLTDFTSNSTETIAVISNIHKTSSNKDYSLTTTQLKTSGMQTLVLSTVTTTVNGAATEYTTWCPASSIAYTTSISYKTLVLTTEVCSHSECTPTVITSVTATSSTIPLLSTSSSTVLSSTVSEGAKNPAASEVTINTQVSATSEATSTSTQVSATSATATASESSTTSQVSTASETISTLGTQNFTTTGSLLFPALSTEMINTTVVSRKTLIISTEVCSHSKCVPTVITEVVTSKGTPSNGHSSQTLQTEAVEVTLSSHQTVTMSTEVCSNSICTPTVITSVQMRSTPFPYLTSSTSSSSLASTKKSSLEASSEMSTFSVSTQSLPLAFTSSEKRSTTSVSQWSNTVLTNTIMSSSSNVISTNEKPSSTTSPYNFSSGYSLPSSSTPSQYSLSTATTTINGIKTVYTTWCPLAEKSTVAASSQSSRSVDRFVSSSKPSSSLSQTSIQYTLSTATTTISGLKTVYTTWCPLTSKSTLGATTQTSSTAKVRITSASSATSTSISLSTSTESESSSGYLSKGVCSGTECTQDVPTQSSSPASTLAYSPSVSTSSSSSFSTTTASTLTSTHTSVPLLPSSSSISASSPSSTSLLSTSLPSPAFTSSTLPTATAVSSSTFIASSLPLSSKSSLSLSPVSSSILMSQFSSSSSSSSSLASLPSLSISPTVDTVSVLQPTTSIATLTCTDSQCQQEVSTICNGSNCDDVTSTATTPPSTVTDTMTCTGSECQKTTSSSCDGYSCKVSETYKSSATISACSGEGCQASATSELNSQYVTMTSVITPSAITTTSVEVHSTESTISITTVKPVTYTSSDTNGELITITSSSQTVIPSVTTIITRTKVAITSAPKPTTTTYVEQRLSSSGIATSFVAAASSTWITTPIVSTYAGSASKFLCSKFFMIMVMVINFI

SEQ ID NO: 13 - protein, 320 aa, Saccharomyces cerevisiae
QFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 14 - protein, 338 aa, Saccharomyces cerevisiae
MKLSTVLLSAGLASTTLAQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 15 - protein, 513 aa, Saccharomyces cerevisiae
SMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 16 - protein, 532 aa, Saccharomyces cerevisiae
MLLQAFLFLLAGFAAKISASMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 17 - protein, 564 aa, unidentified
MTIESQEPWWKSAVVYQVWPASFKDSNGDGIGDLNGITSELDHIKSLGTDVIWLSPHYASPLDDMGYDISDYNAINPQFGTMEDMDRLLAEIKKRDMRLILDLVINHTSSEHAWFKESRSSRDNPKRDWYIWKDNANNWLSFFSGSAWSYDEKTKQYYLRLFAETQPDLNWENPKTREAIYKSALEFWYEKGVSGFRIDTAGLYSKVQTFEDAPVTFPGEKYQPAGPLINSGPRIHEFHKEMYEKVTSRYDAMTVGEVGHCSKADALKYVSAKEKEMNMMFLFDTVDVGSDKSDRFRYKGFTLTDFKDAIINQSNFIFDDETGELNDAWSTVFIENHDQPRCVTRFGNTSNKLFWSRSAKMLALLQTTLTGTLFVYQGQEIGMTNVSPKWDISEYLDINTINYWNAFNETEHSDEEKAELLKIINLLARDNARTPVQWDSSENGGFGGKPWMRINDNYKDINVASQKEDPDSVLNFYRNAIKTRKHYSETLIFGRFEVQDYDNQEIFYYTKTSNKGQKKMAVVLNFTDREVEYPIPQGKLLLSNIANNITGKLQPYEGRLIEVN

SEQ ID NO: 18 - protein, 517 aa, unidentified
MKKVIKKYFFLALAIIMYSCNEDEKYDILERYTPETITSDEIAPVLNLQAQYMDSNSEIVLVTWMNPEDDFLSKVEISCCSANDNLLGEPVLLDAVSTKVGSYQTSLSVEERGYVKIVAINEKGVRSEARTAEILSSQQDFVYRADCLMSSVIELFFGGRYNAWNENYPNATGPYWDGIAAVWGQGAAYSGFVTMYKVTKETNNEKLRAKYAEKEETFLNSIDIFLNNGSGRKSFAYGTYIGPNDERYYDDNVWIGIEMANLYELTGNEVYLQHANTVWNFILEGIDDVTGGGVYWKEGAVSKHTCSTAPAAVMALKLYQLSKNESYLEIAKSLYSYCKDVLQDPNDYLFYDNVRLSDPSDKNSELKVSKDKFTYNSGQPMLAAAMLYRITKEEQFLKDAQNIAQSIYKKWFKNYHSSILDRDIMILSDPNTWFNAVMFRGFVELYKIDKNDVYVKAVKNTMEHAWQSNCRNRLTNLMSDDYAGDKKEGKWNIKTQGAFVEIFSLIGELEQLGCFQE

SEQ ID NO: 19 - protein, 271 aa, unidentified
APAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTP

SEQ ID NO: 20 - protein, 313 aa, unidentified
MFTPVRRRVRTAALALSAAAALVLGSTAASGASATPSPAPAPAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTP

SEQ ID NO: 21 - protein, 698 aa, synthetic construct
APAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSQINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVASSSSEVASSSVASTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 22 - protein, 787 aa, synthetic construct
MRFPSIFTAVLFAASSALAAPVNTTTEDETAQIPAEAVIGYSDLEGDFDVAVLPFSNSTNNGLLFINTTIASIAAKEEGVSLDKREAEAAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSQINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVASSSSEVASSSVASTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 23 - protein, 609 aa, synthetic construct
APAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSASVTTTLSPYDERVNLIELAVYVSDIGAHLSEYYAFQALHKTETYPPEIAKAVFAGGDFTTMLTGISGDEVTRMITGVPWYSTRLMGAISEALANEGIATAVPASTTEASSTSTSEASSAATESSSSSESSAETSSNAASTQATVSSESSSAASTIASSAESSVASSVASSVASSASFANTTAPVSSTSSISVTPVVQNGTDSTVTKTQASTVETTITSCSNNVCSTVTKPVSSKAQSTATSVTSSASRVIDVTTNGANKFNNGVFGAAAIAGAAALLL

SEQ ID NO: 24 - protein, 698 aa, synthetic construct
MRFPSIFTAVLFAASSALAAPVNTTTEDETAQIPAEAVIGYSDLEGDFDVAVLPFSNSTNNGLLFINTTIASIAAKEEGVSLDKREAEAAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSASVTTTLSPYDERVNLIELAVYVSDIGAHLSEYYAFQALHKTETYPPEIAKAVFAGGDFTTMLTGISGDEVTRMITGVPWYSTRLMGAISEALANEGIATAVPASTTEASSTSTSEASSAATESSSSSESSAETSSNAASTQATVSSESSSAASTIASSAESSVASSVASSVASSASFANTTAPVSSTSSISVTPVVQNGTDSTVTKTQASTVETTITSCSNNVCSTVTKPVSSKAQSTATSVTSSASRVIDVTTNGANKFNNGVFGAAAIAGAAALLL

SEQ ID NO: 25 - protein, 650 aa, synthetic construct
APAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 26 - protein, 739 aa, synthetic construct
MRFPSIFTAVLFAASSALAAPVNTTTEDETAQIPAEAVIGYSDLEGDFDVAVLPFSNSTNNGLLFINTTIASIAAKEEGVSLDKREAEAAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 27 - protein, 4 aa, synthetic construct
EAEA

SEQ ID NO: 28 - protein, 5 aa, synthetic construct
GGGGS

SEQ ID NO: 29 - skipped

SEQ ID NO: 30 - protein, 20 aa, synthetic construct
EAAAREAAAREAAAREAAAR

SEQ ID NO: 31 - protein, 59 aa, synthetic construct
GSSGSSGSSGSSGSSGSSGSSGSSEAAAREAAAREAAAREAAARGGGGSGGGGSGGGGS

SEQ ID NO: 32 - DNA, 941 bp, synthetic construct
  source 1..941: mol_type = other DNA
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattggatcccga

SEQ ID NO: 33 - DNA, 559 bp, synthetic construct
  source 1..559: mol_type = other DNA
aaataagcatgtttgtttcagatcaaagattagcgtttcaaagttgtggaaaagtgaccatgcaacaatatgcaacacattcggattatctgataagtttcaaagctactaagtaagcccgtttcaagtctccagaccgacatctgccatccagtgattttcttagtcctgaaaaatacgatgtgtaaacataaaccacaaagatcggcctccgaggttgaacccttacgaaagagacatctggtagcgccaatgccaaaaaaaaatcacaccagaaggacaattcccttcccccccagcccattaaagcttaccatttcctattccaatacgttccatagagggcatcgctcggctcattttcgcgtgggtcatactagagcggctagctagtcggctgtttgagctctctaatcgaggggtaaggatgtctaatatgtcataatggctcactatataaagaacccgcttgctcaaccttcgactcctttcccgatcctttgcttgttgcttcttcttttataacaggaaacaaaggaatttatacactttaagaatt

SEQ ID NO: 34 - DNA, 750 bp, synthetic construct
  source 1..750: mol_type = other DNA
cttccccatttcactgacagtttgtagaaatagggcaacaattgatgcaaatcgattttcaacgcattggttttgatagcattgatgatcttggagctgtaaaagtccggctggataagctcaatgaaataggttggttgatctggatcttcttttgggtcattttgttcgctctgtatttcacaaattgccagaatctctgccaaccacagtggtaggtccaacttggtgttctgaatcacaggcttccccgggttgttctctaaataaccgaggcccggcacagaaatcgtaaaccgacacggtatcttttgtccgtccgccagtatctcatcaaggtcgtagtagcccatgatgagtatcaaaggggatttggttatgcgatgcaacgagagattgtttatcccagatgctgatgtaaaaaccttaaccagcgtgacagtagaaataagacacgttaaaattacccgcgcttccctaacaattggctctgcctttcggcaagtttctaactgccctcccctctcacatgcaccacgaacttaccgttcgctcctagcagaaccaccccaaagtttaatcaggaccgcattttagcctattgctgtagaaccccacaacataacctggtccagagccagccctttatatatggtaaatcccgtttgaacttcgaagtggaatcggaatttttacatcaaagaaactgatactgaaacttttggcttcgacttggactttctcttaatc

SEQ ID NO: 35 - DNA, 1004 bp, synthetic construct
  source 1..1004: mol_type = other DNA
aaatcagccattaatctcacctcagtttttgaatcagtagaattttcaatgaaacaaacggttggtatattatttgatagggtagccaaatttccaaaaatgaacttttcatcaggtaatatcttgaataccgtaatgtagtgactattggaagaaactgctatcaaattatatttcggatagaaatccaaaccccagactgatctcttgagtctcaactctaagtcagccgcgactctaattatctgtggattaggagttagtgtggacaaagcatcagtatagtataactttacggttccattatcagacgctattgcaagaacttcctttccattgatctctccaattcgacagtaattgatatcataaggtaggtctggaaacacactggcgcttgtatcccattctgcaggaatttctggaacggtggtaatggtagttatccaacggagttggggtagttggtatatctggatatgccgcctataggataaaaacaggagagagtgaaccttgcttacggctactagattgttcttgtactcggaattgtcgttatcggaaactagactaatctcatctgtgtgttgcagtactattgagtcgttgtagtatctaccaggagggcattccatgaactagtgagacaaatgagttggattttctcaatagacatatgcaagaatgctacacaacggatgtcgcactctttttcttagttgataatatcatccaatcagaagacacgggctagaaggacttgctcccgaaggataatccactgctactatctcccttcctcacatatagtcttgcagggctcatgcccctttctccttcgaactgcccgatgaggaagtctttagcctatcaaggaattcgggaccatcatcaatttttagagccttacctgatcgcaatcaggatttcactactcatataaatacatcactcaaactccaactttgcttgttcatacaattcttgatattcacaggatc

SEQ ID NO: 36 - DNA, 758 bp, synthetic construct
  source 1..758: mol_type = other DNA
gtgaatttgtcacggaattgaccaagaggtcagacgatcctgtatcccattgagccgttatgctttgtgggggaaaccctatttctatcgtactaagaaaaccaatggtgaactcatattcggtatcaatggcgacgattccagcatagcctgtagacagtaacaacactagggcaacagcaactaacatatcttcattgatgaaacgttgtgatcggtgtgacttttatagtaaaagctacaactgtttgaaataccaagatatcattgtgaatggctcaaaagggtaatacatctgaaaaacctgaagtgtggaaaattccgatggagccaactcatgataacgcagaagtcccattttgccatcttctcttggtatgaaacggtagaaaatgatccgagtatgccaattgatactcttgattcatgccctatagtttgcgtagggtttaattgatctcctggtctatcgatctgggacgcaatgtagaccccattagtggaaacactgaaagggatccaacactctaggcggacccgctcacagtcatttcaggacaatcaccacaggaatcaactacttctcccagtcttccttgcgtgaagcttcaagcctacaacataacacttcttacttaatctttgattctcgaattgtttacccaatcttgacaacttagcctaagcaatactctggggttatatatagcaattgctcttcctcgctgtagcgttcattccatctttctagaattcgt

SEQ ID NO: 37 - DNA, 991 bp, synthetic construct
  source 1..991: mol_type = other DNA
cctgttgataagacgcattctagagttgtttcatgaaagggttacgggtgttgattggtttgagatatgccagaggacagatcaatctgtggtttgctaaactggaagtctggtaaggactctagcaagtccgttactcaaaaagtcataccaagtaagattacgtaacacctgggcatgactttctaagttagcaagtcaccaagagggtcctatttaacgtttggcggtatctgaaacacaagacttgcctatcccatagtacatcatattacctgtcaagctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaaagaattcgcg

SEQ ID NO: 38 - DNA, 500 bp, synthetic construct
  source 1..500: mol_type = other DNA
taatcgaactccgaatgcggttctcctgtaaccttaattgtagcatagatcacttaaataaactcatggcctgacatctgtacacgttcttattggtcttttagcaatcttgaagtctttctattgttccggtcggcattacctaataaattcgaatcgagattgctagtacctgatatcatatgaagtaatcatcacatgcaagttccatgataccctctactaatggaattgaacaaagtttaagcttctcgcacgagaccgaatccatactatgcacccctcaaagttgggattagtcaggaaagctgagcaattaacttccctcgattggcctggacttttcgcttagcctgccgcaatcggtaagtttcattatcccagcggggtgatagcctctgttgctcatcaggccaaaatcatatataagctgtagacccagcacttcaattacttgaaattcaccataacacttgctctagtcaagacttacaattaaa

SEQ ID NO: 39 - DNA, 975 bp, synthetic construct
  source 1..975: mol_type = other DNA
tagcttgggtaggacttgacaagtacggcttccgtggtcataccaaacgcctttgttaccgttggctatacctaatgaccaaggcatttgtggattataacggtatcgtagttgaaaaatatgacgtaaccactggtactagcccccacaaggttgatgctgaatacgggaatcaaggtgccgattttaaaggagtagccactgaagggtttggctgggtcaatgcctcttttattttgggattaacctacttagatgtccaaggcatccgtgcgataggcgccgttacgtcccctgatgtatttttcaggaagctcaaaccttgggaacgcgcaagttatggcctaaggccatgtaacgagatagtcaagtcaaactagaagtatacggtttccccgcagaaatagcagaaataggcgacaaatacatacaacattttcattgtgatagggggcggcggttcctaggagggacaacccccagaaaccttgtagactacgttttcacgacgatgggttattactgtaaaggaagaatatactacccaccagttgaatgtttgaacggatcaaaggtcgaagggagtacacggcccaaccaacgtagctaccggagaaagcaagactttcccaaaccaaatagctccgggtttcttctccggcaacccgtcagtttttgtgtggccggacaaaaattcgcaccctcagtctaattgaaaggtcgggctccgagctctaggcgtttgcgcatgtaatattgcatcccctcccatagataatactgcgcgaacacagggtgcaaattatgatgaccacacatgccagtgaccaaaacagttttttagtctttaaaaaccctcggaacttctgagtatataaaggcttctcatttcctacaagcaaacaaagaagaaacttccactttctaactttttatctatagactttagagttacaaccaacgaacaataacaaa

SEQ ID NO: 40 - DNA, 960 bp, synthetic construct
  source 1..960: mol_type = other DNA
tgaagcttatctgctgagcaagttgtttgaccaaacttgagtcaacagtggttaactatatcctctattattttagatgggagcacatcaagtgtacgggaacaatgcaatcgacaacctgtagcctgacatacatagccatcttgaattgacaaaacttagaatgtcttgaatgtgatagatatgagttcccaaaaatctcttttacgatttcccagttgcggtgtactattacacagaggatatcatagcagacttacaatcctcaggcataaaacgagctttcttatcaaagtgtattcaaatggaccatttgattgcaccaaggcattagccccaaaccataccacacagtaacttgatattctcagcatgcatggaaattccactcataacgcgctattcaccgcgaatacttatctatgaaactgggttctttagtattctttgccaaatttcaccgattagaaattattaggtaatataatttctttggggaaccccttcccgttacgcccgctgcggctttgtggttcttttccagtcttgagcaaattacatctggtctagacagttcttccgtgccccagtatgcgagcgcaaactttcaatcaaacctcgtagcaaattggtacttgaacttcgtatttaaccgctattaaatgtactgactcttacattatgaaaaattttgataaagattttatatttcatctcagttaatctcctaataataatagtctgcataactcaaacggtacttccttttcggaacgcgaagagtagtctctatgtcattctcacactatccgcagcgcaatagagaacgagcatgttacccgactcatcccttgtcgattcggaaacgatttataaatacaattagatcgccaccgatcttcttttgtcaatattataaaaatagtacagattttccttagtcgaatcagatcgcagaaa

SEQ ID NO: 41 - DNA, 876 bp, synthetic construct
  source 1..876: mol_type = other DNA
agatctgagggtgtatacgatgtatcgtgccgaacacatgcacttgacggcacagcaaatggtattcaagaagaccactttagaatgggagttaatagggatggtttcatggaggttaaaacacttcaaggaggcatctgaagcattcaagtatgcactaggtctgaggttttcggtcaaggcatgcaagaaattaattgtattctatctgaacgaacgctccagaatgaaccagccagaaacctcaattgccctcaacaacttaaatcaatccacattatccatccaagagattctcaagtatcgttcgttcctcgatatcaacctaatttcaaacttggtcaaactaggagtttggaatcaccgctggtatgctgagttttctccaaaactcatagaaagccttgcggttgttgtggagaacggagggcttatcaaggtagaaaacgaggttaaggctacctatttcgattcacaagatggagtttacgacttgatgaacgaggtattcaagttcatgaagcattacgattatcctgggactgacaactaagagctcctagtgaagacttgagatggacatgataaacaattatagtgaaaatagaaaccataatacaatattctaatagaggaaccgtttacctgtggttcctattgtggcctactgttactagctagtgtaatacacccttgcctcagctttgcaagttgacaactcagccaaatgatctttgaatgcgcgaaacctcaaggtccatcgaattttctcgaattttcagtgttttcatacagcgtgtcatcttctttcgcgtacttattaaaatcgtacccagatcccttcttcttccttaatttcaattccaacactcaaga

SEQ ID NO: 42 - DNA, 727 bp, synthetic construct
  source 1..727: mol_type = other DNA
agatcttgcaaaatacctttccagctttccagcttcctagcactcatcttgaagatatcaaatattctccattcaaaccaacatcaaaaaatagaataattataatcagtttgaagagcaagagtaattttaaaggaaacacattcatggtcagctagaaggttgactgaagagtcgcaagatatctgagaataaaaaagagcatagctaacaagatgagtaaacacggcaaacagatttaggaacaggtgaagggtttctggctcttcaatgtatatcctgctagccacccattcagaaataacacaaagtaggaccctactgaaaaataaatttaatacatcttcatcctctcattaaaccaccgaccactcaaaccataccagccttgtccaattccatgcatcgtgctatccgtcagaattttcagtgttaatcgaatcggtcattatagctccgtctggggcgacaacttgtcatcacagaatagcacaattatgcgttggaatcgtcaaaaaatcacctccaggtctgtatacatacagaactggttgtaacgacaaccttgtttgattgaggtgactggaaggtggaaagaaagggaggaaataaatattgcaaggaaagaaaaaaaaattgttcacagtcacctcttcaccttcgcgatttcatgtttctttcatgtgctaactgatcccagggcttctccagcgcccttatctgttag

SEQ ID NO: 43 - DNA, 872 bp, synthetic construct
  source 1..872: mol_type = other DNA
ctgcccatctatgactgaatgtggagaagtatcggaacaacccttcactaaggatatctaggctaaactcattcgcgccttagatttctccaaggtatcggttaagtttcctctttcgtactggctaacgatggtgttgctcaacaaagggatggaacggcagctaaagggagtgcatggaatgactttaattggctgagaaagtgttctatttgtccgaatttcttttttctattatctgttcgtttgggcggatctctccagtggggggtaaatggaagatttctgttcatggggtaaggaagctgaaatccttcgtttcttataggggcaagtatactaaatctcggaacattgaatggggtttactttcattggctacagaaattattaagtttgttatggggtgaagttaccagtaattttcattttttcacttcaacttttggggtatttctgtggggtagcatagcttgacaggtaatatgatgtactatgggataggcaagtcttgtgtttcagataccgccaaacgttaaataggaccctcttggtgacttgctaacttagaaagtcatgcccaggtgttacgtaatcttacttggtatgactttttgagtaacggacttgctagagtccttaccagacttccagtttagcaaaccacagattgatctgtcctctggcatatctcaaaccaatcaacacccgtaaccctttcatgaaacaactctagaatgcgtcttatcaacaggattgcccaaaacagtaattggggcggtggaatctacatgggagttccatcgttgtctcggtttttctccctataagctactctggagacgaagtaactaacaccctcaaatatcatt

SEQ ID NO: 44 - DNA, 684 bp, synthetic construct
  source 1..684: mol_type = other DNA
tctgaatccgacctcctctaatctaccactgaagagaagcagtgtattgttcgtctacgtaaatttgaatgtgtaaatggcaaacatggcttcggggatgatttggcatatatattattgtagcatcgtctgtggctctatgagttgtgtggcggatgatgaaaagtttcgtgctgatcccacaatgcggcatttaccaaatggggaaagaccagatttcttcgctgcgccagctagggacagcataatgttccaagaagaagcgattacaggtggattacaaagcgttcgtctgcagttgatgttctacgtgatgggtatgagttgtagtgctacgctccatgaatacttctaatttgtcgttgacaatccatgaataatttaagtttgcttcccaagagtctattgcgaagggtgagccgaatctcttggcgtatgcacccgactcgtcggcttttgtgcgttccttgcaaagctcggtagcaatccgttggtgggagaaatttgtctcacgaatttcagttgggagtagctgttcctggtagcaagttcgaggggatctgtgctcataaaacgtgctcacgccaaaaatattcttacaaaatcttcgcggggtgtttgtcttacataatcgattggatattttcttcaaatttttttttcttactgaagtcccctatagag

SEQ ID NO: 45 - DNA, 842 bp, synthetic construct
  source 1..842: mol_type = other DNA
tcttgccagttgtctcctaagatgtcatcggagtaggctcggctaaagagtagtaatgcatcaagaccaaccaaaacaccttccacgagttcagatgaaccttttaataacttcaggtcactttgatgccggcacaactgggcgagtttcgtatagttaactctgatcttgcactccagaacgggaataggattgactttttgcttccgagaaacgatttgctctctcttcgtctggcttttcactttatatcgcacggaatcaatggatggaactcctaaagctcctaacttcgatgatttgctagccatgactctgtgggacattttcttgcatctcgtttgtaacctgtctgttcctacactaagtttatgagaggctactttggattctagcctcggtggtaaagtgggagataacaacggcataaggcaagaaccagaagtaccataacggtctggtaaagttggtgataacttaattggaagagtgtaagtaagacgtggcttgtaataaggctttccatcaaaaaggttctccgggttggagtttgtgaggctcacatctttgatcagtctttcaatataaattggtaacgttgatgacaatgccggaggtaatttctgtagttgttgatatacgcagataacagattcaaatctccattggttttcatcattgtggcttaaattagatcagaacatggtagtatttaaaaatggatctctttgcagatttactcaatatagcgaaaaaaggagacattcgttacaaaatatgaagataattcgcctcataactcgattaatcaaaacagacggtccagttcttcttttggtagt

SEQ ID NO: 46 - DNA, 689 bp, synthetic construct
  source 1..689: mol_type = other DNA
atctgtactggtactgacaaaggttatccagaatccgagacatttcaacaacagagattccaggcttcaaaacatccattttatcaccaatatctagtaatgcttgcaacaattctggatacttcttctgtgtaaccaaatctcttataaactgaacagctttctgtacgttgtcgtcagtagttggatcaacctcagtggtgacctggcctatcggttttccaaaagacttgtttatcacgtccgaaagctcccatttttgcagatgcgcaactttaaaaggcctggcttgaacatttgcatctcttgttgtgtgttctttgagaaaatattcatcgatctgggtgcttccaacgacagaagatactcttctgagaccagaaagtccccagccatgcttcctaattacaaaatatttgtaggaagatccctgattaggacaaagttgtcttctcatgagttcaactgaaactggggctcaaacggattatgaaaggggtgattaaaggttttcctagccttactttccaaatgtcgaccgagacgaacatttaaaatcctaacatcagaaatttctatccttaatctcattgatggttagtacacttcgcagagtctccacatttgcagaccctcctggataaccaaagcttatctaacagcggcattggacctttgaaaagaccctc

SEQ ID NO: 47 - DNA, 1048 bp, synthetic construct
  source 1..1048: mol_type = other DNA
aaatctgaacacgatgaaacctccccgtagattccaccgccccgttacttttttgggcaatcccgttgataagatccattttagagttgtttctgaaaggattacaggcgttgaagggtcagagagatgccagagaacagaccaattggtagtttgctaaagtggacgtctggcaggtgctctatcgtgttctttatttagggcgttacacttagtaggattacgtaacaatttggcttaaccttctaagttagaaagaaaccaagaggggtcctctttaacgttcagcagtatctaaaacacaaaacctgccctcataatacatcattctatctgtcaagctgtgctaccccacagaaatacccccaagagttaaagtgaaaagaaaagctaaatctgttagacttcaccccataacaaacttgatagttcctgtagccaatgaaagttaaccccattcaatgttccgagatctagtatgcttgctcctataaggaacgaagggttccagcttccttaccccatcaatggaaatctcctatttaccccccactggaaagatccgtccgaacgaacggataatagaaaaaagaaattcggacaaaatagaacacttatttagccaatgaaatccatttccagcatctccttcaactgccgttccatcccctttgttgagctacaccatcgtcagccagtaccgaataggaaacttaaccgatatcttggagaattctaatgcgcgaatgagtttagcctagatatccttagtgaagggttgttccgatacttctccacattcagtcatttcagatgggcagcattgttatcatgaagaaacggaaacgggcagtaagggttaaccgccaaattatataaagacaacatgtccccagtttaaagtttttctttcctattcttgtatcctgagtgaccgttgtgtttaaaataacaagttcgttttaacttaagaccaaaaccagttacaacaaattattccccaactaaacactaaagttcactcttatcaaactatcaaacatcaaag

SEQ ID NO: 48 - DNA, 758 bp, synthetic construct
  source 1..758: mol_type = other DNA
cttccccatttcactgacagtttgtagaaatagggcaacaattgatgcaaatcgattttcaacgcattggttttgatagcattgatgatcttggagctgtaaaagtccggctggataagctcaatgaaataggttggttgatctggatcttcttttgggtcattttgttcgctctgtatttcacaaattgccagaatctctgccaaccacagtggtaggtccaacttggtgttctgaatcacaggcttccccgggttgttctctaaataaccgaggcccggcacagaaatcgtaaaccgacacggtatcttttgtccgtccgccagtatctcatcaaggtcgtagtagcccatgatgagtatcaaaggggatttggttatgcgatgcaacgagagattgtttatcccagatgctgatgtaaaaaccttaaccagcgtgacagtagaaataagacacgttaaaattacccgcgcttccctaacaattggctctgcctttcggcaagtttctaactgccctcccctctcacatgcaccacgaacttaccgttcgctcctagcagaaccaccccaaagtttaatcaggaccgcattttagcctattgctgtagaaccccacaacataacctggtccagagccagccctttatatatggtaaatcccgtttgaacttcgaagtggaatcggaatttttacatcaaagaaactgatactgaaacttttggcttcgacttggactttctcttaatcgaattcgt

SEQ ID NO: 49 - DNA, 798 bp, synthetic construct
  source 1..798: mol_type = other DNA
caggtgaacccacctaactatttttaactggcatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgt

SEQ ID NO: 50 - DNA, 1009 bp, synthetic construct
  source 1..1009: mol_type = other DNA
aaataaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacagaatt

SEQ ID NO: 51 - DNA, 686 bp, synthetic construct
  source 1..686: mol_type = other DNA
tgcttaagtaattgaaaacagtgttgtgattatataagcatggtatttgaatagaactactggggttaacttatctagtaggatggaagttgagggagatcaagatgcttaaagaaaaggattggccaatatgaaagccataattagcaatacttatttaatcagataattgtggggcattgtgacttgacttttaccaggacttcaaacctcaaccatttaaacagttatagaagacgtaccgtcacttttgcttttaatgtgatctaaatgtgatcacatgaactcaaactaaaatgatatcttttactggacaaaaatgttatcctgcaaacagaaagctttcttctattctaagaagaacatttacattggtgggaaacctgaaaacagaaaataaatactccccagtgaccctatgagcaggatttttgcatccctattgtaggcctttcaaactcacacctaatatttcccgccactcacactatcaatgatcacttcccagttctcttcttcccctattcgtaccatgcaacccttacacgccttttccatttcggttcggatgcgacttccagtctgtggggtacgtagcctattctcttagccggtatttaaacatacaaattcacccaaattctaccttgataaggtaattgattaatttcataaatgaattcgcg

SEQ ID NO: 52 - DNA, 477 bp, synthetic construct
  source 1..477: mol_type = other DNA
tttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactat

SEQ ID NO: 53 - DNA, 1018 bp, synthetic construct
  source 1..1018: mol_type = other DNA
aaatagcagtttgcggtttcttgatttcatggggggaacaaacaatagtgttgccttaattctaattggcattgttgcttggaatcgaaattgggggataacgtcatatctgaaaagtaaacaacttcgggaaatcaggctgtttgaatggcttggaagcgagatagaaaggggatagcgagatagagggggcggagtagacgaagggtgttaaactgctgaaatctctcaatctggaagaaacggaataaattaactccttgcgataataaaatccgagtccgttatgaccccacaccgtgttgaccacggcataccccatggaatctggtacaaagcgtcagtcttgaagacaccatcacgtgtaggagactgattgtctgaccgtccagcaaaaagggcattataaatcttgctgttaaaggggtgaggggagatgcaggttgttcttttattcgccttgaactttttaattttcccggggttgcggagcgtgaacagttagcccgatctgatagcttgcaagattcaacagtttatccactacaggtcagagagatcgccgcagaagaaatgctcgtctcgtgttccagcacacatactggtgaagtcgttattttgccgaagggggggtaataaggttatgcaccccctctccacaccccagaatcattttttagctgggttcaaggcattagactttgcacatttttcccttaaacacccttgaaacgcggataaacagttgcatgtgcatcctaaaactaggtgagatgcgtactccgtgctccgataataacagtggtgttggggttgctgctagctcacgcactccgttcttttttttcaaccagcaaaattcgatggggagaaacttggggtactttgccgactcctccaccatgctggtatataaataatactcgcccacttttcgtttgctgcttttatatttcatagactgaaaaagactcttcttctactttttcataatatatctcagatatcactactatag

SEQ ID NO: 54 - DNA, 508 bp, synthetic construct
  source 1..508: mol_type = other DNA
gcgatttaaattcgcgaaagaacagcctaataaactccgaagcatgatggcctctatccggaaaacgttaagagatgtggcaacaggagggcacatagaatttttaaagacgctgaagaatgctatcatagtccgtaaaaatgtgatagtactttgtttagtgcgtacgccacttattcggggccaatagctaaacccaggtttgctggcagcaaattcaactgtagattgaatctctctaacaataatggtgttcaatcccctggctggtcacggggaggactatcttgcgtgatccgcttggaaaatgttgtgtatccctttctcaattgcggaaagcatctgctacttcccataggcaccagttacccaattgatatttccaaaaaagattaccatatgttcatctagaagtataaatacaagtggacattcaatgaatatttcattcaattagtcattgacactttcatcaacttactacgtcttattcaacaatgaattcgcg

SEQ ID NO: 55 - DNA, 507 bp, synthetic construct
  source 1..507: mol_type = other DNA
acacagttattattcatttaaatgtcaaaacagtagtgataaaaggctatgaaggaggttgtctaggggctcgcggaggaaagtgattcaaacagacctgccaaaaagagaaaaaagagggaatccctgttctttccaatggaaatgacgtaactttaacttgaaaaataccccaaccagaagggttcaaactcaacaaggattgcgtaattcctacaagtagcttagagctgggggagagacaactgaaggcagcttaacgataacgcggggggattggtgcacgactcgaaaggaggtatcttagtcttgtaacctcttttttccagaggctattcaagattcataggcgatatcgatgtggagaagggtgaacaatataaaaggctggagagatgtcaatgaagcagctggatagatttcaaattttctagatttcagagtaatcgcacaaaacgaaggaatcccaccaagacaaaaaaaaaaattctaaggaattccgaaacg

SEQ ID NO: 56 - DNA, 759 bp, synthetic construct
  source 1..759: mol_type = other DNA
aaattctttttacgtggtgcgcatactggacagaggcagagtctcaatttcttcttttgagacaggctactacagcctgtgattcctcttggtacttggatttgcttttatctggctccgttgggaactgtgcctgggttttgaagtatcttgtggatgtgtttctaacactttttcaatcttcttggagtgagaatgcaggactttgaacatcgtctagctcgttggtaggtgaaccgttttaccttgcatgtggttaggagttttctggagtaaccaagaccgtcttatcatcgccgtaaaatcgctcttactgtcgctaataatcccgctggaagagaagttcgaacagaagtagcacgcaaagctcttgtcaaatgagaattgttaatcgtttgacaggtcacactcgtgggctatgtacgatcaacttgccggctgttgctggagagatgacaccagttgtggcatggccaattggtattcagccgtaccactgtatggaaaatgagattatcttgttcttgatctagtttcttgccattttagagttgccacattcgtaggtttcagtaccaataatggtaacttccaaacttccaacgcagataccagagatctgccgatccttccccaacaataggagcttactacgccatacatatagcctatctattttcactttcgcgtgggtgcttctatataaacggttccccatcttccgtttcatactacttgaattttaagcactaaagaatt

SEQ ID NO: 57 - DNA, 260 bp, synthetic construct
  source 1..260: mol_type = other DNA
aaattaaccagtgttttcttatctatttgtctttttacactaaagtgaagtacgaatccatgcgattgattcctcctcagatatcagctgaattcttgcttatgtaatacttgcgcgaactacatgtgaacttaggattcgataaggctggggggtcaaccaaccccacttcaaagagccgacccgtataaatagcctctgcgtcctcagatcaacaagacgaagcaatttttttttacctatcttcaggtgcctgttag

SEQ ID NO: 58 - DNA, 223 bp, synthetic construct
  source 1..223: mol_type = other DNA
agggaggcaattagttgtccttgtggaatcaaaagagcacaagaaacctgtgattgaaagtctgggctgtctggggttggcaagaaaatcataaagtttatatagtacatttgttagttgcttctttgaatgacaccttgatctacatgttgttcttcccagttcccaccgcgaagtttctctaactctcaatctctctttccccacttgataatccaaagaa

SEQ ID NO: 59 - DNA, 425 bp, synthetic construct
  source 1..425: mol_type = other DNA
gtcgaggaaagggtcgtttcggggagttaaatatttttggctatgtagcagacatgtttcgacgctggcgtcgcgtcgatcggaaaatattaccccaggaacaagcacttgcttgggttagccaccaccctgcgcaagcctttttgccggctctacacagggccaatgaaatctgggcggaatctgaaaccgatgaaacggacgacactggcaacaagctcactgcactattttttttttctagtgaaatagcctatcctcgtctcgctcccctcatacctgtaaaggggtgcaatttagcctcgttccagccattcacgggccactcaacaacacgtcggctaccatggggtgcttgggcaccaaaaggcctataaataggcccccatccgtctgctacacagtcatctctgtcttttcttccc

SEQ ID NO: 60 - DNA, 261 bp, synthetic construct
  source 1..261: mol_type = other DNA
tcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcg

SEQ ID NO: 61 - DNA, 202 bp, synthetic construct
  source 1..202: mol_type = other DNA
tcgatttgtatgtgaaatagctgaaattcgaaaatttcattatggctgtatctactttagcgtattaggcatttgagcattggcttgaacaatgcgggctgtagtgtgtcaccaaagaaaccattcgggttcggatctggaagtcctcatcacgtgatgccgatctcgtgtattttattttcagataacacctgaagacttt

SEQ ID NO: 62 - DNA, 486 bp, synthetic construct
  source 1..486: mol_type = other DNA
attagtgtacatctgataatatagtactaccacgtatgataatgtagagaatagtcttccttgtcgagtgtgtttgcagttttcttgagtttcaaggtttaaatgctggtatattagttcatcgaaggtttcagccaatagcaccttaaatcaatcaaactaattcgactcttacgaaagagcctactgtgtttagtatcgaagtcgtttacctttcatgttgaatagcttcctctctgaccctaacatttcaagatcctcctaaagttacccggattgtgaaattctaatgatccacctgcccaatgcattttttctttattcagtttaccttttttacctaatatacgagcttgttaaagtaagtggcactgcaatactaggcttattgttgatattatgatgaatcgttttcacaaacttgatttcctgtgaactcaccatgtactaaggaaaaaaacatgcatcaccatctgaatatttgac

SEQ ID NO: 63 - DNA, 497 bp, synthetic construct
  source 1..497: mol_type = other DNA
actatgtaactaacgaaacagcatgtactaatagaaccgtatcgagaatatttatttaggtgagtagtaggagtgaaccagacagtcaatttagtgagctgtcccagcttttgtgcattccagaattgccggtcaaattggttatgggttatggggcttttccgattgaggttcagtttctgcggttatctctttcttgacctggtcttttacaggctgttctttctccccatgattattctttagctgaagataccgcttagcctgataatgtcgtcgttttgtaatcaaaatctttagttgggcatcgtctgaggtttcctttggcttctggggttgttagtaggaacgtaggaaccatagtaacttttacacatacattcttatgattgcgaagtaagctgagtctgctgcttggctcccgaagtactttctctttctctaccggttgattctccttctggtgctcctaaacgattgtgttagaagggattgac

SEQ ID NO: 64 - protein, 42 aa, synthetic construct
MFTPVRRRVRTAALALSAAAALVLGSTAASGASATPSPAPAP

SEQ ID NO: 65 - protein, 18 aa, synthetic construct
MKLSTVLLSAGLASTTLA

SEQ ID NO: 66 - protein, 19 aa, synthetic construct
MRFPSIFTAVLFAASSALA

SEQ ID NO: 67 - protein, 22 aa, synthetic construct
MVSLRSIFTSSILAAGLTRAHG

SEQ ID NO: 68 - protein, 21 aa, synthetic construct
MKFPVPLLFLLQLFFIIATQG

SEQ ID NO: 69 - protein, 18 aa, synthetic construct
MQVKSIVNLLLACSLAVA

SEQ ID NO: 70 - protein, 22 aa, synthetic construct
MQFNWNIKTVASILSALTLAQA

SEQ ID NO: 71 - protein, 59 aa, synthetic construct
MYRNLIIATALTCGAYSAYVPSEPWSTLTPDASLESALKDYSQTFGIAIKSLDADKIKR

SEQ ID NO: 72 - protein, 22 aa, synthetic construct
MNLYLITLLFASLCSAITLPKR

SEQ ID NO: 73 - protein, 25 aa, synthetic construct
MFEKSKFVVSFLLLLQLFCVLGVHG

SEQ ID NO: 74 - protein, 21 aa, synthetic construct
MQFNSVVISQLLLTLASVSMG

SEQ ID NO: 75 - protein, 31 aa, synthetic construct
MKSQLIFMALASLVASAPLEHQQQHHKHEKR

SEQ ID NO: 76 - protein, 19 aa, synthetic construct
MKFAISTLLIILQAAAVFA

SEQ ID NO: 77 - protein, 23 aa, synthetic construct
MKLLNFLLSFVTLFGLLSGSVFA

SEQ ID NO: 78 - protein, 20 aa, synthetic construct
MIFNLKTLAAVAISISQVSA

SEQ ID NO: 79 - protein, 40 aa, synthetic construct
MKISALTACAVTLAGLAIAAPAPKPEDCTTTVQKRHQHKR

SEQ ID NO: 80 - protein, 18 aa, synthetic construct
MSYLKISALLSVLSVALA

SEQ ID NO: 81 - protein, 20 aa, synthetic construct
MLSTILNIFILLLFIQASLQ

SEQ ID NO: 82 - protein, 36 aa, synthetic construct
MKLSTNLILAIAAASAVVSAAPVAPAEEAANHLHKR

SEQ ID NO: 83 - protein, 19 aa, synthetic construct
MFKSLCMLIGSCLLSSVLA

SEQ ID NO: 84 - protein, 19 aa, synthetic construct
MKLAALSTIALTILPVALA

SEQ ID NO: 85 - protein, 23 aa, synthetic construct
MSFSSNVPQLFLLLVLLTNIVSG

SEQ ID NO: 86 - protein, 46 aa, synthetic construct
MQLQYLAVLCALLLNVQSKNVVDFSRFGDAKISPDDTDLESRERKR

SEQ ID NO: 87 - protein, 19 aa, synthetic construct
MKIHSLLLWNLFFIPSILG

SEQ ID NO: 88 - protein, 19 aa, synthetic construct
MSTLTLLAVLLSLQNSALA

SEQ ID NO: 89 - protein, 38 aa, synthetic construct
MINLNSFLILTVTLLSPALALPKNVLEEQQAKDDLAKR

SEQ ID NO: 90 - protein, 16 aa, synthetic construct
MFSLAVGALLLTQAFG

SEQ ID NO: 91 - protein, 16 aa, synthetic construct
MKILSALLLLFTLAFA

SEQ ID NO: 92 - protein, 20 aa, synthetic construct
MKVSTTKFLAVFLLVRLVCA

SEQ ID NO: 93 - protein, 19 aa, synthetic construct
MQFGKVLFAISALAVTALG

SEQ ID NO: 94 - protein, 18 aa, synthetic construct
MWSLFISGLLIFYPLVLG

SEQ ID NO: 95 - protein, 21 aa, synthetic construct
MRNHLNDLVVLFLLLTVAAQA

SEQ ID NO: 96 - protein, 18 aa, synthetic construct
MFLKSLLSFASILTLCKA

SEQ ID NO: 97 - protein, 20 aa, synthetic construct
MFVFEPVLLAVLVASTCVTA

SEQ ID NO: 98 - protein, 22 aa, synthetic construct
MFSPILSLEIILALATLQSVFA

SEQ ID NO: 99 - protein, 16 aa, synthetic construct
MIINHLVLTALSIALA

SEQ ID NO: 100 - protein, 19 aa, synthetic construct
MLALVRISTLLLLALTASA

SEQ ID NO: 101 - protein, 17 aa, synthetic construct
MRPVLSLLLLLASSVLA

SEQ ID NO: 102 - protein, 24 aa, synthetic construct
MVLIQNFLPLFAYTLFFNQRAALA

SEQ ID NO: 103 - protein, 20 aa, synthetic construct
MVSLTRLLITGIATALQVNA

SEQ ID NO: 104 - protein, 24 aa, synthetic construct
MIFDGTTMSIAIGLLSTLGIGAEA

SEQ ID NO: 105 - protein, 31 aa, synthetic construct
MVLVGLLTRLVPLVLLAGTVLLLVFVVLSGG

SEQ ID NO: 106 - protein, 16 aa, synthetic construct
MLSILSALTLLGLSCA

SEQ ID NO: 107 - protein, 20 aa, synthetic construct
MRLLHISLLSIISVLTKANA

SEQ ID NO: 108 - protein, 89 aa, synthetic construct
MRFPSIFTAVLFAASSALAAPVNTTTEDETAQIPAEAVIGYLDLEGDFDVAVLPFSNSTNNGLLFINTTIASIAAKEEGVSLDKREAEA

SEQ ID NO: 109 - protein, 17 aa, synthetic construct
MFKSVVYSILAASLANA

SEQ ID NO: 110 - protein, 19 aa, synthetic construct
MLLQAFLFLLAGFAAKISA

SEQ ID NO: 111 - protein, 21 aa, synthetic construct
MASSNLLSLALFLVLLTHANS

SEQ ID NO: 112 - protein, 29 aa, synthetic construct
MNIFYIFLFLLSFVQGLEHTHRRGSLVKR

SEQ ID NO: 113 - protein, 48 aa, synthetic construct
MLIIVLLFLATLANSLDCSGDVFFGYTRGDKTDVHKSQALTAVKNIKR

SEQ ID NO: 114 - protein, 49 aa, synthetic construct
MESVSSLFNIFSTIMVNYKSLVLALLSVSNLKYARGMPTSERQQGLEER

SEQ ID NO: 115 - protein, 18 aa, synthetic construct
MFAFYFLTACISLKGVFG

SEQ ID NO: 116 - protein, 23 aa, synthetic construct
MRFSTTLATAATALFFTASQVSA

SEQ ID NO: 117 - protein, 23 aa, synthetic construct
MKFAYSLLLPLAGVSASVINYKR

SEQ ID NO: 118 - protein, 22 aa, synthetic construct
MKFFAIAALFAAAAVAQPLEDR

SEQ ID NO: 119 - protein, 15 aa, synthetic construct
MQFFAVALFATSALA

SEQ ID NO: 120 - protein, 24 aa, synthetic construct
MKWVTFISLLFLFSSAYSRGVFRR

SEQ ID NO: 121 - protein, 18 aa, synthetic construct
MRSLLILVLCFLPLAALG

SEQ ID NO: 122 - protein, 15 aa, synthetic construct
MKVLILACLVALALA

SEQ ID NO: 123 - protein, 18 aa, synthetic construct
MFNLKTILISTLASIAVA

SEQ ID NO: 124 - protein, 20 aa, synthetic construct
MYRKLAVISAFLATARAQSA

SEQ ID NO: 125 - protein, 85 aa, synthetic construct
MRFPSIFTAVLFAASSALAAPVNTTTEDETAQIPAEAVIGYLDLEGDFDVAVLPFSNSTNNGLLFINTTIASIAAKEEGVQLDKR

SEQ ID NO: 126 - protein, 85 aa, synthetic construct
MRFPPIFTAALFAASSALAAPANTTTEDETAQIPAEAVIGYLDSEGDSDVAVLPFSNSTNNGLSFINTTIASIAAKEEGVQLDKR

SEQ ID NO: 127 - protein, 85 aa, synthetic construct
MRFPSIFTAVLFAASSALAAPANTTTEDETAQIPAEAVISYSDLEGDFDAAALPLSNSTNNGLSSTNTTIASIAAKEEGVQLDKR

SEQ ID NO: 128 - protein, 85 aa, synthetic construct
MRPPSIFTAVLFAASSALAAPANTTTEDETTQIPAEAVATYLDLEGDVDVAVLPFSSSTNNGLSFINTTIASIAAKEEGVQLDKR

SEQ ID NO: 129 - protein, 85 aa, synthetic construct
MRFPSIFTAALFAASSALAAPANTTTEGETAQTPAEAVIGYRDLEGDFDVAVLPFPNSTNNGLLFTNTTTASIAAKEEGVQLDKR

SEQ ID NO: 130 - protein, 85 aa, synthetic construct
MRFPSIFTAVLLAAPSALAAPANATTEDEAAQIPAEAVIGYLDLEGDFDAAVLPFSNSTNNGLLSINTTIASIAAKEEGVQLDKR

SEQ ID NO: 131 - protein, 85 aa, synthetic construct
MRFPSIFTAVVFAASSALAAPANTTAEDETAQIPAEAVIGYLGLEGDSDVAALPLSDSTNNGSLSTNTTIASIAAKEEGVQLDKR

SEQ ID NO: 132 - protein, 85 aa, synthetic construct
MRLPSIFTAAVFAASSALAAPANTTTEDETAQIPAEAAIGYLDLEGDSDVAVLPLSNSTNNGLLFINTTIASIAAKEEGVQLDKR

SEQ ID NO: 133 - protein, 85 aa, synthetic construct
MRFPSIFTAVLFAASSALAAPANTTTEDETAQIPAEAVIGYLDLEGDFDVAVLPFSNSTNDGLSFINTTTASIAAKEEGVQLDKR

SEQ ID NO: 134 - protein, 35 aa, synthetic construct
MRFPSIFTAVLFAASSALAAPVNTTTEDETAQIPA

SEQ ID NO: 135 - protein, 37 aa, synthetic construct
MLSTILNIFILLLFIQASLQAPIPVVTKYVTEGIAVV

SEQ ID NO: 136 - protein, 36 aa, synthetic construct
MSFSSNVPQLFLLLVLLTNIVSGAVISVWSTSKVTK

SEQ ID NO: 137 - protein, 35 aa, synthetic construct
MNLYLITLLFASLCSAITLPKRDIIWDYSSEKIMG

SEQ ID NO: 138 - protein, 35 aa, synthetic construct; identical to SEQ ID NO: 134

SEQ ID NO: 139 - protein, 35 aa, synthetic construct
MLSTILNIFILLLFIQASLQEFDYKDDDDKMVSKG

SEQ ID NO: 140 - protein, 35 aa, synthetic construct
MSFSSNVPQLFLLLVLLTNIVSGEFDYKDDDDKMV

SEQ ID NO: 141 - protein, 35 aa, synthetic construct
MNLYLITLLFASLCSAEFDYKDDDDKMVSKGEELF

SEQ ID NO: 142 - protein, 35 aa, synthetic construct; identical to SEQ ID NO: 134

SEQ ID NO: 143 - protein, 35 aa, synthetic construct
MLSTILNIFILLLFIQASLQEFLPSGSDPAFSQPK

SEQ ID NO: 144 - protein, 35 aa, synthetic construct
MSFSSNVPQLFLLLVLLTNIVSGEFLPSGSDPAFS

SEQ ID NO: 145 - protein, 35 aa, synthetic construct
MNLYLITLLFASLCSAEFLPSGSDPAFSQPKSVLD

SEQ ID NO: 146 - protein, 206 aa, synthetic construct
MVAWWSLFLYGLQVAAPALAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 147 - protein, 243 aa, synthetic construct
MRFPSIFTAVLFAASSALAAPVNTTTEDELEGDFDVAVLPFSASIAAKEEGVSLEKRAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 148 - protein, 205 aa, synthetic construct
MRFPSIFTAVLFAASSALAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 149 - protein, 212 aa, synthetic construct
MLGKNDPMCLVLVLLGLTALLGICQGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 150 - protein, 212 aa, synthetic construct
MTKPTQVLVRSVSILFFITLLHLVVAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 151 - protein, 205 aa, synthetic construct
MLLQAFLFLLAGFAAKISAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 152 - protein, 204 aa, synthetic construct
MKWVTFISLLFLFSSAYSAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 153 - protein, 204 aa, synthetic construct
MSFRSLLALSGLVCSGLAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 154 - protein, 202 aa, synthetic construct
MKLAYSLLLPLAGVSAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 155 - protein, 247 aa, synthetic construct
MRFPSIFTAVLFAASSALAAPVNTTTEDELEGDFDVAVLPFSASIAAKEEGVSLEKREAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 156 - protein, 24 aa, synthetic construct
MAMAGVFVLFSFVLCGFLPDAAFG

SEQ ID NO: 157 - protein, 18 aa, synthetic construct
MRSLLILVLCFLPLAALG

SEQ ID NO: 158 - protein, 89 aa, synthetic construct
MRFPSIFTAVLFAASSALAAPVNTTTEDETAQIPAEAVIGYSDLEGDFDVAVLPFSNSTNNGLLFINTTIASIAAKEEGVSLDKREAEA

SEQ ID NO: 159 - protein, 19 aa, synthetic construct
MKLILCTVLSLGIAAVCFA

SEQ ID NO: 160 - protein, 19 aa, synthetic construct
MKLFVPALLSLGALGLCLA

SEQ ID NO: 161 - protein, 19 aa, synthetic construct
MKLFIPALLFLGTLGLCLA

SEQ ID NO: 162 - protein, 23 aa, synthetic construct
MESKALLLLALSVWLQSLTVSHG

SEQ ID NO: 163 - protein, 16 aa, synthetic construct
MLLIWTLSLLLGAVLG

SEQ ID NO: 164 - protein, 186 aa, synthetic construct
AEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 165 - protein, 186 aa, synthetic construct
AEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 166 - protein, 186 aa, synthetic construct
AEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYMNKCNACNAVVESNGTLTLSHFGKC

SEQ ID NO: 167 - protein, 210 aa, synthetic construct
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 168 - protein, 210 aa, synthetic construct
MAMAGVFVLFSFVLCGFLPDAVFGAEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 169 - protein, 208 aa, synthetic construct
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVDCSEYPKPDCTAEDRPLCGSDNKTYGNKCNFCNAVVESNGTLTLSHFGKC

SEQ ID NO: 170 - protein, 186 aa, synthetic construct
AEVDCSRFPNATDKEGKDVLVCNKDLRPICGTDGVTYNNECLLCAYSIEFGTNISKEHDGECKETVPMNCSSYANTTSEDGKVMVLCNRAFNPVCGTDGVTYDNECLLCAHKVEQGASVDKRHDGECRKELAAVSVDCSEYPKPDCTAEDRPLCGSDNKTYGNKCNFCNAVVESNGTLTLSHFGKC

SEQ ID NO: 171 - protein, 208 aa, synthetic construct
MAMAGVFVLFSFALCGFLPDAAFGVEVDCSRFPNATNEEGKDVLVCTEDLRPICGTDGVTYSNDCLLCAYNIEYGTNISKEHDGECREAVPVDCSRYPNMTSEEGKVLILCNKAFNPVCGTDGVTYDNECLLCAHNVEQGTSVGKKHDGECRKELAAVDCSEYPKPACTMEYRPLCGSDNKTYDNKCNFCNAVVESNGTLTLSHFGKC

SEQ ID NO: 172 - protein, 239 aa, synthetic construct
MQTITWRQPQGDHLRSRAPAATCRAGQYLTMAMAGIFVLFSFALCGFLPDAAFGVEVDCSRFPNTTNEEGKDVLVCTEDLRPICGTDGVTHSECLLCAYNIEYGTNISKEHDGECREAVPMDCSRYPNTTNEEGKVMILCNKALNPVCGTDGVTYDNECVLCAHNLEQGTSVGKKHDGGCRKELAAVSVDCSEYPKPACTLEYRPLCGSDNKTYGNKCNFCNAVVESNGTLTLSHFGKC

SEQ ID NO: 173 - protein, 185 aa, synthetic construct
VEVDCSRFPNTTNEEGKDVLVCTEDLRPICGTDGVTHSECLLCAYNIEYGTNISKEHDGECREAVPMDCSRYPNTTSEEGKVMILCNKALNPVCGTDGVTYDNECVLCAHNLEQGTSVGKKHDGECRKELAAVSVDCSEYPKPACTLEYRPLCGSDNKTYGNKCNFCNAVVESNGTLTLSHFGKC

SEQ ID NO: 174 - protein, 237 aa, synthetic construct
MQTITWRQPQGDHLRSRAPAATCRAGQYLTMAMAGIFVLFSFALCGFLPDAAFGVEVDCSRFPNTTNEEGKDVLVCTEDLRPICGTDGVTHSECLLCAYNIEYGTNISKEHDGECREAVPMDCSRYPNTTNEEGKVMILCNKALNPVCGTDGVTYDNECVLCAHNLEQGTSVGKKHDGGCRKELAAVDCSEYPKPACTLEYRPLCGSDNKTYGNKCNFCNAVVESNGTLTLSHFGKC

SEQ ID NO: 175 - protein, 128 aa, synthetic construct
EYGTNISIKHNGECKETVPMDCSRYANMTNEEGKVMMPCDRTYNPVCGTDGVTYDNECQLCAHNVEQGTSVDKKHDGVCGKELAAVSVDCSEYPKPECTAEERPICGSDNKTYGNKCNFCNAVVYVQP

SEQ ID NO: 176 - protein, 195 aa, synthetic construct
VDCSRFPNTTNEEGKDVLACTKELHPICGTDGVTYSNECLLCYYNIEYGTNISKEHDGECTEAVPVDCSRYPNTTSEEGKVLIPCNRDFNPVCGSDGVTYENECLLCAHNVEQGTSVGKKHDGGCRKEFAAVSVDCSEYPKPDCTLEYRPLCGSDNKTYASKCNFCNAVVIWEQEKNTRHHASHSVFFISARLVC

SEQ ID NO: 177 - protein, 339 aa, synthetic construct
MLPLGLREYGTNTSKEHDGECTEAVPVDCSRYPNTTSEEGKVRILCKKDINPVCGTDGVTYDNECLLCSHSVGQGASIDKKHDGGCRKEFAAVSVDCSEYPKPACMSEYRPLCGSDNKTYVNKCNFCNAVVYVQPWLHSRCRLPPTGTSFLGSEGRETSLLTSRATDLQVAGCTAISAMEATRAAALLGLVLLSSFCELSHLCFSQASCDVYRLSGSRNLACPRIFQPVCGTDNVTYPNECSLCRQMLRSRAVYKKHDGRCVKVDCTGYMRATGGLGTACSQQYSPLYATNGVIYSNKCTFCSAVANGEDIDLLAVKYPEEESWISVSPTPWRMLSAGA

SEQ ID NO: 178 - protein, 238 aa, synthetic construct
MSWWGIKPALERPSQEQSTSGQPVDSGSTSTTTMAGIFVLLSLVLCCFPDAAFGVEVDCSRFPNTTNEEGKEVLLCTKDLSPICGTDGVTYSNECLLCAYNIEYGTNISKDHDGECKEAVPVDCSTYPNMTNEEGKVMLVCNKMFSPVCGTDGVTYDNECMLCAHNVEQGTSVGKKYDGKCKKEVATVDCSDYPKPACTVEYMPLCGSDNKTYDNKCNFCNAVVDSNGTLTLSHFGKC

SEQ ID NO: 179 - protein, 284 aa, synthetic construct
MSSQNQLHRRRRPLPGGQDLNKYYWPHCTSDRFSWLLHVTAEQFRHCVCIYLQPALERPSQEQSTSGQPVDSGSTSTTTMAGIFVLLSLVLCCFPDAAFGVEVDCSRFPNTTNEEGKEVLLCTKDLSPICGTDGVTYSNECLLCAYNIEYGTNISKDHDGECKEAVPVDCSTYPNMTNEEGKVMLVCNKMFSPVCGTDGVTYDNECMLCAHNVEQGTSVGKKYDGKCKKEVATVDCSDYPKPACTVEYMPLCGSDNKTYDNKCNFCNAVVDSNGTLTLSHFGKC

SEQ ID NO: 180 - protein, 186 aa, synthetic construct
VEVDCSRFPNTTNEEGKDEVVCPDELRLICGTDGVTYNHECMLCFYNKEYGTNISKEQDGECGETVPMDCSRYPNTTSEDGKVTILCTKDFSFVCGTDGVTYDNECMLCAHNVVQGTSVGKKHDGECRKELAAVSVDCSEYPKPACPKDYRPVCGSDNKTYSNKCNFCNAVVESNGTLTLNHFGKC

SEQ ID NO: 181 - protein, 210 aa, synthetic construct
MAMAGVFLLFSFALCGFLPDAAFGVEVDCSRFPNTTNEEGKDEVVCPDELRLICGTDGVTYNHECMLCFYNKEYGTNISKEQDGECGETVPMDCSRYPNTTSEDGKVTILCTKDFSFVCGTDGVTYDNECMLCAHNIVQGTSVGKKHDGECRKELAAVSVDCSEYPKPACPKDYRPVCGSDNKTYSNKCNFCNAVVESNGTLTLNHFGKC

SEQ ID NO: 182 - protein, 205 aa, synthetic construct
MAGVFVLLSLVLCCFPDAAFGVEVDCSRFPNTTNEEGKDVLLCTKELSPVCGTDGVTYSNECLLCAYNIEYGTNISKDHDGECKEAVPADCSMYPNMTNEEGKMTLLCNKMFSPVCGTDGVTYDNECMLCAHNVEQGTSVGKKYDGKCKKEVATVDCSGYPKPACTMEYMPLCGSDNKTYGNKCNFCNAVVDSNGTLTLSHFGEC

SEQ ID NO: 183 - protein, 171 aa, synthetic construct
QVDCSRFPNTTNEEGKEVLLCTKELSPVCGTDGVTYSNECLLCAYNIEYGTNISKDHDGECKEAVPADCSMYPNMTNEEGKMTLLCNKMFSPVCGTDGVTYDNECMLCAHNVEQGTSVGKKYDGKCKKEVATVSVDCSGYPKPACTMEYMPLCGSDNKTYGNKCNFCNAVV

SEQ ID NO: 184 - protein, 207 aa, synthetic construct
MTMPGAFVVLSFVLCCFPDATFGVEVDCSTYPNTTNEEGKEVLVCSKILSPICGTDGVTYSNECLLCANNIEYGTNISKYHDGECKEFVPVNCSRYPNTTNEEGKVMLICNKDLSPVCGTDGVTYDNECLLCAHNLEPGTSVGKKYDGECKKEIATVDCSDYPKPVCSLESMPLCGSDNKTYSNKCNFCNAVVDSNETLTLSHFGKC

SEQ ID NO: 185 - protein, 207 aa, synthetic construct
MTMAGVFVLLSFALCCFPDAAFGVEVDCSTYPNTTNEEGKEVLVCTKILSPICGTDGVTYSNECLLCAYNIEYGTNVSKDHDGECKEVVPVDCSRYPNSTNEEGKVVMLCSKDLNPVCGTDGVTYDNECVLCAHNVESGTSVGKKYDGECKKETATVDCSDYPKPACTLEYMPFCGSDSKTYSNKCNFCNAVVDSNGTLTLSHFGKC

SEQ ID NO: 186 - protein, 207 aa, synthetic construct
MTTAGVFVLLSFALCSFPDAAFGVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 187 - protein, 207 aa, synthetic construct
MTTAGVFVLLSFTLCSFPDAAFGVEVDCSPYPNTTNEEGKEVLVCNKILSPICGTDGVTYSNECLLCAYNLEYGTNISKDYDGECKEVAPVDCSRHPNTTNEEGKVELLCNKDLNPICGTNGVTYDNECLLCARNLESGTSIGKKYDGECKKEIATVDCSDYPKPVCTLEEMPLCGSDNKTYGNKCNFCNAVVDSNGTLTLSHFGKC

SEQ ID NO: 188 - protein, 207 aa, synthetic construct
MTTAVVFVLLSFALCCFPDAAFGVEVDCSTYPNSTNEEGKDVLVCPKILGPICGTDGVTYSNECLLCAYNIQYGTNVSKDHDGECKEIVPVDCSRYPNTTNEEGKVVFLCNKNFDPVCGTDGDTYDNECMLCARSLEPGTTVGKKHDGECKREIATVDCSDYPKPTCSAEDMPLCGSDSKTYSNKCNFCNAVVDSNGTLTLSRFGKC

SEQ ID NO: 189 - protein, 207 aa, synthetic construct
MTMTGVFVLLSFAICCFPDAAFGVEVDCSTYPNTTNEEGKEVLVCTKILSPICGTDGVTYSNECLLCAYNIEYGTNVSKDHDGECKEVVPVDCSKYPNTTNEEGKVVLLCSKDLSPVCGTDGVTYDNECLLCARNLEPGSSVGKKYDGECKKEIATIDCSDYPKPVCSLEYMPLCGSDSKTYDNKCNFCNAVVDSNGTLTLSHFGKC

SEQ ID NO: 190 - protein, 207 aa, synthetic construct
MTTAGVFVLLSFVLCCFPDAVFGVEVDCSTYPNTTNEEGKEVLVCTKILSPICGTDGVTYSNECLLCAYNIEYGTNVSKDHDGECKEVVPVNCSRYPNTTNEEGKVVLRCSKDLSPVCGTDGVTYDNECLMCARNLEPGAVVGKNYDGECKKEIATVDCSDYPKPVCSLEYMPLCGSDSKTYSNKCNFCNAVVDSNGTLTLSHFGKC

SEQ ID NO: 191 - protein, 207 aa, synthetic construct
MTTAGVFVLLSIALCCFPDAAFGVEVDCSAYSNTTSEEGKEVLSCTKILSPICGTDGVTYSNECLLCAYNIEYGTNISKDHDGECKEVVSVDCSRYPNTTNEEGKAVLLCNKDLSPVCGTDGVTYDNECLLCAHNLEPGTSVGKKYDGACKKEIATVDCSDYPKPVCTLEYLPLCGSDSKTYSNKCDFCNAVVDSNGTLTLSHFGKC

SEQ ID NO: 192 - protein, 206 aa, synthetic construct
MTTAGVFVLLSFALCCFPDAAFGVEVDCSTYPNTTNEEGKEVLVCTKILSPICGTDGTTYSNECLLCAYNIEYGTNVSKDHDGECKVVPVDCSKYPNTTNEDGKVVLLCNKALSPICGTDRVTYDNECLMCAHNLEPGTSVGKKHDGECQKEVATVDCSDYPKPVCSLEYMPLCGSDGKTYSNKCNFCNAVVNSNGTLTLSHFEKC

SEQ ID NO: 193 - protein, 213 aa, synthetic construct
MTTAGVFVLLSFVLCCFFPDAAFGVEVDCSTYPNTTNEEGKEVLVCAKILSPVCGTDGVTYSNECLLCAHNIENGTNVGKDHDGKCKEAVPVDCSRYPNTTDEEGKVVLLCNKDVSPVCGTDGVTYDNECLLCAHNLEAGTSVDKKNDSECKTEDTTLAAVSVDCSDYPKPVCTLEYLPLCGSDNKTYSNKCRFCNAVVDSNGTLTLSRFGKC

SEQ ID NO: 194 - protein, 207 aa, synthetic construct
MTTAGVFVLLSFALCCSPDAAFGVEVDCSTYPNTTNEEGKEVLACTKILSPICGTDGVTYSNECLLCAYNMEYGTNVSKDHDGKCKEVVPVDCSRYPNTTNEEGKVVLLCNKDLSPVCGTDGVTYDNECLLCARNLEPGASVGKKYDGECKKEIATVDCSDYPKPVCSLEHMPLCGSDSKTYSNKCTFCNAVVDSNGTLTLSHFGKC

SEQ ID NO: 195 - protein, 207 aa, synthetic construct
MTTAGVFVLLSFALCCFPDAAFGVEVDCSTYPNTTNEEGREVLVCTKILSPICGTDGVTYSNECLLCAYNIEYGTNVSKDHDGECKEVAPVGCSRYPNTTNEEGKVVLLCNKDLSPVCGTDGVTYDNECLLCARHLEPGTSVGKKYDGECKKEIATVDCSDYPKPVCSLEYMPLCGSDSKTYSNKCNFCNAVLDSNGTLTLSHFGKC

SEQ ID NO: 196 - protein, 207 aa, synthetic construct
MTTAGVFVLLSFALCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCNKDLSPVCGTDGVTYDNECLMCARNLEPGAIVGKKYDGECKKEIATVDCSDYPKPVCSLEYMPLCGSDSKTYSNKCNFCNAVVDSNGTLILSHFGKC

SEQ ID NO: 197 - protein, 207 aa, synthetic construct
MTTAGVFVLLSFVLCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCSKDLSPVCGTDGVTYDNECLMCARNLEPGAVVGKNYDGECKKEIATVDCSDYPKPVCSLEYMPLCGSDSKTYSNKCNFCNAVVDSNGTLTLSHFGKC

SEQ ID NO: 198 - protein, 282 aa, synthetic construct
MSSQNQLPSRCRPLPGSQDLNKYYQPHCTGDRFCWLFYVTVEQFRHCICIYLQLALERPSHEQSGQPADSRNTSTMTTAGVFVLLSFALCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCNKDLSPVCGTDGVTYDNECLMCARNLEPGAIVGKKYDGECKKEIATVDCSDYPKPVCSLEYMPLCGSDSKTYSNKCNFCNAVVDSNGTLILSHFGKC

SEQ ID NO: 199 - protein, 193 aa, synthetic construct
MTTAVVFVLLSFALCCFPDAAFGVEVDCSTYPNSTNEEGKDVLVCPKILGPICGTDGVTYSNECLLCAYNIQYGTNVSKDHDGECKEIVPVDCSRYPNTTNEEGKVVFLCNKNFDPVCGTDGDTYDNECMLCARSLEPGTTVGKKHDGECKREIATVDCSDYPKPTCSAEDMPLCGSDSKTYSNKCNFCNAVV

SEQ ID NO: 200 - protein, 190 aa, synthetic construct
EAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 201 - protein, 199 aa, synthetic construct
EEGVSLEKREAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 202 - protein, 275 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 203 - protein, 273 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEAVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 204 - protein, 188 aa, synthetic construct
EAEAVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 205 - protein, 206 aa, synthetic construct
MTMAGVFVLLSFILCCFPDTAFGVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 206 - protein, 272 aa, synthetic construct
MRFPSIFTAVLFAASSALAAPVNTTTEDETAQIPAEAVIGYSDLEGDFDVAVLPFSNSTNNGLLFINTTIASIAAKEEGVSLDKREAEAVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 207 - protein, 187 aa, synthetic construct
EAEAVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 208 - protein, 402 aa, synthetic construct
MFFYNTDFRMGSISAANAEFCFDVFNELKVQHTNENILYSPLSIIVALAMVYMGARGNTEYQMEKALHFDSIAGLGGSTQTKVQKPKCGKSVNIHLLFKELLSDITASKANYSLRIANRLYAEKSRPILPIYLKCVKKLYRAGLETVNFKTASDQARQLINSWVEKQTEGQIKDLLVSSSTDLDTTLVLVNAIYFKGMWKTAFNAEDTREMPFHVTKEESKPVQMMCMNNSFNVATLPAEKMKILELPFASGDLSMLVLLPDEVSGLERIEKTINFEKLTEWTNPNTMEKRRVKVYLPQMKIEEKYNLTSVLMALGMTDLFIPSANLTGISSAESLKISQAVHGAFMELSEDGIEMAGSTGVIEDIKHSPELEQFRADHPFLFLIKHNPTNTIVYFGRYWSP

SEQ ID NO: 209 - protein, 388 aa, synthetic construct
MDSISVTNAKFCFDVFNEMKVHHVNENILYCPLSILTALAMVYLGARGNTESQMKKVLHFDSITGAGSTTDSQCGSSEYVHNLFKELLSEITRPNATYSLEIADKLYVDKTFSVLPEYLSCARKFYTGGVEEVNFKTAAEEARQLINSWVEKETNGQIKDLLVSSSIDFGTTMVFINTIYFKGIWKIAFNTEDTREMPFSMTKEESKPVQMMCMNNSFNVATLPAEKMKILELPYASGDLSMLVLLPDEVSGLERIEKTINFDKLREWTSTNAMAKKSMKVYLPRMKIEEKYNLTSILMALGMTDLFSRSANLTGISSVDNLMISDAVHGVFMEVNEEGTEATGSTGAIGNIKHSLELEEFRADHPFLFFIRYNPTNAILFFGRYWSP

SEQ ID NO: 210 - protein, 386 aa, synthetic construct
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 211 - protein, 474 aa, synthetic construct
MRFPSIFTAVLFAASSALAAPVNTTTEDETAQIPAEAVIGYSDLEGDFDVAVLPFSNSTNNGLLFINTTIASIAAKEEGVSLDKREAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 212 - protein, 389 aa, synthetic construct
EAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 213 - protein, 419 aa, synthetic construct
MRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 214 - protein, 406 aa, synthetic construct
MTSGFANELGPRLMGKLTMGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPSR

SEQ ID NO: 215 - protein, 451 aa, synthetic construct
MGGRRVRWEVYISRAGYVNRQIAWRRHHRSLTMRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 216 - protein, 386 aa, synthetic construct
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAVHAAYAEIYEAGREVIGSAEAGADATSVSEEFRVDHPFLYCIKHNLTNSILFFGRCISP

SEQ ID NO: 217 - protein, 386 aa, synthetic construct
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAAHAAYAEIYEAGREVIGSAEAGADATSVSEEFRVDHPFLYCIKHNLTNSILFFGRCISP

SEQ ID NO: 218 - protein, 417 aa, synthetic construct
YYRVPCMVLCTAFHPYIFIVLLFALDNSEFTMGSIGAVSMEFCFDVFKELRVHHPNENIFFCPFAIMSAMAMVYLGAKDSTRTQINKVIRFDKLPGFGDSTEAQCGKSANVHSSLKDILNQITKPNDVYSFSLASRLYADETYSIQSEYLQCVNELYRGGLESINFQTAADQARELINSWVESQTNGIIRNVLQPSSVDSQTAMVLVNAIVFRGLWEKAFKDEDTQTMPFRVTEQESKPVQMMYQIGSFKVASMASEKMKILELPLASGTMSMLVLLPDEVSGLEQLETTISFEKLTEWTSSNVMEERKIKVYLPRMKMEEKYNLTSVLMAMGITDLFRSSANLSGISLAGNLKISQAVHAAHAEINEAGRKAVSSAEAGVDATSVSEEFRADRPFLFCIKHIATKVVFFFGRYTSP

SEQ ID NO: 219 - protein, 383 aa, synthetic construct
MGSIGAASMEFCFDVFKELKVHHANDNMLYSPFAILSTLAMVFLGAKDSTRTQINKVVHFDKLPGFGDSIEAQCGTSVNVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMYQIGSFKVASMASEKMKILELPFASGTMSMLVLLPDDVSGLEQLESIISFEKLTEWTSSSIMEERKVKVYLPRMKMEEKYNLTSLLMAMGITDLFSSSANLSGISSVGSLKISQAVHAAHAEINEAGRDVVGSAEAGVDATEEFRADHPFLFCVKHIETNAILLFGRCVSP

SEQ ID NO: 220 - protein, 386 aa, synthetic construct
MASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 221 - protein, 411 aa, synthetic construct
MALCKAFHPYIFIVLLFDVDNSAFTMASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 222 - protein, 383 aa, synthetic construct
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 223 - protein, 408 aa, synthetic construct
MGLCTAFHPYIFIVLLFALDNSEFTMGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 224 - protein, 383 aa, synthetic construct
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKIPQAVHAAYAEINEAGRDVVGSAEAGVDATEEFRADHPFLFCVKHIETNAILLFGRCVSP

SEQ ID NO: 225 - protein, 386 aa, synthetic construct
MGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 226 - protein, 386 aa, synthetic construct
MGSIGAASTEFCFDVFRELKVQHVNENIFYSPLSIISALAMVYLGARDNTRTQIDQVVHFDKIPGFGESMEAQCGTSVSVHSSLRDILTEITKPSDNFSLSFASRLYAEETYTILPEYLQCVKELYKGGLESISFQTAADQARELINSWVESQTNGIIKNILQPSSVDSQTTMVLVNAIYFKGMWEKAFKDEDTQTMPFRMTEQESKPVQMMYQVGSFKLATVTSEKVKILELPFASGMMSMCVLLPDEVSGLEQLETTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPSNSILFFGRWISP

SEQ ID NO: 227 - protein, 386 aa, synthetic construct
MGSIGAASTEFCFDVFKELKVQHVNENIFYSPLTIISALSMVYLGARENTRAQIDKVLHFDKMPGFGDTIESQCGTSVSIHTSLKDMFTQITKPSDNYSLSFASRLYAEETYPILPEYLQCVKELYKGGLETISFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITFEKLMAWTSSTTMEERKMKVYLPRMKIEEKYNLTSVLMALGVTDLFSSSANLSGISSAESLKISKAVHEAFVEIYEAGSEVVGSTEAGMEVTSVSEEFRADHPFLFLIKHNPTNSILFFGRCFSP

SEQ ID NO: 228 - protein, 386 aa, synthetic construct
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSVSEEFRADHPFLFLIKHKPTNSILFFGRCFSP

SEQ ID NO: 229 - protein, 386 aa, synthetic construct
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSFSEEFRADHPFLFLIKHKPTNSILFFGRCFSP

SEQ ID NO: 230 - protein, 386 aa, synthetic construct
MGSIGAASTEFCFDVFKELKVQHVNENIFYSPLSIISALSMVYLGARENTRAQIDKVVHFDKITGFGETIESQCGTSVSVHTSLKDMFTQITKPSDNYSLSFASRLYAEETYPILPEYLQCVKELYKGGLETTSFQTAADQARELINSWVESQTNGMIKNILQPGSVDPQTEMVLVNAIYFKGMWEKAFKDEDTQAVPFRMTEQESKTVQMMYQIGSFKVAVMASEKMKILELPYASGELSMLVMLPDDVSGLEQLETAITFEKLMEWTSSNMMEERKMKVYLPRMKMEEKYNLTSVLMALGVTDLFSSSANLSGISSAESLKMSEAVHEAFVEIYEAGSEVVGSTGAGMEVTSVSEEFRADHPFLFLIKHNPTNSILFFGRCFSP

SEQ ID NO: 231 - protein, 386 aa, synthetic construct
MGSIGAASTEFCFDVFKELRVQHVNENVCYSPLIIISALSLVYLGARENTRAQIDKVVHFDKITGFGESIESQCGTSVSVHTSLKDMFNQITKPSDNYSLSVASRLYAEERYPILPEYLQCVKELYKGGLESISFQTAADQAREAINSWVESQTNGMIKNILQPSSVDPQTEMVLVNAIYFKGMWQKAFKDEDTQAVPFRISEQESKPVQMMYQIGSFKVAVMAAEKMKILELPYASGELSMLVLLPDEVSGLEQLENAITVEKLMEWTSSSPMEERIMKVYLPRMKIEEKYNLTSVLMALGITDLFSSSANLSGISAEESLKMSEAVHQAFAEISEAGSEVVGSSEAGIDATSVSEEFRADHPFLFLIKHNATNSILFFGRCFSP

SEQ ID NO: 232 - protein, 386 aa, synthetic construct
MGSISAASTEFCFDVFKELKVQHVNENIFYSPLSIISALSMVYLGARENTRAQIEKVVHFDKITGFGESIESQCSTSVSVHTSLKDMFTQITKPSDNYSLSFASRFYAEETYPILPEYLQCVKELYKGGLETINFRTAADQARELINSWVESQTNGMIKNILQPGSVDPQTDMVLVNAIYFKGMWEKAFKDEDTQALPFRVTEQESKPVQMMYQIGSFKVAVLASEKVKILELPYASGQLSMLVLLPDDVSGLEQLETAITVEKLMEWTSSNNMEERKIKVYLPRIKIEEKYNLTSVLMALGITDLFSSSANLSGISSAESLKVSEAIHEAFVEIYEAGSEVAGSTEAGIEVTSVSEEFRADHPFLFLIKHNATNSILFFGRCFSP

SEQ ID NO: 233 - protein, 386 aa, synthetic construct
MVSIGAASTEFCFDVFKELKVQHVNENIFYSPLSIISALSMVYLGARENTRAQIDKVVHFDKITGFEETIESQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 234 - protein, 386 aa, synthetic construct
MGSIGAASTEFCFDVFKELKVQHVNENIFYSPLSIISALSMVYLGARENTRAQIDKVVHFDKITGFGEPIESQCGISVSVHTSLKDMITQITKPSDNYSLSFASRLYAEETYPILPEYLQCVKELYKGGLETISFQTAADQARELINSWVENQTNGMIKNILQPGSVDPQTEMVLVNAVYFKGMWEKAFKDEDTQAVPFRMTEQESKPVQMMYQIGSFKVAVMASEKIKILELPYASGELSMLVLLPDDVSGLEQLETAITLDKLTEWTSSNAMEERKMKVYLPRMKIEKKYNLTSVLIALGMTDLFSSSANLSGISSAESLKMSEAIHEAFLEIYEAGSEVVGSTEAGMEVTSVSEEFRADHPFLFLIKHNPTNSILFFGRCLSP

SEQ ID NO: 235 - protein, 386 aa, synthetic construct
MGSIGAASTEFCFDVFKELKVQHVNENIFYSPLTIISALSMVYLGARENTRAQIDKVVHFDKIPGFGDTTESQCGTSVSVHTSLKDMFTQITKPSDNYSVSFASRLYAEETYPILPEFLECVKELYKGGLESISFQTAADQARELINSWVESQTNGMIKNILQPGSVDSQTEMVLVNAIYFKGMWEKAFKDEDTQTVPFRMTEQETKPVQMMYQIGTFKVAVMPSEKMKILELPYASGELCMLVMLPDDVSGLEELESSITVEKLMEWTSSNMMEERKMKVFLPRMKIEEKYNLTSVLMALGMTDLFSSSANLSGISSAEPLKMSEAVHEAFIEIYEAGSEVVGSTGAGMEITSVSEEFRADHPFLFLIKHNPTNSILFFGRCVSP

SEQ ID NO: 236 - protein, 386 aa, synthetic construct
MGSIGAVSTEFCFDVFKELKVQHVNENIFYSPLSIISALSMVYLGARENTRAQIDKVVHFDKITGSGETIEAQCGTSVSVHTSLKDMFTQITKPSENYSVGFASRLYADETYPIIPEYLQCVKELYKGGLEMISFQTAADQARELINSWVESQTNGMIKNILQPGSVDPQTEMILVNAIYFKGVWEKAFKDEDTQAVPFRMTEQESKPVQMMYQFGSFKVAAMAAEKMKILELPYASGALSMLVLLPDDVSGLEQLESAITFEKLMEWTSSNMMEEKKIKVYLPRMKMEEKYNFTSVLMALGMTDLFSSSANLSGISSADSLKMSEVVHEAFVEIYEAGSEVVGSTGSGMEAASVSEEFRADHPFLFLIKHNPTNSILFFGRCFSP

SEQ ID NO: 237 - protein, 391 aa, synthetic construct
MVSIGAASTEFCFDVFKELKVQHVNENIFYSPLSIISALSMVYLGARENTRAQIDKVVHFDKITGFEETIESQVQKKQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 238 - protein, 386 aa, synthetic construct
MGSIGAASGEFCFDVFKELKVQHVNENIFYSPLSIISALSMVYLGARENTRAQIDKVVHFDKIIGFGESIESQCGTSVSVHTSLKDMFAQITKPSDNYSLSFASRLYAEETFPILPEYLQCVKELYKGGLETLSFQTAADQARELINSWVESQTNGMIKDILQPGSVDPQTEMVLVNAIYFKGVWEKAFKDEDTQTVPFRMTEQESKPVQMMYQIGSFKVAVVAAEKIKILELPYASGALSMLVLLPDDVSSLEQLETAITFEKLTEWTSSNIMEERKIKVYLPRMKIEEKYNLTSVLMDLGITDLFSSSANLSGISSAESLKVSEAIHEAIVDIYEAGSEVVGSSGAGLEGTSVSEEFRADHPFLFLIKHNPTSSILFFGRCFSP

SEQ ID NO: 239 - protein, 386 aa, synthetic construct
MGSIGAASTEFCFDVFKELKVQHVNENIFYSPLSIISALSMVYLGARENTRAQIDKVVHFDKITGSGEAIESQCGTSVSVHISLKDMFTQITKPSDNYSLSFASRLYAEETYPILPEYLQCVKELYKEGLATISFQTAADQAREFINSWVESQTNGMIKNILQPGSVDPQTQMVLVNAIYFKGVWEKAFKDEDTQAVPFRMTKQESKPVQMMYQIGSFKVAVMASEKMKILELPYASGQLSMLVMLPDDVSGLEQIENAITFEKLMEWTNPNMMEERKMKVYLPRMKMEEKYNLTSVLMALGMTDLFSSSANLSGISSAESLKMSEAVHEAFVEIYEAGSEVVGSTGAGIEVTSVSEEFRADHPFLFLIKHNPTNSILFFGRCFSP

SEQ ID NO: 240 - protein, 386 aa, synthetic construct
MGSIGEASTEFCIDVFRELKVQHVNENIFYSPLSIISALSMVYLGARENTRAQIDQVVHFDKITGFGDTVESQCGSSLSVHSSLKDIFAQITQPKDNYSLNFASRLYAEETYPILPEYLQCVKELYKGGLETISFQTAADQARELINSWVESQTNGMIKNILQPSSVDPQTEMVLVNAIYFKGVWEKAFKDEETQAVPFRITEQENRPVQIMYQFGSFKVAVVASEKIKILELPYASGQLSMLVLLPDEVSGLEQLENAITFEKLTEWTSSDIMEEKKIKVFLPRMKIEEKYNLTSVLVALGIADLFSSSANLSGISSAESLKMSEAVHEAFVEIYEAGSEVVGSSGAGIEAASDSEEFRADHPFLFLIKHKPTNSILFFGRCFSP

SEQ ID NO: 241 - protein, 386 aa, synthetic construct
MGSIGAASTEFCFDIFNELKVQHVNENIFYSPLSIISALSMVYLGARENTKAQIDKVVHFDKITGFGESIESQCSTSASVHTSFKDMFTQITKPSDNYSLSFASRLYAEETYPILPEYSQCVKELYKGGLESISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASGELSMLVLLPDDVSGLEQLETAITFEKLMEWTSSNMMEERKVKVYLPRMKIEEKYNLTSVLMALGMTDLFSPSANLSGISSAESLKMSEAIHEAFVEIYEAGSEVVGSTEAGMEVTSVSEEFRADHPFLFLIKCNLTNSILFFGRCFSP

SEQ ID NO: 242 - protein, 385 aa, synthetic construct
MGSISTASTEFCFDVFKELKVQHVNENIFYSPLSIISALSMVYLGARENTRAQIEKVVHFDKITGFGESIESQCGTSVSVHTSLKDMLIQISKPSDNYSLSFASKLYAEETYPILPEYLQCVKELYKGGLESINFQTAADQARQLINSWVESQTNGMIKDILQPSSVDPQTEMVLVNAIYFKGIWEKAFKDEDTQEVPFRITEQESKPVQMMYQIGSFKVAVIASEKIKILELPYASGELSMLIVLPDDVSGLEQLETAITFEKLIEWTSPSIMEERKTKVYLPRMKIEEKYNLTSVLMALGMTDLFSPSANLSGISSAESLKMSEAIHEAFVEIYEAGSEVVGSAEAGMEATSVSEFRVDHPFLFLIKHNPANIILFFGRCVSP

SEQ ID NO: 243 - protein, 386 aa, synthetic construct
MGSIGAASTEFCFDVFKELKVQHVNENIFYSPLTIISALSLVYLGARENTRAQIDKVFHFDKISGFGETTESQCGTSVSVHTSLKEMFTQITKPSDNYSVSFASRLYAEDTYPILPEYLQCVKELYKGGLETISFQTAADQAREVINSWVESQTNGMIKNILQPGSVDSQTEMVLVNAIYFKGMWEKAFKDEDTQTMPFRITEQERKPVQMMYQAGSFKVAVMASEKMKILELPYASGEFCMLIMLPDDVSGLEQLENSFSFEKLMEWTTSNMMEERKMKVYIPRMKMEEKYNLTSVLMALGMTDLFSSSANLSGISSAETLKMSEAVHEAFMEIYEAGSEVVGSTGSGAEVTGVYEEFRADHPFLFLVKHKPTNSILFFGRCVSP

SEQ ID NO: 244 - protein, 386 aa, synthetic construct
MGSIGAASTEFCFDIFNELKVQHVNENIFYSPLSIISALSMVYLGARENTKAQIDKVVHFDKITGFGETIESQCSTSVSVHTSLKDTFTQITKPSDNYSLSFASRLYAEETYPILPEYSQCVKELYKGGLETISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASRELSMLVLLPDDVSGLEQLETAITFEKLMEWTSSNMMEERKVKVYLPRMKIEEKYNLTSVLMALGMTDLFSPSANLSGISSAESLKMSEAVHEAFVEIYEAGSEVVGSTGAGMEVTSVSEEFRADHPFLFLIKCNPTNSILFFGRCFSP

SEQ ID NO: 245 - protein, 386 aa, synthetic construct
MGSISAASAEFCLDVFKELKVQHVNENIFYSPLSIISALSMVYLGARENTRAQIDKVVHFDKITGSGETIEFQCGTSANIHPSLKDMFTQITRLSDNYSLSFASRLYAEERYPILPEYLQCVKELYKGGLETISFQTAADQARELINSWVESQTNGMIKNILQPGSVNPQTEMVLVNAIYFKGLWEKAFKDEDTQTVPFRMTEQESKPVQMMYQVGSFKVAVMASDKIKILELPYASGELSMLVLLPDDVTGLEQLETSITFEKLMEWTSSNVMEERTMKVYLPHMRMEEKYNLTSVLMALGVTDLFSSSANLSGISSAESLKMSEAVHEAFVEIYESGSQVVGSTGAGTEVTSVSEEFRVDHPFLFLIKHNPTNSILFFGRCFSP

SEQ ID NO: 246 - protein, 385 aa, synthetic construct
MGSIGAASVEFCFDVFKELKVQHVNENIFYSPLSIISALSMVYLGARENTKAQIDKVVHFDKIAGFGEAIESQCVTSASIHSLKDMFTQITKPSDNYSLSFASRLYAEEAYSILPEYLQCVKELYKGGLETISFQTAADQARDLINSWVESQTNGMIKNILQPGAVDLETEMVLVNAIYFKGMWEKAFKDEDTQTVPFRMTEQESKPVQMMYQVGSFKVAVMASDKIKILELPYASGQLSMVVVLPDDVSGLEQLEASITSEKLMEWTSSSIMEEKKIKVYFPHMKIEEKYNLTSVLMALGMTDLFSSSANLSGISSAEKLKVSEAVHEAFVEISEAGSEVVGSTEAGTEVTSVSEEFKADHPFLFLIKHNPTNSILFFGRCFSP

SEQ ID NO: 247 - protein, 386 aa, synthetic construct
MGSIGAASSEFCFDIFKELKVQHVNENIFYSPLSIISALSMVYLGARENTRAQIDKVVPFDKITASGESIESQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 248 - protein, 386 aa, synthetic construct
MGSIGAASTEFCFDVFKELKAQYVNENIFYSPMTIITALSMVYLGSKENTRAQIAKVAHFDKITGFGESIESQCGASASIQFSLKDLFTQITKPSGNHSLSVASRIYAEETYPILPEYLECMKELYKGGLETINFQTAANQARELINSWVERQTSGMIKNILQPSSVDSQTEMVLVNAIYFRGLWEKAFKVEDTQATPFRITEQESKPVQMMHQIGSFKVAVVASEKIKILELPYASGRLTMLVVLPDDVSGLKQLETTITFEKLMEWTTSNIMEERKIKVYLPRMKIEEKYNLTSVLMALGLTDLFSSSANLSGISSAESLKMSEAVHEAFVEIYEAGSEVVASAEAGMDATSVSEEFRADHPFLFLIKDNTSNSILFFGRCFSP

SEQ ID NO: 249 - protein, 386 aa, synthetic construct
MGSIGAASTEFCFDVFKELKGQHVNENIFFCPLSIVSALSMVYLGARENTRAQIVKVAHFDKIAGFAESIESQCGTSVSIHTSLKDMFTQITKPSDNYSLNFASRLYAEETYPIIPEYLQCVKELYKGGLETISFQTAADQAREIINSWVESQTNGMIKNILRPSSVHPQTELVLVNAVYFKGTWEKAFKDEDTQAVPFRITEQESKPVQMMYQIGSFKVAAVTSEKMKILEVPYASGELSMLVLLPDDVSGLEQLETAITAEKLIEWTSSTVMEERKLKVYLPRMKIEEKYNLTTVLTALGVTDLFSSSANLSGISSAQGLKMSNAVHEAFVEIYEAGSEVVGSKGEGTEVSSVSDEFKADHPFLFLIKHNPTNSIVFFGRCFSP

SEQ ID NO: 250 - protein, 386 aa, synthetic construct
MGSIGAASTEFCFDVFKELKVHHVNENILYSPLAIISALSMVYLGAKENTRDQIDKVVHFDKITGIGESIESQCSTAVSVHTSLKDVFDQITRPSDNYSLAFASRLYAEKTYPILPEYLQCVKELYKGGLETIDFQTAADQARQLINSWVEDETNGMIKNILRPSSVNPQTKIILVNAIYFKGMWEKAFKDEDTQEVPFRITEQETKSVQMMYQIGSFKVAEVVSDKMKILELPYASGKLSMLVLLPDDVYGLEQLETVITVEKLKEWTSSIVMEERITKVYLPRMKIMEKYNLTSVLTAFGITDLFSPSANLSGISSTESLKVSEAVHEAFVEIHEAGSEVVGSAGAGIEATSVSEEFKADHPFLFLIKHNPTNSILFFGRCFSP

SEQ ID NO: 251 - protein, 386 aa, synthetic construct
MGSIGAASTEFCLDVFKELKVQHVNENIFYSPLSIISALSMVYLGARENTRAQIDKVVHFDKITGFEDSIESQCGTSVSVHTSLKDMFTQITKPSDNYSVGFASRLYAAETYQILPEYSQCVKELYKGGLETINFQKAADQATELINSWVESQTNGMIKNILQPSSVDPQTQIFLVNAIYFKGMWQRAFKEEDTQAVPFRISEKESKPVQMMYQIGSFKVAVIPSEKIKILELPYASGLLSMLVILPDDVSGLEQLENAITLEKLMQWTSSNMMEERKIKVYLPRMRMEEKYNLTSVFMALGITDLFSSSANLSGISSAESLKMSDAVHEASVEIHEAGSEVVGSTGSGTEASSVSEEFRADHPYLFLIKHNPTDSIVFFGRCFSP

SEQ ID NO: 252 - protein, 386 aa, synthetic construct
MGSIGAASTEFCFDVFKELKFQHVDENIFYSPLTIISALSMVYLGARENTRAQIDKVVHFDKIAGFEETVESQCGTSVSVHTSLKDMFAQITKPSDNYSLSFASRLYAEETYPILPEYLQCVKELYKGGLETISFQTAADQARDLINSWVESQTNGMIKNILQPSSVGPQTELILVNAIYFKGMWQKAFKDEDTQEVPFRMTEQQSKPVQMMYQTGSFKVAVVASEKMKILALPYASGQLSLLVMLPDDVSGLKQLESAITSEKLIEWTSPSMMEERKIKVYLPRMKIEEKYNLTSVLMALGITDLFSPSANLSGISSAESLKMSQAVHEAFVEIYEAGSEVVGSTGAGMEDSSDSEEFRVDHPFLFFIKHNPTNSILFFGRCFSP

SEQ ID NO: 253 - protein, 386 aa, synthetic construct
MGSIGPLSVEFCCDVFKELRIQHPRENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPINFQTAAEQARELINSWVESQTNGMIKNILQPSSVNPETDMVLVNAIYFKGLWEKAFKDEDIQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAESLKVSSAFHEASVEIYEAGSKVVGSTGAEVEDTSVSEEFRADHPFLFLIKHNPSNSIFFFGRCFSP

SEQ ID NO: 254 - protein, 386 aa, synthetic construct
MGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 255 - protein, 386 aa, synthetic construct
MGSIGAVSTEFSCDVFKELRIHHVQENIFYSPVTIISALSMIYLGARDSTKAQIEKAVHFDKIPGFGESIESQCGTSLSIHTSIKDMFTKITKASDNYSIGIASRLYAEEKYPILPEYLQCVKELYKGGLESISFQTAAEQAREIINSWVESQTNGMIKNILQPSSVDPQTDIVLVNAIYFKGLWEKAFRDEDTQTVPFKITEQESKPVQMMYQIGSFKVAEITSEKIKILEVPYASGQLSLWVLLPDDISGLEKLETAITFENLKEWTSSTKMEERKIKVYLPRMKIEEKYNLTSVLTALGITDLFSSSANLSGISSAESLKVSEAFHEAIVEISEAGSKVVGSVGAGVDDTSVSEEFRADHPFLFLIKHNPTSSIFFFGRCFSP

SEQ ID NO: 256 - protein, 381 aa, synthetic construct
MGSIGAASTEFCFDVFKELKVQHVNENIFYSPLSIISALSMVYLGARENTRAQIDKVVHFDKIAGFGESTESQCGTSVSAHTSLKDMSNQITKLSDNYSLSFASRLYAEETYPILPEYSQCVKELYKGGLESISFQTAAYQARELINAWVESQTNGMIKDILQPGSVDSQTKMVLVNAIYFKGIWEKAFKDEDTQEVPFRMTEQETKPVQMMYQIGSFKVAVIAAEKIKILELPYASGQLSMLVILPDDVSGLEQLETAITFEKLTEWTSASVMEERKIKVYLPRMSIEEKYNLTSVLIALGVTDLFSSSANLSGISSAESLRMSEAIHEAFVETYEAGSTESGTEVTSASEEFRVDHPFLFLIKHKPTNSILFFGRCFSP

SEQ ID NO: 257 - protein, 391 aa, synthetic construct
MGSIGAASSEFCFDIFKELKVQHVNENIFYSPLSIISALSMVYLGARENTRAQIDKVVPFDKITASGESIESQVQKIQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 258 - protein, 383 aa, synthetic construct
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGIIKNILQPSSVNPETDMVLVNAIYFKGLWEKAFKDEGTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEINEAGSKVVGAGVDDTSVSEEFRVDRPFLFLIKHNPSNSIFFFGRCFSP

SEQ ID NO: 259 - protein, 386 aa, synthetic construct
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFP

SEQ ID NO: 260 - protein, 392 aa, synthetic construct
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFPHHHHHH

SEQ ID NO: 261 - protein, 386 aa, synthetic construct
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSAVNPETDMVLVNAIYFKGLWEKAFKDEGTQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSKVVGSTGAGVDDTSVSEEFRVDRPFLFLIKHNPSNSIFFFGRCFSP

SEQ ID NO: 262 - protein, 395 aa, synthetic construct
  VARIANT 99: note = Any amino acid
  VARIANT 233: note = Any amino acid
  VARIANT 341: note = Any amino acid
  VARIANT 379: note = Any amino acid
MEDQRGNTGFTMGSIGAASTEFCIDVFRELRVQHVNENIFYSPLTIISALSMVYLGARENTRAQIDQVVHFDKIAGFGDTVESQCGSSPSVHNSLKTVXAQITQPRDNYSLNLASRLYAEESYPILPEYLQCVKELYNGGLETVSFQTAADQARELINSWVESQTNGIIKNILQPSSVDPQTEMVLVNAIYFKGLWEKAFKDEETQAVPFRITEQENRPVQMMYQFGSFKVAXVASEKIKILELPYASGQLSMLVLLPDEVSGLEQNAITFEKLTEWTSSDLMEERKIKVFFPRVKIEEKYNLTAVLVSLGITDLFSSSANLSGISSAENLKMSEAVHEAXVEIYEAGSEVAGSSGAGIEVASDSEEFRVDHPFLFLIXHNPTNSILFFGRCFSP

SEQ ID NO: 263 - protein, 386 aa, synthetic construct
MGSIGAASTEFCIDVFRELRVQHVNENIFYSPLSIISALSMVYLGARENTRAQIDEVFHFDKIAGFGDTVDPQCGASLSVHKSLQNVFAQITQPKDNYSLNLASRLYAEESYPILPEYLQCVKELYNEGLETVSFQTGADQARELINSWVENQTNGVIKNILQPSSVDPQTEMVLVNAIYFKGLWQKAFKDEETQAVPFRITEQENRPVQMMYQFGSFKVAVVASEKVKILELPYASGQLSMWVLLPDEVSGLEQLENAITFEKLTEWTSSDLTEERKIKVFLPRVKIEEKYNLTAVLMALGVTDLFSSSANFSGISAAENLKMSEAVHEAFVEIYEAGSEVVGSSGAGIEAPSDSEEFRADHPFLFLIKHNPTNSILFFGRCFSP

SEQ ID NO: 264 - protein, 386 aa, synthetic construct
MGSIGPLSVEFCCDVFKELRIQHARDNIFYSPVTIISALSMVYLGARDNTKAQIEKAVHFDKIPGFGESIESQCGTSLSVHTSLKDIFTQITKPRENYTVGIASRLYAEEKYPILPEYLQCIKELYKGGLEPISFQTAAEQARELINSWVESQTNGMIKNILQPSSVNPETDMVLVNAIYFKGLWKKAFKDEGTQTVPFRITEQESKPVQMMFQIGSFRVAEITSEKIRILELPYASGQLSLWVLLPDDISGLEQLESAITFENLKEWTSSTKMEERKIKVYLPRMKIEEKYNLTSVLTSLGITDLFSSSANLSGISSAEKLKVSSAFHEASMEIYEAGNKVVGSTGAGVDDTSVSEEFRVDRPFLFLIKHNPSNSIFFFGRCFSP

SEQ ID NO: 265 - protein, 385 aa, synthetic construct
MGSIGAASAEFCVDVFKELKDQHVNNIVFSPLMIISALSMVNIGAREDTRAQIDKVVHFDKITGYGESIESQCGTSIGIYFSLKDAFTQITKPSDNYSLSFASKLYAEETYPILPEYLKCVKELYKGGLETISFQTAADQARELINSWVESQTNGMIKNILQPSSVDPQTEMVLVNAIYFKGLWEKAFKDEDTQAVPFRITEQESKPVQMMYQIGSFKVAVIASEKIKILELPYASGQLSLLVLLPDDVSGLEQLESAITSEKLLEWTNPNIMEERKTKVYLPRMKIEEKYNLTSVLVALGITDLFSSSANLSGISSAEGLKLSDAVHEAFVEIYEAGREVVGSSEAGVEDSSVSEEFKADRPFIFLIKHNPTNGILYFGRYISP

SEQ ID NO: 266 - protein, 386 aa, synthetic construct
MGSIGAANTDFCFDVFKELKVHHANENIFYSPLSIVSALAMVYLGARENTRAQIDKALHFDKILGFGETVESQCDTSVSVHTSLKDMLIQITKPSDNYSFSFASKIYTEETYPILPEYLQCVKELYKGGVETISFQTAADQAREVINSWVESHTNGMIKNILQPGSVDPQTKMVLVNAVYFKGIWEKAFKEEDTQEMPFRINEQESKPVQMMYQIGSFKLTVAASENLKILEFPYASGQLSMMVILPDEVSGLKQLETSITSEKLIKWTSSNTMEERKIRVYLPRMKIEEKYNLKSVLMALGITDLFSSSANLSGISSAESLKMSEAVHEAFVEIYEAGSEVTSSTGTEMEAENVSEEFKADHPFLFLIKHNPTDSIVFFGRCMSP

SEQ ID NO: 267 - protein, 383 aa, synthetic construct
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSSVNPETDMVLVNAIYFKGLWEKAFKDESTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSRVVEAGVDDTSVSEEFRVDRPFLFLIKHNPSNSIFFFGRCFSP

SEQ ID NO: 268 - protein, 386 aa, synthetic construct
MGSIGPVSTEFCCDIFKELRIQHARENIIYSPVTIISALSMVYLGARDNTKAQIEKAVHFDKIPGFGESIESQCGTSLSIHTSLKDILTQITKPSDNYTVGIASRLYAEEKYPILSEYLQCIKELYKGGLEPISFQTAAEQARELINSWVESQTNGMIKNILQPSSVNPETDMVLVNAIYFKGLWEKAFKDEGTQTVPFRITEQESKPVQMMFQIGSFKVAEITSEKIRILELPYASGKLSLWVLLPDDISGLEQLETAITFENLKEWTSSTRMEERKIKVYLPRMKIEEKYNLTSVLTSLGITDLFSSSANLSGISSAERLKVSSAFHEVFVEIYEAGSKVEGSTGAGVDDTSVSEEFRADHPFLFLVKHNPSNSIIFFGRCYLP

SEQ ID NO: 269 - protein, 386 aa, synthetic construct
MGSTGAASMEFCFALFRELKVQHVNENIFFSPVTIISALSMVYLGARENTRAQLDKVAPFDKITGFGETIGSQCSTSASSHTSLKDVFTQITKASDNYSLSFASRLYAEETYPILPEYLQCVKELYKGGLESISFQTAADQARELINSWVESQTNGMIKDILRPSSVDPQTKIILITAIYFKGMWEKAFKEEDTQAVPFRMTEQESKPVQMMYQIGSFKVAVIPSEKLKILELPYASGQLSMLVILPDDVSGLEQLETAITTEKLKEWTSPSMMKERKMKVYFPRMRIEEKYNLTSVLMALGITDLFSPSANLSGISSAESLKVSEAVHEASVDIDEAGSEVIGSTGVGTEVTSVSEEIRADHPFLFLIKHKPTNSILFFGRCFSP

SEQ ID NO: 270 - protein, 2174 aa, synthetic construct
MEHAQLTQLVNSNMTSNTCHEADEFENIDFRMDSISVTNTKFCFDVFNEMKVHHVNENILYSPLSILTALAMVYLGARGNTESQMKKALHFDSITGAGSTTDSQCGSSEYIHNLFKEFLTEITRTNATYSLEIADKLYVDKTFTVLPEYINCARKFYTGGVEEVNFKTAAEEARQLINSWVEKETNGQIKDLLVPSSVDFGTMMVFINTIYFKGIWKTAFNTEDTREMPFSMTKQESKPVQMMCLNDTFNMATLPAEKMRILELPYASGELSMLVLLPDEVSGLEQIEKAINFEKLREWTSTNAMEKKSMKVYLPRMKIEEKYNLTSTLMALGMTDLFSRSANLTGISSVENLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTVISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAMPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQVTAWDNSWIVAHPAAIEPDLCYQIMEQKWKPFDWPDFRLPMRVSCRFRTMEALNKANTSFALDFFKHECQEDDDENILFSPFSISSALATVYLGAKGNTADQMAKTEIGKSGNIHAGFKALDLEINQPTKNYLLNSVNQLYGEKSLPFSKEYLQLAKKYYSAEPQSVDFLGKANEIRREINSRVEHQTEGKIKNLLPPGSIDSLTRLVLVNALYFKGNWATKFEAEDTRHRPFRINMHTTKQVPMMYLRDKFNWTYVESVQTDVLELPYVNNDLSMFILLPRDITGLQKLINELTFEKLSAWTSPELMEKMKMEVYLPRFTVEKKYDMKSTLSKMGIEDAFTKVDSCGVTNVDEITTHIVSSKCLELKHIQINKKLKCNKAVAMEQVSASIGNFTIDLFNKLNETSRDKNIFFSPWSVSSALALTSLAAKGNTAREMAEDPENEQAENIHSGFKELMTALNKPRNTYSLKSANRIYVEKNYPLLPTYIQLSKKYYKAEPYKVNFKTAPEQSRKEINNWVEKQTERKIKNFLSSDDVKNSTKSILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALKSMGMASAFNSNADFSGMTGFQAVPMESLSASTNSFTLDLYKKLDETSKGQNIFFASWSIATALAMVHLGAKGDTATQVAKGPEYEETENIHSGFKELLSAINKPRNTYLMKSANRLFGDKTYPLLPKFLELVARYYQAKPQAVNFKTDAEQARAQINSWVENETESKIQNLLPAGSIDSHTVLVLVNAIYFKGNWEKRFLEKDTSKMPFRLSKTETKPVQMMFLKDTFLIHHERTMKFKIIELPYVGNELSAFVLLPDDISDNTTGLELVERELTYEKLAEWSNSASMMKAKVELYLPKLKMEENYDLKSVLSDMGIRSAFDPAQADFTRMSEKKDLFISKVIHKAFVEVNEEDRIVQLASGRLTGRCRTLANKELSEKNRTKNLFFSPFSISSALSMILLGSKGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 271 - protein, 386 aa, synthetic construct
MGSIGTASTEFCFDMFKEMKVQHANQNIIFSPLTIISALSMVYLGARDNTKAQMEKVIHFDKITGFGESVESQCGTSVSIHTSLKDMLSEITKPSDNYSLSLASRLYAEETYPILPEYLQCMKELYKGGLETVSFQTAADQARELINSWVESQTNGVIKNFLQPGSVDPQTEMVLVNAIYFKGMWEKAFKDEDTQEVPFRITEQESKPVQMMYQVGSFKVATVAAEKMKILEIPYTHRELSMFVLLPDDISGLEQLETTISFEKLTEWTSSNMMEERKVKVYLPHMKIEEKYNLTSVLMALGMTDLFSPSANLSGISTAQTLMMSEAIHGAYVEIYEAGREMASSTGVQVEVTSVLEEVRADKPFLFFIRHNPTNSMVVFGRYMSP

SEQ ID NO: 272 - protein, 2104 aa, synthetic construct
MTSNTCHEADEFENIDFRMDSISVTNTKFCFDVFNEMKVHHVNENILYSPLSILTALAMVYLGARGNTESQMKKALHFDSITGGGSTTDSQCGSSEYIHNLFKEFLTEITRTNATYSLEIADKLYVDKTFTVLPEYINCARKFYTGGVEEVNFKTAAEEARQLMNSWVEKETNGQIKDLLVPSSVDFGTMMVFINTIYFKGIWKTAFNTEDTREMPFSMTKQESKPVQMMCLNDTFNMVTLPAEKMRILELPYASGELSMLVLLPDEVSGLERIEKAINFEKLREWTSTNAMEKKSMKVYLPRMKIEEKYNLTSTLMALGMTDLFSRSANLTGISSVDNLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTIISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAIPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQATAWDNSWIVAHPPAIEPDLYYQIMEQKWKPFDWPDFRLPMRVSCRFRTMEALNKANTSFALDFFKHECQEDDSENILFSPFSISSALATVYLGAKGNTADQMAKVLHFNEAEGARNVTTTIRMQVYSRTDQQRLNRRACFQKTEIGKSGNIHAGFKGLNLEINQPTKNYLLNSVNQLYGEKSLPFSKEYLQLAKKYYSAEPQSVDFVGTANEIRREINSRVEHQTEGKIKNLLPPGSIDSLTRLVLVNALYFKGNWATKFEAEDTRHRPFRINTHTTKQVPMMYLSDKFNWTYVESVQTDVLELPYVNNDLSMFILLPRDITGLQKLINELTFEKLSAWTSPELMEKMKMEVYLPRFTVEKKYDMKSTLSKMGIEDAFTKVDNCGVTNVDEITIHVVPSKCLELKHIQINKELKCNKAVAMEQVSASIGNFTIDLFNKLNETSRDKNIFFSPWSVSSALALTSLAAKGNTAREMAEDPENEQAENIHSGFNELLTALNKPRNTYSLKSANRIYVEKNYPLLPTYIQLSKKYYKAEPHKVNFKTAPEQSRKEINNWVEKQTERKIKNFLSSDDVKNSTKLILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALRSMGMASAFNSNADFSGMTGERDLVISKVCHQSFVAVDEKGTEAAAATAVIAEAVPMESLSASTNSFTLDLYKKLDETSKGQNIFFASWSIATALTMVHLGAKGDTATQVAKGPEYEETENIHSGFKELLSALNKPRNTYSMKSANRLFGDKTYPLLPTKTKPVQMMFLKDTFLIHHERTMKFKIIELPYMGNELSAFVLLPDDISDNTTGLELVERELTYEKLAEWSNSASMMKVKVELYLPKLKMEENYDLKSALSDMGIRSAFDPAQADFTRMSEKKDLFISKVIHKAFVEVNEEDRIVQLASGRLTGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 273 - protein, 386 aa, synthetic construct
MASIGAASTEFCFDVFKELKTQHVKENIFYSPMAIISALSMVYIGARENTRAEIDKVVHFDKITGFGNAVESQCGPSVSVHSSLKDLITQISKRSDNYSLSYASRIYAEETYPILPEYLQCVKEVYKGGLESISFQTAADQARENINAWVESQTNGMIKNILQPSSVNPQTEMVLVNAIYLKGMWEKAFKDEDTQTMPFRVTQQESKPVQMMYQIGSFKVAVIASEKMKILELPYTSGQLSMLVLLPDDVSGLEQVESAITAEKLMEWTSPSIMEERTMKVYLPRMKMVEKYNLTSVLMALGMTDLFTSVANLSGISSAQGLKMSQAIHEAFVEIYEAGSEAVGSTGVGMEITSVSEEFKADLSFLFLIRHNPTNSIIFFGRCISP

SEQ ID NO: 274 - protein, 420 aa, synthetic construct
MGSIGAASTEFCFDVFRELRVQHVNENIFYSPFSIISALAMVYLGARDNTRTQIDKISQFQALSDEHLVLCIQQLGEFFVCTNRERREVTRYSEQTEDKTQDQNTGQIHKIVDTCMLRQDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 275 - protein, 386 aa, synthetic construct
MGSIGAASAEFCLDIFKELKVQHVNENIIFSPMTIISALSLVYLGAKEDTRAQIEKVVPFDKIPGFGEIVESQCPKSASVHSSIQDIFNQIIKRSDNYSLSLASRLYAEESYPIRPEYLQCVKELDKEGLETISFQTAADQARQLINSWVESQTNGMIKNILQPSSVNSQTEMVLVNAIYFRGLWQKAFKDEDTQAVPFRITEQESKPVQMMQQIGSFKVAEIASEKMKILELPYASGQLSMLVLLPDDVSGLEKLESSITVEKLIEWTSSNLTEERNVKVYLPRLKIEEKYNLTSVLAALGITDLFSSSANLSGISTAESLKLSRAVHESFVEIQEAGHEVEGPKEAGIEVTSALDEFRVDRPFLFVTKHNPTNSILFLGRCLSP

SEQ ID NO: 276 - protein, 386 aa, synthetic construct
MGSISAASGEFCLDIFKELKVQHVNENIFYSPMVIVSALSLVYLGARENTRAQIDKVIPFDKITGSSEAVESQCGTPVGAHISLKDVFAQIAKRSDNYSLSFVNRLYAEETYPILPEYLQCVKELYKGGLETISFQTAADQAREIINSWVESQTDGKIKNILQPSSVDPQTKMVLVSAIYFKGLWEKSFKDEDTQAVPFRVTEQESKPVQMMYQIGSFKVAAIAAEKIKILELPYASEQLSMLVLLPDDVSGLEQLEKKISYEKLTEWTSSSVMEEKKIKVYLPRMKIEEKYNLTSILMSLGITDLFSSSANLSGISSTKSLKMSEAVHEASVEIYEAGSEASGITGDGMEATSVFGEFKVDHPFLFMIKHKPTNSILFFGRCISP

SEQ ID NO: 277 - protein, 386 aa, synthetic construct
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILPEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETAITFENLKEWTSSSKMEERKIRVYLPRMKIEEKYNLTSVLKSLGITDLFSSSANLSGISSAESLKVSAAFHEASVEIYEAGSKGVGSSEAGVDGTSVSEEIRADHPFLFLIKHNPSDSILFFGRCFSP

SEQ ID NO: 278 - protein, 386 aa, synthetic construct
MGSIGAASTEFCFDVFKELKVQHVNENIIISPLSIISALSMVYLGAREDTRAQIDKVVHFDKITGFGEAIESQCPTSESVHASLKETFSQLTKPSDNYSLAFASRLYAEETYPILPEYLQCVKELYKGGLETINFQTAAEQARQVINSWVESQTDGMIKSLLQPSSVDPQTEMILVNAIYFRGLWERAFKDEDTQELPFRITEQESKPVQMMSQIGSFKVAVVASEKVKILELPYASGQLSMLVLLPDDVSGLEQLESSITVEKLIEWISSNTKEERNIKVYLPRMKIEEKYNLTSVLVALGITDLFSSSANLSGISSAESLKISEAVHEAFVEIQEAGSEVVGSPGPEVEVTSVSEEWKADRPFLFLIKHNPTNSILFFGRYISP

SEQ ID NO: 279 - protein, 386 aa, synthetic construct
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILQEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETSITFENLKEWTSSSKMEERKIRVYLPRMKIEEKYNLTSVLKSLGITDLFSSSANLSGISSAESLKVSAVFHEASVEIYEAGSKGVGSSEAGVDGTSVSEEIRADHPFLFLIKHNPSDSILFFGRCFSP

SEQ ID NO: 280 - protein, 399 aa, synthetic construct
MLNLMHPKQFCCTMGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIASRLYAEEKYPILPEYIQCVKELYKGGLESISFQTAAEKSRELINSWVESQTNGTIKNILQPSSVSSQTDMVLVSAIYFKGLWEKAFKEEDTQTVPFRITEQESKPVQMMSQIGTFKVAEIPSEKCRILELPYASGRLSLWVLLPDDISGLEQLETAITSENLKEWTSSSKMEERKIKVYLPRMKIEEKYNLTSVLKSLGITDLFSSSANLSGISSAESLKVSGAFHEAFVEIYEAGSKAVGSSGAGVEDTSVSEEIRADHPFLFFIKHNPSDSILFFGRCFSP

SEQ ID NO: 281 - protein, 389 aa, synthetic construct
EAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 282 - protein, 474 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 283 - protein, 389 aa, synthetic construct
EAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 284 - protein, 474 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 285 - protein, 426 aa, synthetic construct
TRAPDCGGILTPLGLSYLAEVSKPHAEVVLRQDLMAQRASDLFLGSMEPSRNRITSVKVADLWLSVIPEAGLRLGIEVELRIAPLHAVPMPVRISIRADLHVDMGPDGNLQLLTSACRPTVQAQSTREAESKSSRSILDKVVDVDKLCLDVSKLLLFPNEQLMSLTALFPVTPNCQLQYLPLAAPVFSKQGIALSLQTTFQVAGAVVPVPVSPVPFSMPELASTSTSHLILALSEHFYTSLYFTLERAGAFNMTIPSMLTTATLAQKITQVGSLYHEDLPITLSAALRSSPRVVLEEGRAALKLFLTVHIGAGSPDFQSFLSVSADVTAGLQLSVSDTRMMISTAVIEDAELSLAASNVGLVRAALLEELFLAPVCQQVPAWMDDVLREGVHLPHLSHFTYTDVNVVVHKDYVLVPCKLKLRSTMA

SEQ ID NO: 286 - protein, 388 aa, synthetic construct; identical to SEQ ID NO: 209

SEQ ID NO: 287 - protein, 1185 aa, synthetic construct
CSTWGGGHFSTFDKYQYDFTGTCNYIFATVCDESSPDFNIQFRRGLDKKIARIIIELGPSVIIVEKDSISVRSVGVIKLPYASNGIQIAPYGRSVRLVAKLMEMELVVMWNNEDYLMVLTEKKYMGKTCGMCGNYDGYELNDFVSEGKLLDTYKFAALQKMDDPSEICLSEEISIPAIPHKKYAVICSQLLNLVSPTCSVPKDGFVTRCQLDMQDCSEPGQKNCTCSTLSEYSRQCAMSHQVVFNWRTENFCSVGKCSANQIYEECGSPCIKTCSNPEYSCSSHCTYGCFCPEGTVLDDISKNRTCVHLEQCPCTLNGETYAPGDTMKAACRTCKCTMGQWNCKELPCPGRCSLEGGSFVTTFDSRSYRFHGVCTYILMKSSSLPHNGTLMAIYEKSGYSHSETSLSAIIYLSTKDKIVISQNELLTDDDELKRLPYKSGDITIFKQSSMFIQMHTEFGLELVVQTSPVFQAYVKVSAQFQGRTLGLCGNYNGDTTDDFMTSMDITEGTASLFVDSWRAGNCLPAMERETDPCALSQLNKISAETHCSILTKKGTVFETCHAVVNPTPFYKRCVYQACNYEETFPYICSALGSYARTCSSMGLILENWRNSMDNCTITCTGNQTFSYNTQACERTCLSLSNPTLECHPTDIPIEGCNCPKGMYLNHKNECVRKSHCPCYLEDRKYILPDQSTMTGGITCYCVNGRLSCTGKLQNPAESCKAPKKYISCSDSLENKYGATCAPTCQMLATGIECIPTKCESGCVCADGLYENLDGRCVPPEECPCEYGGLSYGKGEQIQTECEICTCRKGKWKCVQKSRCSSTCNLYGEGHITTFDGQRFVFDGNCEYILAMDGCNVNRPLSSFKIVTENVICGKSGVTCSRSISIYLGNLTIILRDETYSISGKNLQVKYNVKKNALHLMFDIIIPGKYNMTLIWNKHMNFFIKISRETQETICGLCGNYNGNMKDDFETRSKYVASNELEFVNSWKENPLCGDVYFVVDPCSKNPYRKAWAEKTCSIINSQVFSACHNKVNRMPYYEACVRDSCGCDIGGDCECMCDAIAVYAMACLDKGICIDWRTPEFCPVYCEYYNSHRKTGSGGAYSYGSSVNCTWHYRPCNCPNQYYKYVNIEGCYNCSHDEYFDYEKEKCMPCAMQPTSVTLPTATQPTSPSTSSASTVLTETTNPPV

SEQ ID NO: 288 - protein, 129 aa, synthetic construct [sequence omitted]

SEQ ID NO: 289 - protein, 129 aa, synthetic construct
KVFGRCELAAAMKRHGLDNYRGYSLGNWVCVAKFESNFNTQATNRNTDGSTDYGILQINSRWWCNDGRTPGSRNLCNIPCSALLSSDITASVNCAKKIVSDGNGMSAWVAWRNRCKGTDVQAWIRGCRL

SEQ ID NO: 290 - protein, 130 aa, synthetic construct
KVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 291 - protein, 129 aa, synthetic construct
KVFERCELARTLKKLGLDGYKGVSLANWLCLTKWESSYNTKATNYNPSSESTDYGIFQINSKWWCNDGKTPNAVDGCHVSCRELMENDIAKAVACAKHIVSEQGITAWVAWKSHCRDHDVSSYVEGCTL

SEQ ID NO: 292 - protein, 449 aa, synthetic construct
IEVNCSLYASGIGKDGTSWVACPRNLKPVCGTDGSTYSNECGICLYNREHGANVEKEYDGECRPKHVMIDCSPYLQVVRDGNTMVACPRILKPVCGSDSFTYDNECGICAYNAEHHTNISKLHDGECKLEIGSVDCSKYPSTVSKDGRTLVACPRILSPVCGTDGFTYDNECGICAHNAEQRTHVSKKHDGKCRQEIPEIDCDQYPTRKTTGGKLLVRCPRILLPVCGTDGFTYDNECGICAHNAQHGTEVKKSHDGRCKERSTPLDCTQYLSNTQNGEAITACPFILQEVCGTDGVTYSNDCSLCAHNIELGTSVAKKHDGRCREEVPELDCSKYKTSTLKDGRQVVACTMIYDPVCATNGVTYASECTLCAHNLEQRTNLGKRKNGRCEEDITKEHCREFQKVSPICTMEYVPHCGSDGVTYSNRCFFCNAYVQSNRTLNLVSMAAC

SEQ ID NO: 293 - protein, 139 aa, synthetic construct
MAGARGCVVLLAAALMLVGAVLGSEDRSRLLGAPVPVDENDEGLQRALQFAMAEYNRASNDKYSSRVVRVISAKRQLVSGIKYILQVEIGRTTCPKSSGDLQSCEFHDEPEMAKYTTCTFVVYSIPWLNQIKLLESKCQ

SEQ ID NO: 294 - protein, 450 aa, synthetic construct
SEVCFPRLGCFSDDAPWAGIVQRPLKILPWSPKDVDTRFLLYTNQNQNNYQELVADPSTITNSNFRMDRKTRFIIHGFIDKGEEDWLSNICKNLFKVESVNCICVDWKGGSRTGYTQASQNIRIVGAEVAYFVEVLKSSLGYSPSNVHVIGHSLGSHAAGEAGRRTNGTIERITGLDPAEPCFQGTPELVRLDPSDAKFVDVIHTDAAPIIPNLGFGMSQTVGHLDFFPNGGKQMPGCQKNILSQIVDIDGIWEGTRDFVACNHLRSYKYYADSILNPDGFAGFPCDSYNVFTANKCFPCPSEGCPQMGHYADRFPGKTNGVSQVFYLNTGDASNFARWRYKVSVTLSGKKVTGHILVSLFGNEGNSRQYEIYKGTLQPDNTHSDEFDSDVEVGDLQKVKFIWYNNNVINPTLPRVGASKITVERNDGKVYDFCSQETVREEVLLTLNPC

SEQ ID NO: 295 - protein, 455 aa, synthetic construct
GLVAADRITGGKDFRDIESKFALRTPEDTAEDTCHLIPGVTESVANCHFNHSSKTFVVIHGWTVTGMYESWVPKLVAALYKREPDSNVIVVDWLSRAQQHYPVSAGYTKLVGQDVAKFMNWMADEFNYPLGNVHLLGYSLGAHAAGIAGSLTSKKVNRITGLDPAGPNFEYAEAPSRLSPDDADFVDVLHTFTRGSPGRSIGIQKPVGHVDIYPNGGTFQPGCNIGEALRVIAERGLGDVDQLVKCSHERSVHLFIDSLLNEENPSKAYRCNSKEAFEKGLCLSCRKNRCNNMGYEINKVRAKRSSKMYLKTRSQMPYKVFHYQVKIHFSGTESNTYTNQAFEISLYGTVAESENIPFTLPEVSTNKTYSFLLYTEVDIGELLMLKLKWISDSYFSWSNWWSSPGFDIGKIRVKAGETQKKVIFCSREKMSYLQKGKSPVIFVKCHDKSLNRKSG

SEQ ID NO: 296 - protein, 685 aa, synthetic construct
APKKGVRWCVISTAEYSKCRQWQSKIRRTNPMFCIRRASPTDCIRAIAAKRADAVTLDGGLVFEADQYKLRPVAAEIYGTEENPQTYYYAVAVVKKGFNFQLNQLQGRKSCHTGLGRSAGWNIPIGLLRRFLDWAGPPEPLQKAVAKFFSQSCVPCADGNAYPNLCQLCIGKGKDKCACSSQEPYFGYSGAFNCLHKGIGDVAFVKESTVFENLPQKADRDKYELLCPDNTRKPVEAFRECHLARVPSHAVVARSVNGKENSIWELLYQSQKKFGKSNPQEFQLFGSPGQQKDLLFRDATIGFLKIPSKIDSKLYLGLPYLTAIQGLRETAAEVEARQAKVVWCAVGPEELRKCRQWSSQSSQNLNCSLASTTEDCIVQVLKGEADAMSLDGGFIYTAGKCGLVPVLAENQKSRQSSSSDCVHRPTQGYFAVAVVRKANGGITWNSVRGTKSCHTAVDRTAGWNIPMGLLVNQTGSCKFDEFFSQSCAPGSQPGSNLCALCVGNDQGVDKCVPNSNERYYGYTGAFRCLAENAGDVAFVKDVTVLDNTNGQNTEEWARELRSDDFELLCLDGTRKPVTEAQNCHLAVAPSHAVVSRKEKAAQVEQVLLTEQAQFGRYGKDCPDKFCLFRSETKNLLFNDNTEVLAQLQGKTTYEKYLGSEYVTAIANLKQCSVSPLLEACAFMMR

SEQ ID NO: 297 - protein, 689 aa, synthetic construct
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 298 - protein, 66 aa, synthetic construct
APVNTTTEDETAQIPAEAVIGYSDLEGDFDVAVLPFSNSTNNGLLFINTTIASIAAKEEGVSLDKR

SEQ ID NO: 299 - protein, 70 aa, synthetic construct
APVNTTTEDETAQIPAEAVIGYSDLEGDFDVAVLPFSNSTNNGLLFINTTIASIAAKEEGVSLDKREAEA

SEQ ID NO: 300 - protein, 1405 aa, synthetic construct
MTIAHHCIFLVILAFLALINVASGAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSATEACLPAGQRKSGMNINFYQYSLKDSSTYSNAAYMAYGYASKTKLGSVGGQTDISIDYNIPCVSSSGTFPCPQEDSYGNWGCKGMGACSNSQGIAYWSTDLFGFYTTPTNVTLEMTGYFLPPQTGSYTFSFATVDDSAILSVGGSIAFECCAQEQPPITSTNFTINGIKPWDGSLPDNITGTVYMYAGYYYPLKVVYSNAVSWGTLPISVELPDGTTVSDNFEGYVYSFDDDLSQSNCTIPDPSIHTTSTITTTTEPWTGTFTSTSTEMTTITDTNGQLTDETVIVIRTPTTASTITTTTEPWTGTFTSTSTEMTTVTGTNGQPTDETVIVIRTPTSEGLITTTTEPWTGTFTSTSTEMTTVTGTNGQPTDETVIVIRTPTSEGLITTTTEPWTGTFTSTSTEVTTITGTNGQPTDETVIVIRTPTSEGLITTTTEPWTGTFTSTSTEMTTVTGTNGQPTDETVIVIRTPTSEGLISTTTEPWTGTFTSTSTEVTTITGTNGQPTDETVIVIRTPTSEGLITTTTEPWTGTFTSTSTEMTTVTGTNGQPTDETVIVIRTPTSEGLITRTTEPWTGTFTSTSTEVTTITGTNGQPTDETVIVIRTPTTAISSSLSSSSGQITSSITSSRPIITPFYPSNGTSVISSSVISSSVTSSLVTSSSFISSSVISSSTTTSTSIFSESSTSSVIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHSSSVVSVSETGNTMSLTSSGLSTMSQQPRSTPASSMVGSSTASLEISTYAGSANSLLAGSGLSVFIASLLLAII

SEQ ID NO: 301 - protein, 24 aa, synthetic construct
GSSGSSGSSGSSGSSGSSGSSGSS

SEQ ID NO: 302 - protein, 15 aa, synthetic construct
GGGGSGGGGSGGGGS

SEQ ID NO: 303 - DNA, 3059 bp, synthetic construct
  source 1..3059: mol_type = other DNA
tgtcaaaacagtagtgataaaaggctatgaaggaggttgtctaggggctcgcggaggaaagtgattcaaacagacctgccaaaaagagaaaaaagagggaatccctgttctttccaatggaaatgacgtaactttaacttgaaaaataccccaaccagaagggttcaaactcaacaaggattgcgtaattcctacaagtagcttagagctgggggagagacaactgaaggcagcttaacgataacgcggggggattggtgcacgactcgaaaggaggtatcttagtcttgtaacctcttttttccagaggctattcaagattcataggcgatatcgatgtggagaagggtgaacaatataaaaggctggagagatgtcaatgaagcagctggatagatttcaaattttctagatttcagagtaatcgcacaaaacgaaggaatcccaccaagacaaaaaaaaaaattctaaggaattccgaaacgatgagattcccttctattttcactgctgttttgttcgctgcttcttctgctttggctgctccagttaacactactaccgaagacgaaactgctcaaattcctgctgaagctgttattggttactctgacttggaaggtgacttcgacgttgctgttttgccattctctaactctactaacaacggtttgttgttcattaacactactattgcttctattgctgctaaggaagaaggtgtttctttggacaagagagaggccgaagctgctccagcccccgtaaagcagggaccaacctctgtagcttacgtagaggtaaataacaactccatgttaaacgtaggaaaatacacactggccgacggaggaggcaacgcatttgacgtagctgtgatattcgccgctaatattaactatgacacgggcaccaagacagcatatctacatttcaacgagaacgtacaaagagtgcttgacaacgccgtaacccaaatccgtcctcttcagcaacaaggaattaaggtcttgttgtctgttttaggaaatcatcagggcgctggattcgctaactttccctctcagcaagcagcctccgcatttgctaaacaattgtcagacgccgtcgccaagtacggcttagacggagttgacttcgatgacgagtatgctgaatacggaaacaatggcaccgcacagccaaacgactcctcttttgttcatcttgtcaccgctctacgtgccaatatgccagataaaattatttccctttataatataggtcctgctgcttctcgtttgtcctacggcggtgtagatgtctctgacaagtttgattacgcctggaatccttattacggcacgtggcaggtccccggaatcgcacttcctaaggcccagctatctcctgcagcagttgaaatcggcagaacgtctcgttccaccgtggcagatttagcaaggagaacagtcgatgaaggatatggagtgtacttaacgtataatcttgatggcggcgataggaccgcagacgtatctgcctttactagagagttgtatggaagtgaggccgttcgtactcccggttcatcagggtcctcaggatcatccggtagtagtggttcatccggttcatccggatcaagtggctcctctgaagctgcagcaagggaggctgcagcccgtgaggcagccgctagagaagccgccgctaggggtggtggcggctctggcggaggcggttccggtggcggaggctctcaaatcaacgaattgaacgttgttttagatgatgttaagaccaacattgccgactacatcaccctatcctacactccaaattcaggtttttccttggaccaaatgccagctggtattatggatattgctgcgcaattggttgcaaatccaagtgatgactcctacaccactttgtactctgaagtggacttttctgctgttgagcatatgttgactatggtcccatggtactcttctagactgcttccagaattagaagcaatggatgcttctctaactacctcaagttctgctgccacatcttcaagtgaagttgctagctcttctattgcttcatccactagctcttctgttgcaccatcctcaagtgaagttgtcagctcttccgttgcttcatcctcaagtgaagttgccagctcctctgttgcgtctacaagcgaagctactagttcttctgctgtcacatcttcctccgctgtttcctcttcgaccgagtctgttagctcttcctctgtcagttcttcctcagccgtttcctcttctgaagctgtcagttcctctccagtttcctcagttgtttcatcttcggccggacctgctagctcaagcgttgctccttacaactcaaccattgctagctcttcttccactgcccagacttctatctcgaccattgctccttacaactccacaaccaccaccaccccagctagttctgcttccagcgttattatctcaaccagaaacggtaccactgttactgaaactgacaacactcttgtcaccaaagaaaccactgtctgtgactactcttcaacatctgccgttccagcttccaccaccggttacaacaattctactaaggtttcaaccgctactatctgcagtacatgcaaagaaggtacctctactgcaactgacttctctacactaaagactacagttaccgtatgtgactccgcctgtcaagctaagaagtctgctaccgttgttagcgttcaatctaaaactaccggtatcgttgaacaaaccgaaaacggtgctgccaaggctgttatcggtatgggtgccggtgctttagctgctgttgccgccatgctactatgagcggccgctcgatttgtatgtgaaatagctgaaattcgaaaatttcattatggctgtatctactttagcgtattaggcatttgagcattggcttgaacaatgcgggctgtagtgtgtcaccaaagaaaccattcgggttcggatctggaagtcctcatcacgtgatgccgatctcgtgtattttattttcagataacacctgaagacttt

SEQ ID NO: 304 - DNA, 2792 bp, synthetic construct
  source 1..2792: mol_type = other DNA
tgtcaaaacagtagtgataaaaggctatgaaggaggttgtctaggggctcgcggaggaaagtgattcaaacagacctgccaaaaagagaaaaaagagggaatccctgttctttccaatggaaatgacgtaactttaacttgaaaaataccccaaccagaagggttcaaactcaacaaggattgcgtaattcctacaagtagcttagagctgggggagagacaactgaaggcagcttaacgataacgcggggggattggtgcacgactcgaaaggaggtatcttagtcttgtaacctcttttttccagaggctattcaagattcataggcgatatcgatgtggagaagggtgaacaatataaaaggctggagagatgtcaatgaagcagctggatagatttcaaattttctagatttcagagtaatcgcacaaaacgaaggaatcccaccaagacaaaaaaaaaaattctaaggaattccgaaacgatgagattcccttctattttcactgctgttttgttcgctgcttcttctgctttggctgctccagttaacactactaccgaagacgaaactgctcaaattcctgctgaagctgttattggttactctgacttggaaggtgacttcgacgttgctgttttgccattctctaactctactaacaacggtttgttgttcattaacactactattgcttctattgctgctaaggaagaaggtgtttctttggacaagagagaggccgaagctgctccagcccccgtaaagcagggaccaacctctgtagcttacgtagaggtaaataacaactccatgttaaacgtaggaaaatacacactggccgacggaggaggcaacgcatttgacgtagctgtgatattcgccgctaatattaactatgacacgggcaccaagacagcatatctacatttcaacgagaacgtacaaagagtgcttgacaacgccgtaacccaaatccgtcctcttcagcaacaaggaattaaggtcttgttgtctgttttaggaaatcatcagggcgctggattcgctaactttccctctcagcaagcagcctccgcatttgctaaacaattgtcagacgccgtcgccaagtacggcttagacggagttgacttcgatgacgagtatgctgaatacggaaacaatggcaccgcacagccaaacgactcctcttttgttcatcttgtcaccgctctacgtgccaatatgccagataaaattatttccctttataatataggtcctgctgcttctcgtttgtcctacggcggtgtagatgtctctgacaagtttgattacgcctggaatccttattacggcacgtggcaggtccccggaatcgcacttcctaaggcccagctatctcctgcagcagttgaaatcggcagaacgtctcgttccaccgtggcagatttagcaaggagaacagtcgatgaaggatatggagtgtacttaacgtataatcttgatggcggcgataggaccgcagacgtatctgcctttactagagagttgtatggaagtgaggccgttcgtactcccggttcatcagggtcctcaggatcatccggtagtagtggttcatccggttcatccggatcaagtggctcctctgaagctgcagcaagggaggctgcagcccgtgaggcagccgctagagaagccgccgctaggggtggtggcggctctggcggaggcggttccggtggcggaggctctgcttctgtgacgacaacgctgtctccctacgatgagagagtcaacctaatagagttagcagtatatgtatcagacattggagcacacttaagtgagtattacgccttccaagccttgcataaaacagaaacatatccaccagagattgcaaaagccgtttttgctggaggagactttaccacaatgcttacaggtataagtggtgatgaggtaacccgtatgattactggagttccctggtactccacaaggcttatgggagccatatcagaagctttggcaaacgaaggaatagccacggctgtacccgcctctaccacggaggctagttctaccagtacgtccgaagcttcatctgccgccaccgaatcctcctcatccagtgagtctagtgcagaaacgtcttctaatgcagcatctacacaagctactgtgtcttccgaatcatccagtgcagcctctaccatagcatcatccgccgaatcttctgttgcatcaagtgtagcatcttcagttgcatctagtgcttcttttgcaaacaccactgctcctgtttcttctacctcatccatcagtgtgaccccagtggtccaaaacggcactgattccaccgtgacaaagacacaagcctcaacagtagagactacgataacgtcttgctccaacaacgtgtgctcaacagtcacgaagcccgtaagtagtaaggcacagagtacagccacttcagtcacttcatcagctagtagagttatagatgttactacaaatggcgcaaataagtttaataatggcgtgttcggcgcagccgctattgctggtgccgcagcattattactatgagcggccgctcgatttgtatgtgaaatagctgaaattcgaaaatttcattatggctgtatctactttagcgtattaggcatttgagcattggcttgaacaatgcgggctgtagtgtgtcaccaaagaaaccattcgggttcggatctggaagtcctcatcacgtgatgccgatctcgtgtattttattttcagataacacctgaagacttt

SEQ ID NO: 305 - DNA, 2915 bp, synthetic construct
  source 1..2915: mol_type = other DNA
tgtcaaaacagtagtgataaaaggctatgaaggaggttgtctaggggctcgcggaggaaagtgattcaaacagacctgccaaaaagagaaaaaagagggaatccctgttctttccaatggaaatgacgtaactttaacttgaaaaataccccaaccagaagggttcaaactcaacaaggattgcgtaattcctacaagtagcttagagctgggggagagacaactgaaggcagcttaacgataacgcggggggattggtgcacgactcgaaaggaggtatcttagtcttgtaacctcttttttccagaggctattcaagattcataggcgatatcgatgtggagaagggtgaacaatataaaaggctggagagatgtcaatgaagcagctggatagatttcaaattttctagatttcagagtaatcgcacaaaacgaaggaatcccaccaagacaaaaaaaaaaattctaaggaattccgaaacgatgagattcccttctattttcactgctgttttgttcgctgcttcttctgctttggctgctccagttaacactactaccgaagacgaaactgctcaaattcctgctgaagctgttattggttactctgacttggaaggtgacttcgacgttgctgttttgccattctctaactctactaacaacggtttgttgttcattaacactactattgcttctattgctgctaaggaagaaggtgtttctttggacaagagagaggccgaagctgctccagcccccgtaaagcagggaccaacctctgtagcttacgtagaggtaaataacaactccatgttaaacgtaggaaaatacacactggccgacggaggaggcaacgcatttgacgtagctgtgatattcgccgctaatattaactatgacacgggcaccaagacagcatatctacatttcaacgagaacgtacaaagagtgcttgacaacgccgtaacccaaatccgtcctcttcagcaacaaggaattaaggtcttgttgtctgttttaggaaatcatcagggcgctggattcgctaactttccctctcagcaagcagcctccgcatttgctaaacaattgtcagacgccgtcgccaagtacggcttagacggagttgacttcgatgacgagtatgctgaatacggaaacaatggcaccgcacagccaaacgactcctcttttgttcatcttgtcaccgctctacgtgccaatatgccagataaaattatttccctttataatataggtcctgctgcttctcgtttgtcctacggcggtgtagatgtctctgacaagtttgattacgcctggaatccttattacggcacgtggcaggtccccggaatcgcacttcctaaggcccagctatctcctgcagcagttgaaatcggcagaacgtctcgttccaccgtggcagatttagcaaggagaacagtcgatgaaggatatggagtgtacttaacgtataatcttgatggcggcgataggaccgcagacgtatctgcctttactagagagttgtatggaagtgaggccgttcgtactcccggttcatcagggtcctcaggatcatccggtagtagtggttcatccggttcatccggatcaagtggctcctctgaagctgcagcaagggaggctgcagcccgtgaggcagccgctagagaagccgccgctaggggtggtggcggctctggcggaggcggttccggtggcggaggctctcaattctcaaattcaacatcagcctcctccactgatgttacttcaagttcctctatcagtacatcatctggttcagtgacaatcacctctagtgaagccccagaatcagataacggaacaagtacggctgcacccacagaaacctctacggaggcaccaacaactgccatccctactaacggcacctctactgaagcacctacgactgcaatcccaacaaatggtacgtccactgaggctcctaccgataccaccactgaagctccaaccactgctttgcctacaaacggcacttccacagaggctcctactgatacaactactgaagctccaaccacaggccttcctactaatggcactacatccgcattcccccctacgacctccctgccacctagtaacactactacgactcccccttacaatcctagtaccgactacactacggattacacggttgttactgagtacacaacctactgccccgagcccactacattcacaacgaacggcaaaacgtacactgtaacggaacctacgacgctgacaattaccgattgtccttgtacaatagaaaaacccacaaccacgagtacaacagaatataccgtcgtcacagaatatactacttattgtccagaaccaactaccttcaccacgaacggtaaaacttataccgttactgagcccacgactcttacaataactgattgtccctgcactatcgaaaaatctgaagcaccagagtcatctgtccctgtgactgaatctaagggaacgaccactaaggaaaccggagtaacaacaaagcagactacggccaatccctcactgactgtctccacggtcgtacccgtgtcatcatccgccagttctcatagtgtcgttatcaactcaaacggagctaatgtggttgtacctggcgcattaggacttgctggtgtggccatgttgttcctgtaagcggccgctcgatttgtatgtgaaatagctgaaattcgaaaatttcattatggctgtatctactttagcgtattaggcatttgagcattggcttgaacaatgcgggctgtagtgtgtcaccaaagaaaccattcgggttcggatctggaagtcctcatcacgtgatgccgatctcgtgtattttattttcagataacacctgaagacttt

SEQ ID NO: 306 - protein, 409 aa, synthetic construct
MLTKVISLAILTASAFADSGEFTLWNLSPGDPYDSTFWGVSEGLIVPVEPGVTFVITDDLQLKTTDDQFVTVGEDSALGLGAEGSVEFSIINEDGITSLYYNGELVTAYICEGAEPQIYLTGSEEDPECVSYTVAVIGVDGEAPPTFPEEDDETTTTDDPTDEPTDEPTDEPTDEPTDEPTDEPTDEPTDEPTDEPTDEPTDEPTDEPTDEPTDEPTDEPTDEPTEEPTEEPTEEPTDEPTPPPPHWGNETVTATKTEYETTKVTITSCEETKCYETTSDAWVSTCTTEIGGKVTKIVTWCPIPSTPGPKPPKPTKPTETKPTTVPAPTTKKPETPTTKKPETPAPEKPEKTTTVIPPPTTEKPSTLSTSSVTGSVTIPTITATGGAGSNFNLGGLTVGVAGIAMALFV

SEQ ID NO: 307 - protein, 471 aa, synthetic construct
MFEKSKFVVSFLLLLQLFCVLGVHGQESGNGTTSDTAYACDIGATPFDGFNATIYQYQASDDNSIQDPVFMSTGYLQRNQLHSTTGVTNPGFNIFTAGVATTTLYGIPNVNYQNMLLELKGYFRADASGNYGLSLRNIDDSAILFFGRETAFECCNENLIPLDEAPTDYSLFTIKEGEASTNPDSYTYTQYLEAGRYYPVRTFFANIRTRAVFNFTMTLPDGSELTDFQNYIFQFGALNQQQCQAEIVTRENYTTTTEPWTGTFEATTTVIPSGTEPGTVIVQTPYSTIDSTSTWTGTFTTFTTDADGSTIAVVPSSTIDDHFASTETVLTDTAISTTVITVTSCGTSKCTKTTALTGVTQRTLTIDDRTTVVTTYCPLPTDVATIKTASVSGSEVVQTIYTAKHSQAVSYVHPSTVTITREVCDAQTCTQATIVTGEILQTTVVDSGSTTVVPKYVPVETHEPTFELSTL

SEQ ID NO: 308 - protein, 233 aa, synthetic construct
MQFTFASTSVVVSLIAALAKPAVATPPACLLACAAEVVKESSDCDALNNIQCICENEGSAIHACLESTCPDGLSSTALQSFEDVCESVGTEANLDESSSSQSSSSSSSSESSSSSVSSSSSSASSSSETSSSVTSSSVTSSSTAVSSSTESSSSVEPSTSHSSSHSSSEVSSTVAPTTSVAPTTSSITTSSTSLTSATTSSVTISIEPTSDAADKVIIPGLAGLVGALAVGLI

SEQ ID NO: 309 - protein, 203 aa, synthetic construct
MQYRSLFLGSALLAAANAAVYNTTVTDVVSELETTVLTITSCAEDKCITSKSTGLITTSTLTKHGVVTVVTTVCDLPSTTKSYVPPAKTTTIPPPEKTTTTVPPPAKTTTTVPPPAKTTSTVPPPAKTSSHHESTITVTVPSSTSTKKIETESTTYHFVTQTTTARNITPPAITTQSHGAAGMNAANFVGLGAAAVAAAALVL

SEQ ID NO: 310 - protein, 439 aa, synthetic construct
MSLLLFLVLGAFLLSSVKAADIGAFRLRVYTPGRFTNGALNFNNWGYQYLDASSSNGQLFAGYATVTSVTTFLAPDDEGFVWGSSLGGYPGFLGIGAGATAFHLTGIPGDALSWYIEDNILKTSSPTYVCSRNDGDVVVGIEANTRWLAMHDTSQLPPNYYCFQADYEIVALWYIPDTTSTWTGTETSTTTDDDGSVIELVPTPLPDTTSTWTGTFTTFTTDDDGSVIELVPTPLPDSTSTWTGTYTTFTTDEDGSTIAVVPSSTIDSTSTWTGTYTTFTTDEDGSTIAVVPSSTIDSTSTWTGTYTTFTTDEDGSTIAVYHHLLSTPHPPGLVLTPRSLPMRMEVLLLWYHHLLSTLHPPGLVLTPRSLPMRMEVLLLWYHRLLSTPHPGLVLTPRSLPMRMEVLLLYHHLLSTPHPPGLVLTPRSLPMRMEVLLLWY

SEQ ID NO: 311 - protein, 2140 aa, synthetic construct
MKLQLQSFVFFLLSAVNVLADDSYGCSIATSPRSTGFVANLYEFPNMAISNAELKTYVRYRYKEGRLYDTISNIISPYFYYQGQGANSAYGTLYGRPNVYLYNFSMELKGYFRPPITGQYTIDFNGANVDDAAMVFFGKAGAFDCCNSDYILPEQSAEYSLYSVYPHTATDQILSATIYLEAGKYYPLRVTYTNIGNIGSLDLRVVLPSGASITSLGAFVYQFPNNLSPGTCTPDVEYFTTTTQAWTGTYETTYTVPPSGTQPGTVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTEPGTVIIETPESYVTTTQPWTGTYETTYTVPPSGTEPGIVIIETPESYVTTTQPWTGTYETTYTVPPSGTEPGTVVIETPEITDCEAVCCGAVPTSDPLRRRDVCDCETFCCPGDTNCETYVTTTQPWTGTYETTYTVPPSGTEPGTVIIETPESYVTTTQPWTGTYETTYTVPPSGTEPGIVIIETPESYVTTTQPWTGTYETTYTVPPTGTEPGTVIIETPESYVTTTQPWTGTYETTYTVPPSGTEPGIVIIETPESYVTTTQPWTGTYETTYTVPPSGTEPGTVIIETPESYVTTTQPWTGTYETTYTVPPTGTEPGTVIIETPESYVTTTQPWTGTYETTYTVPPSGTEPGIVIIETPESYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTEPGTVVIETPEITDCEAVCCGAVPTSDPLRRRDVCDCETFCCPGDTNCETYVTTTQPWTGTYETTYTVPPSGTEPGTVIIETPESYVTTTQPWTGTYETTYTVPPTGTEPGTVIIETPESYVTTTQPWTGTYETTYTVPPSGTQPGTVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTQPGTVIIETPESYVTTTQPWTGTYETTYTVPPTGTEPGTVIIETPESYVTTTQPWTGTYETTYTVPPSGTEPGIVIIETPESYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTEPGTVVIETPEITDCEAVCCGAVPTSDPLRRRDVCDCETFCCPGDTNCETYVTTTQPWTGTYETTYTVPPSGTEPGTVIIETPESYVTTTQPWTGTYETTYTVPPTGTEPGTVIIETPESYVTTTQPWTGTYETTYTVPPSGTQPGTVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTQPGTVIIETPESYVTTTQPWTGTYETTYTVPPTGTEPGTVIIETPESYVTTTQPWTGTYETTYTVPPSGTEPGIVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTQPGTVIIETPESYVTTTQPWTGTYETTYTVPPSGTEPGTVIVETPDVPGSYVTTTQPWTGTYETTHTVPPTGTEPGTVVVETPDVPGSYVTTTQPWTGTYETTHTVPPTGTEPGTVVVETPDVPGSYVTTTQPWTGTYETTYTVPPSGTEPGTVIVETPDVPGSYVTTTQPWTGTYETTHTVPPTGTEPGTVVVETPDVPGSYVTTTQPWTGVYKTTYTVPPSGTIPGTVIIETPFGYFNTSSISTKTDKRTITSVVPCSQCSESKTQYITPTGPGDVTVIISQPPSKITLSSPEDKTKTDFITSTGSIGGGSPPSHPNDKPGIITTPTQPIGGGNPSDIPSAISSVSSGGNSRASVPSFSTSSAISVQVSSLYDENSGSTFEVSLLFSVVSGFFLTLMV

SEQ ID NO: 312 - protein, 1149 aa, synthetic construct
MKFPVPLLFLLQLFFIIATQGDESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCYETTVSKITEWTTYTTPWTGTFETTRTITPTGTEGTVVIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVVIETPEIVDCEAYCCASVAIKKRELCQCENFCCSWDQSCQTYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIINCEAVCCGPFLTAFSFRKREECQCENICCPGDTNCETYVTTTQPWTGTYETTYTVPPTGTEPGTVIIETPESYVTTTQPWTGTYETTYTVPSTGTEPGTVIIETPESYVTTTQPWTGTYETTFTVPPTGTEPGTVVIETPESYVTTTQPWTGTYETTYSVPPSGTEPGTVVIETPEASTARTKFTTVTSSWTGVFTTTKTLPASGTEPATIVIQTPTGYFNTSSLVSTRTKTNVDTVTRVIPCPICTAPKTITVVPEEPNESVSVIISQPQSSSTDTTLSKPDSVRVISQPETASQMDTSLSKTDSAVISTETAGNNIIPLAGSHSYNTIVTTVTDSPQVAQSTTATSSSNVHLTISTQTTTPSLVYSSSLSTVHQVSPSNGGFRSSITVHPLLSVIGAIFGALFM

SEQ ID NO: 313 - protein, 715 aa, synthetic construct
MTKFTILLLVLLKFYSILAIEVDGSANGQPLAHPIVVEVHEATKWITHTSPWTGTPEAIRTVTGETPYEQKIARYDEFNPRLANREIIDCVAFCCGDATSSPSITEPESTATELPESYVTINRPWSLSWIPDVPPGSPYWSTSTIPPSGTEPGTVIIYFYLYDDARKRREINFGSTQPYHGRPKLLGSIEKRELCQCDAVCCLGDLSCEVYVTTTQPWTGTYETTYTITPTGSEPGTVIIETPELYVTTTQPWTGTYETTYTITPTGSEPGTVIIETPESYVTTTQPWTGTYETTYTITPTGSEPGTVIIETPESYVTTTQPWTGTYETTYTITPTGSEPGTVIIETPESYVTTTQPWTGTYETTYTVPPSGTEPGAVIIETPELYVTTTQPWTGTYETTYTITPTGSEPGTVIIETPESYVTTTQPWTGTYETTYTVPPSGTEPGTVIIETPELYVTTTQPWTGTYETTYTITPTGSEPGTVIVEIPVSYVNSTQISTSTYDTTDTVLSSGVEPGTIAIETPIVYLNTSVSAFSRPWTKIDTVTQFSSCAVCSKPETITVTPENPIDTVTIIISQPQSTSQSNTPTSFKANSTSAFSRFDEDSIPVFGSYSYEITVNIDVNTEDDTTTNLNADTTIIIGSLSAIRTVAGSSSNYHASNISPTINSQKTASSVVVHSDSSATVYQFSPSNGAPWLSVQISTLLSVVGTLLAAVLL

SEQ ID NO: 314 - protein, 2498 aa, synthetic construct
MNFRYLLILPIYASIVLGQVGDFQLLLNAKEPIRNSPSLLSSNYGNLTLPAMANGALESHFDYGNAYVGDDQITVVYHLPDEHGQINAYRQDTDEYIGYLGLVTDDYGEYTYLSVIMPGVQYDQTTSVNWYIENEELKSTSINVQPLLGCYYKNPPQYSWYWASIDEPGNIASSNFVCEPCKVYVDFVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADTTSVWTGSYTTWTTDEDGTVIEQVPTPSADTPSADTTSVWTGSYTTWTTDEDGTVIEQVPTPSADTTSVWTGSYTTWTTDEDGTVIEQVPTPSADTPSADTTSVWTGSYTTWTTEVGDGGSSTVVELVPTESSTSTNVMQTPVPSSGVSDGVSVFNGFNVEVFHYPADNYELANEISFLSYGYENLGLVTTVTGVSDINFDTDSNWPYYIDRDALGNTGSYVNATIEYEGFFRAPVDGEYVFSFSSTDYNSILFVGSPAAADQALQKREVQFLKPETSPDYVLLFNNTRDLGKTVSTTQYLLADQYYPLRVVIAAISQHALLDFQIKLPNGASLTQYQGYVYNFALEGSESTTVIGDKTSTWTGSYTTWTTDSDGSTIVVVPPATITADKTSTWTGSYTTWTTDSDGSTVVICPSITSDHNDKPSESTLTDSSISTTVVTVTSCDIEKCTKTTALTGVRETTLTTGGTTTVVTTYCPLPTDIVTVKTTSIDGSEVLQTIYTAKPNHVVPDVQTSTVTITREVCDAFTCTHATIVTGEILKTTTLADTHYTTVVPVYVPLETYQPAVELSTLETVLKSSDLASGPVVTAGSVQPSYQSGGVAESSLTVSEFEAHSTSDTVSQPSTISLQTGEANALKWSSFFGAALVPLVNVFFV

SEQ ID NO: 315 - protein, 2812 aa, synthetic construct
MQNTNDKLIIRTFYSISTIHGLLSINIFSDTRVYKFAIYSTDAVSLEPRTKNNMSLVTVLACFIIFAAHAFGQDTFYMLKVRTLTPNGYPLADSLSNPMQYWDLYYVPGGPRRLESSFVNWQPTTAAPINQFYCRLGTDGHMTGYNRVTGSVIGKLSFGTNAATALAFGSYDGDPSYPPQAFSISSSVSGTMTYLNVHYVNARSITWYSTTTATGETNVYINVASTGYTGDRTTYQAELWVEPFVPNIPVDTTTSIWTGSQTSYTTEVGENGGSTVIELIPTPPADATSTWTGTYTTRTTDADGSVIEQIPTPSADTTSVWTGTYTTWTTDADGSVIEQIPTPSADTTSVWTGTYTTWTTDADGSVIEQIPTPSADTTATWTGTETSYTTDVGEDGSSTVIELVPTPSADTTATWTGTETSYTTDVGEDGSSTVVELVPTPSADTTATWTGTETSYTTDVGEDGSSTVIELVPTPSADTTATWTGTETSYTTDVGEDGSSTVIELVPTPSADTTATWTGTETSYTTDVGEDGSSTVVELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPTPSADTTATWTGTETSYTTDVGEDGSSTVIELVPTPSADTTATWTGTETSYTTDVGEDGSSTVIELVPTPSADTTATWTGTETSYTTDVGEDGSSTVVELVPTPTPSADTTATWTGTETSYTTDVGEDGSSTVVELVPTPSADTTATWTGTETSYTTDVGEDGSSTVIELVPTPSADTTATWTGTETSYTTDVGEDGSSTVVELVPTPSADTTATWTGTETSYTTDVGEDGSSTVVELVPTPTADTTATWTGTETSYTTDVGEDGSSTVIELVPTPSADTTATWTGTETSYTTDVGEDGSSTVVELVPTPSADTTATWTGTETSYTTDVGEDGSSTVVELVPTPSADTTATWTGTETSYTTDVGEDGSSTVIELVPTPSADTTATWTGTETSYTTDVGEDGSSTVVELVPTPSADTTATWTGTETSYTTDVGEDGSSTVVELVPTPTADTTATWTGTETSYTTDVGEDGSSTVIELVPTPSADTTATWTGTETSYTTDVGEDGSSTVVELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPTPSADTTATWTGTETSYTTDVGEDGSSTVIELVPTPSADTTATWTGTETSYTTDVGEDGSSTVIELVPTPTPSADTTATWTGTETSYTTDVGEDGSSTVVELVPTPSADTTATWTGTETSYTTDVGEDGSSTVIELVPTPSADTTATWTGTETSYTTDVGEDGSSTVVELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVVELVPTPTPSADTTATWTGTETSYTTDVGEDGSSTVIELVPSDTETATNIVETPVPSSGVSDGVSVFDGFNVEVFHYPADNYELANEIGFLSYGYENLGLVTNATGVSDINFDTDSNWPYYIDRDALGNTGSYVNATIEYEGFFRAPVDGEYVFSFSNTDYNSILFVGSPAAAGQALQKRRVQFLKPETSPDHVLLFNNTRDLGQTISTTQYLLADQYYPLRVVIAAISQHALLDFQIKLPNGALLTQYQGYVYNFALEGSESTTVIGDKTSTWTGSYTTWTTDSDGSTVVVVPSATITADKTSTWTGSYTTWTTDSDGSTIVICPSITSDHNDKPSESTLTDGSISTTVVTVTSCDIEKCTKTTALTGVTETTLTTGGTTTVVTTYCPLPTDIVTVKTTSISGSEVLQTIYTAKPSHVVPNVHTLTVTITREVCDAFTCTQATIVTGEILKTTTLADTHSTTVVPVYVPLESYQSAVELSTLETVLKSSDFASGSAVTAGSAQPSYQSGGVAESSLTGSELEAHSTSDTVSQPSTISPQTGEANALRWSSFFGAALVPLVNVFFV

SEQ ID NO: 316 - protein, 860 aa, synthetic construct
MTKLTILLSVLLQLFSVLAEVPKKTEWSSHTTYWTSTLEALRTVTPTGTERAVIGEAPYEYKLIGNDQFDPGLNAKREIIDCEAVCCGAVPTSDPLKRRDVCECENVCCPGDDCETYVTTTQPWTGTYETTYTVPPSGTEPGTVVIETPEITDCEAVCCGAVPTSDPLRRRDVCECENVCCPGDDCETYVTTTQPWTGTYETTYTVPPSGTEPGTVVIETPEITDCEAVCCGAVPTSDPLRRRDVCECENVCCPGDDCETYVTTTQPWTGTYETTYTVPPTGTEPGTVVIETPVTYVTTTQPWTGTYETTYTVPPTGTEPGTVVIETPEITDCEAVCCGAVPTSDPLRRRDVCECENVCCPGDDCETYVTTTQPWTGTYETTYTVPPTGTEPGTVVIETPVTYVTTTQPWTGTYETTYTVPPTGTEPGTVVIETPVTYVTTTQPWTGTYETTYTVPPTGTEPGTVVIETPVTYVTTTQPWTGTYETTYTIPPTGTEPGTVVIETPEITDCEAVCCGAVPTSDPLRRRDVCECENVCCPGDDCETYVTTTQPWTGTYETTYTVPPTGTEPGTVVIETPVTYVTTTQPWTGTYETTYTVPPTGTEPGTVVIETPVTYVTTTQPWTGTYETTYTVPPTGTEPGTVVIETPVTYVTTTKPWTGTYETTHTVPASGTEPGTVIIETPIKYLNTSISASTSTWTKINTVTQFISCPVCTIPKTITVTPKISNETVTIIISQPHGTSSRTTTVVKTDGASVSSHSYKTALTTDVKPEEKTSTKLGTVTTVSGSHSAIDTVTGSLSDYHASSIPHTVKSEEKASSTVTHTISSSTVYQVSPSNGASWLSVRLNTALSIIGTLFAAVFI

SEQ ID NO: 317 - protein, 1436 aa, synthetic construct
MSKTKNGGSEFVHIAYVFHIEASTPSDYINMIQIVLFPHQAQITKRMNLVTLLVCNLLCVSLTLGQGVYRLKFPALVVTGRESVGTTVVNYDFLVGNTGQYGDLGEFFYDGEPYYCWNSTDSQPLSCSSSSSLLISTQNVTISHPDEDGTVYAYAERDGGLLGRFTVGSVSADWPQWAVIVYSTSSSAHPSSWYVDDNKLKLTSGLGPNNSTTLQACYFTQSSGRDRYAISLEGSPAYTGQVSCQATEFDLEFIPPSADTTSIWDGSYTTWTTDSNGIVVEQIPTPSADTTSIWTGSETSWTTDSDGTVIELVPTPSADATSIWTGDHTTWTTDSEGNVIEQIPTPSADTTSIWTGSETSWTTDSDGTVIELVPTPSADATSIWTGDHTTWTTDSEGNVIEQIPTPSADATSIWTGDHTTWTTDREGNVIEQIPTPSADTTSIWTGSETSWTTDSDGTVIELVPTPSADATSIWTGDHTTWTTDSEGNVIEQIPTPSADATSIWTGSETSWTTDSDGTVIELVPTPSADATSIWTGDHTTWTTDSEGNVIEQIPTPSADATSIWTGDHTTWTTDSEGNVIEQIPTPSADTTSIWTGSETSWTTDSDGTVIELVPTPSADATSIWTGDHTTWTTDSEGNVIEQIPTPSADATSIWTGDHTTWTTDSEGNVIEQIPTPSADTTSIWTGSETSWTTDSDGTVIELVPTPSADATSIWTGDHTTWTTDSEGNVIEQIPTPSADATSIWTGDHTTWTTDSEGNVIEQIPTPSADTTSIWTGSETSWTTDSDGTVIELVPTPSADATSIWTGDHTTWTTDSEGNVIEQIPTPSADATSIWTGSETSWTTDSDGTVIELVPTPSADATSIWTGDHTTWTTDSEGNVIEQIPTPSADATSIWTGDHTTWTTDSEGNVIEQIPTPSADTTSIWTGDHTTWTTEVGGDGSSIVVELVPSETGTATNVVQTPVPSSGISDGVSALDGFNVEVFHYPADNYELANEISFLSYGYENLGLVTTATGVSDINFDTDSNWPSYIDRNALGNTGSYVNATIKYEGFFRAPVDGDYEFSFSNIDYNSILFVGSAAADQALRKREAQFLKPETSPNHILFFNNSRDVGQTISTTQYLSADSYYPLRVVIAAVSQHALLDFQIKLPNGVSLTQFQGYVYNFALEGAESTTVIGDKTSTWTGTYTTWTTDSEGSTIVLCPSIISDHNGKPADTTLTDGSISTTVVTVTSCDIKKCTKTTALTGVTQKTLTVKGTTTVVTAYCPLPTDVATVKTISVGGSEVLQTVYTAKPSHIVPDVQTLTVTITREVCDALTCIPATIVTGEILKTTTLADTHSTTVIPVYVPLETHQPALDLITLETVLKSSDFANGPAITSVSVESLSHQSGVVVSEFDSDSTSGAVSQPSSAVSLQTGKASALKWSPFLGAAVISLFNVFFV

SEQ ID NO: 318 - protein, 2435 aa, synthetic construct
MNLFTILAWGFLYVPLVLGEGYYSLNFDARVPIALGILGSSYQKYTIMADRSLLGGSNIDLDVTFSGIIELLTNRVHIVVSLPDADGRVSVYDMYSGTSLGYLSFVCSLTTCEVHAVSSSSGATTWTLDGNQLIPTSPSTVYACYRSLVGLLAQYTLNDRTSITAQCEQTNLYVELAIPAFPETTAVWTGTYTTWTTDESGSVIEQMPTPSADTTTTWTGTYTTWTTDADGSVIEQIPTPPADTTSVWTGTYTTRTTDADGSVIEQIPTPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSIWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSIWTGTYTTWTTDADGSVIEQIPTPSADTTSVWTGTYTTWTTDADGSVIEQIPTPSADTTLAPSADTTSIWTGTYTTWTTDADGSVIEQIPTPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQSPTPSAYTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQSPTPSAYTTSVWTGTYTTWTTDADGSVIEQIPTPSADTTLAPSADTTSIWTGTYTTWTTDADGSVIEQIPTPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTLAPSADTTSIWTGTYTTWTTDADGSVIEQIPTPSADTTSVWTGTYTTWTTDAAGTVIEVIPSGTSISSDVIPTPLPTSGVDIDTIPYDAFNVAVYHYPADNYELANNLGFLTSGYEGLGQVTTATSVGNINFDTSSGWPYYIESNALGNTGSYVNATIEYVGFFQAPANGNYELSFSNIDYNAILFLGSPATDSSLAKREVQFLKPETSSEYVLFFDHGKDAGQTVSTTQYLSAGLYYPLRIVLAAVSERAQLDFQITLPDGRVLDQYQGYVYNFAHEGIESATSSAHETSWSRFTNSTIYSHSSTIGIITSSTDAPHSVINPTAIETTSTDTSISTVAVTTSICDTKDCVKTTVITPNSPLPTQTVSLTTTTIDRSEVVQTAHSAVPSQFAPDAHPSAVTITREQCDAYSCSQATIVSGKVLQTTTVSDSTTVVPLDTPQLSVEASTLETRLKSTQSSRAPTVTVQTSQSSRHSEDVTESSVHVSEFDAQSTSATSASALQAPSSISLQTGGANTLRLSAFLGTALLPMLNVLFI

SEQ ID NO: 319 - protein, 135 aa, synthetic construct
MQFSIVATLALAGSALAAYSNVTYTYETTITDVVTELTTYCPEPTTFVHKNKTITVTAPTTLTITDCPCTISKTTKITTDVPPTTHSTPHTTTTHVPSTSTPAPTHSVSTISHGGAAKAGVAGLAGVAAAAAYFL

SEQ ID NO: 320 - protein, 219 aa, synthetic construct
MLLTSLLQVFACCLVLPAQVTAFYYYTSGAERKCFHKELSKGTLFQATYKAQIYDDQLQNYRDAGAQDFGVLIDIEETFDDNHLVVHQKGSASGDLTFLASDSGEHKICIQPEAGGWLIKAKTKIDVEFQVGSDEKLDSKGKATIDILHAKVNVLNSKIGEIRREQKLMRDREATFRDASEAVNSRAMWWIVIQLIVLAVTCGWQMKHLGKFFVKQKIL

SEQ ID NO: 321 - protein, 215 aa, synthetic construct
MIKSTIALPSFFIVLILALVNSVAASSSYAPVAISLPAFSKECLYYDMVTEDDSLAVGYQVLTGGNFEIDFDITAPDGSVITSEKQKKYSDFLLKSFGVGKYTFCFSNNYGTALKKVEITLEKEKTLTDEHEADVNNDDIIANNAVEEIDRNLNKITKTLNYLRAREWRNMSTVNSTESRLTWLSILIIIIIAVISIAQVLLIQFLFTGRQKNYV

SEQ ID NO: 322 - protein, 203 aa, synthetic construct [sequence omitted]

SEQ ID NO: 323 - protein, 211 aa, synthetic construct [sequence omitted]

SEQ ID NO: 324 - protein, 225 aa, synthetic construct
MSNLCVLFFQFFFLAQFFAEASPLTFELNKGRKECLYTLTPEIDCTISYYFAVQQGESNDFDVNYEIFAPDDKNKPIIERSGERQGEWSFIGQHKGEYAICFYGGKAHDKIVDLDFKYNCERQDDIRNERRKARKAQRNLRDSKTDPLQDSVENSIDTIERQLHVLERNIQYYKSRNTRNHHTVCSTEHRIVMFSIYGILLIIGMSCAQIAILEFIFRESRKHNV

SEQ ID NO: 325 - protein, 212 aa, synthetic construct
MKYNIVHGICLLFAITQAVGAVHFYAKSGETKCFYEHLSRGNLLIGDLDLYVEKDGLFEEDPESSLTITVDETFDNDHRVLNQKNSHTGDVTFTALDTGEHRFCFTPFYSKKSATLRVFIELEIGNVEALDSKKKEDMNSLKGRVGQLTQRLSSIRKEQDAIREKEAEFRNQSESANSKIMTWSVFQLLILLGTCAFQLRYLKNFFVKQKVV

SEQ ID NO: 326 - protein, 1163 aa, synthetic construct
DESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCYETTVSKITEWTTYTTPWTGTFETTRTITPTGTEGTVVIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVVIETPEIVDCEAYCCASVAIKKRELCQCENFCCSWDQSCQTYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIINCEAVCCGPFLTAFSFRKREECQCENICCPGDTNCETYVTTTQPWTGTYETTYTVPPTGTEPGTVIIETPESYVTTTQPWTGTYETTYTVPSTGTEPGTVIIETPESYVTTTQPWTGTYETTFTVPPTGTEPGTVVIETPESYVTTTQPWTGTYETTYSVPPSGTEPGTVVIETPESYVTTTQPWTGTYETTYSVPPSGTEPGTVVIETPEASTARTKFTTVTSSWTGVFTTTKTLPASGTEPATIVIQTPTGYFNTSSLVSTRTKTNVDTVTRVIPCPICTAPKTITVVPEEPNESVSVIISQPQSSSTDTTLSKPDSVRVISQPETASQMDTSLSKTDSAVISTETAGNNIIPLAGSHSYNTIVTTVTDSPQVAQSTTATSSSNVHLTISTQTTTPSLVYSSSLSTVHQVSPSNGGFRSSITVHPLLSVIGAIFGALFM

SEQ ID NO: 327 - protein, 1184 aa, synthetic construct
MKFPVPLLFLLQLFFIIATQGDESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCYETTVSKITEWTTYTTPWTGTFETTRTITPTGTEGTVVIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVVIETPEIVDCEAYCCASVAIKKRELCQCENFCCSWDQSCQTYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIINCEAVCCGPFLTAFSFRKREECQCENICCPGDTNCETYVTTTQPWTGTYETTYTVPPTGTEPGTVIIETPESYVTTTQPWTGTYETTYTVPSTGTEPGTVIIETPESYVTTTQPWTGTYETTFTVPPTGTEPGTVVIETPESYVTTTQPWTGTYETTYSVPPSGTEPGTVVIETPESYVTTTQPWTGTYETTYSVPPSGTEPGTVVIETPEASTARTKFTTVTSSWTGVFTTTKTLPASGTEPATIVIQTPTGYFNTSSLVSTRTKTNVDTVTRVIPCPICTAPKTITVVPEEPNESVSVIISQPQSSSTDTTLSKPDSVRVISQPETASQMDTSLSKTDSAVISTETAGNNIIPLAGSHSYNTIVTTVTDSPQVAQSTTATSSSNVHLTISTQTTTPSLVYSSSLSTVHQVSPSNGGFRSSITVHPLLSVIGAIFGALFM

SEQ ID NO: 328 - protein, 1092 aa, synthetic construct
SSGKTCPTSEVSPACYANQWETTFPPSDIKITGATWVQDNIYDVTLSYEAESLELENLTELKIIGLNSPTGGTKLVWSLNSKVYDIDNPAKWTTTLRVYTKSSADDCYVEMYPFQIQVDWCEAGASTDGCSAWKWPKSYDYDIGCDNMQDGVSRKHHPVYKWPKKCSSNCGVEPTTSDEPEEPTTSEEPEEPTTSEEPEEPTSSDEEPTTSEEPEEPTTSDEPEEPTTSEEPEEPTTSEEPEEPTTSEEPTTSEEPEEPTSSDEEPTTSDEPEEPTTSDEPEEPTTSEEPTTSEEPEEPTTSSEEPTPSEEPEGPTCPTSEVSPACYADQWETTFPPSDIKITGATWVEDNIYDVTLSYEAESLELENLTELKIIGLNSPTGGTKVVWSLNSGIYDIDNPAKWTTTLRVYTKSSADDCYVEMYPFQIQVDWCEAGASTDGCSAWKWPKSYDYDIGCDNMQDGVSRKHHPVYKWPKKCSSDCGVEPTTSDEPEEPTTSEEPVEPTSSDEEPTTSEEPTTSEEPEEPTTSDEPEEPTTSEEPEEPTTSEEPEEPTTSEEPTTSEEPEEPTSSDEEPTTSDEPEEPTTSEEPEEPTTSEEPEEPTTSEEPEEPTTSDEPEEPTTSEEPEEPTTSEEPEEPTSSDEEPTTSEEPEEPTTSEEPEEPTTSEEPEEPTTSEEPEEPTSSDEEPTTSEEPEEPTTSDEPEEPTTSEEPEEPTTSEEPEEPTSSDEEPTTSEEPEEPTTSDEPEEPTTSEEPEEPTTSEEPEEPTTSEEPEEPTTSEEPEEPTSSDEEPTTSEEPEEPTTSDEPEEPTTSEEPEEPTTSEEPEEPTTSEEPEEPTTSDEEPGTTEEPLVPTTKTETDVSTTLLTVTDCGTKTCTKSLVITGVTKETVTTHGKTTVITTYCPLPTETVTPTPVTVTSTIYADESVTKTTVYTTGAVEKTVTVGGSSTVVVVHTPLTTAVVQSQSTDEIKTVVTARPSTTTIVRDVCYNSVCSVATIVTGVTEKTITFSTGSITVVPTYVPLVESEEHQRTASTSETRATSVVVPTVVGQSSSASATSSIFPSVTIHEGVANTVKNSMISGAVALLFNALFL

SEQ ID NO: 329 - protein, 1114 aa, synthetic construct
MVSLRSIFTSSILAAGLTRAHGSSGKTCPTSEVSPACYANQWETTFPPSDIKITGATWVQDNIYDVTLSYEAESLELENLTELKIIGLNSPTGGTKLVWSLNSKVYDIDNPAKWTTTLRVYTKSSADDCYVEMYPFQIQVDWCEAGASTDGCSAWKWPKSYDYDIGCDNMQDGVSRKHHPVYKWPKKCSSNCGVEPTTSDEPEEPTTSEEPEEPTTSEEPEEPTSSDEEPTTSEEPEEPTTSDEPEEPTTSEEPEEPTTSEEPEEPTTSEEPTTSEEPEEPTSSDEEPTTSDEPEEPTTSDEPEEPTTSEEPTTSEEPEEPTTSSEEPTPSEEPEGPTCPTSEVSPACYADQWETTFPPSDIKITGATWVEDNIYDVTLSYEAESLELENLTELKIIGLNSPTGGTKVVWSLNSGIYDIDNPAKWTTTLRVYTKSSADDCYVEMYPFQIQVDWCEAGASTDGCSAWKWPKSYDYDIGCDNMQDGVSRKHHPVYKWPKKCSSDCGVEPTTSDEPEEPTTSEEPVEPTSSDEEPTTSEEPTTSEEPEEPTTSDEPEEPTTSEEPEEPTTSEEPEEPTTSEEPTTSEEPEEPTSSDEEPTTSDEPEEPTTSEEPEEPTTSEEPEEPTTSEEPEEPTTSDEPEEPTTSEEPEEPTTSEEPEEPTSSDEEPTTSEEPEEPTTSEEPEEPTTSEEPEEPTTSEEPEEPTSSDEEPTTSEEPEEPTTSDEPEEPTTSEEPEEPTTSEEPEEPTSSDEEPTTSEEPEEPTTSDEPEEPTTSEEPEEPTTSEEPEEPTTSEEPEEPTTSEEPEEPTSSDEEPTTSEEPEEPTTSDEPEEPTTSEEPEEPTTSEEPEEPTTSEEPEEPTTSDEEPGTTEEPLVPTTKTETDVSTTLLTVTDCGTKTCTKSLVITGVTKETVTTHGKTTVITTYCPLPTETVTPTPVTVTSTIYADESVTKTTVYTTGAVEKTVTVGGSSTVVVVHTPLTTAVVQSQSTDEIKTVVTARPSTTTIVRDVCYNSVCSVATIVTGVTEKTITFSTGSITVVPTYVPLVESEEHQRTASTSETRATSVVVPTVVGQSSSASATSSIFPSVTIHEGVANTVKNSMISGAVALLFNALFL

SEQ ID NO: 330 - protein, 217 aa, synthetic construct
DESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSC

SEQ ID NO: 331 - protein, 551 aa, synthetic construct
EAEADESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCGSSGSSGSSGSSGSSGSSGSSGSSEAAAREAAAREAAAREAAARGGGGSGGGGSGGGGSAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTP

SEQ ID NO: 332 - protein, 636 aa, synthetic construct
MRFPSIFTAVLFAASSALAAPVNTTTEDETAQIPAEAVIGYSDLEGDFDVAVLPFSNSTNNGLLFINTTIASIAAKEEGVSLDKREAEADESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCGSSGSSGSSGSSGSSGSSGSSGSSEAAAREAAAREAAAREAAARGGGGSGGGGSGGGGSAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTP

SEQ ID NO: 333 - protein, 829 aa, synthetic construct
EAEADESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCGSSGSSGSSGSSGSSGSSGSSGSSEAAAREAAAREAAAREAAARGGGGSGGGGSGGGGSAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSDESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCGS

SEQ ID NO: 334 - protein, 914 aa, synthetic construct
MRFPSIFTAVLFAASSALAAPVNTTTEDETAQIPAEAVIGYSDLEGDFDVAVLPFSNSTNNGLLFINTTIASIAAKEEGVSLDKREAEADESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCGSSGSSGSSGSSGSSGSSGSSGSSEAAAREAAAREAAAREAAARGGGGSGGGGSGGGGSAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSDESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCGS

SEQ ID NO: 335 - protein, 1075 aa, Saccharomyces cerevisiae
MTIAHHCIFLVILAFLALINVASGATEACLPAGQRKSGMNINFYQYSLKDSSTYSNAAYMAYGYASKTKLGSVGGQTDISIDYNIPCVSSSGTFPCPQEDSYGNWGCKGMGACSNSQGIAYWSTDLFGFYTTPTNVTLEMTGYFLPPQTGSYTFSFATVDDSAILSVGGSIAFECCAQEQPPITSTNFTINGIKPWDGSLPDNITGTVYMYAGYYYPLKVVYSNAVSWGTLPISVELPDGTTVSDNFEGYVYSFDDDLSQSNCTIPDPSIHTTSTITTTTEPWTGTFTSTSTEMTTITDTNGQLTDETVIVIRTPTTASTITTTTEPWTGTFTSTSTEMTTVTGTNGQPTDETVIVIRTPTSEGLITTTTEPWTGTFTSTSTEMTTVTGTNGQPTDETVIVIRTPTSEGLITTTTEPWTGTFTSTSTEVTTITGTNGQPTDETVIVIRTPTSEGLITTTTEPWTGTFTSTSTEMTTVTGTNGQPTDETVIVIRTPTSEGLISTTTEPWTGTFTSTSTEVTTITGTNGQPTDETVIVIRTPTSEGLITTTTEPWTGTFTSTSTEMTTVTGTNGQPTDETVIVIRTPTSEGLITRTTEPWTGTFTSTSTEVTTITGTNGQPTDETVIVIRTPTTAISSSLSSSSGQITSSITSSRPIITPFYPSNGTSVISSSVISSSVTSSLVTSSSFISSSVISSSTTTSTSIFSESSTSSVIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHSSSVVSVSETGNTMSLTSSGLSTMSQQPRSTPASSMVGSSTASLEISTYAGSANSLLAGSGLSVFIASLLLAII

SEQ ID NO: 336 - protein, 634 aa, synthetic construct
EAEAAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSN

SEQ ID NO: 337 - protein, 739 aa, synthetic construct; identical to SEQ ID NO: 26

SEQ ID NO: 338 - protein, 1493 aa, synthetic construct
APAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSDESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCYETTVSKITEWTTYTTPWTGTFETTRTITPTGTEGTVVIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVVIETPEIVDCEAYCCASVAIKKRELCQCENFCCSWDQSCQTYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIINCEAVCCGPFLTAFSFRKREECQCENICCPGDTNCETYVTTTQPWTGTYETTYTVPPTGTEPGTVIIETPESYVTTTQPWTGTYETTYTVPSTGTEPGTVIIETPESYVTTTQPWTGTYETTFTVPPTGTEPGTVVIETPESYVTTTQPWTGTYETTYSVPPSGTEPGTVVIETPESYVTTTQPWTGTYETTYSVPPSGTEPGTVVIETPEASTARTKFTTVTSSWTGVFTTTKTLPASGTEPATIVIQTPTGYFNTSSLVSTRTKTNVDTVTRVIPCPICTAPKTITVVPEEPNESVSVIISQPQSSSTDTTLSKPDSVRVISQPETASQMDTSLSKTDSAVISTETAGNNIIPLAGSHSYNTIVTTVTDSPQVAQSTTATSSSNVHLTISTQTTTPSLVYSSSLSTVHQVSPSNGGFRSSITVHPLLSVIGAIFGALFM

SEQ ID NO: 339 - protein, 1514 aa, synthetic construct
MKFPVPLLFLLQLFFIIATQGAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSDESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCYETTVSKITEWTTYTTPWTGTFETTRTITPTGTEGTVVIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVVIETPEIVDCEAYCCASVAIKKRELCQCENFCCSWDQSCQTYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIINCEAVCCGPFLTAFSFRKREECQCENICCPGDTNCETYVTTTQPWTGTYETTYTVPPTGTEPGTVIIETPESYVTTTQPWTGTYETTYTVPSTGTEPGTVIIETPESYVTTTQPWTGTYETTFTVPPTGTEPGTVVIETPESYVTTTQPWTGTYETTYSVPPSGTEPGTVVIETPESYVTTTQPWTGTYETTYSVPPSGTEPGTVVIETPEASTARTKFTTVTSSWTGVFTTTKTLPASGTEPATIVIQTPTGYFNTSSLVSTRTKTNVDTVTRVIPCPICTAPKTITVVPEEPNESVSVIISQPQSSSTDTTLSKPDSVRVISQPETASQMDTSLSKTDSAVISTETAGNNIIPLAGSHSYNTIVTTVTDSPQVAQSTTATSSSNVHLTISTQTTTPSLVYSSSLSTVHQVSPSNGGFRSSITVHPLLSVIGAIFGALFM

SEQ ID NO: 340 - protein, 1422 aa, synthetic construct
APAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSSSGKTCPTSEVSPACYANQWETTFPPSDIKITGATWVQDNIYDVTLSYEAESLELENLTELKIIGLNSPTGGTKLVWSLNSKVYDIDNPAKWTTTLRVYTKSSADDCYVEMYPFQIQVDWCEAGASTDGCSAWKWPKSYDYDIGCDNMQDGVSRKHHPVYKWPKKCSSNCGVEPTTSDEPEEPTTSEEPEEPTTSEEPEEPTSSDEEPTTSEEPEEPTTSDEPEEPTTSEEPEEPTTSEEPEEPTTSEEPTTSEEPEEPTSSDEEPTTSDEPEEPTTSDEPEEPTTSEEPTTSEEPEEPTTSSEEPTPSEEPEGPTCPTSEVSPACYADQWETTFPPSDIKITGATWVEDNIYDVTLSYEAESLELENLTELKIIGLNSPTGGTKVVWSLNSGIYDIDNPAKWTTTLRVYTKSSADDCYVEMYPFQIQVDWCEAGASTDGCSAWKWPKSYDYDIGCDNMQDGVSRKHHPVYKWPKKCSSDCGVEPTTSDEPEEPTTSEEPVEPTSSDEEPTTSEEPTTSEEPEEPTTSDEPEEPTTSEEPEEPTTSEEPEEPTTSEEPTTSEEPEEPTSSDEEPTTSDEPEEPTTSEEPEEPTTSEEPEEPTTSEEPEEPTTSDEPEEPTTSEEPEEPTTSEEPEEPTSSDEEPTTSEEPEEPTTSEEPEEPTTSEEPEEPTTSEEPEEPTSSDEEPTTSEEPEEPTTSDEPEEPTTSEEPEEPTTSEEPEEPTSSDEEPTTSEEPEEPTTSDEPEEPTTSEEPEEPTTSEEPEEPTTSEEPEEPTTSEEPEEPTSSDEEPTTSEEPEEPTTSDEPEEPTTSEEPEEPTTSEEPEEPTTSEEPEEPTTSDEEPGTTEEPLVPTTKTETDVSTTLLTVTDCGTKTCTKSLVITGVTKETVTTHGKTTVITTYCPLPTETVTPTPVTVTSTIYADESVTKTTVYTTGAVEKTVTVGGSSTVVVVHTPLTTAVVQSQSTDEIKTVVTARPSTTTIVRDVCYNSVCSVATIVTGVTEKTITFSTGSITVVPTYVPLVESEEHQRTASTSETRATSVVVPTVVGQSSSASATSSIFPSVTIHEGVANTVKNSMISGAVALLFNALFL

SEQ ID NO: 341 - protein, 1444 aa, synthetic construct
MVSLRSIFTSSILAAGLTRAHGAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPGSSGSSGSSGSSGSSGSSGSSGSSEAAAREAAAREAAAREAAARGGGGSGGGGSGGGGSSSGKTCPTSEVSPACYANQWETTFPPSDIKITGATWVQDNIYDVTLSYEAESLELENLTELKIIGLNSPTGGTKLVWSLNSKVYDIDNPAKWTTTLRVYTKSSADDCYVEMYPFQIQVDWCEAGASTDGCSAWKWPKSYDYDIGCDNMQDGVSRKHHPVYKWPKKCSSNCGVEPTTSDEPEEPTTSEEPEEPTTSEEPEEPTSSDEEPTTSEEPEEPTTSDEPEEPTTSEEPEEPTTSEEPEEPTTSEEPTTSEEPEEPTSSDEEPTTSDEPEEPTTSDEPEEPTTSEEPTTSEEPEEPTTSSEEPTPSEEPEGPTCPTSEVSPACYADQWETTFPPSDIKITGATWVEDNIYDVTLSYEAESLELENLTELKIIGLNSPTGGTKVVWSLNSGIYDIDNPAKWTTTLRVYTKSSADDCYVEMYPFQIQVDWCEAGASTDGCSAWKWPKSYDYDIGCDNMQDGVSRKHHPVYKWPKKCSSDCGVEPTTSDEPEEPTTSEEPVEPTSSDEEPTTSEEPTTSEEPEEPTTSDEPEEPTTSEEPEEPTTSEEPEEPTTSEEPTTSEEPEEPTSSDEEPTTSDEPEEPTTSEEPEEPTTSEEPEEPTTSEEPEEPTTSDEPEEPTTSEEPEEPTTSEEPEEPTSSDEEPTTSEEPEEPTTSEEPEEPTTSEEPEEPTTSEEPEEPTSSDEEPTTSEEPEEPTTSDEPEEPTTSEEPEEPTTSEEPEEPTSSDEEPTTSEEPEEPTTSDEPEEPTTSEEPEEPTTSEEPEEPTTSEEPEEPTTSEEPEEPTSSDEEPTTSEEPEEPTTSDEPEEPTTSEEPEEPTTSEEPEEPTTSEEPEEPTTSDEEPGTTEEPLVPTTKTETDVSTTLLTVTDCGTKTCTKSLVITGVTKETVTTHGKTTVITTYCPLPTETVTPTPVTVTSTIYADESVTKTTVYTTGAVEKTVTVGGSSTVVVVHTPLTTAVVQSQSTDEIKTVVTARPSTTTIVRDVCYNSVCSVATIVTGVTEKTITFSTGSITVVPTYVPLVESEEHQRTASTSETRATSVVVPTVVGQSSSASATSSIFPSVTIHEGVANTVKNSMISGAVALLFNALFL

SEQ ID NO: 342 - protein, 1001 aa, synthetic construct
MRFPSIFTAVLFAASSALAAPVQTTTEDELEGDFDVAVLPFSASIAAKEEGVSLEKREAEASMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVKGSSGSSGSSGSSGSSGSSGSSGSSEAAAREAAAREAAAREAAARGGGGSGGGGSGGGGSQINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVASSSSEVASSSVASTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 343 - protein, 652 aa, unidentified
MVDLFQWLKFYSMRRLGQVAITLVLLNLFVFLGYKFTPSTVIGSPSWEPAVVPTVFNESYLDSLQFTDINVDSFLSDTNGRISVTCDSLAYKGLVKTSKKKELDCDMAYIRRKIFSSEEYGVLADLEAQDITEEQRIKKHWFTFYGSSVYLPEHEVHYLVRRVLFSKVGRADTPVISLLVAQLYDKDWNELTPHTLEIVNPATGNVTPQTFPQLIHVPIEWSVDDKWKGTEDPRVFLKPSKTGVSEPIVLFNLQSSLCDGKRGMFVTSPFRSDKVNLLDIEDKERPNSEKNWSPFFLDDVEVSKYSTGYVHFVYSFNPLKVIKCSLDTGACRMIYESPEEGRFGSELRGATPMVKLPVHLSLPKGKEVWVAFPRTRLRDCGCSRTTYRPVLTLFVKEGNKFYTELISSSIDFHIDVLSYDAKGESCSGSISVLIPNGIDSWDVSKKQGGKSDILTLTLSEADRNTVVVHVKGLLDYLLVLNGEGPIHDSHSFKNVLSTNHFKSDTTLLNSVKAAECAIFSSRDYCKKYGETRGEPARYAKQMENERKEKEKKEKEAKEKLEAEKAEMEEAVRKAQEAIAQKEREKEEAEQEKKAQQEAKEKEAEEKAAKEKEAKENEAKKKIIVEKLAKEQEEAEKLEAKKKLYQLQEEERS

SEQ ID NO: 344 - protein, 644 aa, unidentified
MRTRLNFLLLCIASVLSVIWIGVLLTWNDNNLGGISLNGGKDSAYDDLLSLGSFNDMEVDSYVTNIYDNAPVLGCTDLSYHGLLKVTPKHDLACDLEFIRAQILDIDVYSAIKDLEDKALTVKQKVEKHWFTFYGSSVFLPEHDVHYLVRRVIFSAEGKANSPVTSIIVAQIYDKNWNELNGHFLDILNPNTGKVQHNTFPQVLPIATNFVKGKKFRGAEDPRVVLRKGRFGPDPLVMFNSLTQDNKRRRIFTISPFDQFKTVMYDIKDYEMPRYEKNWVPFFLKDNQEAVHFVYSFNPLRVLKCSLDDGSCDIVFEIPKVDSMSSELRGATPMINLPQAIPMAKDKEIWVSFPRTRIANCGCSRTTYRPMLMLFVREGSNFFVELLSTSLDFGLEVLPYSGNGLPCSADHSVLIPNSIDNWEVVDSNGDDILTLSFSEADKSTSVIHIRGLYNYLSELDGYQGPEAEDEHNFQRILSDLHFDNKTTVNNFIKVQSCALDAAKGYCKEYGLTRGEAERRRRVAEERKKKEKEEEEKKKKKEKEEEEKKRIEEEKKKIEEKERKEKEKEEAERKKLQEMKKKLEEITEKLEKGQRNKEIDPKEKQREEEERKERVRKIAEKQRKEAEKKEAEKKANDKKDLKIRQ

SEQ ID NO: 345 - protein, 659 aa, unidentified
MFGKRRQVRKLLIWVVLLLIVYFFGLQFRAKNSAHQSSIRSFYADNKEFFDRQYSRYDEYDIIDNMNSHNELLQEQFRNGKLAAGLRGVAEEPNSDEVTDDTAIEEDEQAAMINFPKRSPQREKSLVELRKFYKNVLSIIINNKPAMPIENPRDPTPNENALKRKFGKSGIINIALHDTDPSLPILSEAYLRDSLQLSPSFIASLSKSHSAVVKAFPPSFPANAYNGTGIVFIGGQKFSWLSLLSIENLRKTGSKVPVELIIPFAHEYEPQLCEEILPKLNATCVLLQETVGIDLLKSGHLKGYQFKSLALLASSFEQVLLVDSDNIIVENPDPIFDSEVFQRTGLVLWPDFWRRVTHPDYYKIAGIKLGSERVRHVVDSYTDPSLYTSSSEDPFTDIPLHDREGAIPDGSTESGQILISKTKHCQTILLSLYYNFFGPDYYYPLFTQGASGEGDKETFLAAANYYKLPFYNIKKGVDVIGYWKPDQSAYQGCGMLQYDPIVDYQNLQTFLKTHKGSRVNKLEQSELDKPGLLSRLIPKFFFRKTFDEHQLQSHFTKDRSKIMFIHSNFPKLDPFGLKLHNYLFVDQDTHKPRIRMYADQTGLSFDFELRQWIIIHEYFCEYPDFNLKYLENANVKPQDLCMFIKEELNFLQNNPIQLT

SEQ ID NO: 346 - protein, 683 aa, unidentified
MLFGLIRHSRRQLLFLGALVTVIVLIFTLPNTSPIEANGVKSEEGSITPIIPVLESPANSLEKIVDTASEERIGGATLEEGHENNKEEQALENAERAKEKEKTEAIAAEEEKLKAAELLRQQETTREKEAAKEDDSKKPNQELVEQDTYLDDIPDDVEDNIIISEQDRKKIILPSYTPKTDPAYSKRATALKIFYNDFFIKVADSGPNTAPITKKTRKKGKSKLKGDVSSGDKYEGPVLTEDFLRFMEIYSDEFIDAVSESHSKIVNLMPESFPKGMYQGDGIVIIGGGVYSWYGLLAIRNLRDGGNTLPVELMLPSDNEYEPQLCEQILPSLNAKCIMLSDIVDQDVLKKLDFKGYQFKALSLLASSFENVLSLDSDNIPVANVSHLFDHEPFSETGLVSWPDFWRRTTNPRYYEAAGIKIGEYQVRNCLDGFVPESDFVHIGLKDIPLHDRNGTIPDASTESGQLLVNKNKHAKTLMLMFYYNFYGPGYYYPLLSQGMAGEGDKETFLAAANFFGLPFYQVKAGPGILGHHDSTGAFTGVAIVQYDPIADYELTKENFVGEKRKGIEAPKAFYGNNNKSPLFHHCNFPKLDPVKLIKEKKLIDNKTHKFNRMYGPNTKLKYDFEERQWKYTKEYLCEKKYNLLYFTEQYKNYGQGYSQERICKFSDRFLKFLSDNPIRIEG

SEQ ID NO: 347 - protein, 579 aa, unidentified
MFNSLAPMRLKKLLKVFCASVVLLAATSVVLFFHFGGQIIIPIPERTVTLSTPPANDTWQFQQFFNGYLDALLENNLSYPIPERWNHEVTNVRFFNRIGELLSESRLQELIHFSPEFIEDTSDKFDNIVEQIPAKWPYENMYRGDGYVIVGGGRHTFLALLNINALRRAGNKLPVEVVLPTYDDYEEDFCENHFPLLNARCVILEERFGDQVYPRLQLGGYQFKIFAIAASSFKNCFLLDSDNIPLRKMDKIFSSELYKNKTMITWPDFWLRSTSPHYYHNITKTPIGDKRVRYFNDFYTNPNEYYYGDEDPRSEIPFHDREGTIPDWTTESGQLVINKEVHFPAILLGLFYNFNGPMGFYPLLSQGGAGEGDKDTFVAASHYYNLPYYQVYKNCEMLYGWVDHANSGRIEHSAIVQYNPIVDYENLQSVKAKAEIILKNHEPDSRKKSSKPKSYSKTRLSTHVKGSIYSYRRLFRDSFNKANSDEMFLHCHTPKIEPYRIMEDDLTLGRNKEAKQRWYGGRKNRVRFGYDVELYIWELIDQYICDKNIQYKIFEGKDRDALCGSFMREQLGFLRSTGD